SEQUENCE LISTING (105 sequences)

SEQ ID NO: 1 - DNA, 1725 bp, synthetic construct
  misc_feature 1..1725: note = PDI-H3 HK DNA
  source 1..1725: mol_type = other DNA
atggcgaaaaacgttgcgattttcggcttattgttttctcttcttgtgttggttccttctcagatcttcgcgcaaaaaattcctggaaatgacaatagcacggcaacgctgtgccttgggcatcatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattggactggagtcactcaaaacggaacaagttctgcttgcataaggagatctagtagtagtttctttagtagattaaattggttgacccacttaaactacacatacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggtgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaaggaagaattcaggaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaataagaaatggaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttga

SEQ ID NO: 2 - protein, 574 aa, synthetic construct
  REGION 1..574: note = PDI-H3 HK AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 3 - DNA, 1725 bp, synthetic construct
  misc_feature 1..1725: note = PDI-H3 Minn DNA
  source 1..1725: mol_type = other DNA
atggcgaaaaacgttgcgattttcggcttattgttttctcttcttgtgttggttccttctcagatcttcgcgcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtgaaatatgcgacagtcctcatcagatccttgatggagagaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattggactggagtcactcaaaacggaacaagttctgcttgcataaggagatctagtagtagtttctttagtagattaaattggttgacccacttaaactacacatatccagcattgaacgtgactatgccaaacaaggaacaatttgacaaattgtacatttggggggttcaccacccgggtacggacaaggaccaaatcttcctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcggttgatcgggaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatgaaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattagatgcaacatttgcatttga

SEQ ID NO: 4 - protein, 574 aa, synthetic construct
  REGION 1..574: note = PDI-H3 Minn AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYTYPALNVTMPNKEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMWACQKGNIRCNICI

SEQ ID NO: 5 - DNA, 1725 bp, synthetic construct
  misc_feature 1..1725: note = PDI-H3 SAus DNA
  source 1..1725: mol_type = other DNA
atggcgaaaaacgttgcgattttcggcttattgttttctcttcttgtgttggttccttctcagatcttcgcgcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtgaaatatgcgacagtcctcatcagatccttgatggagagaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaaaaatgaaagcttcaattggactggagtcactcaaaacggaacaagttctgcttgcataaggagatctagtagtagtttctttagtagattaaattggttgacccacttaaactacacatatccagcattgaacgtgactatgccaaacaaggaacaatttgacaaattgtacatttggggggttcaccacccgggtacggacaaggaccaaatcttcctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcggttgatcgggaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatgaaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattagatgcaacatttgcatttga

SEQ ID NO: 6 - protein, 574 aa, synthetic construct
  REGION 1..574: note = PDI-H3 SAus AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGTSSACIRRSSSSFFSRLNWLTHLNYTYPALNVTMPNKEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMWACQKGNIRCNICI

SEQ ID NO: 7 - DNA, 1725 bp, synthetic construct
  misc_feature 1..1725: note = PDI-H3 Bang DNA
  source 1..1725: mol_type = other DNA
atggcgaaaaacgttgcgattttcggcttattgttttctcttcttgtgttggttccttctcagatcttcgcgcaaaaaattcctggaaatgacaatagcacggcaacgctgtgccttgggcaccatgcagtaccaaacggaacgatagtgaaaacaatcacaaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattggactggagtcactcaaaacggaacaagttctgcttgcataaggaaatctagtagtagtttctttagtagattaaattggttgacccacttaaactacacatacccagcattgaacgtgactgtgccaaacaatgaacaatttgacaaattgtacatttggggggttcaccacccgggtacggacaaggaccaaatcttcctgtatgctcgatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaaggaagaattcaggaccttgagaaatatgttgaggacactaaaatagatctctggtcatacaacgcggagcttcttgttgccctggagaatcaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatggaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttga

SEQ ID NO: 8 - protein, 574 aa, synthetic construct
  REGION 1..574: note = PDI-H3 Bang AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRKSSSSFFSRLNWLTHLNYTYPALNVTVPNNEQFDKLYIWGVHHPGTDKDQIFLYARSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 9 - DNA, 1725 bp, synthetic construct
  misc_feature 1..1725: note = PDI-H3 Switz DNA
  source 1..1725: mol_type = other DNA
atggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaatagtagtttctttagtagattaaattggttgacccacttaaactccaaatacccagcattaaacgtgactatgccaaacaatgaacaatttgacaaattgtacatttggggggttcaccacccgggtacggacaaggaccaaatcttcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttga

SEQ ID NO: 10 - protein, 574 aa, synthetic construct
  REGION 1..574: note = PDI-H3 Switz AA
MAKNVAIFGLLFSLLVLVPSQIFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWAGVTQNGTSSSCIRGSNSSFFSRLNWLTHLNSKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 11 - DNA, 1725 bp, synthetic construct
  misc_feature 1..1725: note = PDI-H3 Miss DNA
  source 1..1725: mol_type = other DNA
atggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcagagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaatagtagtttctttagtagattaaattggttgacccacttaaactccaaatacccagcattaaacgtgactatgccaaacaatgaacaatttgacaaattgtacatttggggggttcaccacccgggtacggacaaggaccaaatcttcctgtatgcaaaaccatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaaacactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttga

SEQ ID NO: 12 - protein, 574 aa, synthetic construct
  REGION 1..574: note = PDI-H3 Miss AA
MAKNVAIFGLLFSLLVLVPSQIFAQKLPGNDNSTATLCLGHHAVPNGTIVKTITNDRIEVTNATELVQNSSIGEICDSPHQILDGENCTLIDALLGDPQCDGFQNKKWDLFVERSRAYSNCYPYDVPDYASLRSLVASSGTLEFNNESFNWAGVTQNGTSSSCIRGSNSSFFSRLNWLTHLNSKYPALNVTMPNNEQFDKLYIWGVHHPGTDKDQIFLYAKPSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQNTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 13 - DNA, 1725 bp, synthetic construct
  misc_feature 1..1725: note = PDI-H3 Penn DNA
  source 1..1725: mol_type = other DNA
atggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggaaaaaactgcactctaatagatgctctattgggagaccctcagtgtgatggctttcaaaataagagatgggacctttttgttgaacgaagcaaagcctacagcaactgttacccttatgatgtgccggattatgcctcccttaggtcactagttgcctcatccggcacactggagtttaacgatgaaagcttcaattgggctggagtcactcaaaacggaacaagttctgcttgcataaggggatctaatagtagtttctttagtagattaaattggttgacccactcaaacttcaaatacccagcattgaacgtgactatgccaaacaatgaacaatttgacaaattgtacatttggggggttcaccacccgggtacagacaaggaccaaatcttcctgtacgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacaaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatgaaggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagctttaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttga

SEQ ID NO: 14 - protein, 574 aa, synthetic construct
  REGION 1..574: note = PDI-H3 Penn AA
MAKNVAIFGLLFSLLVLVPSQIFAQKLPGNDNSTATLCLGHHAVPNGTIVKTITNDRIEVTNATELVQNSSIGEICDSPHQILDGKNCTLIDALLGDPQCDGFQNKRWDLFVERSKAYSNCYPYDVPDYASLRSLVASSGTLEFNDESFNWAGVTQNGTSSACIRGSNSSFFSRLNWLTHSNFKYPALNVTMPNNEQFDKLYIWGVHHPGTDKDQIFLYAQSSGRITVSTKRSQQAVIPNIGSRPRIRNIPSRISIYWTIVKPGDILLINSTGNLIAPRGYFKIQSGKSSIMRSDAPIGKCKSECITPNGSIPNDKPFQNVNRITYGACPRYVKQSTLKLATGMRNVPEKQTRGIFGAIAGFIENGWEGMKDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 15 - DNA, 49 bp, synthetic construct
  misc_feature 1..49: note = Primer IF-CPMV(fl5'UTR)_SpPDI.c
  source 1..49: mol_type = other DNA
tcgtgcttcggcaccagtacaatggcgaaaaacgttgcgattttcggct

SEQ ID NO: 16 - DNA, 46 bp, synthetic construct
  misc_feature 1..46: note = Primer IF-H1cTMCT.S1-4r
  source 1..46: mol_type = other DNA
actaaagaaaataggcctttaaatacatattctacactgtagagac

SEQ ID NO: 17 - DNA, 55 bp, synthetic construct
  misc_feature 1..55: note = Primer IF-H3A-Ala.r
  source 1..55: mol_type = other DNA
actaaagaaaataggccttcaaatgcaaatgttgcacctaatgttgcccttttgg

SEQ ID NO: 18 - DNA, 51 bp, synthetic construct
  misc_feature 1..51: note = Primer H3_Swi_SLVLL.r
  source 1..51: mol_type = other DNA
agcaactaacagtacaagggatgatatggcaaaggaaatccataggatcca

SEQ ID NO: 19 - DNA, 53 bp, synthetic construct
  misc_feature 1..53: note = H3_Swi_SLVLL.c
  source 1..53: mol_type = other DNA
tcctttgccatatcatcccttgtactgttagttgctttgttggggttcatcat

SEQ ID NO: 20 - DNA, 1725 bp, synthetic construct
  misc_feature 1..1725: note = PDI-H3 HK-CysTm DNA
  source 1..1725: mol_type = other DNA
atggcgaaaaacgttgcgattttcggcttattgttttctcttcttgtgttggttccttctcagatcttcgcgcaaaaaattcctggaaatgacaatagcacggcaacgctgtgccttgggcatcatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattggactggagtcactcaaaacggaacaagttctgcttgcataaggagatctagtagtagtttctttagtagattaaattggttgacccacttaaactacacatacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggtgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaaggaagaattcaggaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaataagaaatggaacttatgaccacaatgtgtacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 21 - protein, 574 aa, synthetic construct
  REGION 1..574: note = PDI-H3 HK-CysTm AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISSLVLLVALLGFIMWACQKGNIRCNICI

SEQ ID NO: 22 - DNA, 48 bp, synthetic construct
  misc_feature 1..48: note = Primer H3_HK480114(N382A+L384V).r
  source 1..48: mol_type = other DNA
ttggttttcccgatcactcgagccagcttcccattgatttgatcgatt

SEQ ID NO: 23 - DNA, 48 bp, synthetic construct
  misc_feature 1..48: note = Primer H3_HK480114(N382A+L384V).c
  source 1..48: mol_type = other DNA
gggaagctggctcgagtgatcgggaaaaccaacgagaaattccatcag

SEQ ID NO: 24 - DNA, 1725 bp, synthetic construct
  misc_feature 1..1725: note = PDI-H3 HK-N382A+L384V-CysTm DNA
  source 1..1725: mol_type = other DNA
atggcgaaaaacgttgcgattttcggcttattgttttctcttcttgtgttggttccttctcagatcttcgcgcaaaaaattcctggaaatgacaatagcacggcaacgctgtgccttgggcatcatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattggactggagtcactcaaaacggaacaagttctgcttgcataaggagatctagtagtagtttctttagtagattaaattggttgacccacttaaactacacatacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggtgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctggctcgagtgatcgggaaaaccaacgagaaattccatcagattgaaaaagaattctcagaagtagaaggaagaattcaggaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaataagaaatggaacttatgaccacaatgtgtacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 25 - protein, 574 aa, synthetic construct
  REGION 1..574: note = PDI-H3 HK-N382A+L384V-CysTm AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISSLVLLVALLGFIMWACQKGNIRCNICI

SEQ ID NO: 26 - DNA, 1725 bp, synthetic construct
  misc_feature 1..1725: note = PDI-H3 Minn-CysTm DNA
  source 1..1725: mol_type = other DNA
atggcgaaaaacgttgcgattttcggcttattgttttctcttcttgtgttggttccttctcagatcttcgcgcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtgaaatatgcgacagtcctcatcagatccttgatggagagaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattggactggagtcactcaaaacggaacaagttctgcttgcataaggagatctagtagtagtttctttagtagattaaattggttgacccacttaaactacacatatccagcattgaacgtgactatgccaaacaaggaacaatttgacaaattgtacatttggggggttcaccacccgggtacggacaaggaccaaatcttcctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcggttgatcgggaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatgaaacttatgaccacaatgtgtacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattagatgcaacatttgcatttga

SEQ ID NO: 27 - protein, 574 aa, synthetic construct
  REGION 1..574: note = PDI-H3 Minn-CysTm AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYTYPALNVTMPNKEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISSLVLLVALLGFIMWACQKGNIRCNICI

SEQ ID NO: 28 - DNA, 60 bp, synthetic construct
  misc_feature 1..60: note = Primer H3Minn(N382A+L384V).r
  source 1..60: mol_type = other DNA
gttggttttcccgatcacccgagccagcttcccattgatttgatcgattgctgcttgagt

SEQ ID NO: 29 - DNA, 56 bp, synthetic construct
  misc_feature 1..56: note = Primer H3Minn(N382A+L384V).c
  source 1..56: mol_type = other DNA
aatcaatgggaagctggctcgggtgatcgggaaaaccaacgagaaattccatcaga

SEQ ID NO: 30 - DNA, 1725 bp, synthetic construct
  misc_feature 1..1725: note = PDI-H3 Minn-N382A+L384V-CysTm DNA
  source 1..1725: mol_type = other DNA
atggcgaaaaacgttgcgattttcggcttattgttttctcttcttgtgttggttccttctcagatcttcgcgcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtgaaatatgcgacagtcctcatcagatccttgatggagagaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattggactggagtcactcaaaacggaacaagttctgcttgcataaggagatctagtagtagtttctttagtagattaaattggttgacccacttaaactacacatatccagcattgaacgtgactatgccaaacaaggaacaatttgacaaattgtacatttggggggttcaccacccgggtacggacaaggaccaaatcttcctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctggctcgggtgatcgggaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatgaaacttatgaccacaatgtgtacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattagatgcaacatttgcatttga

SEQ ID NO: 31 - protein, 574 aa, synthetic construct
  REGION 1..574: note = PDI-H3 Minn-N382A+L384V-CysTm AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYTYPALNVTMPNKEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLARVIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISSLVLLVALLGFIMWACQKGNIRCNICI

SEQ ID NO: 32 - DNA, 1725 bp, synthetic construct
  misc_feature 1..1725: note = PDI-H3 SAus-CysTm DNA
  source 1..1725: mol_type = other DNA
atggcgaaaaacgttgcgattttcggcttattgttttctcttcttgtgttggttccttctcagatcttcgcgcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtgaaatatgcgacagtcctcatcagatccttgatggagagaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaaaaatgaaagcttcaattggactggagtcactcaaaacggaacaagttctgcttgcataaggagatctagtagtagtttctttagtagattaaattggttgacccacttaaactacacatatccagcattgaacgtgactatgccaaacaaggaacaatttgacaaattgtacatttggggggttcaccacccgggtacggacaaggaccaaatcttcctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcggttgatcgggaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatgaaacttatgaccacaatgtgtacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattagatgcaacatttgcatttga

SEQ ID NO: 33 - protein, 574 aa, synthetic construct
  REGION 1..574: note = PDI-H3 SAus-CysTm AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGTSSACIRRSSSSFFSRLNWLTHLNYTYPALNVTMPNKEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISSLVLLVALLGFIMWACQKGNIRCNICI

SEQ ID NO: 34 - DNA, 1725 bp, synthetic construct
  misc_feature 1..1725: note = PDI-H3 SAus-N382A+L384V-CysTm DNA
  source 1..1725: mol_type = other DNA
atggcgaaaaacgttgcgattttcggcttattgttttctcttcttgtgttggttccttctcagatcttcgcgcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtgaaatatgcgacagtcctcatcagatccttgatggagagaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaaaaatgaaagcttcaattggactggagtcactcaaaacggaacaagttctgcttgcataaggagatctagtagtagtttctttagtagattaaattggttgacccacttaaactacacatatccagcattgaacgtgactatgccaaacaaggaacaatttgacaaattgtacatttggggggttcaccacccgggtacggacaaggaccaaatcttcctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctggctcgggtgatcgggaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatgaaacttatgaccacaatgtgtacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattagatgcaacatttgcatttga

SEQ ID NO: 35 - protein, 574 aa, synthetic construct
  REGION 1..574: note = PDI-H3 SAus-N382A+L384V-CysTm AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGTSSACIRRSSSSFFSRLNWLTHLNYTYPALNVTMPNKEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLARVIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISSLVLLVALLGFIMWACQKGNIRCNICI

SEQ ID NO: 36 - DNA, 1725 bp, synthetic construct
  misc_feature 1..1725: note = PDI-H3 Bang-CysTm DNA
  source 1..1725: mol_type = other DNA
atggcgaaaaacgttgcgattttcggcttattgttttctcttcttgtgttggttccttctcagatcttcgcgcaaaaaattcctggaaatgacaatagcacggcaacgctgtgccttgggcaccatgcagtaccaaacggaacgatagtgaaaacaatcacaaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattggactggagtcactcaaaacggaacaagttctgcttgcataaggaaatctagtagtagtttctttagtagattaaattggttgacccacttaaactacacatacccagcattgaacgtgactgtgccaaacaatgaacaatttgacaaattgtacatttggggggttcaccacccgggtacggacaaggaccaaatcttcctgtatgctcgatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaaggaagaattcaggaccttgagaaatatgttgaggacactaaaatagatctctggtcatacaacgcggagcttcttgttgccctggagaatcaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatggaacttatgaccacaatgtgtacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 37 - protein, 574 aa, synthetic construct
  REGION 1..574: note = PDI-H3 Bang-CysTm AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRKSSSSFFSRLNWLTHLNYTYPALNVTVPNNEQFDKLYIWGVHHPGTDKDQIFLYARSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISSLVLLVALLGFIMWACQKGNIRCNICI

SEQ ID NO: 38 - DNA, 1725 bp, synthetic construct
  misc_feature 1..1725: note = PDI-H3 Bang-N382A+L384V-CysTm DNA
  source 1..1725: mol_type = other DNA
atggcgaaaaacgttgcgattttcggcttattgttttctcttcttgtgttggttccttctcagatcttcgcgcaaaaaattcctggaaatgacaatagcacggcaacgctgtgccttgggcaccatgcagtaccaaacggaacgatagtgaaaacaatcacaaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattggactggagtcactcaaaacggaacaagttctgcttgcataaggaaatctagtagtagtttctttagtagattaaattggttgacccacttaaactacacatacccagcattgaacgtgactgtgccaaacaatgaacaatttgacaaattgtacatttggggggttcaccacccgggtacggacaaggaccaaatcttcctgtatgctcgatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctggctcgagtgatcgggaaaaccaacgagaaattccatcagattgaaaaagaattctcagaagtagaaggaagaattcaggaccttgagaaatatgttgaggacactaaaatagatctctggtcatacaacgcggagcttcttgttgccctggagaatcaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatggaacttatgaccacaatgtgtacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 39 - protein, 574 aa, synthetic construct
  REGION 1..574: note = PDI-H3 Bang-N382A+L384V-CysTm AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRKSSSSFFSRLNWLTHLNYTYPALNVTVPNNEQFDKLYIWGVHHPGTDKDQIFLYARSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISSLVLLVALLGFIMWACQKGNIRCNICI

SEQ ID NO: 40 - DNA, 54 bp, synthetic construct
  misc_feature 1..54: note = Primer H3_Swi(F392D).r
  source 1..54: mol_type = other DNA
ttttcaatctgatggtctttctcgttggttttcccgatcaatcgattcagcttc

SEQ ID NO: 41 - DNA, 48 bp, synthetic construct
  misc_feature 1..48: note = Primer H3_Swi(F392D).c
  source 1..48: mol_type = other DNA
tcgggaaaaccaacgagaaagaccatcagattgaaaaagaattctcag

SEQ ID NO: 42 - DNA, 1725 bp, synthetic construct
  misc_feature 1..1725: note = PDI-H3 Switz-F392D DNA
  source 1..1725: mol_type = other DNA
atggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaatagtagtttctttagtagattaaattggttgacccacttaaactccaaatacccagcattaaacgtgactatgccaaacaatgaacaatttgacaaattgtacatttggggggttcaccacccgggtacggacaaggaccaaatcttcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctgaatcgattgatcgggaaaaccaacgagaaagaccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttga

SEQ ID NO: 43 - protein, 574 aa, synthetic construct
  REGION 1..574: note = PDI-H3 Switz-F392D AA
MAKNVAIFGLLFSLLVLVPSQIFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWAGVTQNGTSSSCIRGSNSSFFSRLNWLTHLNSKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKDHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 44 - DNA, 50 bp, synthetic construct
  misc_feature 1..50: note = Primer H3_Swi(L431M).r
  source 1..50: mol_type = other DNA
ttgtatgttggttctccatggcaacaagaagctccgcgttgtatgaccag

SEQ ID NO: 45 - DNA, 54 bp, synthetic construct
  misc_feature 1..54: note = Primer H3_Swi(L431M).c
  source 1..54: mol_type = other DNA
ggagcttcttgttgccatggagaaccaacatacaattgatctaactgactcaga

SEQ ID NO: 46 - DNA, 1725 bp, synthetic construct
  misc_feature 1..1725: note = PDI-H3 Switz-L431M DNA
  source 1..1725: mol_type = other DNA
atggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaatagtagtttctttagtagattaaattggttgacccacttaaactccaaatacccagcattaaacgtgactatgccaaacaatgaacaatttgacaaattgtacatttggggggttcaccacccgggtacggacaaggaccaaatcttcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaagggagaattcaggaccttgagaaatatgttgaggacacaaaaatagatctctggtcatacaacgcggagcttcttgttgccatggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttga

SEQ ID NO: 47 - protein, 574 aa, synthetic construct
  REGION 1..574: note = PDI-H3 Switz-L431M AA
MAKNVAIFGLLFSLLVLVPSQIFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWAGVTQNGTSSSCIRGSNSSFFSRLNWLTHLNSKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVAMENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 48 - DNA, 1725 bp, synthetic construct
  misc_feature 1..1725: note = PDI-H3 Switz-CysTm DNA
  source 1..1725: mol_type = other DNA
atggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaatagtagtttctttagtagattaaattggttgacccacttaaactccaaatacccagcattaaacgtgactatgccaaacaatgaacaatttgacaaattgtacatttggggggttcaccacccgggtacggacaaggaccaaatcttcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 49 - protein, 574 aa, synthetic construct
  REGION 1..574: note = PDI-H3 Switz-CysTm AA
MAKNVAIFGLLFSLLVLVPSQIFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWAGVTQNGTSSSCIRGSNSSFFSRLNWLTHLNSKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISSLVLLVALLGFIMWACQKGNIRCNICI

SEQ ID NO: 50 - DNA, 48 bp, synthetic construct
  misc_feature 1..48: note = Primer H3_Swi(N382A).r
  source 1..48: mol_type = other DNA
tttcccgatcaatcgagccagcttcccattgatttgatcgattgctgc

SEQ ID NO: 51 - DNA, 54 bp, synthetic construct
  misc_feature 1..54: note = Primer H3_Swi(N382A).c
  source 1..54: mol_type = other DNA
tcaaatcaatgggaagctggctcgattgatcgggaaaaccaacgagaaattcca

SEQ ID NO: 52 - DNA, 1725 bp, synthetic construct
  misc_feature 1..1725: note = PDI-H3 Switz-N382A-CysTm DNA
  source 1..1725: mol_type = other DNA
atggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaatagtagtttctttagtagattaaattggttgacccacttaaactccaaatacccagcattaaacgtgactatgccaaacaatgaacaatttgacaaattgtacatttggggggttcaccacccgggtacggacaaggaccaaatcttcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgattgatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 53 - protein, 574 aa, synthetic construct
  REGION 1..574: note = PDI-H3 Switz-N382A-CysTm AA
MAKNVAIFGLLFSLLVLVPSQIFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWAGVTQNGTSSSCIRGSNSSFFSRLNWLTHLNSKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISSLVLLVALLGFIMWACQKGNIRCNICI

SEQ ID NO: 54 - DNA, 48 bp, synthetic construct
  misc_feature 1..48: note = Primer H3_Swi(L384V).r
  source 1..48: mol_type = other DNA
ttggttttcccgatcactcgattcagcttcccattgatttgatcgatt

SEQ ID NO: 55 - DNA, 48 bp, synthetic construct
  misc_feature 1..48: note = Primer H3_Swi(L384V).c
  source 1..48: mol_type = other DNA
gggaagctgaatcgagtgatcgggaaaaccaacgagaaattccatcag

SEQ ID NO: 56 - DNA, 1725 bp, synthetic construct
  misc_feature 1..1725: note = PDI-H3 Switz-L384V-CysTm DNA
  source 1..1725: mol_type = other DNA
atggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaatagtagtttctttagtagattaaattggttgacccacttaaactccaaatacccagcattaaacgtgactatgccaaacaatgaacaatttgacaaattgtacatttggggggttcaccacccgggtacggacaaggaccaaatcttcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctgaatcgagtgatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 57 - protein, 574 aa, synthetic construct
  REGION 1..574: note = PDI-H3 Switz-L384V-CysTm AA
MAKNVAIFGLLFSLLVLVPSQIFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWAGVTQNGTSSSCIRGSNSSFFSRLNWLTHLNSKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRVIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISSLVLLVALLGFIMWACQKGNIRCNICI

SEQ ID NO: 58 - DNA, 1725 bp, synthetic construct
  misc_feature 1..1725: note = PDI-H3 Switz-F392D-CysTm DNA
  source 1..1725: mol_type = other DNA
atggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaatagtagtttctttagtagattaaattggttgacccacttaaactccaaatacccagcattaaacgtgactatgccaaacaatgaacaatttgacaaattgtacatttggggggttcaccacccgggtacggacaaggaccaaatcttcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctgaatcgattgatcgggaaaaccaacgagaaagaccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 59 - protein, 574 aa, synthetic construct
  REGION 1..574: note = PDI-H3 Switz-F392D-CysTm AA
MAKNVAIFGLLFSLLVLVPSQIFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWAGVTQNGTSSSCIRGSNSSFFSRLNWLTHLNSKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKDHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISSLVLLVALLGFIMWACQKGNIRCNICI

SEQ ID NO: 60 - DNA, 1725 bp, synthetic construct
  misc_feature 1..1725: note = PDI-H3 Switz-L431M-CysTm DNA
  source 1..1725: mol_type = other DNA
atggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaatagtagtttctttagtagattaaattggttgacccacttaaactccaaatacccagcattaaacgtgactatgccaaacaatgaacaatttgacaaattgtacatttggggggttcaccacccgggtacggacaaggaccaaatcttcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaagggagaattcaggaccttgagaaatatgttgaggacacaaaaatagatctctggtcatacaacgcggagcttcttgttgccatggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 61 - protein, 574 aa, synthetic construct
  REGION 1..574: note = PDI-H3 Switz-L431M-CysTm AA
MAKNVAIFGLLFSLLVLVPSQIFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWAGVTQNGTSSSCIRGSNSSFFSRLNWLTHLNSKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVAMENQHTIDLTDSEMNKLFEKTKKQLRENAEDMGNGCFKIYHKCDNACIGSIRNGTYDHDVYRDEALNNRFQIKGVELKSGYKDWILWISFAISSLVLLVALLGFIMWACQKGNIRCNICI

SEQ ID NO: 62 - DNA, 4540 bp, synthetic construct
  misc_feature 1..4540: note = Cloning vector 1190 from left to right T-DNA
  source 1..4540: mol_type = other DNA
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccgcggatggcgaaaaacgttgcgattttcggcttattgttttctcttcttgtgttggttccttctcagatcttcgcctgcaggctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagcgatcgctcaccatcaccatcaccatcaccatcaccattaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 63 - DNA, 3105 bp, synthetic construct
  misc_feature 1..3105: note = Construct 1314 from 2X35S prom to NOS term
  source 1..3105: mol_type = other DNA
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgctgacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacacctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatgcatatgtttttgtggggtcatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcaaacacccaagggtgctataaacaccagcctcccatttcagaatatacatccgatcacaattggaaaatgtccaaaatatgtaaaaagcacaaaattgagactggccacaggattgaggaatatcccgtctattcaatctagaggactatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagctaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 64 - DNA, 3105 bp, synthetic construct
  misc_feature 1..3105: note = Construct 2980 from 2X35S prom to NOS term
  source 1..3105: mol_type = other DNA
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgcggacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacacctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatgcatatgtttttgtggggtcatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcaaacacccaagggtgctataaacaccagcctcccatttcagaatatacatccgatcacaattggaaaatgtccaaaatatgtaaaaagcacaaaattgagactggccacaggattgaggaatatcccgtctattcaatctagaggactatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacaggacacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagctaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 65 - DNA, 3105 bp, synthetic construct
  misc_feature 1..3105: note = Construct 2995 from 2X35S prom to NOS term
  source 1..3105: mol_type = other DNA
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgcggacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacacctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatgcatatgtttttgtggggtcatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcaaacacccaagggtgctataaacaccagcctcccatttcagaatatacatccgatcacaattggaaaatgtccaaaatatgtaaaaagcacaaaattgagactggccacaggattgaggaatatcccgtctattcaatctagaggactatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacaggacacagcagtaggtaaagagttcaaccacctggaaaaaagaatagagaatttaaataaaaaagttgatgatggtttcctggacatttggacttacaatgccgaactgttggttctaatggaaaatgaaagaactttggactaccacgattcaaatgtgaagaacttatatgaaaaggtaagaagccagctaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 66 - DNA, 6360 bp, synthetic construct
  misc_feature 1..6360: note = Cloning vector 3556 from left to right T-DNA
  source 1..6360: mol_type = other DNA
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacacgcgtggcgcgccctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatgagtcttctaaccgaggtcgaaacgcctatcagaaacgaatgggggtgcagatgcaacgattcaagtgatcctcttgttgttgccgcaagtataattgggattgtgcacctgatattgtggattattgatcgccttttttccaaaagcatttatcgtatctttaaacacggtttaaaaagagggccttctacggaaggagtaccagagtctatgagggaagaatatcgagaggaacagcagaatgctgtggatgctgacgatggtcattttgtcagcatagagctggagtaagagctctaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggaactaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccgcggatggcgaaaaacgttgcgattttcggcttattgttttctcttcttgtgttggttccttctcagatcttcgcctgcaggctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagcgatcgctcaccatcaccatcaccatcaccatcaccattaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 67 - DNA, 50 bp, synthetic construct
  misc_feature 1..50: note = Primer IF-H5ITMCT.s1-4r
  source 1..50: mol_type = other DNA
actaaagaaaataggcctttaaatgcaaattctgcattgtaacgatccat

SEQ ID NO: 68 - DNA, 1731 bp, synthetic construct
  misc_feature 1..1731: note = PDI-H5 Indo DNA
  source 1..1731: mol_type = other DNA
atggcgaaaaacgttgcgattttcggcttattgttttctcttcttgtgttggttccttctcagatcttcgccgatcagatttgcattggttaccatgcaaacaattcaacagagcaggttgacacaatcatggaaaagaacgttactgttacacatgcccaagacatactggaaaagacacacaacgggaagctctgcgatctagatggagtgaagcctctaattttaagagattgtagtgtagctggatggctcctcgggaacccaatgtgtgacgaattcatcaatgtaccggaatggtcttacatagtggagaaggccaatccaaccaatgacctctgttacccagggagtttcaacgactatgaagaactgaaacacctattgagcagaataaaccattttgagaaaattcaaatcatccccaaaagttcttggtccgatcatgaagcctcatcaggagttagctcagcatgtccatacctgggaagtccctccttttttagaaatgtggtatggcttatcaaaaagaacagtacatacccaacaataaagaaaagctacaataataccaaccaagaggatcttttggtactgtggggaattcaccatcctaatgatgcggcagagcagacaaggctatatcaaaacccaaccacctatatttccattgggacatcaacactaaaccagagattggtaccaaaaatagctactagatccaaagtaaacgggcaaagtggaaggatggagttcttctggacaattttaaaacctaatgatgcaatcaacttcgagagtaatggaaatttcattgctccagaatatgcatacaaaattgtcaagaaaggggactcagcaattatgaaaagtgaattggaatatggtaactgcaacaccaagtgtcaaactccaatgggggcgataaactctagtatgccattccacaacatacaccctctcaccatcggggaatgccccaaatatgtgaaatcaaacagattagtccttgcaacagggctcagaaatagccctcaaagagagagcagaagaaaaaagagaggactatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcaatcattgacaaaatgaacactcagtttgaggccgttggaagggaatttaataacttagaaaggagaatagagaatttaaacaagaagatggaagacgggtttctagatgtctggacttataatgccgaacttctggttctcatggaaaatgagagaactctagactttcatgactcaaatgttaagaacctctacgacaaggtccgactacagcttagggataatgcaaaggagctgggtaacggttgtttcgagttctatcacaaatgtgataatgaatgtatggaaagtataagaaacggaacgtacaactatccgcagtattcagaagaagcaagattaaaaagagaggaaataagtggggtaaaattggaatcaataggaacttaccaaatactgtcaatttattcaacagtggcgagttccctagcactggcaatcatgatggctggtctatctttatggatgtgctccaatggatcgttacaatgcagaatttgcatttaa

SEQ ID NO: 69 - protein, 576 aa, synthetic construct
  REGION 1..576: note = PDI-H5 Indo AA
MAKNVAIFGLLFSLLVLVPSQIFADQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQILSIYSTVASSLALAIMMAGLSLWMCSNGSLQCRICI

SEQ ID NO: 70 - DNA, 49 bp, synthetic construct
  misc_feature 1..49: note = Primer H5Ind(F393D).r
  source 1..49: mol_type = other DNA
cttccaacggcctcgtcctgagtgttcattttgtcaatgattgagttga

SEQ ID NO: 71 - DNA, 49 bp, synthetic construct
  misc_feature 1..49: note = H5Ind(F393D).c
  source 1..49: mol_type = other DNA
caaaatgaacactcaggacgaggccgttggaagggaatttaataactta

SEQ ID NO: 72 - DNA, 1731 bp, synthetic construct
  misc_feature 1..1731: note = PDI-H5 Indo-F393D DNA
  source 1..1731: mol_type = other DNA
atggcgaaaaacgttgcgattttcggcttattgttttctcttcttgtgttggttccttctcagatcttcgcggatcagatttgcattggttaccatgcaaacaattcaacagagcaggttgacacaatcatggaaaagaacgttactgttacacatgcccaagacatactggaaaagacacacaacgggaagctctgcgatctagatggagtgaagcctctaattttaagagattgtagtgtagctggatggctcctcgggaacccaatgtgtgacgaattcatcaatgtaccggaatggtcttacatagtggagaaggccaatccaaccaatgacctctgttacccagggagtttcaacgactatgaagaactgaaacacctattgagcagaataaaccattttgagaaaattcaaatcatccccaaaagttcttggtccgatcatgaagcctcatcaggagttagctcagcatgtccatacctgggaagtccctccttttttagaaatgtggtatggcttatcaaaaagaacagtacatacccaacaataaagaaaagctacaataataccaaccaagaggatcttttggtactgtggggaattcaccatcctaatgatgcggcagagcagacaaggctatatcaaaacccaaccacctatatttccattgggacatcaacactaaaccagagattggtaccaaaaatagctactagatccaaagtaaacgggcaaagtggaaggatggagttcttctggacaattttaaaacctaatgatgcaatcaacttcgagagtaatggaaatttcattgctccagaatatgcatacaaaattgtcaagaaaggggactcagcaattatgaaaagtgaattggaatatggtaactgcaacaccaagtgtcaaactccaatgggggcgataaactctagtatgccattccacaacatacaccctctcaccatcggggaatgccccaaatatgtgaaatcaaacagattagtccttgcaacagggctcagaaatagccctcaaagagagagcagaagaaaaaagagaggactatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcaatcattgacaaaatgaacactcaggacgaggccgttggaagggaatttaataacttagaaaggagaatagagaatttaaacaagaagatggaagacgggtttctagatgtctggacttataatgccgaacttctggttctcatggaaaatgagagaactctagactttcatgactcaaatgttaagaacctctacgacaaggtccgactacagcttagggataatgcaaaggagctgggtaacggttgtttcgagttctatcacaaatgtgataatgaatgtatggaaagtataagaaacggaacgtacaactatccgcagtattcagaagaagcaagattaaaaagagaggaaataagtggggtaaaattggaatcaataggaacttaccaaatactgtcaatttattcaacagtggcgagttccctagcactggcaatcatgatggctggtctatctttatggatgtgctccaatggatcgttacaatgcagaatttgcatttaa

SEQ ID NO: 73 - protein, 576 aa, synthetic construct
  REGION 1..576: note = PDI-H5 Indo-F393D AA
MAKNVAIFGLLFSLLVLVPSQIFADQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQDEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQILSIYSTVASSLALAIMMAGLSLWMCSNGSLQCRICI

SEQ ID NO: 74 - DNA, 51 bp, synthetic construct
  misc_feature 1..51: note = Primer IF-H5_Egy.r
  source 1..51: mol_type = other DNA
actaaagaaaataggcctttaaatgcaaattctgcattgtagcgatccatt

SEQ ID NO: 75 - DNA, 1728 bp, synthetic construct
  misc_feature 1..1728: note = PDI-H5 Egypt DNA
  source 1..1728: mol_type = other DNA
atggcgaaaaacgttgcgattttcggcttattgttttctcttcttgtgttggttccttctcagatcttcgcggatcagatttgcattggttaccatgcaaacaactcgacagagcaggttgacacaataatggaaaagaatgtcactgttacacacgcccaagacatactggaaaagacacacaacgggaaactctgcaatctagatggagtgaagcctctcattttgagagattgtagtgtagctggatggctcctcgggaacccaatgtgcgatgaattcctcaatgtgccggaatggtcttacatagtggagaaaatcaatccagccaatgacctctgttatccagggaatttcaacgactatgaagaactgaaacacctattgagcagaataaaccattttgagaaaattcagatcattcccaaagattcttggtcagatcatgaagcctcgggagtgagctcagcatgcccataccaaggaagatcctccttttttagaaatgttgtatggcttaccaaaaagaacgatgcatacccaacaataaagaaaagttacaataatactaaccaagaagatcttttggtactatgggggattcaccatccaaatgatgctgcagagcagacaaggctttatcaaaacccaactacctatatctccgttgggacatcaacactaaaccagagattggtacccaaaatagctactagatctaaggtaaacgggcaaagtggaaggatggagttcttttggacaattttaaaatcgaatgatgcaataaactttgagagcaatggaaacttcattgctccagaaaatgcatacaaaattgtcaagaaaggagattcaacaattatgaaaagtgagttggaatatagtaactgcaacaccaagtgtcagactccaataggggcgataaactccagtatgccattccacaacatccaccctctcaccatcggggaatgccccaaatatgtgaaatcaaacagattagtccttgctactgggctcaggaatagccctcaaggagagaaaagaagaaaaaagagaggactattcggagccatagcaggctttatagagggaggatggcagggaatggtagatggttggtatgggtaccaccatagcaacgagcaggggagtgggtacgctgcagacaaagaatccactcaaagggctatagatggagtcaccaataaggtcaattcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaatctttatgacaaggtccgactacagcttagggataatgcaaaggagcttggtaacggttgtttcgagttctatcacagatgtgataatgaatgtatggaaagtgtaagaaacggaacgtatgactaccctcaatattcagaagaagcaagattaaaaagagaggaaataagtggagtaaaattggagtcaataggaacttaccaaatactgtcaatttattcaacagtggcgagctccctagcactggcaatcatggtggctggtctatctttatggatgtgctccaatggatcgctacaatgcagaatttgcatttaa

SEQ ID NO: 76 - protein, 575 aa, synthetic construct
  REGION 1..575: note = PDI-H5 Egypt AA
MAKNVAIFGLLFSLLVLVPSQIFADQICIGYHANNSTEQVDTIMEKNVTVTHAQDILEKTHNGKLCNLDGVKPLILRDCSVAGWLLGNPMCDEFLNVPEWSYIVEKINPANDLCYPGNFNDYEELKHLLSRINHFEKIQIIPKDSWSDHEASGVSSACPYQGRSSFFRNVVWLTKKNDAYPTIKKSYNNTNQEDLLVLWGIHHPNDAAEQTRLYQNPTTYISVGTSTLNQRLVPKIATRSKVNGQSGRMEFFWTILKSNDAINFESNGNFIAPENAYKIVKKGDSTIMKSELEYSNCNTKCQTPIGAINSSMPFHNIHPLTIGECPKYVKSNRLVLATGLRNSPQGEKRRKKRGLFGAIAGFIEGGWQGMVDGWYGYHHSNEQGSGYAADKESTQRAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLSLWMCSNGSLQCRICI

SEQ ID NO: 77 - DNA, 49 bp, synthetic construct
  misc_feature 1..49: note = Primer H5Egy(F392D).r
  source 1..49: mol_type = other DNA
cttccaacagcctcgtcctgagtgttcattttgtcaatgatcgaattga

SEQ ID NO: 78 - DNA, 49 bp, synthetic construct
  misc_feature 1..49: note = Primer H5Egy(F392D).c
  source 1..49: mol_type = other DNA
caaaatgaacactcaggacgaggctgttggaagggaatttaataactta

SEQ ID NO: 79 - DNA, 1728 bp, synthetic construct
  misc_feature 1..1728: note = PDI-H5 Egypt-F392D DNA
  source 1..1728: mol_type = other DNA
atggcgaaaaacgttgcgattttcggcttattgttttctcttcttgtgttggttccttctcagatcttcgcggatcagatttgcattggttaccatgcaaacaactcgacagagcaggttgacacaataatggaaaagaatgtcactgttacacacgcccaagacatactggaaaagacacacaacgggaaactctgcaatctagatggagtgaagcctctcattttgagagattgtagtgtagctggatggctcctcgggaacccaatgtgcgatgaattcctcaatgtgccggaatggtcttacatagtggagaaaatcaatccagccaatgacctctgttatccagggaatttcaacgactatgaagaactgaaacacctattgagcagaataaaccattttgagaaaattcagatcattcccaaagattcttggtcagatcatgaagcctcgggagtgagctcagcatgcccataccaaggaagatcctccttttttagaaatgttgtatggcttaccaaaaagaacgatgcatacccaacaataaagaaaagttacaataatactaaccaagaagatcttttggtactatgggggattcaccatccaaatgatgctgcagagcagacaaggctttatcaaaacccaactacctatatctccgttgggacatcaacactaaaccagagattggtacccaaaatagctactagatctaaggtaaacgggcaaagtggaaggatggagttcttttggacaattttaaaatcgaatgatgcaataaactttgagagcaatggaaacttcattgctccagaaaatgcatacaaaattgtcaagaaaggagattcaacaattatgaaaagtgagttggaatatagtaactgcaacaccaagtgtcagactccaataggggcgataaactccagtatgccattccacaacatccaccctctcaccatcggggaatgccccaaatatgtgaaatcaaacagattagtccttgctactgggctcaggaatagccctcaaggagagaaaagaagaaaaaagagaggactattcggagccatagcaggctttatagagggaggatggcagggaatggtagatggttggtatgggtaccaccatagcaacgagcaggggagtgggtacgctgcagacaaagaatccactcaaagggctatagatggagtcaccaataaggtcaattcgatcattgacaaaatgaacactcaggacgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaatctttatgacaaggtccgactacagcttagggataatgcaaaggagcttggtaacggttgtttcgagttctatcacagatgtgataatgaatgtatggaaagtgtaagaaacggaacgtatgactaccctcaatattcagaagaagcaagattaaaaagagaggaaataagtggagtaaaattggagtcaataggaacttaccaaatactgtcaatttattcaacagtggcgagctccctagcactggcaatcatggtggctggtctatctttatggatgtgctccaatggatcgctacaatgcagaatttgcatttaa

SEQ ID NO: 80 - protein, 575 aa, synthetic construct
  REGION 1..575: note = PDI-H5 Egypt-F392D AA
MAKNVAIFGLLFSLLVLVPSQIFADQICIGYHANNSTEQVDTIMEKNVTVTHAQDILEKTHNGKLCNLDGVKPLILRDCSVAGWLLGNPMCDEFLNVPEWSYIVEKINPANDLCYPGNFNDYEELKHLLSRINHFEKIQIIPKDSWSDHEASGVSSACPYQGRSSFFRNVVWLTKKNDAYPTIKKSYNNTNQEDLLVLWGIHHPNDAAEQTRLYQNPTTYISVGTSTLNQRLVPKIATRSKVNGQSGRMEFFWTILKSNDAINFESNGNFIAPENAYKIVKKGDSTIMKSELEYSNCNTKCQTPIGAINSSMPFHNIHPLTIGECPKYVKSNRLVLATGLRNSPQGEKRRKKRGLFGAIAGFIEGGWQGMVDGWYGYHHSNEQGSGYAADKESTQRAIDGVTNKVNSIIDKMNTQDEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLSLWMCSNGSLQCRICI

SEQ ID NO: 81 - DNA, 1725 bp, synthetic construct
  misc_feature 1..1725: note = PDI-H3 Switz-N382A DNA
  source 1..1725: mol_type = other DNA
atggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaatagtagtttctttagtagattaaattggttgacccacttaaactccaaatacccagcattaaacgtgactatgccaaacaatgaacaatttgacaaattgtacatttggggggttcaccacccgggtacggacaaggaccaaatcttcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgattgatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttga

SEQ ID NO: 82 - protein, 574 aa, synthetic construct
  REGION 1..574: note = PDI-H3 Switz-N382A AA
MAKNVAIFGLLFSLLVLVPSQIFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWAGVTQNGTSSSCIRGSNSSFFSRLNWLTHLNSKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 83 - DNA, 1725 bp, synthetic construct
  misc_feature 1..1725: note = PDI-H3 Switz-L384V DNA
  source 1..1725: mol_type = other DNA
atggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaatagtagtttctttagtagattaaattggttgacccacttaaactccaaatacccagcattaaacgtgactatgccaaacaatgaacaatttgacaaattgtacatttggggggttcaccacccgggtacggacaaggaccaaatcttcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctgaatcgagtgatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttga

SEQ ID NO: 84 - protein, 574 aa, synthetic construct
  REGION 1..574: note = PDI-H3 Switz-L384V AA
MAKNVAIFGLLFSLLVLVPSQIFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWAGVTQNGTSSSCIRGSNSSFFSRLNWLTHLNSKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRVIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 85 - DNA, 1725 bp, synthetic construct
  misc_feature 1..1725: note = PDI-H3 Penn-CysTm DNA
  source 1..1725: mol_type = other DNA
atggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggaaaaaactgcactctaatagatgctctattgggagaccctcagtgtgatggctttcaaaataagagatgggacctttttgttgaacgaagcaaagcctacagcaactgttacccttatgatgtgccggattatgcctcccttaggtcactagttgcctcatccggcacactggagtttaacgatgaaagcttcaattgggctggagtcactcaaaacggaacaagttctgcttgcataaggggatctaatagtagtttctttagtagattaaattggttgacccactcaaacttcaaatacccagcattgaacgtgactatgccaaacaatgaacaatttgacaaattgtacatttggggggttcaccacccgggtacagacaaggaccaaatcttcctgtacgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacaaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatgaaggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagctttaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 86 - protein, 574 aa, synthetic construct
  REGION 1..574: note = PDI-H3 Penn-CysTm AA
MAKNVAIFGLLFSLLVLVPSQIFAQKLPGNDNSTATLCLGHHAVPNGTIVKTITNDRIEVTNATELVQNSSIGEICDSPHQILDGKNCTLIDALLGDPQCDGFQNKRWDLFVERSKAYSNCYPYDVPDYASLRSLVASSGTLEFNDESFNWAGVTQNGTSSACIRGSNSSFFSRLNWLTHSNFKYPALNVTMPNNEQFDKLYIWGVHHPGTDKDQIFLYAQSSGRITVSTKRSQQAVIPNIGSRPRIRNIPSRISIYWTIVKPGDILLINSTGNLIAPRGYFKIQSGKSSIMRSDAPIGKCKSECITPNGSIPNDKPFQNVNRITYGACPRYVKQSTLKLATGMRNVPEKQTRGIFGAIAGFIENGWEGMKDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISSLVLLVALLGFIMWACQKGNIRCNICI

SEQ ID NO: 87 - DNA, 1725 bp, synthetic construct
  misc_feature 1..1725: note = PDI-H3 Penn-N382A-CysTm DNA
  source 1..1725: mol_type = other DNA
atggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggaaaaaactgcactctaatagatgctctattgggagaccctcagtgtgatggctttcaaaataagagatgggacctttttgttgaacgaagcaaagcctacagcaactgttacccttatgatgtgccggattatgcctcccttaggtcactagttgcctcatccggcacactggagtttaacgatgaaagcttcaattgggctggagtcactcaaaacggaacaagttctgcttgcataaggggatctaatagtagtttctttagtagattaaattggttgacccactcaaacttcaaatacccagcattgaacgtgactatgccaaacaatgaacaatttgacaaattgtacatttggggggttcaccacccgggtacagacaaggaccaaatcttcctgtacgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacaaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatgaaggatggttggtacggtttcaggcatcaaaattctgagggaagaggacaagcagcagatctcaaaagcactcaagcagcaatcgatcaaatcaatgggaagctggctcgattgatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagctttaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 88 - protein, 574 aa, synthetic construct
  REGION 1..574: note = PDI-H3 Penn-N382A-CysTm AA
MAKNVAIFGLLFSLLVLVPSQIFAQKLPGNDNSTATLCLGHHAVPNGTIVKTITNDRIEVTNATELVQNSSIGEICDSPHQILDGKNCTLIDALLGDPQCDGFQNKRWDLFVERSKAYSNCYPYDVPDYASLRSLVASSGTLEFNDESFNWAGVTQNGTSSACIRGSNSSFFSRLNWLTHSNFKYPALNVTMPNNEQFDKLYIWGVHHPGTDKDQIFLYAQSSGRITVSTKRSQQAVIPNIGSRPRIRNIPSRISIYWTIVKPGDILLINSTGNLIAPRGYFKIQSGKSSIMRSDAPIGKCKSECITPNGSIPNDKPFQNVNRITYGACPRYVKQSTLKLATGMRNVPEKQTRGIFGAIAGFIENGWEGMKDGWYGFRHQNSEGRGQAADLKSTQAAIDQINGKLARLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISSLVLLVALLGFIMWACQKGNIRCNICI

SEQ ID NO: 89 - DNA, 1725 bp, synthetic construct
  misc_feature 1..1725: note = PDI-H3 Penn-L384V-CysTm DNA
  source 1..1725: mol_type = other DNA
atggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggaaaaaactgcactctaatagatgctctattgggagaccctcagtgtgatggctttcaaaataagagatgggacctttttgttgaacgaagcaaagcctacagcaactgttacccttatgatgtgccggattatgcctcccttaggtcactagttgcctcatccggcacactggagtttaacgatgaaagcttcaattgggctggagtcactcaaaacggaacaagttctgcttgcataaggggatctaatagtagtttctttagtagattaaattggttgacccactcaaacttcaaatacccagcattgaacgtgactatgccaaacaatgaacaatttgacaaattgtacatttggggggttcaccacccgggtacagacaaggaccaaatcttcctgtacgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacaaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatgaaggatggttggtacggtttcaggcatcaaaattctgagggaagaggacaagcagcagatctcaaaagcactcaagcagcaatcgatcaaatcaatgggaagctgaatcgagtgatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagctttaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 90 - protein, 574 aa, synthetic construct
  REGION 1..574: note = PDI-H3 Penn-L384V-CysTm AA
MAKNVAIFGLLFSLLVLVPSQIFAQKLPGNDNSTATLCLGHHAVPNGTIVKTITNDRIEVTNATELVQNSSIGEICDSPHQILDGKNCTLIDALLGDPQCDGFQNKRWDLFVERSKAYSNCYPYDVPDYASLRSLVASSGTLEFNDESFNWAGVTQNGTSSACIRGSNSSFFSRLNWLTHSNFKYPALNVTMPNNEQFDKLYIWGVHHPGTDKDQIFLYAQSSGRITVSTKRSQQAVIPNIGSRPRIRNIPSRISIYWTIVKPGDILLINSTGNLIAPRGYFKIQSGKSSIMRSDAPIGKCKSECITPNGSIPNDKPFQNVNRITYGACPRYVKQSTLKLATGMRNVPEKQTRGIFGAIAGFIENGWEGMKDGWYGFRHQNSEGRGQAADLKSTQAAIDQINGKLNRVIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISSLVLLVALLGFIMWACQKGNIRCNICI

SEQ ID NO: 91 - protein, 550 aa, Alpha influenza A virus
QKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRKSSSSFFSRLNWLTHLNYTYPALNVTVPNNEQFDKLYIWGVHHPGTDKDQIFLYARSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 92 - protein, 550 aa, Alpha influenza A virus
QKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 93 - protein, 550 aa, Alpha influenza A virus
QKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYTYPALNVTMPNKEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMWACQKGNIRCNICI

SEQ ID NO: 94 - protein, 550 aa, Alpha influenza A virus
QKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGTSSACIRRSSSSFFSRLNWLTHLNYTYPALNVTMPNKEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMWACQKGNIRCNICI

SEQ ID NO: 95 - protein, 550 aa, Alpha influenza A virus
QKLPGNDNSTATLCLGHHAVPNGTIVKTITNDRIEVTNATELVQNSSIGEICDSPHQILDGKNCTLIDALLGDPQCDGFQNKRWDLFVERSKAYSNCYPYDVPDYASLRSLVASSGTLEFNDESFNWAGVTQNGTSSACIRGSNSSFFSRLNWLTHSNFKYPALNVTMPNNEQFDKLYIWGVHHPGTDKDQIFLYAQSSGRITVSTKRSQQAVIPNIGSRPRIRNIPSRISIYWTIVKPGDILLINSTGNLIAPRGYFKIQSGKSSIMRSDAPIGKCKSECITPNGSIPNDKPFQNVNRITYGACPRYVKQSTLKLATGMRNVPEKQTRGIFGAIAGFIENGWEGMKDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 96 - protein, 550 aa, Alpha influenza A virus
QKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWAGVTQNGTSSSCIRGSNSSFFSRLNWLTHLNSKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 97 - protein, 550 aa, Alpha influenza A virus
QKLPGNDNSTATLCLGHHAVPNGTIVKTITNDRIEVTNATELVQNSSIGEICDSPHQILDGENCTLIDALLGDPQCDGFQNKKWDLFVERSRAYSNCYPYDVPDYASLRSLVASSGTLEFNNESFNWAGVTQNGTSSSCIRGSNSSFFSRLNWLTHLNSKYPALNVTMPNNEQFDKLYIWGVHHPGTDKDQIFLYAKPSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQNTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 98 - protein, 5 aa, synthetic construct
  REGION 1..5: note = CysTM sequence (aa)
SLVLL

SEQ ID NO: 99 - protein, 5 aa, synthetic construct
  REGION 1..5: note = Native TMCT (aa)
CFLLC

SEQ ID NO: 100 - DNA, 15 bp, synthetic construct
  misc_feature 1..15: note = CysTM sequence (nt)
  source 1..15: mol_type = other DNA
tcccttgtactgtta

SEQ ID NO: 101 - DNA, 15 bp, synthetic construct
  misc_feature 1..15: note = Native TMCT (nt)
  source 1..15: mol_type = other DNA
tgttttttgctttgt

SEQ ID NO: 102 - DNA, 1701 bp, Alpha influenza A virus
  source 1..1701: mol_type = genomic DNA
atgaagactatcattgctttgagctacattctatgtctggttttcgctcaaaaaattcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattggactggagtcactcaaaacggaacaagttctgcttgcataaggagatctagtagtagtttctttagtagattaaattggttgacccacttaaactacacatacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaaggaagaattcaggaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaataagaaatggaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttga

SEQ ID NO: 103 - DNA, 3108 bp, synthetic construct
  misc_feature 1..3108: note = Construct 2801 from 2X35S prom to NOS term
  source 1..3108: mol_type = other DNA
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaatagtagtttctttagtagattaaattggttgacccacttaaactccaaatacccagcattaaacgtgactatgccaaacaatgaacaatttgacaaattgtacatttggggggttcaccacccgggtacggacaaggaccaaatcttcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 104 - DNA, 3108 bp, synthetic construct
  misc_feature 1..3108: note = Construct 3023 from 2X35S prom to NOS term
  source 1..3108: mol_type = other DNA
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaatagtagtttctttagtagattaaattggttgacccacttaaactccaaatacccagcattaaacgtgactatgccaaacaatgaacaatttgacaaattgtacatttggggggttcaccacccgggtacggacaaggaccaaatcttcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgattgatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 105 - DNA, 3108 bp, synthetic construct
  misc_feature 1..3108: note = Construct 2811 from 2X35S prom to NOS term
  source 1..3108: mol_type = other DNA
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgcgcaaaaacttcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaatagtagtttctttagtagattaaattggttgacccacttaaactccaaatacccagcattaaacgtgactatgccaaacaatgaacaatttgacaaattgtacatttggggggttcaccacccgggtacggacaaggaccaaatcttcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

(19) United States
(12) Patent Application Publication
(10) Pub. No.: US 2025/0122549 A1
(43) Pub. Date: Apr. 17, 2025

(54) INFLUENZA VIRUS HEMAGGLUTININ MUTANTS

(71) Applicant: ARAMIS BIOTECHNOLOGIES INC., Quebec (CA)

(72) Inventors: Pierre-Olivier LAVOIE, Quebec (CA); Aurelien LORIN, Quebec (CA); Alain DOUCET, Quebec (CA); Marc-Andre D'AOUST, Quebec (CA); Manon COUTURE, Quebec (CA)

(73) Assignee: ARAMIS BIOTECHNOLOGIES INC., Quebec (CA)

(21) Appl. No.: 18/909,318

(22) Filed: Oct. 8, 2024

Related U.S. Application Data
(62) Division of application No. 17/255,520, filed on Dec. 23, 2020, now Pat. No. 12,139,511.
(60) Provisional application No. 62/690,780, filed on Jun. 27, 2018.

(51) Int. Cl. C12P 21/02; A61K 38/00; A61K 39/00; A61K 39/145; A61P 31/16; A61P 37/04; C07K 14/005; C07K 14/11; C07K 16/10; C12N 7/00; C12N 15/82

(52) U.S. Cl. CPC C12P 21/02; A61K 39/145; A61P 31/16; A61P 37/04; C07K 14/005; C07K 14/11; C07K 16/10; C07K 16/1018; C12N 7/00; C12N 15/8257; C12N 15/8258; A61K 38/00; A61K 2039/5258; C12N 2760/16023; C12N 2760/16033; C12N 2760/16122; C12N 2760/16123; C12N 2760/16134; C12N 2760/16152

(57) ABSTRACT

The present invention relates to the production of modified influenza viral proteins in plants. More specifically, the present invention relates to producing and increasing influenza virus-like particle (VLP) production in plants, wherein the VLPs comprise the modified influenza viral proteins, such as modified influenza hemagglutinin (HA). The HA protein may comprising an amino acid sequence comprising at least one substitution when compared to a corresponding wildtype amino acid sequence. Further provided are nucleic acid encoding the modified HA protein. Furthermore methods of producing an influenza virus like particle (VLP) and methods of increasing yield of production of an influenza virus like particle (VLP) in a plant, portion of a plant, or a plant cell, are also provided.

### CROSS-REFERENCE TO RELATED APPLICATIONS

This application is a Divisional of U.S. application Ser. No. 17/255,520, filed Dec. 23, 2020; which is a National Stage (371) of PCT International Application No. PCT/CA2019/050892, filed Jun. 27, 2019; which claims priority to U.S. Application No. 62/690,780, filed Jun. 27, 2018, the disclosures of which are all incorporated by reference.

### SEQUENCE LISTING

The Sequence Listing, submitted herewith through Patent Center as an eXtensible Markup Language file (2024-10-01_Sequence_Listing.xml; created on Oct. 1, 2024; 236,637 bytes), is incorporated by reference.

### FIELD OF INVENTION

The present invention relates to producing mutant viral proteins in plants. More specifically, the present invention relates to producing and increasing influenza virus-like particle production in plants.

### BACKGROUND OF THE INVENTION

Influenza viruses are enveloped, single-stranded-RNA viruses of the Orthomyxoviridae family. Influenza viruses are highly contagious and can cause mild to serious illness across all age groups.

Vaccination remains the most effective method to prevent influenza infection. Conventionally, vaccination is accomplished using live attenuated or whole inactivated forms of the virus, which elicit an immune response when administered to a patient. To eliminate the potential risk of live attenuated and whole inactivated viruses re-acquiring the competency to replicate and become infectious, vaccines comprising recombinant viral proteins have also been used to elicit protective immunity to influenza infection.

However, the use of recombinant viral proteins as the immunogenic component of vaccines is subject to a number of limitations. Firstly, in the absence of the full complement of viral proteins and genetic components required for optimal expression and proper protein folding, the yield of recombinant viral proteins in standard in vitro expression systems may be insufficient for the purpose of vaccine production. Second, recombinant viral protein vaccines may exhibit poor immunogenicity, owing to improper folding, poor antigen presentation, and/or the generation of a primarily humoral immune response that is ineffective in conferring long-lasting, protective immunity.

There are four types of influenza virus: A, B, C and D, of which influenza A and B are the causative organism for seasonal disease epidemics in humans.

Influenza A viruses are further divided based on the expression of hemagglutinin (HA) and neuraminidase (NA) glycoprotein subtypes on the surface of the virus. There are 18 different HA subtypes (H1-H18).

HA is a trimeric lectin that facilitates binding of the influenza virus particle to sialic acid-containing proteins on the surface of target cells and mediates release of the viral genome into the target cell. HA proteins comprise two structural elements: the head, which is the primary target of seroprotective antibodies; and the stalk. HA is translated as a single polypeptide, HA0 (assembled as trimers), that must be cleaved by a serine endoprotease between the HA1 (^{˜}40 kDa) and HA2 (^{˜}20 kDa) subdomains. After cleavage, the two disulfide-bonded protein domains adopt the requisite conformation necessary for viral infectivity. HA1 forms the globular head domain containing the receptor-binding site (RBS), and is the least conserved segment of influenza virus. HA2 is a single-pass integral membrane protein with fusion peptide (FP), soluble ectodomain (SE), transmembrane (TM), and cytoplasmic tail (CT) with respective lengths of approximately 25, 160, 25, and 10 residues. HA2 together with the N and C terminal HA1 residues forms a stalk domain, which includes the transmembrane region, and is relatively conserved.

Various mutations in influenza virus proteins, particularly influenza HA, have been investigated.

For example, Castelan-Vega et al. (Adv Appl Bioinform Chem. 2014; 7:37-44) used a stability prediction algorithm to compare 7,479 full-length amino acid sequences of HA from the influenza A (H1N1)pdm09 virus and identified that D104N, A259T, S124N, and E172K mutations resulted in a predicted enhancement of influenza HA stability. In contrast, S206T, K285E, and E47K mutations had a predicted destabilizing effect on HA.

In comparing the sequences of the original influenza A (H1N1)pdm [A/California/7/2009] and a later-emerging influenza strain [A/Brisbane/10/2010], Cotter et al. (PLoS Pathog. 2014; 10(1):e1003831) identified that a E47K mutation in the stalk region of A/California/7/2009 HA stabilized the trimer structure, lowered the pH for membrane fusion, and increased the thermal and acid stability of the virus. Cotter et al. additionally observed that A/California/7/2009 E47K mutant HA was more infectious in ferrets than its wildtype counterpart.

Antanasijevic et al. (J Biol Chem. 2014; 289(32):22237-45) investigated the structure-function properties of H5 HA stem loop region by site directed mutagenesis at 14 different positions. A/Vietnam/1203/04 (H5N1) mutants were expressed in HEK 293T cells and Antanasijevic reported that most mutations in the stem loop region did not disrupt expression, proteolytic processing, viral assembly, or receptor binding. However, Antanasijevic observed that HA1-D26K, HA1-M102L, HA2-V52A and HA2-I55A mutants (based on H3 numbering) exhibited significantly reduced levels of total HA, suggesting reduced expression and/or assembly of HA into viral particles. HA1-D26K, HA2-T49A and HA2-M102L mutants also exhibited lower hemagglutination titers as compared to wildtype virus. Antanasijevic additionally observed that all single mutants exhibited decreased entry into A549 lung cells, with the most pronounced impairment shown in HA1-D26K and HA2-I55A mutants. Antanasijivec further demonstrated that the HA2-L99A mutant was more sensitive to A549 lung cell inhibition by C179 neutralizing antibody as compared to wildtype virus, suggesting that the mutation enhances antibody binding and/or the mode of neutralizing action. In contrast, HA1-I28A, HA1-M31A, HA1-M31L, HA2-I45A, and HA2-I55V mutants were rendered less sensitive to entry inhibition by C179 neutralizing antibody.

WO2013/177444 and its companion publication Lu et al. (Proc Natl Acad Sci USA. 2014; 111(1):125-30) reported a method for the production of properly folded HA stem domain from A/California/05/2009 (H1N1) using an Escherichia coli-based cell-free protein expression system and a simple refolding protocol. For inducing the trimerization of HA stem domain, either a chloramphenicol acetyl transferase (CAT) or foldon domain was fused to the C terminus of the HA. To mitigate newly exposed hydrophobicity and/or intermolecular ion pairing causing aggregation of expressed HA stem protein, five groups of mutations were evaluated: M1 (169T+172E+174T+C77T); M2 (169T+172E+174T+C77T+F164D); M3 (169T+172E+174T+C77T+F164D+L174D); M4 (F164D); and M5 (F164D+L174D). Lu observed that the M5 (F164D+L174D) mutations appeared to be the most influential mutations for improving HA stem protein solubility. Additional deletions (H38 to C43 and C49 to N61) and a C77T mutation were made to M5 mutants to avoid the formation of undesirable disulfide bonds, reduce surface hydrophobicity and pl, and avoid regions with disordered structure.

U.S. application Ser. No. 13/838,796 and its companion publication by Holtz et al. (BMC Biotechnology. 2014; 14:111) teach the improved stability and maintained potency of recombinant HA by the mutation of cysteine residues in the carboxy terminal region of the HA protein including the transmembrane (TM) and cytosolic domain (CT). Specifically, Holtz et al. demonstrate C539A, C546A, C549A, C524S and C528A mutations in recombinant Perth/16/2009 HA (H3N2). Mutation of all five cysteine residues, or different subsets thereof, resulted in HA yields, purities, particle size, hemagglutination activity, and thermostability comparable to recombinant wildtype HA protein. In contrast, C64S and C76S mutations resulted in significantly reduced HA expression, indicating the critical role of these residues in proper HA folding. By using a single radial immune-diffusion assay (SRID), Holtz et al. also show that the five cysteine residue mutations improve potency of recombinant HA as compared to wildtype protein, by preventing disulfide cross-linking in the TM and CT domains. The mutant HA proteins maintain potency for at least 12 months at 25° C., whereas wildtype HA protein exhibited less than 40% potency after only 50 days post purification.

WO2015/020913 teaches the mutation of specific residues at one or more positions selected from the group of 403, 406, 411, 422, 429, 432, 433, and 435 of influenza A/Puerto Rico/8/1934 (H1N1) to tyrosine. These mutations facilitate the formation of di-tyrosine cross-links that stabilize or “lock” the stalk domain of influenza HA in its native trimeric conformation.

WO2013/079473 discloses a modified influenza HA lacking a globular head domain. The polypeptide taught in WO2013/079473 comprises an HA1 domain where amino acids 53 to 620 (with reference to A/Brisbane/59/2007 [H1N1] numbering) are deleted and replaced with covalently linked sequence of 0 to 10 amino acids, and an HA2 domain, wherein the C-terminal amino acid of the HA1 domain is an amino acid other than arginine or lysine, and wherein one or more amino acids at position 406, 409, 413 and 416 are mutated to an amino acid selected from the group consisting of: serine, threonine, asparagine, glutamine, arginine, histidine, lysine, aspartic acid, glutamic acid, and glycine.

WO2014/191435 similarly teaches a modified influenza HA comprising an HA1 domain having a deleted segment replaced with a covalently linked sequence of 0 to 50 amino acids, and an HA2 domain, wherein the HA is resistant to cleavage at the junction between HA1 and HA2 and wherein one or more amino acids at positions 337, 340, 352, 353, 402, 406, 409, 413 and/or 416 have been mutated.

Virus-like particles (VLPs) are potential candidates for inclusion in immunogenic compositions. VLPs closely resemble mature virions, but they do not contain viral genomic material. Therefore, VLPs are non-replicative in nature, which make them safe for administration as a vaccine. In addition, VLPs can be engineered to express viral glycoproteins on the surface of the VLP, which is their most native physiological configuration. Moreover, since VLPs resemble intact virions and are multivalent particulate structures, VLPs may be more effective in inducing neutralizing antibodies to the glycoprotein than soluble envelope protein antigens.

VLPs have been produced in plants (see for example WO2009/076778; WO2009/009876; WO 2009/076778; WO 2010/003225; WO 2010/003235; WO2010/006452; WO2011/03522; WO 2010/148511; and WO2014153674 which are incorporated herein by reference).

WO2009/076778 teaches a method of producing influenza VLPs in plants comprising introducing a nucleic acid having a regulatory region active in the plant operatively linked to a nucleotide sequence encoding an influenza HA from a type A or type B influenza.

WO2009/009876 teaches a method of producing influenza HA VLPs in plants, wherein influenza HA self-assembles into VLPs in plant cells and bud from plant cell membranes.

WO2010/003225 discloses a method of producing influenza HA VLPs in plants comprising introducing a nucleic acid having a regulatory region active in the plant, operatively linked to a nucleotide sequence encoding an influenza HA from A/California/04/09 (H1N1).

WO2010/006452 teaches the production of VLPs comprising modified influenza HA proteins, wherein glycosylation sites at positions 154, 165, 286, or combinations thereof (with reference to A/Vietnam/1194/04 [H5N1] numbering), have been abolished by mutating the residues at said positions to amino acids other than asparagine. WO2010/006452 further teaches that amino acids at positions 156, 167, 288, or combinations thereof, may be mutated to residues other than serine or threonine to similarly abolish the N-linked glycosylation signal triad “N-X-S/T”. By selectively deleting glycosylation sites located in the globular head of the HA protein, WO2010/006452 demonstrates that the resulting HA protein has increased antigenicity and broader cross-reactivity.

WO2011/035422 teaches a method of preparing plant-derived VLPs comprising: obtaining a plant or plant matter comprising apoplast-localized VLPs; producing a protoplast/spheroplast fraction and an apoplast fraction; and recovering the apoplast fraction comprising the plant-derived VLPs.

WO2010/148511 discloses a method for producing influenza VLPs in plants, wherein the VLPs comprise chimeric HA proteins. The chimeric HA proteins comprise a stem domain cluster having an F′1, F′2 and F subdomain; a head domain cluster having an RB, E1 and E2 subdomain; and a transmembrane domain cluster having a transmembrane domain and a C-terminal tail domain, wherein at least one subdomain is derived from a first influenza strain and the other subdomains are derived from one or more second influenza strain.

WO2014/153674 teaches a method of producing influenza VLPs in a plant, wherein the VLPs comprise modified influenza HA having a modified proteolytic loop. The modified proteolytic loop comprises the removal of the proteolytic cleavage site between HA1 and HA2 domains of the HAO precursor. The HA protein is thus stabilized and increased protein yields are achieved as compared to native HA protein.

### SUMMARY OF THE INVENTION

The present invention relates to the production of modified influenza viral proteins in plants. More specifically, the present invention relates to producing and increasing influenza virus-like particle (VLP) production in plants, wherein the VLPs comprise the modified influenza viral proteins for example a modified hemagglutinin (HA) protein.

It is an object of the invention to provide an improved method to increase influenza VLP production in plants.

According to the present invention, there is provided:

A. A nucleic acid comprising a nucleotide sequence encoding a modified influenza H3 hemagglutinin (HA) protein, the HA protein comprising an amino acid sequence comprising at least one substitution when compared to a corresponding wildtype amino acid sequence, said at least one substitution being at one or more than one amino acids corresponding to amino acids at position 382, 384, 392 or 431 of A/Hong Kong/4801/14 HA.

The HA protein may comprise an amino acid sequence with a substitution to a non-asparagine at the amino acid corresponding to the amino acid at position 382 of A/Hong Kong/4801/14 HA. The HA protein may comprise an amino acid sequence with a substitution to an alanine or a conserved substitution of alanine at the amino acid corresponding to the amino acid at position 382 of A/Hong Kong/4801/14 HA.

The HA protein may comprise an amino acid sequence with a substitution to a non-leucine at the amino acid corresponding to the amino acid at position 384 of A/Hong Kong/4801/14 HA. The HA protein may comprise an amino acid sequence with a substitution to a valine or a conserved substitution of valine at the amino acid corresponding to the amino acid at position 384 of A/Hong Kong/4801/14 HA.

The HA protein may comprise an amino acid sequence with a substitution to a non-phenylalanine at the amino acid corresponding to the amino acid at position 392 of A/Hong Kong/4801/14 HA. The HA protein may comprise an amino acid sequence with a substitution to an aspartic acid or a conserved substitution of aspartic acid at the amino acid corresponding to the amino acid at position 392 of A/Hong Kong/4801/14 HA.

The HA protein may comprise an amino acid sequence with a substitution to a non-leucine at the amino acid corresponding to the amino acid at position 431 of A/Hong Kong/4801/14 HA. The HA protein may comprise an amino acid sequence with a substitution to a methionine or a conserved substitution of methionine at the amino acid corresponding to the amino acid at position 431 of A/Hong Kong/4801/14 HA.

It is further provided:

B. A nucleic acid comprising, a nucleotide sequence encoding a modified influenza H3 hemagglutinin (HA) protein, the HA protein comprising an amino acid sequence comprising at least one substitution when compared to a corresponding wildtype amino acid sequence, said at least one substitution being at one or more than one amino acid corresponding to amino acids at positions 382, 384, 392, 431, 524, 525, 526 or 528 of A/Hong Kong/4801/14 HA.

The HA protein may further comprise an amino acid sequence with a first substitution to a non-asparagine at the amino acid corresponding to amino acid at position 382 of A/Hong Kong/4801/14 HA, a second substitution to a non-cysteine at the amino acid corresponding to amino acid at position 524 of A/Hong Kong/4801/14 HA and a third substitution to a non-cysteine at the amino acid corresponding to amino acid at position 528 of A/Hong Kong/4801/14 HA. The HA protein may further comprise an amino acid sequence with a first substitution to an alanine or a conserved substitution of alanine at the amino acid corresponding to amino acid at position 382 of A/Hong Kong/4801/14 HA, a second substitution to a serine or a conserved substitution of serine at the amino acid corresponding to amino acid at position 524 of A/Hong Kong/4801/14 HA and a third substitution to a leucine or a conserved substitution of leucine at the amino acid corresponding to amino acid at position 528 of A/Hong Kong/4801/14 HA.

The HA protein may further comprise an amino acid sequence with a first substitution to a non-leucine at the amino acid corresponding to amino acid at position 384 of A/Hong Kong/4801/14 HA, a second substitution to a non-cysteine at the amino acid corresponding to amino acid at position 524 of A/Hong Kong/4801/14 HA and a third substitution to a non-cysteine at the amino acid corresponding to amino acid at position 528 of A/Hong Kong/4801/14 HA. The HA protein may further comprise an amino acid sequence with a first substitution to a valine or a conserved substitution of valine at the amino acid corresponding to amino acid at position 384 of A/Hong Kong/4801/14 HA, a second substitution to a serine or a conserved substitution of serine at the amino acid corresponding to amino acid at position 524 of A/Hong Kong/4801/14 HA and a third substitution to a leucine or a conserved substitution of leucine at the amino acid corresponding to amino acid at position 528 of A/Hong Kong/4801/14 HA.

The HA protein may further comprise an amino acid sequence with a first substitution to a non-phenylalanine at the amino acid corresponding to amino acid at position 392 of A/Hong Kong/4801/14 HA, a second substitution to a non-cysteine at the amino acid corresponding to amino acid at position 524 of A/Hong Kong/4801/14 HA and a third substitution to a non-cysteine at the amino acid corresponding to amino acid at position 528 of A/Hong Kong/4801/14 HA. The HA protein may further comprise an amino acid sequence with a first substitution to an aspartic acid or a conserved substitution of aspartic acid at the amino acid corresponding to amino acid at position 392 of A/Hong Kong/4801/14 HA, a second substitution to a serine or a conserved substitution of serine at the amino acid corresponding to amino acid at position 524 of A/Hong Kong/4801/14 HA and a third substitution to a leucine or a conserved substitution of leucine at the amino acid corresponding to amino acid at position 528 of A/Hong Kong/4801/14 HA.

The HA protein may further comprise an amino acid sequence with a first substitution to non-leucine at the amino acid corresponding to amino acid at position 431 of A/Hong Kong/4801/14 HA, a second substitution to a non-cysteine at the amino acid corresponding to amino acid at position 524 of A/Hong Kong/4801/14 HA and a third substitution to a non-cysteine at the amino acid corresponding to amino acid at position 528 of A/Hong Kong/4801/14 HA. The HA protein may further comprise an amino acid sequence with a first substitution to a methionine or a conserved substitution of methionine at the amino acid corresponding to amino acid at position 431 of A/Hong Kong/4801/14 HA, a second substitution to a serine or a conserved substitution of serine at the amino acid corresponding to amino acid at position 524 of A/Hong Kong/4801/14 HA and a third substitution to a leucine or a conserved substitution of leucine at the amino acid corresponding to amino acid at position 528 of A/Hong Kong/4801/14 HA.

The HA protein may further comprise an amino acid sequence with a first substitution to a non-asparagine at the amino acid corresponding to amino acid at position 382 of A/Hong Kong/4801/14 HA, a second substitution to a non-leucine at the amino acid corresponding to amino acid at position 384 of A/Hong Kong/4801/14 HA, a third substitution to a non-cysteine at the amino acid corresponding to amino acid at position 524 of A/Hong Kong/4801/14 HA and a fourth substitution to a non-cysteine at the amino acid corresponding to amino acid at position 528 of A/Hong Kong/4801/14 HA. The HA protein may further comprise an amino acid sequence with a first substitution to an alanine or a conserved substitution of alanine at the amino acid corresponding to amino acid at position 382 of A/Hong Kong/4801/14 HA, a second substitution to a valine or a conserved substitution of valine at the amino acid corresponding to amino acid at position 384 of A/Hong Kong/4801/14 HA, a third substitution to a serine or a conserved substitution of serine at the amino acid corresponding to amino acid at position 524 of A/Hong Kong/4801/14 HA and a fourth substitution to a leucine or a conserved substitution of leucine at the amino acid corresponding to amino acid at position 528 of A/Hong Kong/4801/14 HA.

The HA protein may further comprise an amino acid sequence with a substitution at amino acids corresponding to amino acids at positions 525, 526 or 525 and 526 of A/Hong Kong/4801/14 HA, wherein the substitution of the amino acid at position 525 is to a non-phenylalanine and the substitution of the amino acid at position 526 is to a non-leucine. The HA protein may further comprise an amino acid sequence with a substitution at amino acids corresponding to amino acids at positions 525, 526 or 525 and 526 of A/Hong Kong/4801/14 HA, wherein the substitution of the amino acid at position 525 is to a leucine or a conserved substitution of leucine and the substitution of the amino acid at position 526 is to a valine or a conserved substitution of valine.

Further provided are HA protein encoded by the recombinant nucleic acids as described under A or B and virus-like particle (VLP) comprising the HA protein encoded by the recombinant nucleic acids as described under A or B.

Therefore it is further provided a modified HA protein comprising an amino acid sequence comprising at least one substitution when compared to a corresponding wildtype amino acid sequence, said at least one substitution being at one or more than one amino acids corresponding to amino acids at position 382, 384, 392 or 431 of A/Hong Kong/4801/14 HA.

In another aspect it is provided a modified influenza H3 hemagglutinin (HA) protein, the HA protein comprising an amino acid sequence comprising at least one substitution when compared to a corresponding wildtype amino acid sequence, said at least one substitution being at one or more than one amino acid corresponding to amino acids at positions 382, 384, 392, 431, 524, 525, 526 or 528 of A/Hong Kong/4801/14 HA.

Furthermore a method of producing an influenza virus like particle (VLP) in a plant, portion of a plant, or a plant cell, is provided, the method comprising:
a) introducing the recombinant nucleic acid as described under A or B into the plant, portion of the plant, or plant cell; and
b) incubating the plant, portion of the plant, or plant cell under conditions that permit expression of the HA protein encoded by the recombinant nucleic acid, thereby producing the VLP. The method may further comprises a step c) of harvesting the plant, portion of the plant, or plant cell, and purifying the VLP.

It is further provided a method of producing an influenza virus like particle (VLP) in a plant, portion of a plant, or a plant cell, comprising:
a) providing a plant, portion of a plant, or plant cell comprising the recombinant nucleic acid as described under A or B; and
b) incubating the plant, portion of the plant, or plant cell under conditions that permit expression of the HA protein encoded by the recombinant nucleic acid, thereby producing the VLP. The method may further comprises a step c) of harvesting the plant, portion of the plant, or plant cell, and purifying the VLP.

Furthermore, it is provided a method of increasing yield of production of an influenza virus like particle (VLP) in a plant, portion of a plant, or a plant cell, comprising: a) introducing the recombinant nucleic acid of A or B into the plant, portion of the plant, or plant cell; or providing a plant, portion of a plant, or plant cell comprising the recombinant nucleic acid of A or B; and b) incubating the plant, portion of the plant, or plant cell under conditions that permit expression of the HA protein encoded by the recombinant nucleic acid, thereby producing the VLP at a higher yield compared to plant, portion of the plant, or plant cell expressing an unmodified influenza HA protein. The method may further comprises a step c) of harvesting the plant, portion of the plant, or plant cell, and purifying the VLP.

The methods may further comprise introducing a second nucleic acid encoding a proton channel protein; wherein the plant, portion of the plant, or plant cell is incubated under conditions that permit expression of the proton channel protein encoded by the second nucleic acid. The proton channel protein may be an influenza A subtype M2 protein.

It is further provided a VLP produced by the method as described herewith.

The VLP may comprise one or more than one lipid derived from the plant, portion of the plant, or plant cell, plant-specific N-glycans, modified N-glycans or a combination thereof.

In addition a method of producing an antibody or antibody fragment is provided, the method comprising administering the VLP as described to a subject, or a host animal, thereby producing the antibody or the antibody fragment. Antibodies or the antibody fragments produced by the method are also provided.

Furthermore it is provided a plant, portion of the plant, or plant cell comprising the recombinant nucleic acid of A or B or HA protein encoded by the recombinant nucleic acid of A or B. The HA protein may form VLP. Accordingly, a plant, portion of the plant, or plant cell comprising VLP comprising HA protein encoded by the recombinant nucleic acid of A or B are also provided.

In addition, it is provided a composition for inducing an immune response comprising, an effective dose of the VLP as described herewith, and a pharmaceutically acceptable carrier, adjuvant, vehicle or excipient. A method for inducing immunity to an influenza infection in a subject, the method comprising administering the VLP as described is also provided. The VLP may be administered to the subject orally, intranasally, intramuscularly, intraperitoneally, intravenously or subcutaneously.

Furthermore, it is provided a modified influenza hemagglutinin (HA) protein comprising an amino acid sequence having from about 30% to about 100%, sequence identity or sequence similarity with a sequence of the sequences of SEQ ID NO: 21, SEQ ID NO: 25, SEQ ID NO: 27, SEQ ID NO: 31, SEQ ID NO: 33, SEQ ID NO: 35, SEQ ID NO: 37, SEQ ID NO: 39, SEQ ID NO: 43, SEQ ID NO: 47, SEQ ID NO: 49, SEQ ID NO: 53, SEQ ID NO: 57, SEQ ID NO: 59, SEQ ID NO: 61, SEQ ID NO: 82, SEQ ID NO: 84, SEQ ID NO: 86, SEQ ID NO: 88, SEQ ID NO: 90, SEQ ID NO: 98, provided that the influenza HA protein comprises at least on substitution as described herewith and is able to form VLPs, induces an immune response when administered to a subject, induces hemagglutination or a combination thereof.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a sequence alignment of the amino acid sequences of hemagglutinin (HA) of A/Bangkok/3007/15 (H3N2) (SEQ ID NO: 91); A/Hongkong/4801/14 (H3N2) (SEQ ID NO: 92); A/Minnesota/40/15 (H3N2) (SEQ ID NO: 93); A/South Australia/1/16 (H3N2) (SEQ ID NO: 94); A/Pennsylvania/09/15 (H3N2) (SEQ ID NO:95); A/Switzerland/9715293/13 (H3N2)(SEQ ID NO: 96); A/Mississippi/16/16 (H3N2) (SEQ ID NO: 97); Outlined residues align with amino acids N382, L384, F392, L431 of HA from influenza H3 strains (H3N2) for example A/Hongkong/4801/14 (H3N2) (SEQ ID NO: 92).

FIG. 2 shows the hemagglutination titers of wildtype A/Switzerland/9715293/13 H3, N382A A/Switzerland/9715293/13 mutant H3, L384V A/Switzerland/9715293/13 mutant H3, F392D A/Switzerland/9715293/13 mutant H3 and L431M A/Switzerland/9715293/13 mutant H3.

FIG. 3 shows the hemagglutination titers of wildtype A/Indonesia/5/2005 H5, wildtype A/Egypt/N04915/2014 H5, F393D A/Indonesia/5/2005 mutant H5, F393D A/Egypt/N04915/2014 mutant H5, N383A A/Indonesia/5/2005 mutant H5, and N383A A/Egypt/N04915/2014 mutant H5. The numbering is in accordance with A/Indonesia/5/2005.

FIG. 4A shows a schematic representation of vector 3045 (H3 A-Swi-9715293-13 (F392D)). FIG. 4B shows a schematic representation of vector 3022 (H3 A-Swi-9715293-13 (L431M)). FIG. 4C shows a schematic representation of vector 3023 (H3 A-Swi-9715293-13 (N382A)). FIG. 4D shows a schematic representation of vector 3034 (H3 A-Swi-9715293-13 (L384V)).

FIG. 5A shows a schematic representation of vector 3556 (Vector for In-Fusion cloning into CPMV 160-based expression cassette in addition to M2 helper protein under the control of alfalfa plastocyanin promoter and terminator). FIG. 5B shows a schematic representation of vector 1190 (Vector for In-Fusion cloning into CPMV 160-based expression cassette).

FIG. 6A shows the hemagglutination titers of wildtype A/Switzerland/9715293/13 H3 and CysTM A/Switzerland/9715293/13 mutant H3.

FIG. 6B shows the hemagglutination titers of wildtype A/Pennsylvania/09/2015 H3, and CysTM A/Pennsylvania/09/2015 mutant H3.

FIG. 7A shows the hemagglutination titers of wildtype A/Switzerland/9715293/13 H3, N382A A/Switzerland/9715293/13 mutant H3, L384V A/Switzerland/9715293/13 mutant H3, F392D A/Switzerland/9715293/13 mutant H3, L431M A/Switzerland/9715293/13 mutant H3, A/Switzerland/9715293/13 H3 with CysTM modification, N382A+CysTM A/Switzerland/9715293/13 mutant H3, L384V+CysTM A/Switzerland/9715293/13 mutant H3, F392D+CysTM A/Switzerland/9715293/13 mutant H3 and L431M+CysTM A/Switzerland/9715293/13 mutant H3. FIG. 7B shows the post-density gradient VLP yields of CysTM A/Switzerland/9715293/13 mutant H3, N382A+CysTM A/Switzerland/9715293/13 mutant H3, L384V+CysTM A/Switzerland/9715293/13 mutant H3, F392D+CysTM A/Switzerland/9715293/13 mutant H3, and L431M+CysTM A/Switzerland/9715293/13 mutant H3 expressed as percentages relative to CysTM A/Switzerland/9715293/13 mutant H3. FIG. 7C shows the hemagglutination titers of wildtype A/Pennsylvania/09/2015 H3, CysTM A/Pennsylvania/09/2015 mutant H3, N382A+CysTM A/Pennsylvania/09/2015 mutant H3, and L384V+CysTM A/Pennsylvania/09/2015 mutant H3. FIG. 7D shows the hemagglutination titers of CysTM A/S. Australia/1/16 mutant H3 and N382A+L384V+CysTM A/S. Australia/1/16 mutant H3. FIG. 7E shows the hemagglutination titers of CysTM A/Hong Kong/4801/14 mutant H3 and N382A+L384V+CysTM A/Hong Kong/4801/14 mutant H3; CysTM A/Minnesota/40/15 mutant H3 and N382A+L384V+CysTM A/Minnesota/40/15 mutant H3; CysTM A/S. Australia/1/16 mutant H3 and N382A+L384V+CysTM A/S. Australia/1/16 mutant H3; CysTM A/Bangkok/3007/15 mutant H3 and N382A+L384V+CysTM A/Bangkok/3007/15 mutant H3; CysTM A/Switzerland/9715293/13 mutant H3, and L384V+CysTM A/Switzerland/9715293/13 mutant H3; CysTM A/Mississippi/16/16 mutant H3 and N382A+L384V+CysTM A/Mississippi/16/16 mutant H3.

FIG. 8A shows a schematic representation of vector 3340 (H3 A-HK-4801-14). FIG. 8B shows a schematic representation of vector 3341 (H3 A-HK-4801-14). FIG. 8C shows a schematic representation of vector 3375 (H3 A-HK-4801-14 (N382A+L384V)). FIG. 8D shows a schematic representation of vector 3914 (H3 A-Minn-40-15). FIG. 8E shows a schematic representation of vector 3915 (H3 A-Minn-40-15 (N382A+L384V)). FIG. 8F shows a schematic representation of vector 3924 (H3 A-SAus-1-16). FIG. 8G shows a schematic representation of vector 3925 (H3 A-SAus-1-16 (N382A+L384V)). FIG. 8H shows a schematic representation of vector 3904 (H3 A-Bang-3007-15). FIG. 8I shows a schematic representation of vector 3905 (H3 A-Bang-3007-15 (N382A+L384V)). FIG. 8J shows a schematic representation of vector 2801 (H3 A-Swi-9715293-13). FIG. 8K shows a schematic representation of vector 2811 (H3 A-Swi-9715293-13). FIG. 8L shows a schematic representation of vector 3063 (H3 A-Swi-9715293-13 (N382A)). FIG. 8M shows a schematic representation of vector 3074 (H3 A-Swi-9715293-13 (L384V)). FIG. 8N shows a schematic representation of vector 3085 (H3 A-Swi-9715293-13 (F392D)). FIG. 8O shows a schematic representation of vector 3062 (H3 A-Swi-9715293-13 (L431M)). FIG. 8P shows a schematic representation of vector 3312 (H3 A-Penn-09-15). FIG. 8Q shows a schematic representation of vector 3313 (H3 A-Penn-09-15). FIG. 8R shows a schematic representation of vector 3314 (H3 A-Penn-09-15 (N382A)). FIG. 8S shows a schematic representation of vector 3315 (H3 A-Penn-09-15 (L384V)).

FIG. 9A shows a schematic representation of vector 2295 (H5 A-Indo-5-05). FIG. 9B shows a schematic representation of vector 3680 (H5 A-Indo-5-05 (F393D)). FIG. 9C shows a schematic representation of vector 3645 (H5 A-Egypt-N04915-14). FIG. 9D shows a schematic representation of vector 3690 (H5 A-Egypt-N04915-14 (F392D)).

### DETAILED DESCRIPTION

The following description is of a preferred embodiment.

As used herein, the terms “comprising”, “having”, “including”, “containing”, and grammatical variations thereof, are inclusive or open-ended and do not exclude additional, un-recited elements and/or method steps. The term “consisting essentially of” when used herein in connection with a product, use or method, denotes that additional elements and/or method steps may be present, but that these additions do not materially affect the manner in which the recited method or use functions. The term “consisting of” when used herein in connection with a product, use or method, excludes the presence of additional elements and/or method steps. A product, use or method described herein as comprising certain elements and/or steps may also, in certain embodiments, consist essentially of those elements and/or steps, and in other embodiments consist of those elements and/or steps, whether or not these embodiments are specifically referred to. In addition, the use of the singular includes the plural, and “or” means “and/or” unless otherwise stated. Unless otherwise defined herein, all technical and scientific terms used herein have the same meaning as commonly understood by one of ordinary skill in the art. As used herein, the term “about” refers to an approximately +/−10% variation from a given value. It is to be understood that such a variation is always included in any given value provided herein, whether or not it is specifically referred to. The use of the word “a” or “an” when used herein in conjunction with the term “comprising” may mean “one,” but it is also consistent with the meaning of “one or more,” “at least one” and “one or more than one.”

The term “plant”, “portion of a plant”, “plant portion”, “plant matter”, “plant biomass”, “plant material”, plant extract”, or “plant leaves”, as used herein, may comprise an entire plant, tissue, cells, or any fraction thereof, intracellular plant components, extracellular plant components, liquid or solid extracts of plants, or a combination thereof, that are capable of providing the transcriptional, translational, and post-translational machinery for expression of one or more than one nucleic acids described herein, and/or from which an expressed protein or VLP may be extracted and purified. Plants may include, but are not limited to, herbaceous plants. Furthermore, plants may include, but are not limited to, agricultural crops including for example canola, Brassica spp., maize, Nicotiana spp., (tobacco) for example, Nicotiana benthamiana, Nicotiana rustica, Nicotiana, tabacum, Nicotiana alata, Arabidopsis thaliana, alfalfa, potato, sweet potato (Ipomoea batatus), ginseng, pea, oat, rice, soybean, wheat, barley, sunflower, cotton, corn, rye (Secale cereale), sorghum (Sorghum bicolor, Sorghum vulgare), safflower (Carthamus tinctorius).

The term “plant portion”, as used herein, refers to any part of the plant including but not limited to leaves, stem, root, flowers, fruits, a plant cell obtained from leaves, stem, root, flowers, fruits, a plant extract obtained from leaves, stem, root, flowers, fruits, or a combination thereof. The term “plant extract”, as used herein, refers to a plant-derived product that is obtained following treating a plant, a portion of a plant, a plant cell, or a combination thereof, physically (for example by freezing followed by extraction in a suitable buffer), mechanically (for example by grinding or homogenizing the plant or portion of the plant followed by extraction in a suitable buffer), enzymatically (for example using cell wall degrading enzymes), chemically (for example using one or more chelators or buffers), or a combination thereof. A plant extract may be further processed to remove undesired plant components for example cell wall debris. A plant extract may be obtained to assist in the recovery of one or more components from the plant, portion of the plant or plant cell, for example a protein (including protein complexes, protein surprastructures and/or VLPs), a nucleic acid, a lipid, a carbohydrate, or a combination thereof from the plant, portion of the plant, or plant cell. If the plant extract comprises proteins, then it may be referred to as a protein extract. A protein extract may be a crude plant extract, a partially purified plant or protein extract, or a purified product, that comprises one or more proteins, protein complexes, protein suprastructures, and/or VLPs, from the plant tissue. If desired a protein extract, or a plant extract, may be partially purified using techniques known to one of skill in the art, for example, the extract may be subjected to salt or pH precipitation, centrifugation, gradient density centrifugation, filtration, chromatography, for example, size exclusion chromatography, ion exchange chromatography, affinity chromatography, or a combination thereof. A protein extract may also be purified, using techniques that are known to one of skill in the art.

The term “construct”, “vector” or “expression vector”, as used herein, refers to a recombinant nucleic acid for transferring exogenous nucleic acid sequences into host cells (e.g. plant cells) and directing expression of the exogenous nucleic acid sequences in the host cells. “Expression cassette” refers to a nucleotide sequence comprising a nucleic acid of interest under the control of, and operably (or operatively) linked to, an appropriate promoter or other regulatory elements for transcription of the nucleic acid of interest in a host cell. As one of skill in the art would appreciate, the expression cassette may comprise a termination (terminator) sequence that is any sequence that is active the plant host. For example the termination sequence may be derived from the RNA-2 genome segment of a bipartite RNA virus, e.g. a comovirus, the termination sequence may be a NOS terminator, or terminator sequence may be obtained from the 3′UTR of the alfalfa plastocyanin gene.

The constructs of the present disclosure may further comprise a 3′ untranslated region (UTR). A 3′ untranslated region contains a polyadenylation signal and any other regulatory signals capable of effecting mRNA processing or gene expression. The polyadenylation signal is usually characterized by effecting the addition of polyadenylic acid tracks to the 3′ end of the mRNA precursor. Polyadenylation signals are commonly recognized by the presence of homology to the canonical form 5′ AATAAA-3′ although variations are not uncommon. Non-limiting examples of suitable 3′ regions are the 3′ transcribed non-translated regions containing a polyadenylation signal of Agrobacterium tumor inducing (Ti) plasmid genes, such as the nopaline synthase (Nos gene) and plant genes such as the soybean storage protein genes, the small subunit of the ribulose-1, 5-bisphosphate carboxylase gene (ssRUBISCO; U.S. Pat. No. 4,962,028; which is incorporated herein by reference), the promoter used in regulating plastocyanin expression.

By “regulatory region” “regulatory element” or “promoter” it is meant a portion of nucleic acid typically, but not always, upstream of the protein coding region of a gene, which may be comprised of either DNA or RNA, or both DNA and RNA. When a regulatory region is active, and in operative association, or operatively linked, with a nucleotide sequence of interest, this may result in expression of the nucleotide sequence of interest. A regulatory element may be capable of mediating organ specificity, or controlling developmental or temporal gene activation. A “regulatory region” includes promoter elements, core promoter elements exhibiting a basal promoter activity, elements that are inducible in response to an external stimulus, elements that mediate promoter activity such as negative regulatory elements or transcriptional enhancers. “Regulatory region”, as used herein, also includes elements that are active following transcription, for example, regulatory elements that modulate gene expression such as translational and transcriptional enhancers, translational and transcriptional repressors, upstream activating sequences, and mRNA instability determinants. Several of these latter elements may be located proximal to the coding region.

In the context of this disclosure, the term “regulatory element” or “regulatory region” typically refers to a sequence of DNA, usually, but not always, upstream (5′) to the coding sequence of a structural gene, which controls the expression of the coding region by providing the recognition for RNA polymerase and/or other factors required for transcription to start at a particular site. However, it is to be understood that other nucleotide sequences, located within introns, or 3′ of the sequence may also contribute to the regulation of expression of a coding region of interest. An example of a regulatory element that provides for the recognition for RNA polymerase or other transcriptional factors to ensure initiation at a particular site is a promoter element. Most, but not all, eukaryotic promoter elements contain a TATA box, a conserved nucleic acid sequence comprised of adenosine and thymidine nucleotide base pairs usually situated approximately 25 base pairs upstream of a transcriptional start site. A promoter element may comprise a basal promoter element, responsible for the initiation of transcription, as well as other regulatory elements that modify gene expression.

There are several types of regulatory regions, including those that are developmentally regulated, inducible or constitutive. A regulatory region that is developmentally regulated, or controls the differential expression of a gene under its control, is activated within certain organs or tissues of an organ at specific times during the development of that organ or tissue. However, some regulatory regions that are developmentally regulated may preferentially be active within certain organs or tissues at specific developmental stages, they may also be active in a developmentally regulated manner, or at a basal level in other organs or tissues within the plant as well. Examples of tissue-specific regulatory regions, for example see-specific a regulatory region, include the napin promoter, and the cruciferin promoter (Rask et al., 1998, J. Plant Physiol. 152: 595-599; Bilodeau et al., 1994, Plant Cell 14: 125-130). An example of a leaf-specific promoter includes the plastocyanin promoter (see U.S. Pat. No. 7,125,978, which is incorporated herein by reference).

An inducible regulatory region is one that is capable of directly or indirectly activating transcription of one or more DNA sequences or genes in response to an inducer. In the absence of an inducer the DNA sequences or genes will not be transcribed. Typically the protein factor that binds specifically to an inducible regulatory region to activate transcription may be present in an inactive form, which is then directly or indirectly converted to the active form by the inducer. However, the protein factor may also be absent. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory region may be exposed to an inducer by externally applying the inducer to the cell or plant such as by spraying, watering, heating or similar methods. Inducible regulatory elements may be derived from either plant or non-plant genes (e.g. Gatz, C. and Lenk, I. R. P., 1998, Trends Plant Sci. 3, 352-358). Examples, of potential inducible promoters include, but not limited to, tetracycline-inducible promoter (Gatz, C., 1997, Ann. Rev. Plant Physiol. Plant Mol. Biol. 48, 89-108), steroid inducible promoter (Aoyama, T. and Chua, N. H., 1997, Plant J. 2, 397-404) and ethanol-inducible promoter (Salter, M. G., et al, 1998, Plant Journal 16, 127-132; Caddick, M. X., et al, 1998, Nature Biotech. 16, 177-180) cytokinin inducible IB6 and CK11 genes (Brandstatter, I. and Kieber, J. J., 1998, Plant Cell 10, 1009-1019; Kakimoto, T., 1996, Science 274, 982-985) and the auxin inducible element, DR5 (Ulmasov, T., et al., 1997, Plant Cell 9, 1963-1971).

A constitutive regulatory region directs the expression of a gene throughout the various parts of a plant and continuously throughout plant development. Examples of known constitutive regulatory elements include promoters associated with the CaMV 35S transcript. (p35S; Odell et al., 1985, Nature, 313: 810-812; which is incorporated herein by reference), the rice actin 1 (Zhang et al, 1991, Plant Cell, 3: 1155-1165), actin 2 (An et al., 1996, Plant J., 10: 107-121), or tms 2 (U.S. Pat. No. 5,428,147), and triosephosphate isomerase 1 (Xu et. al., 1994, Plant Physiol. 106: 459-467) genes, the maize ubiquitin 1 gene (Cornejo et al, 1993, Plant Mol. Biol. 29: 637-646), the Arabidopsis ubiquitin 1 and 6 genes (Holtorf et al, 1995, Plant Mol. Biol. 29: 637-646), the tobacco translational initiation factor 4A gene (Mandel et al, 1995 Plant Mol. Biol. 29: 995-1004), the Cassava Vein Mosaic Virus promoter, pCAS, (Verdaguer et al., 1996); the promoter of the small subunit of ribulose biphosphate carboxylase, pRbcS: (Outchkourov et al., 2003), the pUbi (for monocots and dicots).

The term “constitutive” as used herein does not necessarily indicate that a nucleotide sequence under control of the constitutive regulatory region is expressed at the same level in all cell types, but that the sequence is expressed in a wide range of cell types even though variation in abundance is often observed.

The expression constructs as described above may be present in a vector. The vector may comprise border sequences which permit the transfer and integration of the expression cassette into the genome of the organism or host. The construct may be a plant binary vector, for example a binary transformation vector based on pPZP (Hajdukiewicz, et al. 1994). Other example constructs include pBin19 (see Frisch, D. A., L. W. Harris-Haller, et al. 1995, Plant Molecular Biology 27: 405-409).

The term “native”, “native protein” or “native domain”, as used herein, refers to a protein or domain having a primary amino acid sequence identical to wildtype. Native proteins or domains may be encoded by nucleotide sequences having 100% sequence similarity to the wildtype sequence. A native amino acid sequence may also be encoded by a human codon (hCod) optimized nucleotide sequence or a nucleotide sequence comprising an increased GC content when compared to the wild type nucleotide sequence provided that the amino acid sequence encoded by the hCod-nucleotide sequence exhibits 100% sequence identity with the native amino acid sequence.

By a nucleotide sequence that is “human codon optimized” or a “hCod” nucleotide sequence, it is meant the selection of appropriate DNA nucleotides for the synthesis of an oligonucleotide sequence or fragment thereof that approaches the codon usage generally found within an oligonucleotide sequence of a human nucleotide sequence. By “increased GC content” it is meant the selection of appropriate DNA nucleotides for the synthesis of an oligonucleotide sequence or fragment thereof in order to approach codon usage that, when compared to the corresponding native oligonucleotide sequence, comprises an increase of GC content, for example, from about 1 to about 30%, or any amount therebetween, over the length of the coding portion of the oligonucleotide sequence. For example, from about 1, 2, 4, 6, 8, 10, 12, 14, 16, 18, 20, 22, 24, 26, 28, 30%, or any amount therebetween, over the length of the coding portion of the oligonucleotide sequence. As described below, a human codon optimized nucleotide sequence, or a nucleotide sequence comprising an increased GC contact (when compared to the wild type nucleotide sequence) exhibits increased expression within a plant, portion of a plant, or a plant cell, when compared to expression of the non-human optimized (or lower GC content) nucleotide sequence.

Modified influenza hemagglutinin (HA) proteins (also termed modified HA protein, modified influenza HA protein, modified HA, modified influenza HA, mutant HA, influenza mutant HA, influenza HA variants or HA variants) and methods of producing modified influenza HA proteins in plants are described herein. The modified influenza HA proteins disclosed herewith comprise modifications or mutations that have been found to result in improved HA characteristics as compared to the wildtype HA or unmodified HA proteins. For example, the modified influenza HA protein may have an amino acid sequence with at least one substitution of an amino acid when compared to a corresponding wildtype amino acid sequence.

Examples of improved characteristics of the modified HA protein include, increased HA protein yield when expressed in plant cells as compared to the wildtype or unmodified HA of the same strain or subtype of influenza that does not comprise the modification(s) or mutation(s); improved hemagglutination titer of the modified HA protein when compared to the wildtype or unmodified HA protein; improved integrity, stability, or both integrity and stability, of virus like particles (VLPs) that are comprised of the modified HA proteins as compared to the integrity, stability or both of VLPs comprising wildtype HA that does not comprise the modification(s) or mutation(s); increased VLP yield when expressed in plant cells as compared to the wildtype level of VLP production that does not comprise the modification(s) or mutation(s); and a combination thereof.

### Influenza Subtypes and Strain

The term “influenza virus subtype” as used herein refers to influenza A virus variants that are characterized by various combinations of the hemagglutinin (H or HA) and neuramidase (N) viral surface proteins. According to the present specification, influenza virus subtypes and hemagglutinin (HA) from such virus subtypes may be referred to by their H number, such as, for example, “HA of the H3 subtype”, “H3 HA” or “H3 influenza”. The term “subtype” specifically includes all individual “strains” within each subtype, which usually result from mutations and may show different pathogenic profiles. Such strains may also be referred to as various “isolates” of a viral subtype. Accordingly, as used herein, the terms “strains” and “isolates” may be used interchangeably.

Traditionally, different strains of influenza have been categorized based upon, e.g., the ability of influenza to agglutinate red blood cells (RBCs or erythrocytes). Antibodies specific for particular influenza strains may bind to the virus and, thus, prevent such agglutination. Assays determining strain types based on such inhibition are typically known as hemagglutinin inhibition assays (HI assays or HAI assays) and are standard and well known methods in the art to characterize influenza strains.

However, HA proteins from different virus strains also show significant sequence similarity at both the nucleic acid and amino acid levels. This level of similarity varies when strains of different subtypes are compared, with some strains clearly displaying higher levels of similarity than others (Air, Proc. Natl. Acad. Sci. USA, 1981, 78:7643). The levels of amino acid similarity vary between virus strains of one subtype and virus strains of other subtypes (Air, Proc. Natl. Acad. Sci. USA, 1981, 78:7643). This variation is sufficient to establish discrete subtypes and the evolutionary lineage of the different strains, but the DNA and amino acid sequences of different strains are still readily aligned using conventional bioinformatics techniques (Air, Proc. Natl. Acad. Sci. USA, 1981, 78:7643; Suzuki and Nei, Mol. Biol. Evol. 2002, 19:501).

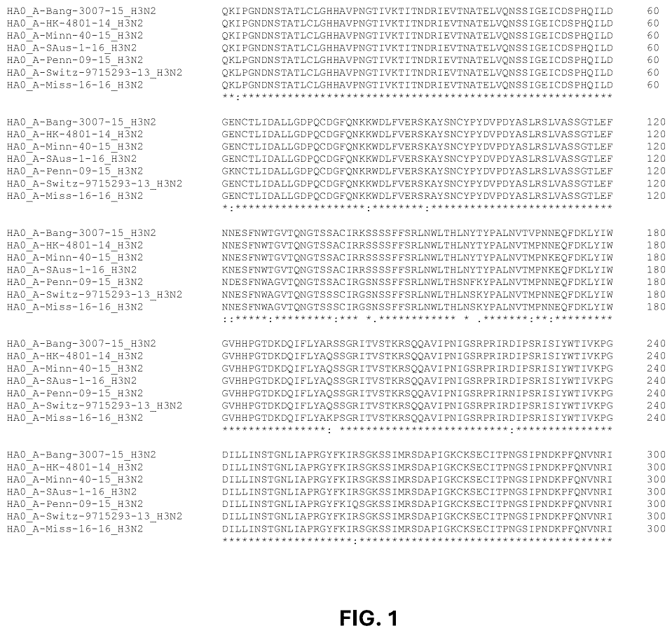

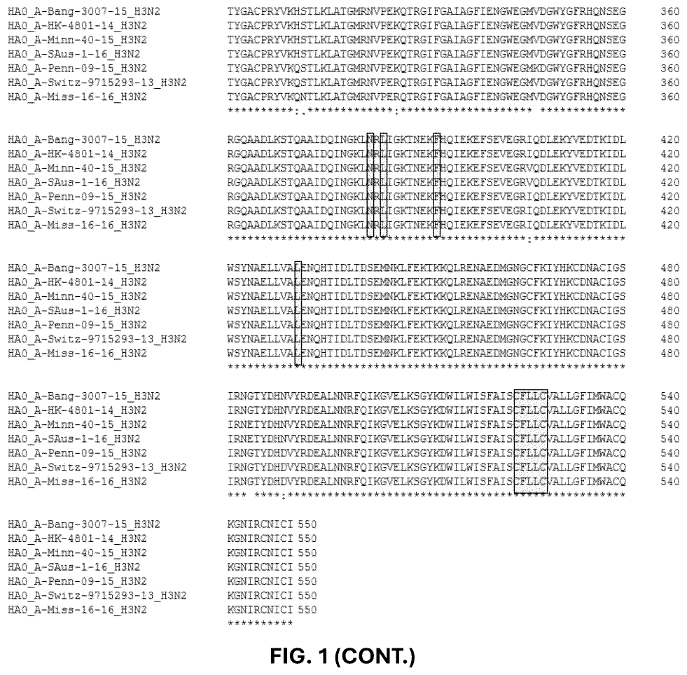

Multiple nucleotide sequences, or corresponding polypeptide sequences of hemagglutinin (HA), may be aligned to determine a “consensus” or “consensus sequence” of a subtype (see FIG. 1).

Based on sequence similarities, influenza virus subtypes can further be classified by reference to their phylogenetic group. Phylogenetic analysis (Fouchier et al., J Virol. 2005 March; 79(5):2814-22) has demonstrated a subdivision of HAs that falls into two main groups (Air, Proc. Natl. Acad. Sci. USA, 1981, 78:7643): inter alia the H1, H2, H5 and H9 subtypes in phylogenetic group 1 and inter alia the H3, H4 and H7 subtypes in phylogenetic group 2.

New influenza HA proteins, HA modifications, HA protein variants and mutants are created by introducing changes to the amino acid sequence of HA protein that results in an improved characteristic of the HA as described above. Isolation of nucleic acids encoding such HA molecules is routine, as is modification of the nucleic acid to introduce changes in the amino acid sequence, e.g., by site-directed mutagenesis.

Modified influenza HA proteins and methods of producing modified influenza HA proteins in plants are described herein. It has been observed that the modification for example by substitution of specific amino acids in HA proteins for example HA from subtype H3 results in improved characteristics of the modified HA protein when compared to the wildtype HA protein or unmodified HA protein.

The one or more than one modification, mutation or substitution of the HA protein as described herein are not located in the globular head domain of the HA protein, are not located in known epitopic regions of the HA protein nor do these modifications, mutations or substitutions add or remove glycosylation sites within the HA protein.

The HA protein, mutant HA protein or modified HA protein as described herein is modified and comprises one or more than one mutation, modification, or substitution in its amino acid sequence at any one or more amino acid that correspond to one or more than one mutation, or modification, at any one or more amino acid that corresponds with positions 382, 384, 392, 431, 524, 525, 526, 527 or 528 of the amino acid sequence of A/Hong Kong/4801/14 (SEQ ID NO: 92; see FIG. 1).

By “correspond to an amino acid” or “corresponding to an amino acid”, it is meant that an amino acid corresponds to an amino acids in a sequence alignment with an influenza reference strain as described below.

The amino acid residue number or residue position of HA is in accordance with the numbering of the HA of an influenza reference strain. For example in the case of influenza H3, the reference strain may be A/Hong Kong/4801/14 [SEQ ID NO: 92 see FIG. 1]. The corresponding amino acid positions may be determined by aligning the sequences of the HA (for example H3 HA) with the sequence of HA of their respective reference strain. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)). An amino acid sequence alignment of several influenza A HA domains, which are not to be considered limiting, is shown in FIG. 1.

When referring to modifications, mutants or variants, the wild type amino acid residue (also referred to as simply ‘amino acid’) is followed by the residue number and the new or substituted amino acid. For example, substitution of Asparagine (N, Asn) for Alanine (A, Ala) in residue or amino acid at position 382 is denominated N382A (see Table 1). 382 (N382A)

The modified HA, HA mutants or variants for example modified H3 HA are designated in the same manner by using the single letter amino acid code for the wild-type residue followed by its position and the single letter amino acid code of the replacement residue. Multiple mutants are indicated by component single mutants separated by slashes (/) or pluses (+). Mutations or modifications in the transmembrane (TM) domain or region are indicated as (CysTM). Thus for example the H3 HA mutant N382A+L384V (CysTM) is a mutant in which Alanine (A, Ala) replaces Asparagine (N, Asp) at residue position 382 and Valine (V, Val) replaces Leucine (L, Leu) at residue position 384 and the H3 HA mutant has further mutations in the transmembrane domain.

TABLE 1
Positions of modification in HA and the corresponding amino
acid/residue position in reference strains of influenza H3
and H5. The exemplified modification is shown in brackets.

| Position of | | |
|---|---|---|
| Modification in HA | | |
| (exemplification) | H3 HA¹ | H5 HA² |
| 382 (N382A) | N382 | N383 |
| 384 (L384V) | L384 | I385 |
| 392 (F392D) | F392 | F393 |
| 431(L431M) | L431 | M432 |
| 524 (C524S) | C524 | S526 |
| 525 (F525L) | F525 | L527 |
| 526 (L526V) | L526 | A528 |
| 527 | L527 | L529 |
| 528 (C528L) | C528 | A530 |

¹A/Hong Kong/4801/14
²A/Indonesia/05/05

The modified influenza hemagglutinin (HA) protein may comprise an amino acid sequence having at least one amino acid substitution when compared to a corresponding wildtype amino acid sequence.

By “amino acid substitution” or “substitution” it is meant the replacement of an amino acid in the amino acid sequence of a protein with a different amino acid. The terms amino acid, amino acid residue or residue are used interchangeably in the disclosure. One or more amino acids may be replaced with one or more amino acids that are different than the original or wildtype amino acid at this position, without changing the overall length of the amino acid sequence of the protein. The substitution or replacement may be experimentally induced by altering the codon sequence in a nucleotide sequence encoding the protein to the codon sequence of a different amino acid compared to the original or wildtype amino acid. The resulting protein is a modified protein, for example a modified influenza HA protein. The modified influenza HA protein does not occur naturally.

The modified HA includes non-naturally occurring HA protein, having at least one modification to naturally occurring HA and having improved characteristics compared to naturally occurring HA protein from which the amino acid sequence of the modified HA is derived. Modified HA proteins have an amino acid sequence, not found in nature, which is derived by replacement of one or more amino acid residues of an HA protein with one or more different amino acids.

Accordingly, modified HA, mutant HA or recombinant HA refers to an HA in which the DNA sequence encoding the naturally-occurring HA is modified to produce a modified or mutant DNA sequence which encodes the modification, mutation or substitution of one or more amino acids in the HA amino acid sequence.

Some of the residues identified for modification, mutation or substitution correspond to conserved residues whereas others are not. In the case of residues which are not conserved, the replacement of one or more amino acids is limited to substitutions which produce a modified HA which has an amino acid sequence that does not correspond to one found in nature. In the case of conserved residues, such modification, substitution or replacements should also not result in a naturally-occurring HA sequences.

### Conservative Substitutions

As described herein, residues in HA proteins may be identified and modified, substituted or mutated to produce modified HA protein or HA protein variants. The substitutions or mutations at specific positions are not limited to the amino acid substitutions described herewith or as given in the examples. For example, the HA variants may contain conserved or conservative substitutions of describes amino acid substitutions.

As used herein, the term “conserved substitution” or “conservative substitution” and grammatical variations thereof, refers to the presence of an amino acid residue in the sequence of the HA protein that is different from, but is in the same class of amino acid as the described substitution or described residue (i.e., a nonpolar residue replacing a nonpolar residue, an aromatic residue replacing an aromatic residue, a polar-uncharged residue replacing a polar-uncharged residue, a charged residue replacing a charged residue). In addition, conservative substitutions can encompass a residue having an interfacial hydropathy value of the same sign and generally of similar magnitude as the residue that is replacing the wildtype residue.

As used herein, the term “nonpolar residue” refers to glycine (G, Gly), alanine (A, Ala), valine (V, Val), leucine (L, Leu), isoleucine (I, lie), and proline (P, Pro); the term “aromatic residue” refers to phenylalanine (F, Phe), tyrosine (Y, Tyr), and tryptophan (W, Trp); the term “polar uncharged residue” refers to serine (S, Ser), threonine (T, Thr), cysteine (C, Cys), methionine (M, Met), asparagine (N, Asn) and glutamine (Q, Gln); the term “charged residue” refers to the negatively charged amino acids aspartic acid (D, Asp) and glutamic acid (E, Glu), as well as the positively charged amino acids lysine (K, Lys), arginine (R, Arg), and histidine (H, His). Other classification of amino acids may be as follows:
amino acids with hydrophobic side chain (aliphatic): Alanine (A, Ala), Isoleucine (I, lie), Leucine (L, Leu), Methionine (M, Met) and Valine (V, Val);
amino acids with hydrophobic side chain (aromatic): Phenylalanine (F, Phe), Tryptophan (W, Trp), Tyrosine (Y, Tyr);
amino acids with polar neutral side chain: Asparagine (N, Asn), Cysteine (C, Cys), Glutamine (Q, Gln), Serine (S, Ser) and Threonine (T, Thr);
amino acids with electrically charged side chains (acidic): Aspartic acid (D, Asp), Glutamic acid (E, Glu);
amino acids with electrically charged side chains (basic): Arginine (R, Arg); Histidine (H, His); Lysine (K, Lys), Glycine G, Gly) and Proline (P, Pro).

Conservative amino acid substitutions are likely to have a similar effect on the activity of the resultant HA protein variant or modified HA protein, as the original substitution or modification. Further information about conservative substitutions can be found, for instance, in Ben Bassat et al. (J. Bacteriol, 169:751-757, 1987), O'Regan et al. (Gene, 77:237-251, 1989), Sahin-Toth et al. (Protein ScL, 3:240-247, 1994), Hochuli et al (Bio/Technology, 6:1321-1325, 1988) and in widely used textbooks of genetics and molecular biology.

The Blosum matrices are commonly used for determining the relatedness of polypeptide sequences. The Blosum matrices were created using a large database of trusted alignments (the BLOCKS database), in which pairwise sequence alignments related by less than some threshold percentage identity were counted (Henikoff et al., Proc. Natl. Acad. Sci. USA, 89:10915-10919, 1992). A threshold of 90% identity was used for the highly conserved target frequencies of the BLOSUM90 matrix. A threshold of 65% identity was used for the BLOSUM65 matrix. Scores of zero and above in the Blosum matrices are considered “conservative substitutions” at the percentage identity selected. The following table shows exemplary conservative amino acid substitutions: Table 2.

TABLE 2
Exemplary conservative amino acid substitutions.

| | Very Highly- | | |
|---|---|---|---|
| Original | Conserved | Highly Conserved Substitutions | Conserved Substitutions |
| Residue | Substitutions | (from the Blosum90 Matrix) | (from the Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, Ser, Thr | Arg, Asp, Gln, Glu, His, Lys, Ser, Thr |
| Asp | Glu | Asn, Glu | Asn, Gln, Glu, Ser |
| Cys | Ser | None | Ala |
| Gln | Asn | Arg, Asn, Glu, His, Lys, Met | Arg, Asn, Asp, Glu, His, Lys, Met, Ser |
| Glu | Asp | Asp, Gln, Lys | Arg, Asn, Asp, Gln, His, Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

The nucleotide sequence encoding the modified HA protein may be optimized for human codon usage, for increased GC content, or a combination thereof. The modified HA protein may be expressed in a plant, portion of a plant, or plant cell.

### A. H3 HA Modifications I

Modified influenza H3 HA proteins and methods of producing modified influenza H3 HA proteins in plants are described herein. It has been observed that the modification of specific amino acids in HA proteins from subtype H3 results in improved characteristics of the modified H3 HA protein when compared to the wildtype H3 HA protein or unmodified H3 HA protein.

A total of 33 single, double and/or triple modifications to residues that are not part of the HA transmembrane (TM) and cytosolic domain (CT) and/or modifications to residues that are part of the transmembrane (TM) and cytosolic domain (CT) of HA were tested to improve the characteristics of the H3 HA protein. As described herewith and as shown in the Examples, only modifications or combinations of modifications at specific positions improved the characteristics of the H3 HA protein. Modifications at 13 positions or combinations of positions had negative effects on the characteristics of the H3 HA protein (data not shown).

Examples of improved characteristics of the H3 HA mutant or modified H3 HA protein include, increased HA protein yield or accumulation when expressed in plant cells as compared to the wildtype or unmodified HA of the same strain or subtype of influenza that does not comprise the modification(s) or mutation(s); improved hemagglutination titer of the modified or mutated HA protein when compared to the wildtype or unmodified HA protein; improved integrity, stability, or both integrity and stability, of VLPs that are comprised of the modified HA proteins as compared to the integrity, stability or both of VLPs comprising wildtype HA that does not comprise the mutation(s); increased VLP yield when expressed in plant cells as compared to the wildtype level of VLP production that does not comprise the modification(s) or mutation(s); and a combination thereof.

The modified H3 HA protein or mutant H3 HA protein as described herein is modified and comprises one or more than one mutation, or modification, at any one or more residues in sequence alignment with positions 382, 384, 392 and/or 431 of reference strain A/Hong Kong/4801/14 (SEQ ID NO: 92; see FIG. 1). It is therefore provided influenza H3 HA polypeptides, proteins, and/or protein complexes such as for example virus-like particle (VLP) that comprise modifications or mutations at one or more of amino acid positions 382, 384, 392 and 431, where such amino acid numbering is based upon the sequence of A/Hong Kong/4801/14 as shown in FIG. 1 (SEQ ID NO: 92), or at amino acid positions that correspond to such amino acid positions, for example as determined by alignment of an HA amino acid sequence to SEQ ID NO: 92. Non-limiting examples of influenza H3 HA amino acid sequences that comprise one or more of such mutations include SEQ ID NOs: 91, 92, 93, 94, 95, 96 and 97.

The modified H3 HA protein described herewith includes H3 HA protein with amino acid sequences that have about 70, 75, 80, 85, 87, 90, 91, 92, 93 94, 95, 96, 97, 98, 99, 100% or any amount therebetween, sequence identity, or sequence similarity, with the amino acid sequence encoding HA from H3 (SEQ ID NO: 91-97), wherein the amino acid sequence has one or more than one mutation, or modification, at any one or more residues in sequence alignment with positions 382, 384, 392 and 431 of A/Hong Kong/4801/14 HA (SEQ ID NO:92).

Furthermore, the H3 HA protein may be encoded by a nucleotide sequences that has about 70, 75, 80, 85, 87, 90, 91, 92, 93 94, 95, 96, 97, 98, 99, 100% or any amount therebetween, sequence identity, or sequence similarity, with the nucleotide sequence encoding HA from H3 (SEQ ID NO: 91-97), wherein the H3 HA protein has one or more than one mutation, or modification, at any one or more residues in sequence alignment with positions 382, 384, 392 and 431 of A/Hong Kong/4801/14 (SEQ ID NO: 92) HA and wherein the nucleotide sequence encodes a hemagglutinin protein that when expressed forms a VLP.

Non-limiting examples of strains from which the H3 HA might be derived are A/Bangkok/3007/15 (H3N2) (SEQ ID NO: 91); A/Hongkong/4801/14 (H3N2) (SEQ ID NO: 92); A/Minnesota/40/15 (H3N2) (SEQ ID NO: 93); A/South Australia/1/16 (H3N2) (SEQ ID NO: 94); A/Pennsylvania/09/15 (H3N2) (SEQ ID NO: 95); A/Switzerland/9715293/13 (H3N2) (SEQ ID NO: 96) and A/Mississippi/16/16 (H3N2) (SEQ ID NO: 97).

### Modification at Position 382

In one aspect it is provided a H3 HA that may have modified residue at position 382 (numbering in accordance with A/Hong Kong/4801/14 HA numbering, SEQ ID NO: 92).

Antanasijevic et al. (J Biol Chem. 2014; 289(32):22237-45) investigated the structure-function properties of H5 HA stem loop region by site directed mutagenesis at 14 different positions. Mutated positions Thr41, Gln42, Ile45, Asn53, and Leu99 of HA2 are highly conserved and were designed to assay the importance of these residues to HA function. Antanasijevic observed that conserved mutations at positions HA1-I28V, HA2-T41A, HA2-T49A, HA2-N53A, and HA2-D57E disrupted entry of the virus. In case of the mutational effect on entry inhibition by the small molecule MBX2329, substitutions to HA2-Ile45, HA2-Val52, HA2-Asn53, and HA2-Ser54 of the outer face and HA2-Leu99 of the inner face had the largest effects. These residues are highly conserved in the Group 1 HA (e.g. H1 and H5). Position 53 in the HA of H5 of Antanasijevic corresponds to position 380 in H1 HA and position 382 in H3 HA of the current disclosure.

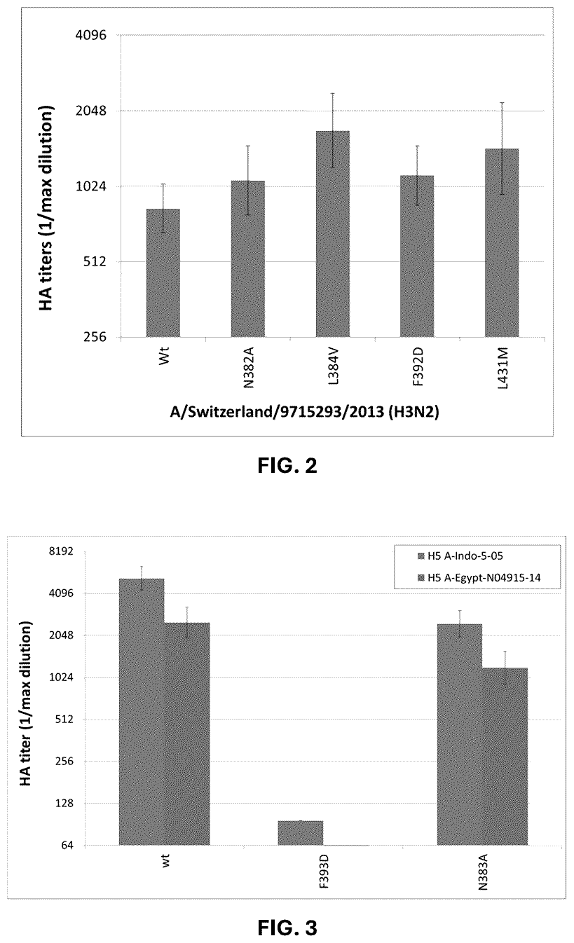

It was unexpectedly found that modification of the conserved residue at position 380 in H1 HA from an Asparagine to Alanine lead to an approximate 80% decrease in hemagglutination titer of the modified H1 HA when compared to wildtype H1 HA (data not shown). The equivalent modification in HA from H5 also lead to a decrease in hemagglutination titer (see FIG. 3, Table 7). However, modification of equivalent position in HA from H3 (N382A) from an Asparagine to Alanine lead to an approximate 130% increase in hemagglutination titer when compared to wildtype H3 HA (see FIG. 2, Table 5).

Accordingly, the residue at position 382 of H3 HA may be modified to be a non-Asparagine. For example the residue at position 382 may be modified to be a hydrophobic amino acid for example Alanine or a conserved substitution of Alanine for example Serine (S, Ser), Glycine (G, Gly), Threonine (T, Thr), Cysteine (C, Cys) or Valine (V, Val).

For example the modified H3 HA protein may have an amino acid sequence that has about 70, 75, 80, 85, 87, 90, 91, 92, 93 94, 95, 96, 97, 98, 99, 100% or any amount therebetween, sequence identity, or sequence similarity, with the amino acid sequence of HA from H3 A/Hong Kong/4801/14 (SEQ ID NO: 92), wherein the amino acid sequence has an Alanine (A, Ala) or a conserved substitution of Alanine (A, Ala) at position 382, and wherein the sequence does not occur naturally. The conserved substitution of Alanine may for example be Serine (S, Ser), Glycine (G, Gly), Threonine (T, Thr), Cysteine (C, Cys) or Valine (V, Val).

The present specification also provides a nucleic acid comprising a nucleotide sequence encoding a modified H3 HA with a substitution at positions 382, as described above operatively linked to a regulatory region active in a plant.

For example the nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence encoding HA from H3 A/Hong Kong/4801/14 (SEQ ID NO: 102), wherein the nucleotide sequence encodes a modified H3 HA protein that has an Alanine (A, Ala) or a conserved substitution of Alanine (A, Ala) at position 382, and wherein the sequence does not occur naturally. The conserved substitution may for example be Serine (S, Ser), Glycine (G, Gly), Threonine (T, Thr), Cysteine (C, Cys) or Valine (V, Val).

The nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence of SEQ ID NO: 102, wherein the nucleotide sequence encodes a modified H3 HA protein that has an Alanine (A, Ala) or a conserved substitution of Alanine (A, Ala) at position 382, and wherein the sequence does not occur naturally. The conserved substitution may for example be Serine (S, Ser), Glycine (G, Gly), Threonine (T, Thr), Cysteine (C, Cys) or Valine (V, Val).

In addition to residue at position 382, residues at positions 384, 524, 525, 526, 527, 528 or any combination thereof may be modified in the H3 HA.

Furthermore, it is provided a method of producing VLPs that comprise a modified H3 HA with a substitution at least at position 382 and optionally at position 384, 524, 525, 526, 527, 528 or any combination thereof in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

In addition, it is provided a method of increasing yield of VLPs that comprise a modified H3 HA with a substitution at least at position 382 and optionally substitutions at positions 384, 524, 525, 526, 527, 528 or any combination thereof in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

The present specification further provides for a VLP comprising a H3 HA with a substitution at least at position 382 and optionally substitutions at positions 384, 524, 525, 526, 527, 528 or any combination thereof. The VLP may be produced by the method as provided by the present specification. The VLP comprising the modified H3 HA show improved characteristics when compared to VLPs that comprise the unmodified H3 HA protein.

### Modification at Position 384

In another aspect it is provided a H3 HA that may have a modified residue at position 384 (A/Hong Kong/4801/14 HA numbering, SEQ ID NO: 92).

As shown for example in FIG. 2 and Table 5 having the residue at position 384 modified from Leucine to Valine lead to an approximately 200% increase in hemagglutination titer as compared to wildtype H3 HA.

Accordingly, the residue at position 384 of H3 HA may be modified to be a non-Leucine. For example the residue at position 384 may be modified to be a for example Valine or a conserved substitution of Valine that is not Leucine for example Isoleucine, Methionine, Alanine, or Threonine.

For example the modified H3 HA protein may have an amino acid sequence that has about 70, 75, 80, 85, 87, 90, 91, 92, 93 94, 95, 96, 97, 98, 99, 100% or any amount therebetween, sequence identity, or sequence similarity, with the amino acid sequence of HA from H3 A/Hong Kong/4801/14 (SEQ ID NO: 92), wherein the amino acid sequence has an Valine (V, Val) or a conserved substitution of Valine that is not Leucine at position 384, wherein the sequence does not occur naturally and wherein the HA protein when expressed form VLP. The conserved substitution of Valine may for example be Isoleucine, Methionine, Alanine, or Threonine.

The present specification also provides a nucleic acid comprising a nucleotide sequence encoding a modified H3 HA with a substitution at positions 384 as described above operatively linked to a regulatory region active in a plant.

For example the nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence encoding HA from H3 A/Hong Kong/4801/14 (SEQ ID NO: 102), wherein the nucleotide sequence encodes a modified H3 HA protein that has a Valine (V, Val) or a conserved substitution of Valine (V, Val) that is not Leucine at position 384, wherein the sequence does not occur naturally and wherein the nucleotide sequence encodes HA protein that when expressed form VLP. The conserved substitution of Valine may for example be Isoleucine, Methionine, Alanine, or Threonine.

The nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence of SEQ ID NO: 102, wherein the nucleotide sequence encodes a modified H3 HA protein that has a Valine (V, Val) or a conserved substitution of Valine (V, Val) that is not Leucine at position 384, wherein the sequence does not occur naturally and wherein the nucleotide sequence encodes HA protein that when expressed form VLP. The conserved substitution of Valine may for example be Isoleucine, Methionine, Alanine, or Threonine.

In addition to residue at position 384, residues at positions 382, 524, 525, 526, 527, 528 or any combination thereof may be modified in the H3 HA.

Furthermore, it is provided a method of producing VLPs that comprise a modified H3 HA with a substitution at least at positions 384 and optionally substitutions at positions 382, 524, 525, 526, 527, 528 or any combination thereof in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

In addition, it is provided a method of increasing yield of VLPs that comprise a modified H3 HA with a substitution at least at 384 and optionally substitutions at positions 382, 524, 525, 526, 527, 528 or any combination thereof, as described above in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

The present specification further provides for a VLP comprising a H3 HA with a substitution at least at position 384 and optionally substitutions at positions 382, 524, 525, 526, 527, 528 or any combination thereof. The VLP may be produced by the method as provided by the present specification. The VLP comprising the modified H3 HA show improved characteristics when compared to VLPs that comprise the unmodified H3 HA protein.

### Modification at Position 392

In another aspect it is provided a H3 HA that may have modified residue at position 392 (H3 A/Hong Kong/4801/14 HA numbering, SEQ ID NO: 92).

As shown for example in FIG. 2 and Table 5 having the residue at position 392 modified from Phenylalanine to Aspartic Acid lead to an approximately 140% increase in hemagglutination titer as compared to wildtype H3 HA.

Accordingly, the residue at position 392 of H3 HA may be modified to be a non-Phenylalanine. For example the residue at position 392 may be modified to be for example Aspartic Acid or a conserved substitution of Aspartic Acid for example Glutamic acid, Asparagine, Glutamine, or Serine.

For example the modified H3 HA protein may have an amino acid sequence that has about 70, 75, 80, 85, 87, 90, 91, 92, 93 94, 95, 96, 97, 98, 99, 100% or any amount therebetween, sequence identity, or sequence similarity, with the amino acid sequence of HA from H3 A/Hong Kong/4801/14 (SEQ ID NO: 92), wherein the amino acid sequence has an Aspartic Acid or a conserved substitution of Aspartic Acid for example Glutamic acid, Asparagine, Glutamine, or Serine at position 392, wherein the sequence does not occur naturally and wherein the HA protein when expressed form VLP. The conserved substitution of Aspartic Acid may for example be Glutamic acid, Asparagine, Glutamine, or Serine.

The present specification also provides a nucleic acid comprising a nucleotide sequence encoding a modified H3 HA with a substitution at positions 392 as described above operatively linked to a regulatory region active in a plant.

For example the nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence encoding HA from H3 A/Hong Kong/4801/14 (SEQ ID NO: 102), wherein the nucleotide sequence encodes a modified H3 HA protein that has an Aspartic Acid or a conserved substitution of Aspartic Acid at position 392, wherein the sequence does not occur naturally and wherein the nucleotide sequence encodes HA protein that when expressed form VLP. The conserved substitution of Aspartic Acid may for example be Glutamic acid, Asparagine, Glutamine, or Serine.

The nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence of SEQ ID NO: 102, wherein the nucleotide sequence encodes a modified H3 HA protein that has an Aspartic Acid or a conserved substitution of Aspartic Acid at position 392, wherein the sequence does not occur naturally and wherein the nucleotide sequence encodes HA protein that when expressed form VLP. The conserved substitution of Aspartic Acid may for example be Glutamic acid, Asparagine, Glutamine, or Serine.

Furthermore, it is provided a method of producing VLPs that comprise a modified H3 HA with a substitution at least at positions 392 as described above in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

In addition, it is provided a method of increasing yield of VLPs that comprise a modified H3 HA with a substitution at least at 392, as described above in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

The present specification further provides for a VLP comprising a H3 HA with a substitution at least at position 392. The VLP may be produced by the method as provided by the present specification. The VLP comprising the modified H3 HA show improved characteristics when compared to VLPs that comprise the unmodified H3 HA protein.

### Modification at Position 431

In another aspect it is provided a H3 HA that may have modified residue at position 431 (H3 A/Hong Kong/4801/14 HA numbering, SEQ ID NO: 92).

As shown for example in FIG. 2 and Table 5 having the residue at position 431 modified from Leucine to Methionine lead to an approximately 170% increase in hemagglutination titer as compared to wildtype H3 HA.

Accordingly, the residue at position 431 of H3 HA may be modified to be a non-Leucine. For example the residue at position 431 may be modified to be for example Methionine or a conserved substitution of Methionine for example Leucine, Isoleucine, Glutamine, Valine or Phenylalanine.

For example the modified H3 HA protein may have an amino acid sequence that has about 70, 75, 80, 85, 87, 90, 91, 92, 93 94, 95, 96, 97, 98, 99, 100% or any amount therebetween, sequence identity, or sequence similarity, with the amino acid sequence of HA from H3 A/Hong Kong/4801/14 (SEQ ID NO: 92), wherein the amino acid sequence has Methionine or a conserved substitution of Methionine at position 431, wherein the sequence does not occur naturally and wherein the HA protein that when expressed form VLP. The conserved substitution of Methionine may for example be Leucine, Isoleucine, Glutamine, Valine or Phenylalanine.

The present specification also provides a nucleic acid comprising a nucleotide sequence encoding a modified H3 HA with a substitution at positions 431 as described above operatively linked to a regulatory region active in a plant.

For example the nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence encoding HA from H3 A/Hong Kong/4801/14 (SEQ ID NO: 102), wherein the nucleotide sequence encodes a modified H3 HA protein that has Methionine or a conserved substitution of Methionine at position 431, wherein the sequence does not occur naturally and wherein the nucleotide sequence encodes HA protein that when expressed form VLP. The conserved substitution of Methionine may for example be Leucine, Isoleucine, Glutamine, Valine or Phenylalanine.

The nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence of SEQ ID NO: 102, wherein the nucleotide sequence encodes a modified H3 HA protein that has Methionine or a conserved substitution of Methionine at position 431, wherein the sequence does not occur naturally and wherein the nucleotide sequence encodes HA protein that when expressed form VLP. The conserved substitution of Aspartic Acid may for example be Leucine, Isoleucine, Glutamine, Valine or Phenylalanine.

Furthermore, it is provided a method of producing VLPs that comprise a modified H3 HA with a substitution at least at positions 431 as described above in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

In addition, it is provided a method of increasing yield of VLPs that comprise a modified H3 HA with a substitution at least at 431, as described above in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

The present specification further provides for a VLP comprising a H3 HA with a substitution at least at position 431. The VLP may be produced by the method as provided by the present specification. The VLP comprising the modified H3 HA show improved characteristics when compared to VLPs that comprise the unmodified H3 HA protein.

### B. H3 HA Modifications II

Modification at Position 382, 384, 392, 431 and/or 524-528 (“CysTM”)

Modified influenza H3 HA proteins and methods of producing modified influenza H3 HA proteins in plants are described herein. It has been observed that the modification of specific amino acids in HA proteins from subtype H3 results in improved characteristics of the modified H3 HA protein when compared to the wildtype H3 HA protein or unmodified H3 HA protein.

In one aspect, cysteine residues at position 524 and/or 528 (H3 A/Hong Kong/4801/14 numbering, SEQ ID NO: 92) or residues equivalent to these positions as determined by alignment may be substituted for non-cysteine residues at these positions. Furthermore, residues at position 525, 526 and/or 527 (H3 A/Hong Kong/4801/14 HA numbering) may also be modified. These modification or substitution may be referred to as the “CysTM modification”, “CysTM”, “CysTM mutation”, “CysTM substitution” or “CysTM replacement” or grammatically equivalent expressions.

U.S. application Ser. No. 13/838,796 and its companion publication by Holtz et al. (BMC Biotechnology. 2014; 14:111) teach the improved stability and maintained potency of recombinant HA by the mutation of cysteine residues in the carboxy terminal region of the HA protein including the transmembrane (TM) and cytosolic domain (CT). Specifically, Holtz et al. demonstrate C539A, C546A, C549A, C524S and C528A mutations in recombinant Perth/16/2009 HA (H3N2). Mutation of all five cysteine residues, or different subsets thereof, resulted in HA yields, purities, particle size, hemagglutination activity, and thermostability comparable to recombinant wildtype HA protein. In contrast, mutations of a pair of conserved cysteine residues in the ectodomain known to form a disulfide bond (C64S and C76S) resulted in significantly reduced HA expression, indicating the critical role of these residues in proper HA folding. By using a single radial immune-diffusion assay (SRID), Holtz et al. also show that the five cysteine residue mutations improve potency of recombinant HA as compared to wildtype protein, by preventing disulfide cross-linking in the TM and CT domains. The mutant HA proteins maintain potency for at least 12 months at 25° C., whereas wildtype HA protein exhibited less than 40% potency after only 50 days post purification.

Xu et al. (Virus Genes (2013) 47:20-26) showed that mutant H3 HAs with mutations of one or two of the TM cysteines (C540/544) could be expressed properly in cells, however the mutant exhibited lower thermal resistance and enhanced fusion activity in comparison with wildtype H3 HA proteins.

A total of 33 single, double or triple modifications in combination with modifications to the transmembrane (TM) as described herein (“CysTM modification”) were tested to improve the characteristics of the H3 HA protein. As described herewith and as shown in the Examples, only modifications or combinations of modifications at certain positions improved the characteristics of the H3 HA protein. Modifications at 13 positions or combinations of positions had negative effects on the characteristics of the H3 HA protein (data not shown).

Examples of improved characteristics of the H3 HA mutant protein include, increased HA protein yield when expressed in plant cells as compared to the wildtype or unmodified HA of the same strain or subtype of influenza that does not comprise the modification(s) or mutation(s); improved hemagglutination titer of the modified or mutated HA protein when compared to the wildtype or unmodified HA protein; improved integrity, stability, or both integrity and stability, of VLPs that are comprised of the modified HA proteins as compared to the integrity, stability or both of VLPs comprising wildtype HA that does not comprise the mutation(s); increased VLP yield when expressed in plant cells as compared to the wildtype level of VLP production that does not comprise the modification(s) or mutation(s); and a combination thereof.

In one aspect, it is provided a modified H3 HA having cysteine residues at position 524 and/or 528 (A/Hong Kong/4801/14 numbering) or residues equivalent to these positions as determined by alignment substituted for non-cysteine residues at these positions. For example a cysteine residues at position 524 may be substituted with a Serine (S, Ser) or a conserved substitution of Serine or a cysteine at position 528 may be substituted by a Leucine (L, Leu) or a conserved substitution of Leucine.

Furthermore, the sequence between the two cysteines at position 524 and 528 (A/Hong Kong/4801/14 numbering, SEQ ID NO: 92) may also be modified. For example in the sequence “C524X525X526X527C528”, C524 (position 524) may be substituted with a Serine (S, Ser) or a conserved substitution of Serine; if X525 (position 525) is not Leucine, the residue X525 may be replaced with Leucine (L, Leu) or a conserved substitution of Leucine; if X526 (position 526) is not Valine, X526 may be replaced Valine (V, Val) or a conserved substitution of Valine; if X527 (position 527) is not Leucine, the residue at position X527 may be replaced with Leucine (L, Leu) or a conserved substitution of Leucine and C528 (position 528) may be substituted with a Leucine (L, Leu) or a conserved substitution of Leucine. For example, in one embodiment the sequence “CFLLC” at position 524 to 528 may be replaced with the sequence “SLVLL” (SEQ ID NO: 98).

HA proteins from other strains than H3 influenza that have cysteine residues at position 524 and/or 528 or residues equivalent to these positions as determined by alignment may also be modified as described herewith. For example HA proteins from influenza B strains that have cysteine residues at positions that are equivalent to position 524 and/or 528 of H3 HA may be modified to non-cysteine residues as described herewith.

In addition to the mutation of the transmembrane domain as described herein, the modified H3 HA proteins may further comprise one or more than one substitutions of amino acids at positions 382, 384, 392, and/or 431, which resulted in an improved characteristic of the modified HA3 protein, or VLP produced using the modified HA protein. It is to be understood that the improved characteristic is not limited to substituting the specific amino acid at the specified sites, as one of skill in the art would understand that amino acids with similar properties may be substituted for the amino acids at the identified positions.

Accordingly, the modified H3 HA protein or mutant H3 HA protein as described herein may comprise one or more than one mutation, or modification, at any one or more residues in sequence alignment with positions 382, 384, 392, 431, 524, 525, 526, 527 or 528 of reference strain H3 A/Hong Kong/4801/14 (SEQ ID NO: 92; see FIG. 1). It is therefore provided influenza H3 HA polypeptides, proteins, and/or protein complexes such as for example virus-like particle (VLP) that comprise modifications or mutations at one or more of amino acid positions 382, 384, 392, 431, 524, 525, 526, 527 or 528, where such amino acid numbering is based upon the sequence of H3 A/Hong Kong/4801/14 as shown in FIG. 1 (SEQ ID NO: 92), or at amino acid positions that correspond to such amino acid positions, for example as determined by alignment of an HA amino acid sequence to SEQ ID NO: 92. Non-limiting examples of influenza H3 HA amino acid sequences that comprise one or more of such mutations include SEQ ID NOs: 91, 92, 93, 94, 95, 96 and 97.

The modified H3 HA protein described herewith includes H3 HA protein with amino acid sequences that have about 70, 75, 80, 85, 87, 90, 91, 92, 93 94, 95, 96, 97, 98, 99, 100% or any amount therebetween, sequence identity, or sequence similarity, with the amino acid sequence encoding HA from H3 (SEQ ID NO: 91-97), wherein the amino acid sequence has one or more than one mutation, or modification, at any one or more residues in sequence alignment with positions 382, 384, 392, 431, 524, 525, 526, 527 or 528 of A/Hong Kong/4801/14 HA (SEQ ID NO: 92).

Furthermore, the H3 HA protein may be encoded by a nucleotide sequences that has about 70, 75, 80, 85, 87, 90, 91, 92, 93 94, 95, 96, 97, 98, 99, 100% or any amount therebetween, sequence identity, or sequence similarity, with the nucleotide sequence encoding HA from H3 (SEQ ID NO: 91-97 sequences of H3 strains), wherein the H3 HA protein has one or more than one mutation, or modification, at any one or more residues in sequence alignment with positions 382, 384, 392, 431, 524, 525, 526, 527 or 528 of H3 A/Hong Kong/4801/14 HA (SEQ ID NO: 92) and wherein the nucleotide sequence encodes a hemagglutinin protein that when expressed forms a VLP.

Non-limiting examples of strains from which the H3 HA might be derived are A/Bangkok/3007/15 (H3N2) (SEQ ID NO: 91); A/Hongkong/4801/14 (H3N2) (SEQ ID NO: 92); A/Minnesota/40/15 (H3N2) (SEQ ID NO: 93); A/South Australia/1/16 (H3N2) (SEQ ID NO: 94); A/Pennsylvania/09/15 (H3N2) (SEQ ID NO: 95); A/Switzerland/9715293/13 (H3N2) (SEQ ID NO: 96) and A/Mississippi/16/16 (H3N2) (SEQ ID NO: 97).

### Modification at Position 524-528 (CysTM) and 382

In another aspect it is provided a H3 HA that may have a modified residue at position 382, and may have been modified to have cysteine residue at positions 524 and/or 528 (H3 A/Hongkong/4801/14 numbering, SEQ ID NO: 92). Furthermore, residues at position 525, 526 and/or 527 (H3 A/Hongkong/4801/14 numbering) may also be modified.

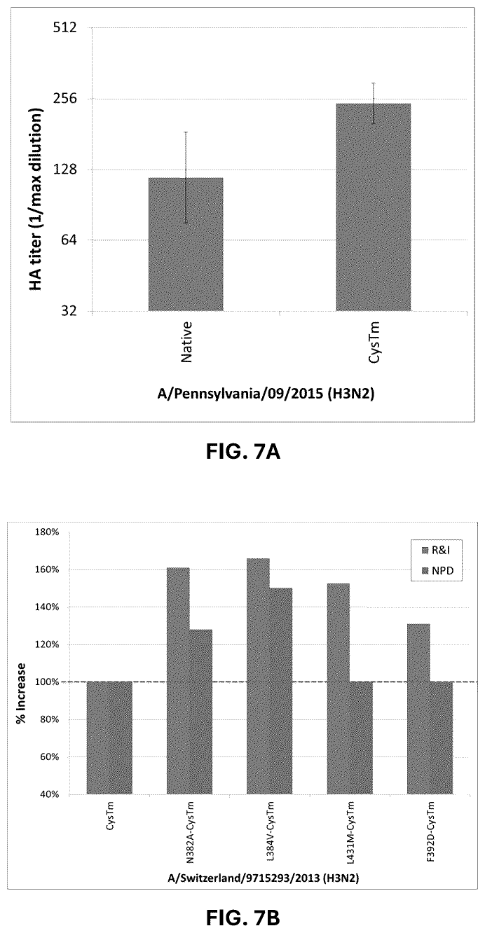

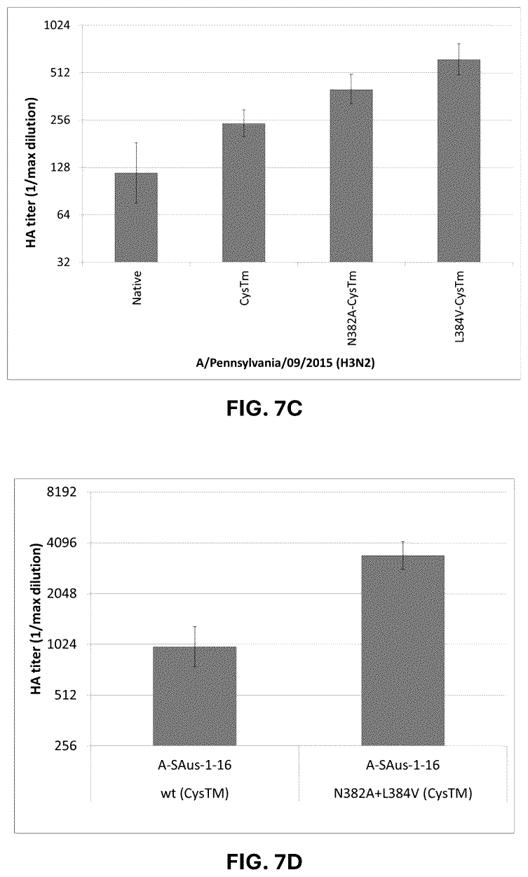

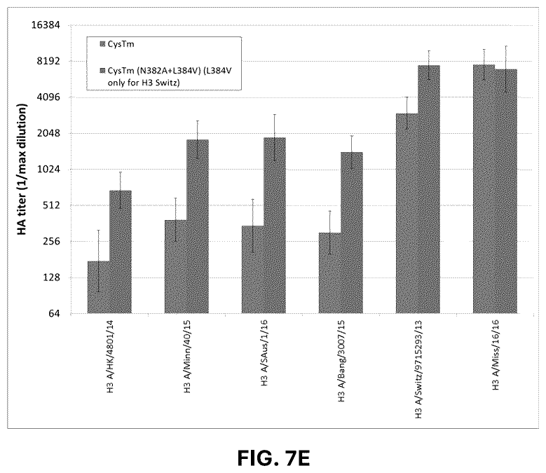

As shown for example in FIGS. 7A, 7B and 7C, an H3 HA having cysteine residues at positions 524 and 528 modified to be non-cysteine and having residue at position 382 modified from Asparagine to Alanine lead to an approximately 260% increase in hemagglutination titer as compared to wildtype H3 HA.

Accordingly, it is provided a modified H3 HA protein that comprise one or more than one modification at position 524, 525, 526, 527 or 528 and a modification at position 382. In a non-limiting example the modified H3 HA has modifications at least at positions 382, 524 and 528.

The residue at position 382 of H3 HA may be modified to be a non-Asparagine. For example the residue at position 382 may be modified to be a hydrophobic amino acid for example Alanine or a conserved substitution of Alanine for example Serine (S, Ser), Glycine (G, Gly), Threonine (T, Thr), Cysteine (C, Cys) or Valine (V, Val). The residue at position 524 may be modified to a non-cysteine, a Serine or a conserved substitution of Serine (S, Ser) and the residue at position 528 may be modified to a non-cysteine, Leucine (L, Leu) or a conserved substitution of Leucine.

For example the modified H3 HA protein may have an amino acid sequence that has about 70, 75, 80, 85, 87, 90, 91, 92, 93 94, 95, 96, 97, 98, 99, 100% or any amount therebetween, sequence identity, or sequence similarity, with the amino acid sequence of HA from H3 A/Hongkong/4801/14 (SEQ ID NO: 92), wherein the amino acid sequence has an Alanine (A, Ala) or a conserved substitution of Alanine (A, Ala) at position 382, no cysteine residue at positions 524 and 528, wherein the sequence does not occur naturally and wherein the HA when expressed form VLP. The conserved substitution of Alanine may for example be Serine (S, Ser), Glycine (G, Gly), Threonine (T, Thr), Cysteine (C, Cys) or Valine (V, Val). The non-cysteine at position 524 may be a Serine (S, Ser) or a conserved substitution of Serine and the non-cysteine at position 528 may be Leucine (L, Leu) or a conserved substitution of Leucine. The conserved substitution of Serine (Se, Ser) may for example be Threonine (T, Thr), Alanine (A, Ala), Asparagine (N, Asn), Aspartic Acid (D, Asp), Glutamine (Q, Gln), Glycine (G, Gly), Glutamic acid (E, Glu) or Lysine (L, Lys). The conserved substitution of Leucine may for example be Isoleucine (I, lie), Valine (V, Val), Methionine (M, Met), Phenylalanine (F, Phe), or Valine (V, Val).

The present specification also provides a nucleic acid comprising a nucleotide sequence encoding a modified H3 HA with a substitution at positions 382, 524 and 528 as described above operatively linked to a regulatory region active in a plant.

For example the nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence encoding HA from H3 A/Hongkong/4801/14 (SEQ ID NO: 102), wherein the nucleotide sequence encodes a modified H3 HA protein that has an Alanine (A, Ala) or a conserved substitution of Alanine (A, Ala) at position 382, no cysteine residue at positions 524 and 528, wherein the sequence does not occur naturally and wherein the HA when expressed form VLP. The conserved substitution for Alanine may for example be Serine (S, Ser), Glycine (G, Gly), Threonine (T, Thr), Cysteine (C, Cys) or Valine (V, Val). The non-cysteine at position 524 may be a Serine (S, Ser) or a conserved substitution of Serine and the non-cysteine at position 528 may be Leucine (L, Leu) or a conserved substitution of Leucine. The conserved substitution of Serine (Se, Ser) may for example be Threonine (T, Thr), Alanine (A, Ala), Asparagine (N, Asn), Aspartic Acid (D, Asp), Glutamine (Q, Gln), Glycine (G, Gly), Glutamic acid (E, Glu) or Lysine (L, Lys). The conserved substitution of Leucine may for example be Isoleucine (I, lie), Valine (V, Val), Methionine (M, Met), Phenylalanine (F, Phe), or Valine (V, Val).

The nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence of SEQ ID NO: 102, wherein the nucleotide sequence encodes a modified H3 HA protein that has an Alanine (A, Ala) or a conserved substitution of Alanine (A, Ala) at position 382, no cysteine residue at positions 524 and 528, wherein the sequence does not occur naturally and wherein the HA when expressed form VLP. The conserved substitution of Alanine may for example be Serine (S, Ser), Glycine (G, Gly), Threonine (T, Thr), Cysteine (C, Cys) or Valine (V, Val).

In addition to residues at position 382, 524 and 528, the modified H3 HA may have further residues modified. For example one or more than one residues at position 384, 525, 526 and 527 may be modified. In a non-limiting example the modified H3 HA may have substituted residues at position 382, 524, 528 and one or more substitutions at positions 384, 525, 526, 527 or a combination thereof.

Furthermore, it is provided a method of producing VLPs that comprise a modified H3 HA with a substitution at least at positions 382, 524 and 528 and optionally substitutions at positions 384, 525, 526, 527 above in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

In addition, it is provided a method of increasing yield of VLPs that comprise a modified H3 HA with a substitution at least at position 382, 524 and 528 and optionally substitutions at positions 384, 525, 526, 527, as described above in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

The present specification further provides for a VLP comprising a H3 HA with a substitution at least at position 382, 524 and 528 and optionally substitutions at positions 384, 525, 526, 527. The VLP may be produced by the method as provided by the present specification. The VLP comprising the modified H3 HA show improved characteristics when compared to VLPs that comprise the unmodified H3 HA protein.

### Modification at Position 524-528 (CysTM) and 384

In another aspect it is provided a H3 HA that may have modified residue at position 384, and may be modified to have no cysteine residue at positions 524 and 528 (H3 A/Hongkong/4801/14 numbering, SEQ ID NO: 92). Furthermore, residues at position 525, 526 and/or 527 (H3 A/Hongkong/4801/14 numbering) may also be modified.

As shown for example in FIGS. 7A, 7B and 7C, an H3 HA having cysteine residues at positions 524 and 528 modified to be non-cysteine residues and having residue at position 384 modified from Leucine to Valine, lead to an approximately 270% increase in hemagglutination titer as compared to wildtype H3 HA.

Accordingly, it is provided a modified H3 HA protein that comprise one or more than one modification at position 524, 525, 526, 527 or 528 and a modification at position 384. In a non-limiting example the modified H3 HA has modifications at least at positions 384, 524 and 528.

The residue at position 384 of H3 HA may be modified to be a non-Leucine. For example the residue at position 384 may be modified to another hydrophobic amino acid for example Valine or a conserved substitution of Valine for example Isoleucine, Leucine, Methionine, Alanine or Threonine.

For example the modified H3 HA protein may have an amino acid sequence that has about 70, 75, 80, 85, 87, 90, 91, 92, 93 94, 95, 96, 97, 98, 99, 100% or any amount therebetween, sequence identity, or sequence similarity, with the amino acid sequence of HA from H3 A/Hongkong/4801/14 (SEQ ID NO: 92), wherein the amino acid sequence has a Valine or a conserved substitution of Valine at position 384, no cysteine residue at positions 524 and 528, wherein the sequence does not occur naturally and wherein the HA when expressed form VLP. The conserved substitution of Valine may for example be Isoleucine, Leucine, Methionine, Alanine or Threonine.

The present specification also provides a nucleic acid comprising a nucleotide sequence encoding a modified H3 HA with a substitution at positions 384, 524 and 528 as described above operatively linked to a regulatory region active in a plant.

For example the nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence encoding HA from H3 A/Hongkong/4801/14 (SEQ ID NO: 102), wherein the nucleotide sequence encodes a modified H3 HA protein that has a Valine or a conserved substitution of Valine at position 384, no cysteine residue at positions 524 and 528 and wherein the sequence does not occur naturally. The conserved substitution may for example be Isoleucine, Leucine, Methionine, Alanine or Threonine.

The nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence of SEQ ID NO: 102, wherein the nucleotide sequence encodes a modified H3 HA protein that has a Valine or a conserved substitution of Valine at position 384, no cysteine residue at positions 524 and 528, wherein the sequence does not occur naturally and wherein the HA when expressed form VLP. The conserved substitution of Valine may for example be Isoleucine, Leucine, Methionine, Alanine or Threonine.

In addition to residues at position 384, 524 and 528, the modified H3 HA may have further residues modified. For example one or more than one residues at position 382, 525, 526 and 527 may be modified. In a non-limiting example the modified H3 HA may have substituted residues at position 384, 524, 528 and one or more substitutions at positions 382, 525, 526, 527 or a combination thereof.

Furthermore, it is provided a method of producing VLPs that comprise a modified H3 HA with a substitution at least at positions 384, 524 and 528 and optionally substitutions at positions 382, 525, 526, 527 above in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

In addition, it is provided a method of increasing yield of VLPs that comprise a modified H3 HA with a substitution at least at position 384, 524 and 528 and optionally substitutions at positions 382, 525, 526, 527, as described above in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

The present specification further provides for a VLP comprising a H3 HA with a substitution at least at position 384, 524 and 528 and optionally substitutions at positions 382, 525, 526, 527. The VLP may be produced by the method as provided by the present specification. The VLP comprising the modified H3 HA show improved characteristics when compared to VLPs that comprise the unmodified H3 HA protein.

### Modification at Position 524-528 (CysTM) and 392

In another aspect it is provided a H3 HA that may have modified residue at position 392, and may have no cysteine residue at positions 524 and 528 (H3 A/Hongkong/4801/14 numbering, SEQ ID NO: 92). Furthermore, residues at position 525, 526 and/or 527 (H3 A/Hongkong/4801/14 numbering) may also be modified.

As shown for example in FIGS. 7A and 7B, an H3 HA having cysteine residues at positions 524 and 528 modified to be no cysteine and having residue at position 392 modified from Phenylalanine to Aspartic Acid, lead to an 200% to 270% increase in hemagglutination titer as compared to wildtype H3 HA.

Accordingly, it is provided a modified H3 HA protein that comprise one or more than one modification at position 524, 525, 526, 527 or 528 and a modification at position 392. In a non-limiting example the modified H3 HA has modifications at least at positions 392, 524 and 528.

The residue at position 392 of H3 HA may be modified to be a non-Phenylalanine. For example the residue at position 392 may be modified to a charged amino acid for example Aspartic Acid or a conserved substitution of Aspartic Acid for example Glutamine, Asparagine, Glutamic Acid or Serine.

For example the modified H3 HA protein may have an amino acid sequence that has about 70, 75, 80, 85, 87, 90, 91, 92, 93 94, 95, 96, 97, 98, 99, 100% or any amount therebetween, sequence identity, or sequence similarity, with the amino acid sequence of HA from H3 A/Hongkong/4801/14 (SEQ ID NO: 92), wherein the amino acid sequence has a Aspartic Acid or a conserved substitution of Aspartic Acid at position 392, no cysteine residue at positions 524 and 528, wherein the sequence does not occur naturally and wherein the HA when expressed form VLP. The conserved substitution of Aspartic Acid may for example be Glutamine, Asparagine, Glutamic Acid or Serine. The non-cysteine at position 524 may be a Serine (S, Ser) or a conserved substitution of Serine and the non-cysteine at position 528 may be Leucine (L, Leu) or a conserved substitution of Leucine. The conserved substitution of Serine (Se, Ser) may for example be Threonine (T, Thr), Alanine (A, Ala), Asparagine (N, Asn), Aspartic Acid (D, Asp), Glutamine (Q, Gln), Glycine (G, Gly), Glutamic acid (E, Glu) or Lysine (L, Lys). The conserved substitution of Leucine may for example be Isoleucine (I, lie), Valine (V, Val), Methionine (M, Met), Phenylalanine (F, Phe), or Valine (V, Val).

The present specification also provides a nucleic acid comprising a nucleotide sequence encoding a modified H3 HA with a substitution at positions 392, 524 and 528 as described above operatively linked to a regulatory region active in a plant.

For example the nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence encoding HA from H3 A/Hongkong/4801/14 (SEQ ID NO: 102), wherein the nucleotide sequence encodes a modified H3 HA protein that has Aspartic Acid or a conserved substitution of Aspartic Acid at position 392, no cysteine residue at positions 524 and 528, wherein the sequence does not occur naturally and wherein the HA when expressed form VLP. The conserved substitution may for example be Glutamine, Asparagine, Glutamic Acid or Serine.

The nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence of SEQ ID NO: 102, wherein the nucleotide sequence encodes a modified H3 HA protein that has Aspartic Acid or a conserved substitution of Aspartic Acid at position 392, no cysteine residue at positions 524 and 528, wherein the sequence does not occur naturally and wherein the HA when expressed form VLP. The conserved substitution may for example be Glutamine, Asparagine, Glutamic Acid or Serine.

In addition to residues at positions 392, 524 and 528, the modified H3 HA may have further residues modified. For example one or more than one residues at position 525, 526 and 527 may be modified. In a non-limiting example the modified H3 HA may have substituted residues at position 392, 524, 528 and one or more substitutions at positions 525, 526, 527 or a combination thereof.

Furthermore, it is provided a method of producing VLPs that comprise a modified H3 HA with a substitution at least at positions 392, 524 and 528 and optionally substitutions at positions 525, 526, 527 above in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

In addition, it is provided a method of increasing yield of VLPs that comprise a modified H3 HA with a substitution at least at position 392, 524 and 528 and optionally substitutions at positions 525, 526, 527, as described above in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

The present specification further provides for a VLP comprising a H3 HA with a substitution at least at position 392, 524 and 528 and optionally substitutions at positions 525, 526, 527. The VLP may be produced by the method as provided by the present specification. The VLP comprising the modified H3 HA show improved characteristics when compared to VLPs that comprise the unmodified H3 HA protein.

### Modification at Position 524-528 (CysTM) and 431

In another aspect it is provided a H3 HA that may have a modified residue at position 431, and may have no cysteine residue at positions 524 and 528 (H3 A/Hongkong/4801/14 numbering, SEQ ID NO: 92). Furthermore, residues at position 525, 526 and/or 527 (H3 A/Hongkong/4801/14 numbering) may also be modified.

As shown for example in FIGS. 7A and 7B, an H3 HA having cysteine residues at positions 524 and 528 modified to be no cysteine and having residue at position 431 modified from Leucine to Methionine, lead to an approximately 250% increase in hemagglutination titer as compared to wildtype H3 HA.

Accordingly, it is provided a modified H3 HA protein that comprise one or more than one modification at position 524, 525, 526, 527 or 528 and a modification at position 431. In a non-limiting example the modified H3 HA has modifications at least at positions 431, 524 and 528.

The residue at position 431 of H3 HA may be modified to be a non-Leucine. For example the residue at position 431 may be modified be Methionine or a conserved substitution of Methionine for example Leucine, Isoleucine, Glutamine, Valine, or Phenylalanine.

For example the modified H3 HA protein may have an amino acid sequence that has about 70, 75, 80, 85, 87, 90, 91, 92, 93 94, 95, 96, 97, 98, 99, 100% or any amount therebetween, sequence identity, or sequence similarity, with the amino acid sequence of HA from H3 A/Hongkong/4801/14 (SEQ ID NO: 92), wherein the amino acid sequence has a Methionine or a conserved substitution of Methionine at position 431, no cysteine residue at positions 524 and 528, wherein the sequence does not occur naturally and wherein the HA when expressed form VLP. The conserved substitution of Methionine may for example be example Leucine, Isoleucine, Glutamine, Valine, or Phenylalanine. The non-cysteine at position 524 may be a Serine (S, Ser) or a conserved substitution of Serine and the non-cysteine at position 528 may be Leucine (L, Leu) or a conserved substitution of Leucine. The conserved substitution of Serine (Se, Ser) may for example be Threonine (T, Thr), Alanine (A, Ala), Asparagine (N, Asn), Aspartic Acid (D, Asp), Glutamine (Q, Gln), Glycine (G, Gly), Glutamic acid (E, Glu) or Lysine (L, Lys). The conserved substitution of Leucine may for example be Isoleucine (I, lie), Valine (V, Val), Methionine (M, Met), Phenylalanine (F, Phe), or Valine (V, Val).

The present specification also provides a nucleic acid comprising a nucleotide sequence encoding a modified H3 HA with a substitution at positions 431, 524 and 528 as described above operatively linked to a regulatory region active in a plant.

For example the nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence encoding HA from H3 A/Hongkong/4801/14 (SEQ ID NO: 102), wherein the nucleotide sequence encodes a modified H3 HA protein that has Methionine or a conserved substitution of Methionine at position 431, no cysteine residue at positions 524 and 528, wherein the sequence does not occur naturally and wherein the HA when expressed form VLP. The conserved substitution may for example be Leucine, Isoleucine, Glutamine, Valine, or Phenylalanine.

The nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence of SEQ ID NO: 102, wherein the nucleotide sequence encodes a modified H3 HA protein that has Methionine or a conserved substitution of Methionine at position 431, no cysteine residue at positions 524 and 528, wherein the sequence does not occur naturally and wherein the HA when expressed form VLP. The conserved substitution may for example be Leucine, Isoleucine, Glutamine, Valine, or Phenylalanine.

In addition to residues at positions 431, 524 and 528, the modified H3 HA may have further residues modified. For example one or more than one residues at position 525, 526 and 527 may be modified. In a non-limiting example the modified H3 HA may have substituted residues at position 431, 524, 528 and one or more substitutions at positions 525, 526, 527 or a combination thereof.

Furthermore, it is provided a method of producing VLPs that comprise a modified H3 HA with a substitution at least at positions 431, 524 and 528 and optionally substitutions at positions 525, 526, 527 above in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

In addition, it is provided a method of increasing yield of VLPs that comprise a modified H3 HA with a substitution at least at position 431, 524 and 528 and optionally substitutions at positions 525, 526, 527, as described above in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

The present specification further provides for a VLP comprising a H3 HA with a substitution at least at position 431, 524 and 528 and optionally substitutions at positions 525, 526, 527. The VLP may be produced by the method as provided by the present specification. The VLP comprising the modified H3 HA show improved characteristics when compared to VLPs that comprise the unmodified H3 HA protein.

### Modification at Position 524-528 (CysTM), 382 and 384

In another aspect it is provided a H3 HA that may have modified residues at position 382 and 384, and may have no cysteine residue at positions 524 and 528 (H3 A/Hongkong/4801/14 numbering, SEQ ID NO: 92). Furthermore, residues at position 525, 526 and/or 527 (H3 A/Hongkong/4801/14 numbering) may also be modified.

As shown for example in FIG. 7E, an H3 HA having cysteine residues at positions 524 and 528 modified to be non-cysteine and having residue at position 382 modified from Asparagine to Alanine and residue at position 384 modified from Leucine to Valine, lead to an approximately 400% to 500% increase in hemagglutination titer as compared to wildtype H3 HA.

Accordingly, it is provided a modified H3 HA protein that comprise one or more than one modification at position 524, 525, 526, 527 or 528 and a modification at position 382 and 384. In a non-limiting example the modified H3 HA has modifications at least at positions 382, 384, 524 and 528.

The residue at position 382 of H3 HA may be modified to be a non-Asparagine. For example the residue at position 382 may be modified to be a hydrophobic amino acid for example Alanine or a conserved substitution of Alanine for example Serine (S, Ser), Glycine (G, Gly), Threonine (T, Thr), Cysteine (C, Cys) or Valine (V, Val). The residue at position 384 of H3 HA may be modified to be a non-Leucine. For example the residue at position 384 may be modified to another hydrophobic amino acid for example Valine or a conserved substitution of Valine for example Isoleucine, Leucine, Methionine, Alanine or Threonine.

For example the modified H3 HA protein may have an amino acid sequence that has about 70, 75, 80, 85, 87, 90, 91, 92, 93 94, 95, 96, 97, 98, 99, 100% or any amount therebetween, sequence identity, or sequence similarity, with the amino acid sequence of HA from H3 A/Hongkong/4801/14 (SEQ ID NO: 92), wherein the amino acid sequence has an Alanine (A, Ala) or a conserved substitution of Alanine (A, Ala) at position 382, a Valine or a conserved substitution of Valine at position 384, no cysteine residue at positions 524 and 528 and wherein the sequence does not occur naturally and wherein the HA when expressed form VLP. The conserved substitution of Alanine may for example be Serine (S, Ser), Glycine (G, Gly), Threonine (T, Thr), Cysteine (C, Cys) or Valine (V, Val). The conserved substitution of Valine may for example be Isoleucine (I, lie), Leucine (L, Leu), Methionine (M, Met), Alanine A, Ala) or Threonine (T, Thr). The non-cysteine at position 524 may be a Serine (S, Ser) or a conserved substitution of Serine and the non-cysteine at position 528 may be Leucine (L, Leu) or a conserved substitution of Leucine. The conserved substitution of Serine (Se, Ser) may for example be Threonine (T, Thr), Alanine (A, Ala), Asparagine (N, Asn), Aspartic Acid (D, Asp), Glutamine (Q, Gln), Glycine (G, Gly), Glutamic acid (E, Glu) or Lysine (L, Lys). The conserved substitution of Leucine may for example be Isoleucine (I, lie), Valine (V, Val), Methionine (M, Met), Phenylalanine (F, Phe), or Valine (V, Val).

The present specification also provides a nucleic acid comprising a nucleotide sequence encoding a modified H3 HA with a substitution at positions 382, 384, 524 and 528 as described above operatively linked to a regulatory region active in a plant.

For example the nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence encoding HA from H3 A/Hongkong/4801/14 (SEQ ID NO: 102), wherein the nucleotide sequence encodes a modified H3 HA protein that has an Alanine (A, Ala) or a conserved substitution of Alanine (A, Ala) at position 382, a Valine or a conserved substitution of Valine at position 384, no cysteine residue at positions 524 and 528 and wherein the sequence does not occur naturally and wherein the HA when expressed form VLP. The conserved substitution of Alanine may for example be Serine (S, Ser), Glycine (G, Gly), Threonine (T, Thr), Cysteine (C, Cys) or Valine (V, Val). The conserved substitution of Valine may for example be Isoleucine (I, lie), Leucine (L, Leu), Methionine (M, Met), Alanine A, Ala) or Threonine (T, Thr). The non-cysteine at position 524 may be a Serine (S, Ser) or a conserved substitution of Serine and the non-cysteine at position 528 may be Leucine (L, Leu) or a conserved substitution of Leucine. The conserved substitution of Serine (Se, Ser) may for example be Threonine (T, Thr), Alanine (A, Ala), Asparagine (N, Asn), Aspartic Acid (D, Asp), Glutamine (Q, Gln), Glycine (G, Gly), Glutamic acid (E, Glu) or Lysine (L, Lys). The conserved substitution of Leucine may for example be Isoleucine (I, lie), Valine (V, Val), Methionine (M, Met), Phenylalanine (F, Phe), or Valine (V, Val).

The nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence of SEQ ID NO: 102, wherein the nucleotide sequence encodes a modified H3 HA protein that has an Alanine (A, Ala) or a conserved substitution of Alanine (A, Ala) at position 382, a Valine or a conserved substitution of Valine at position 384, no cysteine residue at positions 524 and 528 and wherein the sequence does not occur naturally and wherein the HA when expressed form VLP. The conserved substitution of Alanine may for example be Serine (S, Ser), Glycine (G, Gly), Threonine (T, Thr), Cysteine (C, Cys) or Valine (V, Val). The conserved substitution of Valine may for example be Isoleucine (I, lie), Leucine (L, Leu), Methionine (M, Met), Alanine A, Ala) or Threonine (T, Thr). The non-cysteine at position 524 may be a Serine (S, Ser) or a conserved substitution of Serine and the non-cysteine at position 528 may be Leucine (L, Leu) or a conserved substitution of Leucine. The conserved substitution of Serine (Se, Ser) may for example be Threonine (T, Thr), Alanine (A, Ala), Asparagine (N, Asn), Aspartic Acid (D, Asp), Glutamine (Q, Gln), Glycine (G, Gly), Glutamic acid (E, Glu) or Lysine (L, Lys). The conserved substitution of Leucine may for example be Isoleucine (I, lie), Valine (V, Val), Methionine (M, Met), Phenylalanine (F, Phe), or Valine (V, Val).

In addition to residues at position 382, 384, 524 and 528, the modified H3 HA may have further residues modified. For example one or more than one residues at positions 525, 526 and 527 may be modified. In a non-limiting example the modified H3 HA may have substituted residues at positions 382, 384, 524, 528 and one or more substitutions at positions 525, 526, 527 or a combination thereof.

Furthermore, it is provided a method of producing VLPs that comprise a modified H3 HA with a substitution at least at positions 382, 384, 524 and 528 and optionally substitutions at positions 525, 526, 527 above in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

In addition, it is provided a method of increasing yield of VLPs that comprise a modified H3 HA with a substitution at least at position 382, 384, 524 and 528 and optionally substitutions at positions 525, 526, 527, as described above in a plant. The method involves introducing a nucleic acid encoding a modified H3 HA operatively linked to a regulatory region active in the plant, into the plant, or portion of the plant, and incubating the plant or portion of the plant under conditions that permit the expression of the nucleic acid, thereby producing the VLPs.

The present specification further provides for a VLP comprising a H3 HA with a substitution at least at position 382, 384, 524 and 528 and optionally substitutions at positions 525, 526, 527. The VLP may be produced by the method as provided by the present specification. The VLP comprising the modified H3 HA show improved characteristics when compared to VLPs that comprise the unmodified H3 HA protein.

Also provided herein are methods of increasing production or yield of VLPs comprising mutant influenza HAs in plants. For example, a method may involve introducing a nucleic acid encoding a mutant influenza HA, as described herein, into the plant, portion of the plant, or plant cell. The nucleic acid encoding the mutant influenza HA may be optimized for human codon usage, increased GC content, or a combination thereof. One or more than one mutant influenza HA protein may be expressed in a plant, portion of the plant, or plant cell, in order to produce a VLP comprising one or more than one mutant influenza HA protein. Alternatively, the method may comprise providing a plant, portion of the plant, or plant cell that comprises the nucleic acid encoding the mutant influenza HA protein in order to produce a VLP comprising the one or more than one mutant influenza HA protein.

The methods of producing a VLP comprising a mutant influenza HA may further comprise a step of introducing a second nucleic acid sequence into the plant, portion of the plant, or plant cell, wherein the second nucleic acid encodes a proton channel protein that is co-expressed with the mutant influenza HA. For example, the proton channel protein may be an influenza A subtype M2 protein, such as A/New Caledonia/20/99 M2. The co-expression of the proton channel protein may lead to an increased accumulation of mutant influenza HA protein and/or VLP comprising the mutant influenza HA protein as for example described in WO 2013/044390 which is incorporated herein by reference.

Furthermore, the mutant influenza HA might further comprise a modified proteolytic loop or cleavage site as described in WO 2013/044390 and WO 2014/153674 and which are incorporated herein by reference.

By “co-expression”, it is meant the introduction and expression of two or more nucleotide sequences, each of the two or more nucleotide sequences encoding a protein of interest, or a fragment of a protein of interest within a plant, portion of a plant or a plant cell. The two or more nucleotide sequences may be introduced into the plant, portion of the plant or the plant cell within one vector, so that each of the two or more nucleotide sequences is under the control of a separate regulatory region (e.g. comprising a dual construct). Alternatively, the two or more nucleotide sequences may be introduced into the plant, portion of the plant or the plant cell within separate vectors (e.g. comprising single constructs), and each vector comprising appropriate regulatory regions for the expression of the corresponding nucleic acid. For example, two nucleotide sequences, each on a separate vector and introduced into separate Agrobacterium tumefaciens hosts, may be co-expressed by mixing suspensions of each A. tumefaciens host in a desired volume (for example, an equal volume, or the ratios of each A. tumefaciens host may be altered) before vacuum infiltration. In this manner, co-infiltration of multiple A. tumefaciens suspensions permits co-expression of multiple transgenes.

The nucleic acid encoding a mutant influenza HA as described herein may further comprise sequences that enhance expression of the mutant influenza HA in a plant, portion of the plant, or plant cell. Sequences that enhance expression may include, a cowpea mosaic virus (CPMV) enhancer element in operative association with the nucleic acid encoding the mutant influenza HA protein.

The nucleic acid comprising a nucleotide sequence encoding a modified influenza hemagglutinin (HA) protein, as described herein may further comprise sequences that enhance expression of the HA protein in the plant, portion of the plant, or plant cell. Sequences that enhance expression may include, a CPMV enhancer element, or a plant-derived expression enhancer, in operative association with the nucleic acid encoding the modified influenza hemagglutinin (HA) protein. The sequence encoding the modified influenza hemagglutinin (HA) may also be optimized for human codon usage, increased GC content, or a combination thereof.

The term “CPMV enhancer element”, as used herein, refers to a nucleotide sequence encoding the 5UTR regulating the Cowpea Mosaic Virus (CPMV) RNA2 polypeptide or a modified CPMV sequence as is known in the art. For example, a CPMV enhancer element or a CPMV expression enhancer, includes a nucleotide sequence as described in WO2015/14367; WO2015/103704; WO2007/135480; WO2009/087391; Sainsbury F., and Lomonossoff G. P., (2008, Plant Physiol. 148: pp. 1212-1218), each of which is incorporated herein by reference. A CPMV enhancer sequence can enhance expression of a downstream heterologous open reading frame (ORF) to which they are attached. The CPMV expression enhancer may include CPMV HT, CPMVX (where X=160, 155, 150, 114), for example CPMV 160, CPMVX+(where X=160, 155, 150, 114), for example CPMV 160+, CPMV-HT+, CPMV HT+[WT115], or CPMV HT+[511](WO2015/143567; WO2015/103704 which are incorporated herein by reference). The CPMV expression enhancer may be used within a plant expression system comprising a regulatory region that is operatively linked with the CPMV expression enhancer sequence and a nucleotide sequence of interest.

The term “CPMV enhancer element”, as used herein, refers to a nucleotide sequence encoding the 5′UTR regulating the Cowpea Mosaic Virus (CPMV) RNA2 polypeptide or a modified CPMV sequence as is known in the art. For example, a CPMV enhancer element or a CPMV expression enhancer, includes a nucleotide sequence as described in WO2015/14367; WO2015/103704; WO2007/135480; WO2009/087391; Sainsbury F., and Lomonossoff G. P., (2008, Plant Physiol. 148: pp. 1212-1218), each of which is incorporated herein by reference. A CPMV enhancer sequence can enhance expression of a downstream heterologous open reading frame (ORF) to which they are attached. The CPMV expression enhancer may include CPMV HT, CPMVX, CPMVX+, CPMV-HT+, CPMV HT+[WT115], or CPMV HT+[511](WO2015/14367; WO2015/103704 which are incorporated herein by reference). The CPMV expression enhancer may be used within a plant expression system comprising a regulatory region that is operatively linked with the CPMV expression enhancer sequence and a nucleotide sequence of interest.

The term “5′UTR” or “5′ untranslated region” or “5′ leader sequence” refers to regions of an mRNA that are not translated. The 5′UTR typically begins at the transcription start site and ends just before the translation initiation site or start codon of the coding region. The 5′ UTR may modulate the stability and/or translation of an mRNA transcript.

The term “plant-derived expression enhancer”, as used herein, refers to a nucleotide sequence obtained from a plant, the nucleotide sequence encoding a 5′UTR. Examples of a plant derived expression enhancer are described in U.S. Provisional Patent Application No. 62/643,053 (Filed Mar. 14, 2018; which is incorporated herein by reference) or in Diamos A. G. et al. (2016, Front Plt Sci. 7:1-15; which is incorporated herein by reference). The plant-derived expression enhancer may be selected from nbMT78, nbATL75, nbDJ46, nbCHP79, nbEN42, atHSP69, atGRP62, atPK65, atRP46, nb30S72, nbGT61, nbPV55, nbPP143, nbPM64, and nbH2A86 as described in U.S. 62/643,053). The plant derived expression enhancer may be used within a plant expression system comprising a regulatory region that is operatively linked with the plant-derived expression enhancer sequence and a nucleotide sequence of interest.

By “operatively linked” it is meant that the particular sequences interact either directly or indirectly to carry out an intended function, such as mediation or modulation of expression of a nucleic acid sequence. The interaction of operatively linked sequences may, for example, be mediated by proteins that interact with the operatively linked sequences.

When one or more than one mutant influenza HA protein is expressed in a plant, portion of the plant, or plant cell, the one or more than one mutant influenza HA proteins self-assemble into VLPs. The plant, portion of the plant, or plant cell, may be harvested under suitable extraction and purification conditions to maintain the integrity of the VLP, and the VLP comprising the one or more than one mutant influenza HA may be purified.

The present invention also provides the use of a mutant influenza HA, or VLP comprising the mutant influenza HA, as described herein, for inducing immunity to an influenza infection in a subject. Also disclosed herein is an antibody or antibody fragment, prepared by administering the mutant influenza HA or VLP comprising the mutant influenza HA, to a subject or a host animal. Further provided is a composition comprising an effective dose of a mutant influenza HA or VLP comprising the mutant influenza HA, as described herein, and a pharmaceutically acceptable carrier, adjuvant, vehicle, or excipient, for inducing an immune response in a subject. Also provided is a vaccine for inducing an immune response in a subject, wherein the vaccine comprises an effective dose of the mutant influenza HA.

Also provided herein are methods for inducing immunity to an influenza infection in a subject comprising of administering the mutant influenza HA or VLP comprising the mutant influenza HA, to a subject orally, intranasally, intramuscularly, intraperitoneally, intravenously, or subcutaneously.

The term “influenza virus”, as used herein, refers to an enveloped viral strain of the family Orthomyxoviridae that is characterized as having a negative sense single-stranded RNA genome. The influenza virus genome comprises eight gene segments coding for 12-14 proteins depending on the strain.

There are four types of influenza virus: A, B, C and D, of which influenza A or B are the causative organism for seasonal disease epidemics in humans. Influenza A is further classified based on the expression of HA and neuraminidase (NA) glycoprotein subtypes.

The term “hemagglutinin” or “HA”, as used herein, refers to a trimeric lectin that facilitates binding of the influenza virus particle to sialic acid-containing proteins on the surface of target cells and mediates release of the viral genome into the target cell. There are 18 different HA subtypes (H1-H18). HA proteins comprise two structural elements: the head, which is the primary target of seroprotective antibodies; and the stalk. HA is translated as a single polypeptide, HAO (assembled as trimers), that must be cleaved by a serine endoprotease between the HA1 (^{˜}40 kDa) and HA2 (^{˜}20 kDa) subdomains. After cleavage, the two disulfide-bonded protein domains adopt the requisite conformation necessary for viral infectivity.

Influenza A HA proteins or modified influenza A HA proteins as disclosed herein, include any known HA proteins derived from any known influenza A strain, but also modifications to known influenza A strains that develop over time. For example, influenza HA may be derived from A/Hong Kong/4801/14 (H3N2), A/Minnesota/40/15 (H3N2), A/South Australia/1/16 (H3N2), A/Bangkok/3007/15 (H3N2), A/Switzerland/9715293/13 (H3N2), A/Mississippi/16/16 (H3N2), or A/Pennsylvania/09/2015 (H3N2). Influenza A HA may include HA derived from strains, wherein the HA has about 30-100%, or any amount therebetween, amino acid sequence identity to any HA derived from the influenza A strains listed above, provided that the influenza HA protein comprises at least one substitution as described herewith and is able to form VLPs, induces an immune response when administered to a subject, induces hemagglutination or a combination thereof.

For example, influenza HA proteins may have 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100%, or any amount therebetween, amino acid sequence identity (sequence similarity, percent identity, percent similarity) to any HA derived from the influenza A strains listed above and comprises at least one substitution as described herewith and is able to form VLPs, induces an immune response when administered to a subject, induces hemagglutination or a combination thereof. An amino acid sequence alignment of several influenza A HA domains, which are not to be considered limiting, is shown in FIG. 1.

The terms “percent similarity”, “sequence similarity”, “percent identity”, or “sequence identity”, when referring to a particular sequence, are used for example as set forth in the University of Wisconsin GCG software program, or by manual alignment and visual inspection (see, e.g., Current Protocols in Molecular Biology, Ausubel et al., eds. 1995 supplement). Methods of alignment of sequences for comparison are well-known in the art. Optimal alignment of sequences for comparison can be conducted, using for example the algorithm of Smith & Waterman, (1981, Adv. Appl. Math. 2:482), by the alignment algorithm of Needleman & Wunsch, (1970, J. Mol. Biol. 48:443), by the search for similarity method of Pearson & Lipman, (1988, Proc. Natl. Acad. Sci. USA 85:2444), by computerized implementations of these algorithms (for example: GAP, BESTFIT, FASTA, and TFASTA in the Wisconsin Genetics Software Package, Genetics Computer Group (GCG), 575 Science Dr., Madison, Wis.).

An example of an algorithm suitable for determining percent sequence identity and sequence similarity are the BLAST and BLAST 2.0 algorithms, which are described in Altschul et al., (1977, Nuc. Acids Res. 25:3389-3402) and Altschul et al., (1990, J. Mol. Biol. 215:403-410), respectively. BLAST and BLAST 2.0 are used, with the parameters described herein, to determine percent sequence identity for the nucleic acids and proteins of the invention. For example the BLASTN program (for nucleotide sequences) may use as defaults a wordlength (W) of 11, an expectation (E) of 10, M=5, N=−4 and a comparison of both strands. For amino acid sequences, the BLASTP program may use as defaults a word length of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands. Software for performing BLAST analyses is publicly available through the National Center for Biotechnology Information (see URL: ncbi.nlm.nih.gov/).

The term “virus-like particle”, VLP, “virus like particles”, or “VLPs”, as used herein, refers to influenza particles that comprise one or more than one influenza HA protein, and that self-assemble into non-replicating, non-infectious viral capsid structures lacking all parts of the influenza genome.

### Influenza HA Protein Production in Plants

Influenza A HA protein includes any HA protein comprising an amino acid sequence having from about 30 to about 100%, from about 40 to about 100%, from about 50 to about 100%, from about 60 to about 100%, from about 70 to about 100%, from about 80 to about 100%, from about 85 to about 100%, from about 90 to about 100%, from 95 to about 100%, or from about 97 to about 100% from about 98 to about 100%, or any amount therebetween, sequence identity or sequence similarity with influenza A HA sequence from a A/Hong Kong/4801/14 (H3N2, SEQ ID NO: 92), A/Minnesota/40/15 (H3N2, SEQ ID NO: 93), A/South Australia/1/16 (H3N2, SEQ ID NO: 94), A/Bangkok/3007/15 (H3N2, SEQ ID NO: 91), A/Switzerland/9715293/13 (H3N2, SEQ ID NO: 96), A/Mississippi/16/16 (H3N2, SEQ ID NO: 97), and A/Pennsylvania/09/2015 (H3N2, SEQ ID NO: 95), provided that the influenza HA protein comprises at least one substitution as described herewith and is able to form VLPs, induces an immune response when administered to a subject, induces hemagglutination or a combination thereof.

Furthermore the modified influenza HA protein includes any HA protein comprising an amino acid sequence having from about 30% to about 100%, from about 40% to about 100%, from about 50% to about 100%, from about 60% to about 100%, from about 70% to about 100%, from about 80% to about 100%, from about 85% to about 100%, from about 90% to about 100%, from 95% to about 100%, or from about 97% to about 100% from about 98% to about 100%, or any amount therebetween, sequence identity or sequence similarity with a sequence of the sequences of SEQ ID NO: 25, SEQ ID NO: 30, SEQ ID NO: 35, SEQ ID NO: 39, SEQ ID NO: 43, SEQ ID NO: 47, SEQ ID NO: 49, SEQ ID NO: 53, SEQ ID NO: 57, SEQ ID NO: 59, SEQ ID NO: 61, SEQ ID NO: 82, SEQ ID NO: 84, SEQ ID NO: 86, SEQ ID NO: 88, SEQ ID NO: 90, provided that the influenza HA protein comprises at least one substitution as described herewith and is able to form VLPs, induces an immune response when administered to a subject, induces hemagglutination or a combination thereof.

As described herein, one or more than one specific mutation or modification in influenza HA results in increased accumulation of HA protein and increased VLP production in plants, as compared to wildtype influenza HA.

Examples of mutant influenza A HA proteins having enhanced influenza HA and/or VLP production in plants include, but are not limited to the following:

CysTM A/Hong Kong/4801/14 Mutant H3 (Construct #3341, SEQ ID NO: 21), N382A+L384V+CysTM A/Hong Kong/4801/14 Mutant H3 (Construct #3375, SEQ ID NO: 25); CysTM A/Minnesota/40/15 Mutant H3 (Construct #3914, SEQ ID NO:27); N382A+L384V+CysTM A/Minnesota/40/15 Mutant H3 (Construct #3915, SEQ ID NO:30); CysTM A/S. Australia/1/16 Mutant H3 (Construct #3924, SEQ ID NO: 33); N382A+L384V+CysTM A/S. Australia/1/16 Mutant H3 (Construct #3925, SEQ ID NO: 35); N382A+L384V+CysTM A/Bangkok/3007/15 Mutant H3 (Construct #3905, SEQ ID NO: 39); N382A A/Switzerland/9715293/13 Mutant H3 (Construct #3023, SEQ ID NO: 82); L384V A/Switzerland/9715293/13 Mutant H3 (Construct #3034, SEQ ID NO: 84); F392D A/Switzerland/9715293/13 Mutant H3 (Construct #3045, SEQ ID NO: 43; L431M A/Switzerland/9715293/13 Mutant H3 (Construct #3022, SEQ ID NO: 47); CysTM A/Switzerland/9715293/13 Mutant H3 (Construct #2811, SEQ ID NO: 49); N382A+CysTM A/Switzerland/9715293/13 Mutant H3 (Construct #3063, SEQ ID NO:53); L384V+CysTM A/Switzerland/9715293/13 Mutant H3 (Construct #3074, SEQ ID NO:57); F392D+CysTM A/Switzerland/9715293/13 Mutant H3 (Construct #3085, SEQ ID NO: 59); and L431M+CysTM A/Switzerland/9715293/13 Mutant H3 (Construct #3062, SEQ ID NO: 61), CysTM A/Pennsylvania/09/2015 mutant H3 (Construct #3313, SEQ ID NO: 86), N382A+CysTM A/Pennsylvania/09/2015 mutant H3 (Construct #3314, SEQ ID NO: 88), and L384V+CysTM A/Pennsylvania/09/2015 mutant H3 (Construct #3315, SEQ ID NO: 90).

### Induction of Immunity Against Influenza Infection

An “immune response” generally refers to a response of the adaptive immune system of a subject. The adaptive immune system generally comprises a humoral response, and a cell-mediated response. The humoral response is the aspect of immunity that is mediated by secreted antibodies, produced in the cells of the B lymphocyte lineage (B cell). Secreted antibodies bind to antigens on the surfaces of invading microbes (such as viruses or bacteria), which flags them for destruction. Humoral immunity is used generally to refer to antibody production and the processes that accompany it, as well as the effector functions of antibodies, including Th2 cell activation and cytokine production, memory cell generation, opsonin promotion of phagocytosis, pathogen elimination and the like. The terms “modulate” or “modulation” or the like refer to an increase or decrease in a particular response or parameter, as determined by any of several assays generally known or used, some of which are exemplified herein.

A cell-mediated response is an immune response that does not involve antibodies but rather involves the activation of macrophages, natural killer cells (NK), antigen-specific cytotoxic T-lymphocytes, and the release of various cytokines in response to an antigen. Cell-mediated immunity is used generally to refer to some Th cell activation, Tc cell activation and T-cell mediated responses. Cell mediated immunity may be of particular importance in responding to viral infections.

For example, the induction of antigen specific CD8 positive T lymphocytes may be measured using an ELISPOT assay; stimulation of CD4 positive T-lymphocytes may be measured using a proliferation assay. Anti-influenza HA antibody titres may be quantified using an ELISA assay; isotypes of antigen-specific or cross reactive antibodies may also be measured using anti-isotype antibodies (e.g. anti-IgG, IgA, IgE or IgM). Methods and techniques for performing such assays are well-known in the art.

Cytokine presence or levels may also be quantified. For example a T-helper cell response (Th1/Th2) will be characterized by the measurement of IFN-γ and IL-4 secreting cells using by ELISA (e.g. BD Biosciences OptEIA kits). Peripheral blood mononuclear cells (PBMC) or splenocytes obtained from a subject may be cultured, and the supernatant analyzed. T lymphocytes may also be quantified by fluorescence-activated cell sorting (FACS), using marker specific fluorescent labels and methods as are known in the art.

A microneutralization assay may also be conducted to characterize an immune response in a subject, see for example the methods of Rowe et al., 1973. Virus neutralization titers may be quantified in a number of ways, including: enumeration of lysis plaques (plaque assay) following crystal violent fixation/coloration of cells; microscopic observation of cell lysis in in vitro culture; and 2) ELISA and spectrophotometric detection of influenza virus.

The term “epitope” or “epitopes”, as used herein, refers to a structural part of an antigen to which an antibody specifically binds.

Immune responses elicited in response to administration of plant-produced wildtype influenza HA proteins or VLPs, or mutant influenza HA proteins or VLPs may for example be observed in Balb/C mice. Serum samples from blood collected from animals may be analyzed by ELISA for H3-specific total IgG and IgA antibodies. Mice immunized with either plant-produced wildtype influenza HA or mutant influenza HA proteins may exhibit HA-specific IgG antibody titers in sera for each treatment group.

### Plant Expression

The constructs of the present invention may be introduced into plant cells using Ti plasmids, Ri plasmids, plant virus vectors, direct DNA transformation, micro-injection, electroporation, etc. For reviews of such techniques see for example Weissbach and Weissbach, Methods for Plant Molecular Biology, Academy Press, New York Vill, pp. 421-463 (1988); Geierson and Corey, Plant Molecular Biology, 2d Ed. (1988); and Miki and lyer, Fundamentals of Gene Transfer in Plants. In Plant Metabolism, 2d Ed. DT. Dennis, DH Turpin, DD Lefebrvre, DB Layzell (eds), Addison Wesly, Langmans Ltd. London, pp. 561-579 (1997). Other methods include direct DNA uptake, the use of liposomes, electroporation, for example using protoplasts, micro-injection, microprojectiles or whiskers, and vacuum infiltration. See, for example, Bilang, et al. (1991, Gene 100: 247-250), Scheid et al. (1991, Mol. Gen. Genet. 228: 104-112), Guerche et al. (1987, Plant Science 52: 111-116), Neuhause et al. (1987, Theor. Appl Genet. 75: 30-36), Klein et al. (2987, Nature 327: 70-73); Freeman et al. (1984, Plant Cell Physiol. 29: 1353), Howell et al. (1985, Science 227: 1229-1231), DeBlock et al. (1989, Plant Physiology 91: 694-701), Methods for Plant Molecular Biology (Weissbach and Weissbach, eds., Academic Press Inc., 1988), Methods in Plant Molecular Biology (Schuler and Zielinski, eds., Academic Press Inc., 1989), WO 92/09696, WO 94/00583, EP 331083, EP 175966, Liu and Lomonossoff (2002, J Virol Meth, 105:343-348), EP 290395; WO 8706614; U.S. Pat. Nos. 4,945,050; 5,036,006; and 5,100,792, U.S. patent application Ser. No. 08/438,666, filed May 10, 1995, and Ser. No. 07/951,715, filed Sep. 25, 1992, (all of which are hereby incorporated by reference).

Transient expression methods may be used to express the constructs of the present invention (see D'Aoust et al., 2009, Methods in molecular biology, Vol 483, pages 41-50; Liu and Lomonossoff, 2002, Journal of Virological Methods, 105:343-348; which is incorporated herein by reference). Alternatively, a vacuum-based transient expression method, as described by Kapila et al. (1997, Plant Sci. 122, 101-108; which is incorporated herein by reference), or WO 00/063400, WO 00/037663 (which are incorporated herein by reference) may be used. These methods may include, for example, but are not limited to, a method of Agro-inoculation or Agro-infiltration, syringe infiltration, however, other transient methods may also be used as noted above. With Agro-inoculation, Agro-infiltration, or syringe infiltration, a mixture of Agrobacteria comprising the desired nucleic acid enter the intercellular spaces of a tissue, for example the leaves, aerial portion of the plant (including stem, leaves and flower), other portion of the plant (stem, root, flower), or the whole plant. After crossing the epidermis the Agrobacteria infect and transfer t-DNA copies into the cells. The t-DNA is episomally transcribed and the mRNA translated, leading to the production of the protein of interest in infected cells, however, the passage of t-DNA inside the nucleus is transient.

Also considered part of this invention are transgenic plants, plant cells or seeds containing the gene construct of the present invention that may be used as a platform plant suitable for transient protein expression described herein. Methods of regenerating whole plants from plant cells are also known in the art (for example see Guerineau and Mullineaux (1993, Plant transformation and expression vectors. In: Plant Molecular Biology Labfax (Croy RRD ed) Oxford, BIOS Scientific Publishers, pp 121-148). In general, transformed plant cells are cultured in an appropriate medium, which may contain selective agents such as antibiotics, where selectable markers are used to facilitate identification of transformed plant cells. Once callus forms, shoot formation can be encouraged by employing the appropriate plant hormones in accordance with known methods and the shoots transferred to rooting medium for regeneration of plants. The plants may then be used to establish repetitive generations, either from seeds or using vegetative propagation techniques. Transgenic plants can also be generated without using tissue culture. Methods for stable transformation, and regeneration of these organisms are established in the art and known to one of skill in the art. Available techniques are reviewed in Vasil et al. (Cell Culture and Somatic Cell Genetics of Plants, Vol I, II and Ill, Laboratory Procedures and Their Applications, Academic Press, 1984), and Weissbach and Weissbach (Methods for Plant Molecular Biology, Academic Press, 1989). The method of obtaining transformed and regenerated plants is not critical to the present invention.

If plants, plant portions or plant cells are to be transformed or co-transformed by two or more nucleic acid constructs, the nucleic acid construct may be introduced into the Agrobacterium in a single transfection event so that the nucleic acids are pooled, and the bacterial cells transfected. Alternatively, the constructs may be introduced serially. In this case, a first construct is introduced into the Agrobacterium as described, the cells are grown under selective conditions (e.g. in the presence of an antibiotic) where only the singly transformed bacteria can grow. Following this first selection step, a second nucleic acid construct is introduced into the Agrobacterium as described, and the cells are grown under doubly-selective conditions, where only the doubly-transformed bacteria can grow. The doubly-transformed bacteria may then be used to transform a plant, portion of the plant or plant cell as described herein, or may be subjected to a further transformation step to accommodate a third nucleic acid construct.

Alternatively, if plants, plant portions, or plant cells are to be transformed or co-transformed by two or more nucleic acid constructs, the nucleic acid construct may be introduced into the plant by co-infiltrating a mixture of Agrobacterium cells with the plant, plant portion, or plant cell, each Agrobacterium cell may comprise one or more constructs to be introduced within the plant. In order to vary the relative expression levels within the plant, plant portion or plant cell, of a nucleotide sequence of interest within a construct, during the step of infiltration, the concentration of the various Agrobacteria populations comprising the desired constructs may be varied.

TABLE 3
SEQ ID NOs and Description of Sequences

| SEQ ID NO: | Description of Sequence |
|---|---|
| SEQ ID NO: 1 | PDI-H3 HK DNA |
| SEQ ID NO: 2 | PDI-H3 HK AA |
| SEQ ID NO: 3 | PDI-H3 Minn DNA |
| SEQ ID NO: 4 | PDI-H3 Minn AA |
| SEQ ID NO: 5 | PDI-H3 SAus DNA |
| SEQ ID NO: 6 | PDI-H3 SAus AA |
| SEQ ID NO: 7 | PDI-H3 Bang DNA |
| SEQ ID NO: 8 | PDI-H3 Bang AA |
| SEQ ID NO: 9 | PDI-H3 Switz DNA |
| SEQ ID NO: 10 | PDI-H3 Switz AA |
| SEQ ID NO: 11 | PDI-H3 Miss DNA |
| SEQ ID NO: 12 | PDI-H3 Miss AA |
| SEQ ID NO: 13 | PDI-H3 Penn DNA |
| SEQ ID NO: 14 | PDI-H3 Penn AA |
| SEQ ID NO: 15 | IF-CPMV(fl5′UTR)_SpPDI.c |
| SEQ ID NO: 16 | IF-H1cTMCT.S1-4r |
| SEQ ID NO: 17 | IF-H3A-Ala.r |
| SEQ ID NO: 18 | H3_Swi_SLVLL.r |
| SEQ ID NO: 19 | H3_Swi_SLVLL.c |
| SEQ ID NO: 20 | PDI-H3 HK-CysTm DNA |
| SEQ ID NO: 21 | PDI-H3 HK-CysTm AA |
| SEQ ID NO: 22 | H3_HK480114(N382A + L384V).r |
| SEQ ID NO: 23 | H3_HK480114(N382A + L384V).c |
| SEQ ID NO: 24 | PDI-H3 HK-N382A + L384V-CysTm DNA |
| SEQ ID NO: 25 | PDI-H3 HK-N382A + L384V-CysTm AA |
| SEQ ID NO: 26 | PDI-H3 Minn-CysTm DNA |
| SEQ ID NO: 27 | PDI-H3 Minn-CysTm AA |
| SEQ ID NO: 28 | H3Minn(N382A + L384V).r |
| SEQ ID NO: 29 | H3Minn(N382A + L384V).c |
| SEQ ID NO: 30 | PDI-H3 Minn-N382A + L384V-CysTm DNA |
| SEQ ID NO: 31 | PDI-H3 Minn-N382A + L384V-CysTm AA |
| SEQ ID NO: 32 | PDI-H3 SAus-CysTm DNA |
| SEQ ID NO: 33 | PDI-H3 SAus-CysTm AA |
| SEQ ID NO: 34 | PDI-H3 SAus-N382A + L384V-CysTm DNA |
| SEQ ID NO: 35 | PDI-H3 SAus-N382A + L384V-CysTm AA |
| SEQ ID NO: 36 | PDI-H3 Bang-CysTm DNA |
| SEQ ID NO: 37 | PDI-H3 Bang-CysTm AA |
| SEQ ID NO: 38 | PDI-H3 Bang-N382A + L384V-CysTm DNA |
| SEQ ID NO: 39 | PDI-H3 Bang-N382A + L384V-CysTm AA |
| SEQ ID NO: 40 | H3_Swi(F392D).r |
| SEQ ID NO: 41 | H3_Swi(F392D).c |
| SEQ ID NO: 42 | PDI-H3 Switz-F392D DNA |
| SEQ ID NO: 43 | PDI-H3 Switz-F392D AA |
| SEQ ID NO: 44 | H3_Swi(L431M).r |
| SEQ ID NO: 45 | H3_Swi(L431M).c |
| SEQ ID NO: 46 | PDI-H3 Switz-L431M DNA |
| SEQ ID NO: 47 | PDI-H3 Switz-L431M AA |
| SEQ ID NO: 48 | PDI-H3 Switz-CysTm DNA |
| SEQ ID NO: 49 | PDI-H3 Switz-CysTm AA |
| SEQ ID NO: 50 | H3_Swi(N382A).r |
| SEQ ID NO: 51 | H3_Swi(N382A).c |
| SEQ ID NO: 52 | PDI-H3 Switz-N382A-CysTm DNA |
| SEQ ID NO: 53 | PDI-H3 Switz-N382A-CysTm AA |
| SEQ ID NO: 54 | H3_Swi(L384V).r |
| SEQ ID NO: 55 | H3_Swi(L384V).c |
| SEQ ID NO: 56 | PDI-H3 Switz-L384V-CysTm DNA |
| SEQ ID NO: 57 | PDI-H3 Switz-L384V-CysTm AA |
| SEQ ID NO: 58 | PDI-H3 Switz-F392D-CysTm DNA |
| SEQ ID NO: 59 | PDI-H3 Switz-F392D-CysTm AA |
| SEQ ID NO: 60 | PDI-H3 Switz-L431M-CysTm DNA |
| SEQ ID NO: 61 | PDI-H3 Switz-L431M-CysTm AA |
| SEQ ID NO: 62 | Cloning vector 1190 from left to right T-DNA |
| SEQ ID NO: 63 | Construct 1314 from 2X35S prom to NOS term |
| SEQ ID NO: 64 | Construct 2980 from 2X35S prom to NOS term |
| SEQ ID NO: 65 | Construct 2995 from 2X35S prom to NOS term |
| SEQ ID NO: 66 | Cloning vector 3556 from left to right T-DNA |
| SEQ ID NO: 67 | IF-H5ITMCT.s1-4r |
| SEQ ID NO: 68 | PDI-H5 Indo DNA |
| SEQ ID NO: 69 | PDI-H5 Indo AA |
| SEQ ID NO: 70 | H5Ind(F393D).r |
| SEQ ID NO: 71 | H5Ind(F393D).c |
| SEQ ID NO: 72 | PDI-H5 Indo-F393D DNA |
| SEQ ID NO: 73 | PDI-H5 Indo-F393D AA |
| SEQ ID NO: 74 | IF-H5_Egy.r |
| SEQ ID NO: 75 | PDI-H5 Egypt DNA |
| SEQ ID NO: 76 | PDI-H5 Egypt AA |
| SEQ ID NO: 77 | H5Egy(F392D).r |
| SEQ ID NO: 78 | H5Egy(F392D).c |
| SEQ ID NO: 79 | PDI-H5 Egypt-F392D DNA |
| SEQ ID NO: 80 | PDI-H5 Egypt-F392D AA |
| SEQ ID NO: 81 | PDI-H3 Switz-N382A DNA |
| SEQ ID NO: 82 | PDI-H3 Switz-N382A AA |
| SEQ ID NO: 83 | PDI-H3 Switz-L384V DNA |
| SEQ ID NO: 84 | PDI-H3 Switz-L384V AA |
| SEQ ID NO: 85 | PDI-H3 Penn-CysTm DNA |
| SEQ ID NO: 86 | PDI-H3 Penn-CysTm AA |
| SEQ ID NO: 87 | PDI-H3 Penn-N382A-CysTm DNA |
| SEQ ID NO: 88 | PDI-H3 Penn-N382A-CysTm AA |
| SEQ ID NO: 89 | PDI-H3 Penn-L384V-CysTm DNA |
| SEQ ID NO: 90 | PDI-H3 Penn-L384V-CysTm AA |
| SEQ ID NO: 91 | A/Bangkok/3007/15 (H3N2) (aa) |
| SEQ ID NO: 92 | A/Hongkong/4801/14 (H3N2) (aa) |
| SEQ ID NO: 93 | A/Minnesota/40/15 (H3N2) (aa) |
| SEQ ID NO: 94 | A/South Australia/1/16 (H3N2) (aa) |
| SEQ ID NO: 95 | A/Pennsylvania/09/15 (H3N2) (aa) |
| SEQ ID NO: 96 | A/Switzerland/9715293/13 (H3N2) (aa) |
| SEQ ID NO: 97 | A/Mississippi/16/16 (H3N2) (aa) |
| SEQ ID NO: 98 | CysTM mutation (aa) |
| SEQ ID NO: 99 | wt CysTM (aa) |
| SEQ ID NO: 100 | CysTM mutation (nt) |
| SEQ ID NO: 101 | wt CysTM (nt) |
| SEQ ID NO: 102 | A/Hong Kong/4801/14 (nt) |
| SEQ ID NO: 103 | Construct No. 2801 (nt) |
| SEQ ID NO: 104 | Construct No. 3023 (nt) |
| SEQ ID NO: 105 | Construct No. 2811 (nt) |

The present invention will be further illustrated in the following examples.

### EXAMPLES

### Example 1: Influenza HA Constructs

The influenza HA constructs were produced using techniques well known within the art. For example wildtype H3 A-Switzerland/9715293/13 HA, N382A A/Switzerland/9715293/13 H3 HA and CysTM A-Switzerland-9715293-13 were cloned as described below. Other H3 HA mutants were obtained using similar techniques and the HA sequences primers, templates and products are described in Example 3 (Influenza HA and VLP Production in Plants) and Table 4.

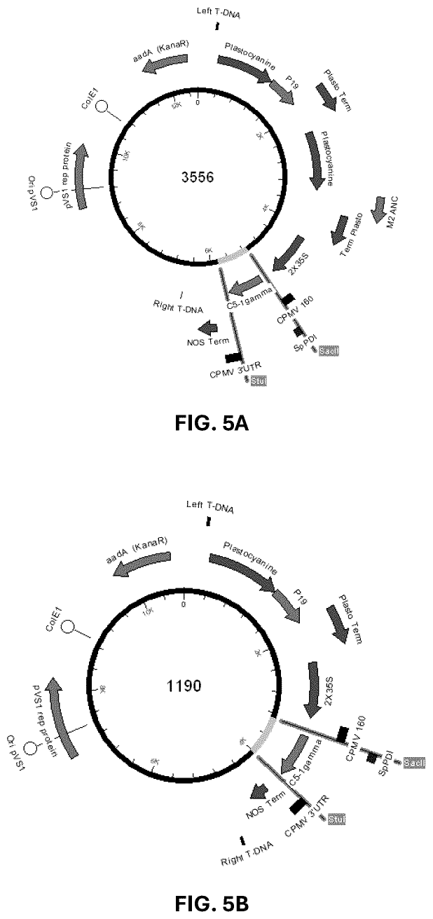

A summary of the wildtype and mutated HA proteins, primers, templates and products is provided in Table 4 below. For Influenza H3 constructs other than H3 A/Switzerland/9715293/13 HA proteins which were cloned into the 1190 cloning vector without M2, the cloning vector used integrates an influenza M2 ion channel gene under the control of Alfalfa Plastocyanin promoter and terminator in addition to the 2X35S promoter/CPMV 160+CPMV 3′UTR/NOS-based expression cassette. Plasmid number 3556 (SEQ ID NO: 66; FIG. 5A) was digested with SacII and StuI restriction enzymes and used for the In-Fusion reaction.

### Modification of H3 HA

### 2X35S/CPMV 160/PDISP-HAO H3 A-Switzerland-9715293-13/NOS (Construct Number 2801)

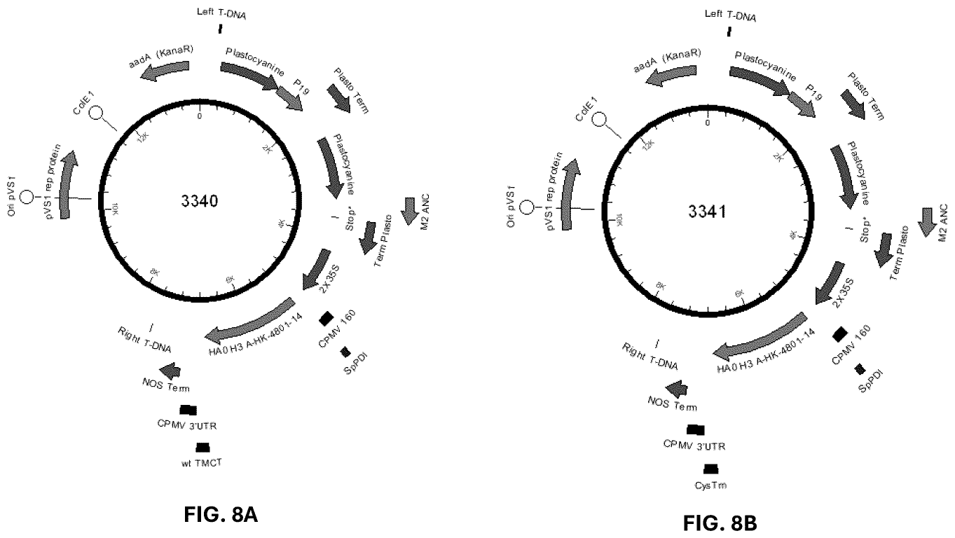

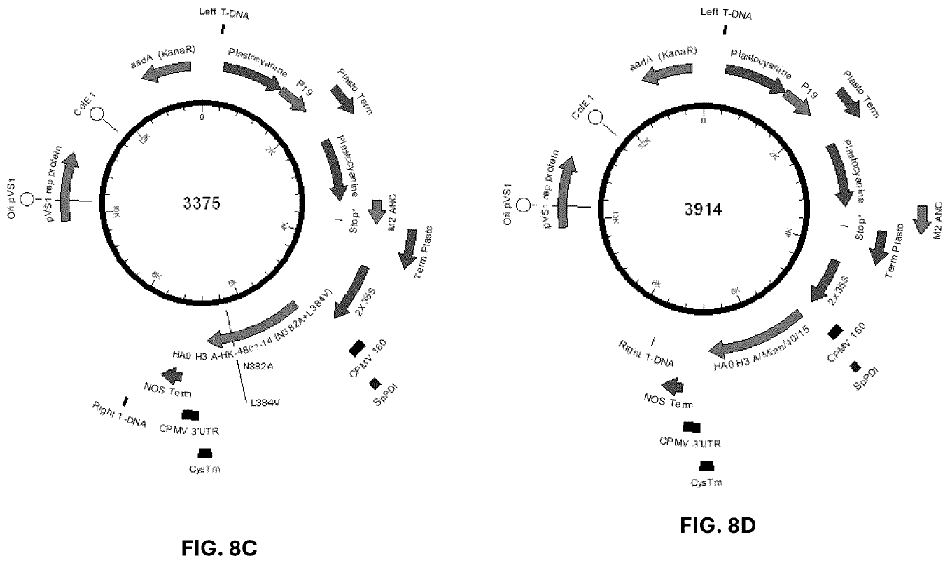

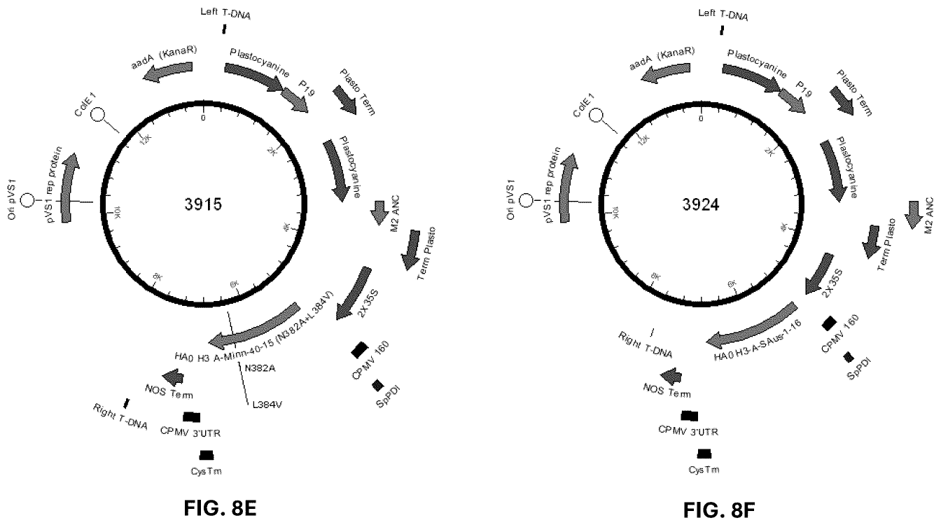

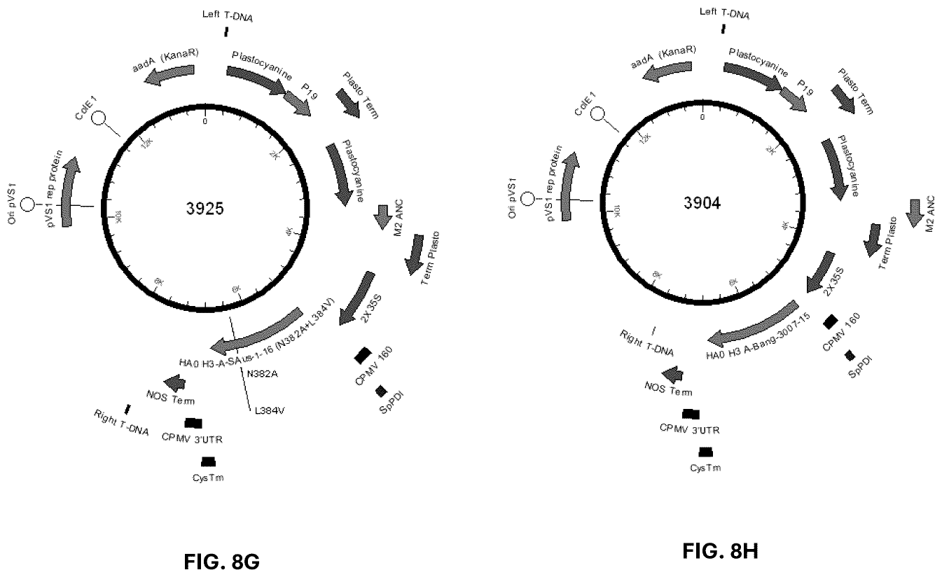

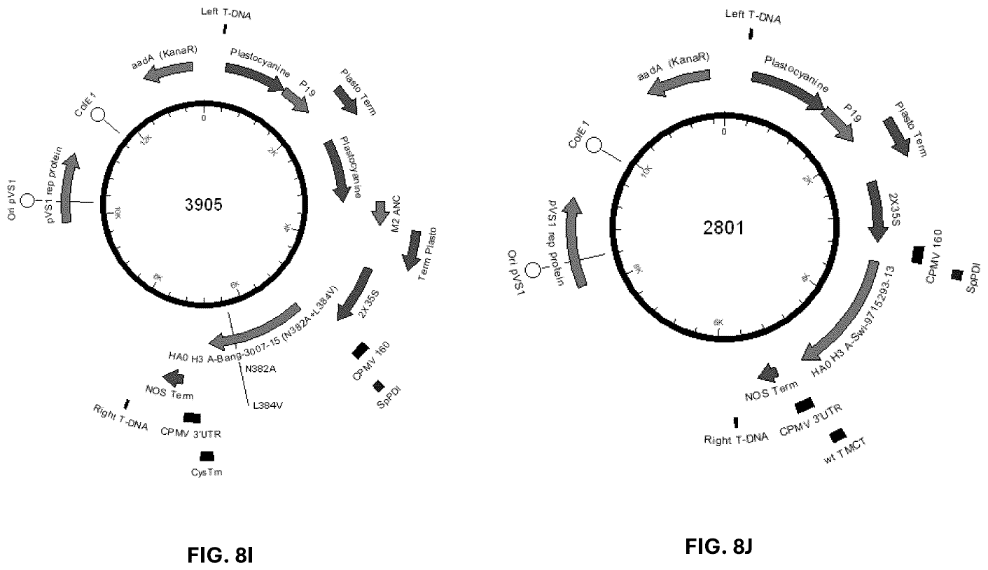

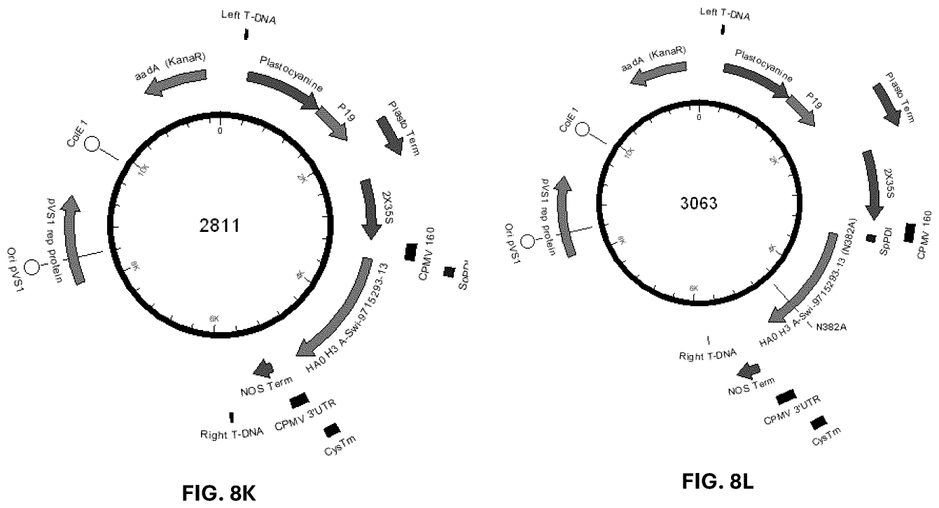

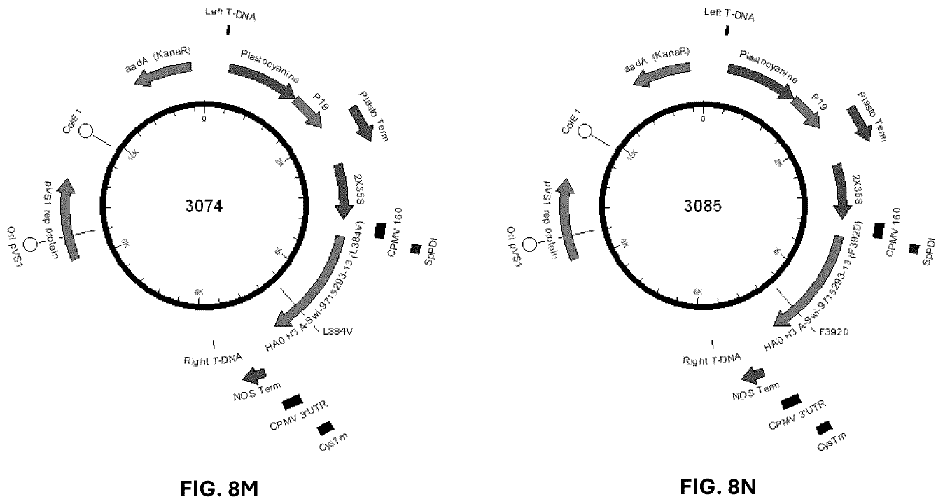

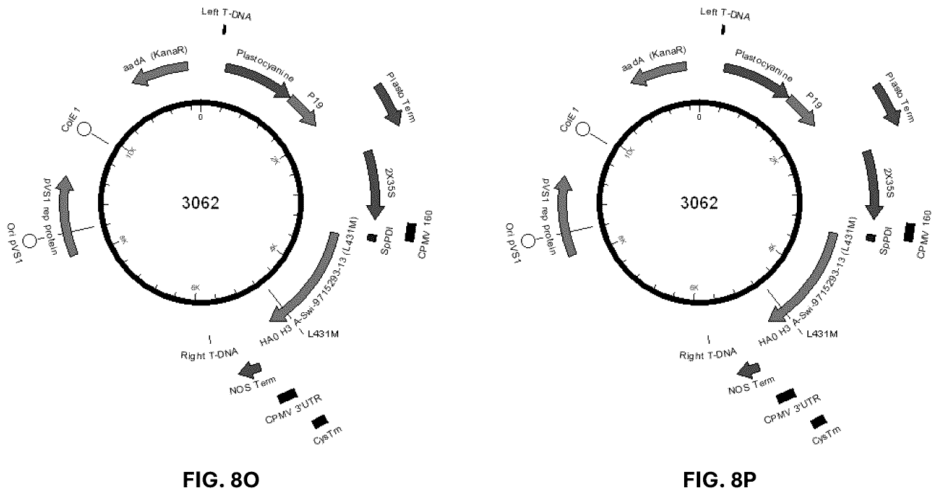

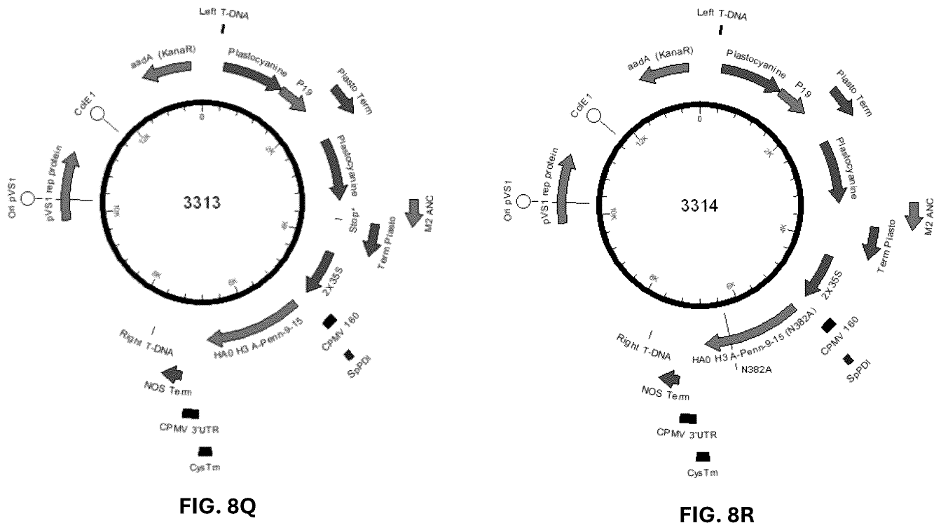

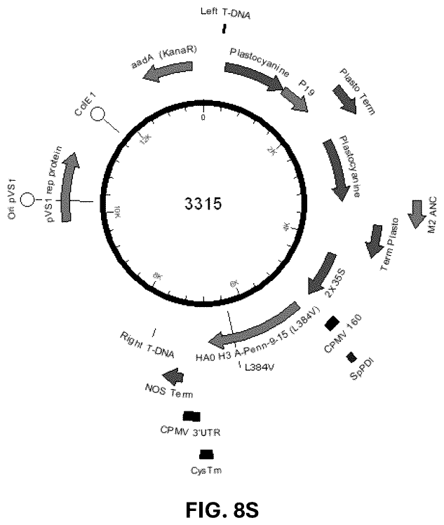

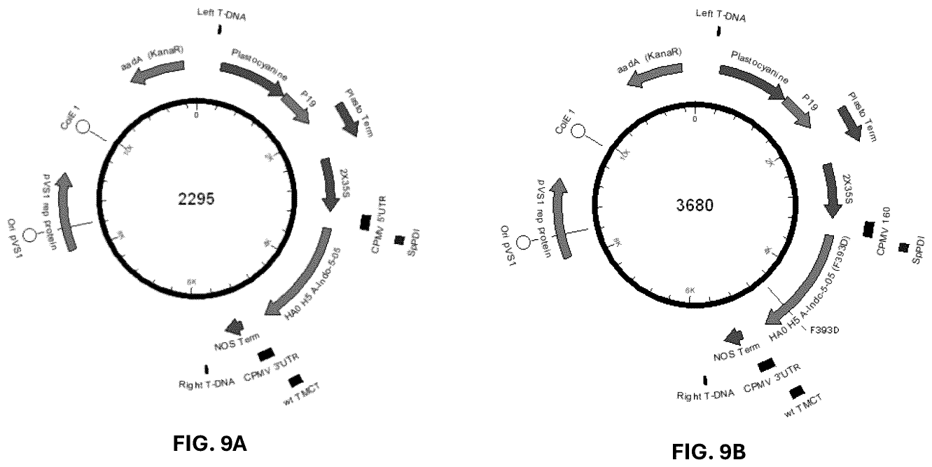

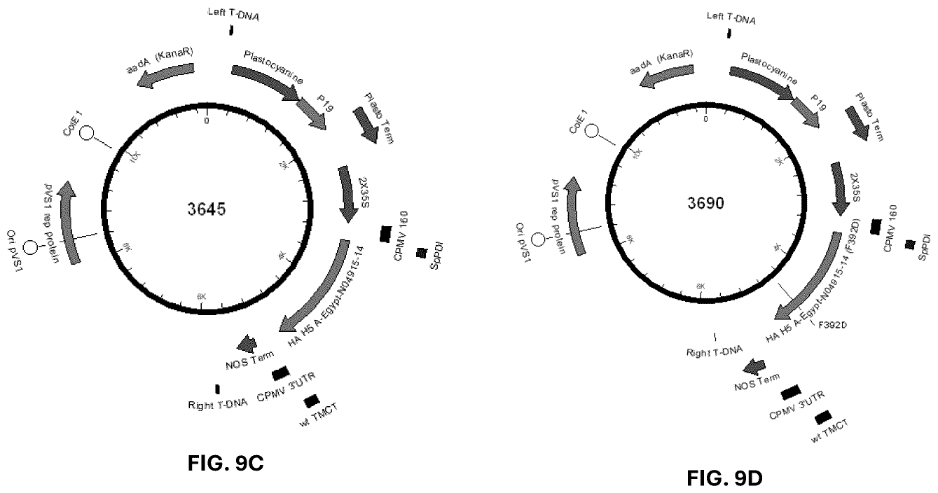

A sequence encoding mature HAO from influenza HA from H3 A/Switzerland/9715293/13 fused to alfalfa PDI secretion signal peptide (PDISP) was cloned into 2X35S/CPMV 160/NOS expression system using the following PCR-based method. A fragment containing the PDISP-H3 A/Switzerland/9715293/13 coding sequence was amplified using primers IF-CPMV(fl5′UTR)_SpPDI.c (SEQ ID NO: 15) and IF-H3A-Ala.r (SEQ ID NO: 17), using PDISP-H1 A/Switzerland/9715293/13 gene sequence (SEQ ID NO: 9) as template. The PCR product was cloned in 2X35S/CPMV 160/NOS expression system using In-Fusion cloning system (Clontech, Mountain View, CA). Construct number 1190 (FIG. 5B) was digested with SacII and StuI restriction enzyme and the linearized plasmid was used for the In-Fusion assembly reaction. Construct number 1190 is an acceptor plasmid intended for “In Fusion” cloning of genes of interest in a 2X35S/CPMV 160/NOS-based expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in SEQ ID NO: 62. The resulting construct was given number 2801 (SEQ ID NO: 103). The amino acid sequence of mature HAO from influenza HA from A/Switzerland/9715293/13 fused to alfalfa PDI secretion signal peptide (PDISP) is presented in SEQ ID NO: 10. A representation of plasmid 2801 is presented in FIG. 8J.

### 2X35S/CPMV 160/PDISP-HAO H3 A-Switzerland-9715293-13 (N382A)/NOS (Construct Number 3023)

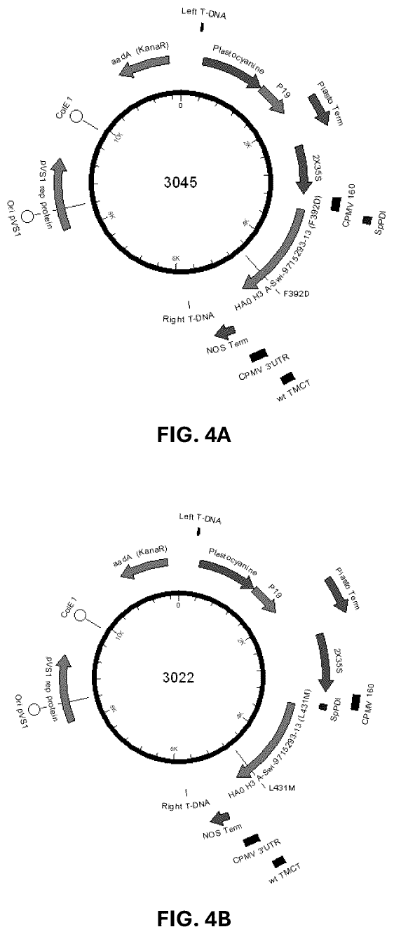

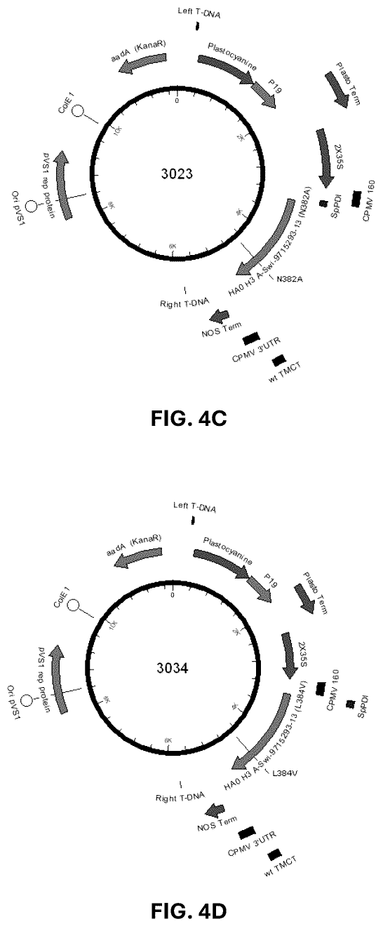

A sequence encoding mature HAO from influenza HA from A/Switzerland/9715293/13 (N382A) fused to alfalfa PDI secretion signal peptide (PDISP) was cloned into 2X35S/CPMV 160/NOS expression system using the following PCR-based method. In a first round of PCR, a fragment containing the PDISP-H3 A/Switzerland/9715293/13 with the mutated N382A amino acid was amplified using primers IF-CPMV(fl5′UTR)_SpPDI.c (SEQ ID NO: 15) and H3_Swi(N382A).r (SEQ ID NO: 50), using PDISP-H3 A/Switzerland/9715293/13 gene sequence (SEQ ID NO: 9) as template. A second fragment containing the N382A mutation with the remaining of the H3 A/Switzerland/9715293/13 was amplified using H3_Swi(N382A).c (SEQ ID NO: 51) and IF-H3A-Ala.r (SEQ ID NO: 17), using PDISP-H3 A/Switzerland/9715293/13 gene sequence (SEQ ID NO: 9) as template. The PCR products from both amplifications were then mixed and used as template for a second round of amplification using IF-CPMV(fl5′UTR)_SpPDI.c (SEQ ID NO: 15) and IF-H3A-Ala.r (SEQ ID NO: 17) as primers. The final PCR product was cloned in 2X35S/CPMV 160/NOS expression system using In-Fusion cloning system (Clontech, Mountain View, CA). Construct number 1190 (FIG. 5B) was digested with SacII and StuI restriction enzyme and the linearized plasmid was used for the In-Fusion assembly reaction. Construct number 1190 is an acceptor plasmid intended for “In Fusion” cloning of genes of interest in a 2X35S/CPMV 160/NOS-based expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in SEQ ID NO: 62. The resulting construct was given number 3023 (SEQ ID NO: 104). The amino acid sequence of mutated PDISP-H3 A-Switzerland-9715293-13 (N382A) is presented in SEQ ID NO: 82. A representation of plasmid 3023 is presented in FIG. 4C.

### 2X35S/CPMV 160/PDISP-HAO H3 A-Switzerland-9715293-13 (CysTM)/NOS (Construct Number 2811)

A sequence encoding mature HAO from influenza HA from A/Switzerland/9715293/13 (CysTM) fused to alfalfa PDI secretion signal peptide (PDISP) was cloned into 2X35S/CPMV 160/NOS expression system using the following PCR-based method. In a first round of PCR, a fragment containing the PDISP-H3 A/Switzerland/9715293/13 with the mutated CysTM amino acids was amplified using primers IF-CPMV(fl5′UTR)_SpPDI.c (SEQ ID NO: 15) and H3_Swi_SLVLL.r (SEQ ID NO: 18), using PDISP-H3 A/Switzerland/9715293/13 gene sequence (SEQ ID NO: 9) as template. A second fragment containing the CysTm mutations with the remaining of the H3 A/Switzerland/9715293/13 was amplified using H3_Swi_SLVLL.c (SEQ ID NO: 19) and IF-H3A-Ala.r (SEQ ID NO: 17), using PDISP-H3 A/Switzerland/9715293/13 gene sequence (SEQ ID NO: 9) as template. The PCR products from both amplifications were then mixed and used as template for a second round of amplification using IF-CPMV(fl5′UTR)_SpPDI.c (SEQ ID NO: 15) and IF-H3A-Ala.r (SEQ ID NO: 17) as primers. The final PCR product was cloned in 2X35S/CPMV 160/NOS expression system using In-Fusion cloning system (Clontech, Mountain View, CA). Construct number 1190 (FIG. 5B) was digested with SacII and StuI restriction enzyme and the linearized plasmid was used for the In-Fusion assembly reaction. Construct number 1190 is an acceptor plasmid intended for “In Fusion” cloning of genes of interest in a 2X35S/CPMV 160/NOS-based expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in SEQ ID NO: 62. The resulting construct was given number 2811 (SEQ ID NO: 105). The amino acid sequence of mutated PDISP-H3 A-Switzerland-9715293-13 (CysTM) is presented in SEQ ID NO: 49. A representation of plasmid 2811 is presented in FIG. 8K.

### Example 2: Methods

Agrobacterium tumefaciens Transfection

Agrobacterium tumefaciens strain AGL1 was transfected by electroporation with the wildtype influenza HA or mutant influenza HA expression vectors using the methods described by D'Aoust et al., 2008 (Plant Biotech. J. 6:930-40). Transfected Agrobacterium were grown in YEB medium supplemented with 10 mM 2-(N-morpholino)ethanesulfonic acid (MES), 20 μM acetosyringone, 50 μg/ml kanamycin and 25 ag/ml of carbenicillin pH5.6 to an OD600 between 0.6 and 1.6. Agrobacterium suspensions were centrifuged before use and resuspended in infiltration medium (10 mM MgCl2 and 10 mM MES pH 5.6).

### Preparation of Plant Biomass, Inoculum and Agroinfiltration

N. benthamiana plants were grown from seeds in flats filled with a commercial peat moss substrate. The plants were allowed to grow in the greenhouse under a 16/8 photoperiod and a temperature regime of 25° C. day/20° C. night. Three weeks after seeding, individual plantlets were picked out, transplanted in pots and left to grow in the greenhouse for three additional weeks under the same environmental conditions.

Agrobacteria transfected with each wildtype influenza HA or mutant influenza HA expression vector were grown in a YEB medium supplemented with 10 mM 2-(N-morpholino)ethanesulfonic acid (MES), 20 μM acetosyringone, 50 μg/ml kanamycin and 25 ag/ml of carbenicillin pH 5.6 until they reached an OD₆₀₀ between 0.6 and 1.6. Agrobacterium suspensions were centrifuged before use and resuspended in infiltration medium (10 mM MgCl₂ and 10 mM MES pH 5.6) and stored overnight at 4° C. On the day of infiltration, culture batches were diluted in 2.5 culture volumes and allowed to warm before use. Whole plants of N. benthamiana were placed upside down in the bacterial suspension in an air-tight stainless steel tank under a vacuum of 20-40 Torr for 2-min. Plants were returned to the greenhouse for a 6 or 9 day incubation period until harvest.

### Leaf Harvest and Total Protein Extraction

Proteins were extracted from fresh biomass cut into ^{˜}1 cm2 pieces by an overnight enzymatic extraction at room temperature using an orbital shaker. The slurry was then filtered through a large pore nylon filter to remove coarse undigested vegetal tissue.

To obtain the “Full Process yields”, the slurry was centrifuged to remove protoplasts and intracellular contaminants. The supernatant was clarified by depth-filtration. The clarified fraction was then loaded over a cation exchange column with a step-elution step with increasing concentrations of NaCl. The purified VLPs were concentrated by TFF, diafiltered against a formulation buffer and passed through a filter. Protein content of purified VLP was analysed by BCA assay and activity was analysed by a hemagglutination assay. Relative yields were obtained by comparing the protein yields from the new construct to the native construct (or to CysTM for H3 strains) used as control.

To obtain the “Post-Density Gradient Yields”, the slurry was centrifuged to remove protoplasts and intracellular contaminants. The supernatant was centrifuged further to remove additional debris. The supernatant was the clarified by depth-filtration using glass fiber filter. The clarified fraction was then loaded on a discontinuous iodixanol density gradient. Separation density gradient centrifugation was performed as follows: 38 ml tubes containing discontinuous iodixanol density gradient in Tris buffer (successive layers of 35%, 30%, 25%, 20%, 15, 10% and 5%) were prepared and overlaid with clarified extract. The gradients were centrifuged at 120 000 g for 2 hours (4° C.). After centrifugation, the first 5 mL collected from the bottom to the top were discarded while the next 5 mL were collected for protein content analysis (BCA), activity measurement (hemagglutination assay) and intensity measurement of the HAO band on a reduced SDS-PAGE (densitometry). Relative yields were obtained by comparing the HAO band intensity from the new construct to the native construct (or CysTM for H3 strains) used as control.

### Hemagglutination Assay

Hemagglutination assay was based on a method described by Nayak and Reichl (2004). Briefly, serial double dilutions of the test samples (100 μL) were made in V-bottomed 96-well microtiter plates containing 100 μL PBS, leaving 100 μL of diluted sample per well. One hundred microliters of 0.5% guinea pig red blood cells suspension (Bio Link Inc., Syracuse, NY) were added to each well, and plates were incubated for 2 h at room temperature. The reciprocal of the highest dilution showing complete hemagglutination was recorded as HA activity. In parallel, a recombinant HA standard (A/Vietnam/1203/2004 H5N1) (Protein Science Corporation, Meriden, CT) was diluted in PBS and run as a control on each plate.

### Protein Analysis and Immunoblotting

Immunoblotting was performed with a first incubation with a primary mAb, diluted 1/500 in 2% skim milk in TBS-Tween 20 0.1%. Peroxydase-conjugated goat anti-mouse (Jackson Immunoresearch, cat #115-035-146) diluted 1/10000 was used as secondary antibody for chemiluminescence detection in 2% skim milk in TBS-Tween 20 0.1% Immunoreactive complexes were detected by chemiluminescence using luminol as the substrate (Roche Diagnostics Corporation). Horseradish peroxidase-enzyme conjugation of human IgG antibody was carried out by using the EZ-Link Plus® Activated Peroxidase conjugation kit (Pierce, Rockford, Ill.).

### Example 3: Influenza HA and VLP Production in Plants

### Modification of H3 HA I

The influenza HA constructs were produced using techniques well known within the art (see Example 1). A summary of the wildtype and mutated HA proteins, primers, templates and products is provided in Table 4 below. The sequences used are provided in Example 4 and in the sequence listing.

### N382A A/Switzerland/9715293/13 Mutant H3

N382A A/Switzerland/9715293/13 Mutant H3 (Construct #3023) was constructed by mutating the asparagine at position 382 of wildtype/Switzerland/9715293/13 H3 to alanine. As shown in FIG. 2, purified extracts from N. benthamiana plants agroinfiltrated with Construct #3023 exhibited an approximate 30% increase in hemagglutination titer as compared to extracts from N. benthamiana plants agroinfiltrated with wildtype A/Switzerland/9715293/13 H3 (Construct #2801).

### L384V A/Switzerland/9715293/13 Mutant H3

L384V A/Switzerland/9715293/13 Mutant H3 (Construct #3034) was constructed by mutating the leucine at position 384 of wildtype/Switzerland/9715293/13 H3 to valine. As shown in FIG. 2, purified extracts from N. benthamiana plants agroinfiltrated with Construct #3034 exhibited an approximate 100% increase in hemagglutination titer as compared to extracts from N. benthamiana plants agroinfiltrated with wildtype A/Switzerland/9715293/13 H3 (Construct #2801).

### F392D A/Switzerland/9715293/13 Mutant H3

F392D A/Switzerland/9715293/13 Mutant H3 was constructed by mutating the phenylalanine at position 392 of wildtype A/Switzerland/9715293/13 H3 to aspartic acid (Construct #3045). As shown in FIG. 2, purified extracts from N. benthamiana plants agroinfiltrated with Construct #3045 exhibited an approximate 40% increase in hemagglutination titer as compared to extracts from N. benthamiana plants agroinfiltrated with wildtype A/Switzerland/9715293/13 H3 (Construct #2801).

### L431M A/Switzerland/9715293/13 Mutant H3

L431M A/Switzerland/9715293/13 Mutant H3 was constructed by mutating the leucine at position 431 of wildtype A/Switzerland/9715293/13 H3 to methionine (Construct #3022). As shown in FIG. 2, purified extracts from N. benthamiana plants agroinfiltrated with Construct #3022 exhibited an approximate 70% increase in hemagglutination titer as compared to extracts from N. benthamiana plants agroinfiltrated with wildtype A/Switzerland/9715293/13 H3 (Construct #2811).

The one or more than one mutations described herein specifically increase influenza HA protein production and VLP yield in plants. It was observed that mutations at other positions significantly reduced, or had no significant effect, on influenza HA protein accumulation or VLP production in plant cells.

The increased hemagglutination titers achieved with the influenza HA proteins comprising the one or more than one mutation described herein was also observed to be specific to influenza H3 HAs. Similar enhancements were not observed in plants agroinfiltrated with constructs encoding mutant influenza HAs derived from non-H3 strains.

For example, F393D A/Indonesia/5/2005 Mutant H5 was constructed by mutating the phenylalanine at position 393 of wildtype A/Indonesia/5/2005 H5 to aspartic acid (Construct #3680). As shown in FIG. 3, purified extracts from N. benthamiana plants agroinfiltrated with Construct #3680 exhibited an approximate 98% reduction in hemagglutination titer as compared to extracts from N. benthamiana plants agroinfiltrated with wildtype A/Indonesia/5/2005 H5 (Construct #2295).

Similarly, purified extracts from N. benthamiana plants agroinfiltrated with F392D A/Egypt/N04915/2014 Mutant H5 (Construct #3690), exhibited an approximate 99% reduction in hemagglutination titer as compared to extracts from N. benthamiana plants agroinfiltrated with wildtype A/Egypt/N04915/2014 H5 (Construct #3645) (see FIG. 3, Table 7).

### Modification of H3 HA II

### CysTM A/Switzerland/9715293/13 Mutant H3

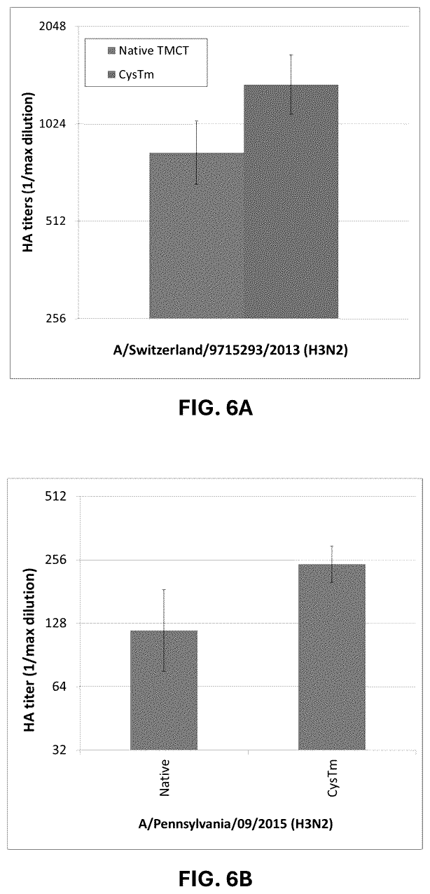

CysTM A/Switzerland/9715293/13 Mutant H3 (Construct #2811) was constructed by replacing the “CFLLC” sequence at positions 524-528 (SEQ ID NO: 99) of wildtype A/Switzerland/9715293/13 H3 to “SLVLL” (SEQ ID NO: 98). As shown in FIG. 6A, purified extracts from N. benthamiana plants agroinfiltrated with Construct #2811 exhibited an approximate 60% increase in hemagglutination titer as compared to extracts from N. benthamiana plants agroinfiltrated with wildtype A/Switzerland/9715293/13 H3 (Construct #2801).

### CysTM A/Pennsylvania/09/2015 Mutant H3

CysTM A/Pennsylvania/09/2015 Mutant H3 (Construct #3313) was constructed by replacing the “CFLLC” sequence at positions 524-528 (SEQ ID NO: 99) of wildtype A/Pennsylvania/09/2015 H3 to “SLVLL” (SEQ ID NO: 98). As shown in FIG. 7C, purified extracts from N. benthamiana plants agroinfiltrated with Construct #3313 exhibited an approximate 100% increase in hemagglutination titer as compared to extracts from N. benthamiana plants agroinfiltrated with wildtype A/Pennsylvania/09/2015 H3 (Construct #3312).

### N382A+CysTM A/Switzerland/9715293/13 Mutant H3

N382A+CysTM A/Switzerland/9715293/13 Mutant H3 (Construct #3063) was constructed by mutating the asparagine at position 382 of wildtype A/Switzerland/9715293/13 H3 to alanine and replacing the “CFLLC” sequence at positions 524-528 (SEQ ID NO: 99) to “SLVLL” (SEQ ID NO: 98). As shown in FIG. 7A, purified extracts from N. benthamiana plants agroinfiltrated with Construct #3063 exhibited an approximate 160% increase in hemagglutination titer as compared to extracts from N. benthamiana plants agroinfiltrated with wildtype A/Switzerland/9715293/13 H3 (Construct #2801). As further shown in FIG. 7B, N. benthamiana plants agroinfiltrated with Construct #3063 exhibited an approximate 30% increase in VLP yield following lodixanol gradient purification, in comparison to plants infiltrated with CysTM A/Switzerland/9715293/13 Mutant H3 (Construct #2811).

### N382A+CysTM A/Pennsylvania/09/2015 Mutant H3

N382A+CysTM A/Pennsylvania/09/2015 Mutant H3 (Construct #3314) was constructed by mutating the asparagine at position 382 of wildtype A/Pennsylvania/09/2015 H3 to alanine and replacing the “CFLLC” sequence at positions 524-528 (SEQ ID NO: 99) to “SLVLL” (SEQ ID NO: 98). As shown in FIG. 7C, purified extracts from N. benthamiana plants agroinfiltrated with Construct #3314 exhibited an approximate 300% increase in hemagglutination titer as compared to extracts from N. benthamiana plants agroinfiltrated with wildtype A/Pennsylvania/09/2015 H3 (Construct #3312).

### L384V+CysTM A/Switzerland/9715293/13 Mutant H3

L384V+CysTM A/Switzerland/9715293/13 Mutant H3 (Construct #3074) was constructed by mutating the leucine at position 384 of wildtype A/Switzerland/9715293/13 H3 to valine and replacing the “CFLLC” sequence at positions 524-528 (SEQ ID NO: 99) to “SLVLL” (SEQ ID NO: 98). As shown in FIG. 7C, purified extracts from N. benthamiana plants agroinfiltrated with Construct #3074 exhibited an approximate 380% increase in hemagglutination titer as compared to extracts from N. benthamiana plants agroinfiltrated with wildtype A/Switzerland/9715293/13 H3 (Construct #2801). Furthermore, as shown in FIG. 7B, N. benthamiana plants agroinfiltrated with Construct #3074 exhibited an approximate 50% increase in VLP yield following sucrose gradient purification, in comparison to plants infiltrated with CysTM A/Switzerland/9715293/13 Mutant H3 (Construct #2811).

### L384V+CysTM A/Pennsylvania/09/2015 Mutant H3

L384V+CysTM A/Pennsylvania/09/2015 Mutant H3 (Construct #3315) was constructed by mutating the leucine at position 384 of wildtype A/Pennsylvania/09/2015 H3 to valine and replacing the “CFLLC” sequence at positions 524-528 (SEQ ID NO: 99) to “SLVLL” (SEQ ID NO: 98). As shown in FIG. 7C, purified extracts from N. benthamiana plants agroinfiltrated with Construct #3315 exhibited an approximate 380% increase in hemagglutination titer as compared to extracts from N. benthamiana plants agroinfiltrated with wildtype A/Pennsylvania/09/2015 H3 (Construct #3312).

### F392D+CysTM A/Switzerland/9715293/13 Mutant H3

F392D+CysTM A/Switzerland/9715293/13 Mutant H3 (Construct #3085) was constructed by mutating the phenylalanine at position 392 of wildtype/Switzerland/9715293/13 H3 to aspartic acid and replacing the “CFLLC” sequence at positions 524-528 (SEQ ID NO: 99) to “SLVLL” (SEQ ID NO: 98). As shown in FIG. 7A, purified extracts from N. benthamiana plants agroinfiltrated with Construct #3085 exhibited an approximate 170% increase in hemagglutination titer as compared to extracts from N. benthamiana plants agroinfiltrated with wildtype A/Switzerland/9715293/13 H3 (Construct #2801).

### L431M+CysTM A/Switzerland/9715293/13 Mutant H3

L431M+CysTM A/Switzerland/9715293/13 Mutant H3 (Construct #3062) was constructed by mutating the leucine at position 431 of wildtype/Switzerland/9715293/13 H3 to methionine and replacing the “CFLLC” sequence at positions 524-528 (SEQ ID NO: 99) to “SLVLL” (SEQ ID NO: 98). As shown in FIG. 7A, purified extracts from N. benthamiana plants agroinfiltrated with Construct #3062 exhibited an approximate 150% increase in hemagglutination titer as compared to extracts from N. benthamiana plants agroinfiltrated with wildtype A/Switzerland/9715293/13 H3 (Construct #2801).

### N382A+L384V+CysTM A/Hong Kong/4801/14 Mutant H3

N382A+L384V+CysTM A/Hong Kong/4801/14 Mutant H3 (Construct #3375) was constructed by mutating the asparagine at position 382 of wildtype A/Hong Kong/4801/14 to an alanine residue, the leucine at position 384 to a valine residue, and the “CFLLC” sequence at positions 524-528 (SEQ ID NO: 99) to “SLVLL” (SEQ ID NO: 98). As shown in FIG. 7E, purified extracts from N. benthamiana plants agroinfiltrated with Construct #3375 exhibited an approximate 300% increase in hemagglutination titer as compared to extracts from N. benthamiana plants agroinfiltrated with CysTM A/Hong Kong/4801/14 Mutant H3 (Construct #3341).

### N382A+L384V+CysTM A/Minnesota/40/15 Mutant H3

N382A+L384V+CysTM A/Minnesota/40/15 Mutant H3 (Construct #3915) was constructed by mutating the asparagine at position 382 of wildtype A/Minnesota/40/15 to an alanine residue, the leucine at position 384 to a valine residue, and the “CFLLC” sequence at positions 524-528 (SEQ ID NO: 99) to “SLVLL” (SEQ ID NO: 98). As shown in FIG. 7E, purified extracts from N. benthamiana plants agroinfiltrated with Construct #3915 exhibited an approximate 400% increase in hemagglutination titer as compared to extracts from N. benthamiana plants agroinfiltrated with CysTM A/Minnesota/40/15 Mutant H3 (Construct #3914).

### N382A+L384V+CysTM A/S. Australia/1/16 Mutant H3

N382A+L384V+CysTM A/S. Australia/1/16 Mutant H3 (Construct #3925) was constructed by mutating the asparagine at position 382 of wildtype A/S. Australia/1/16 to an alanine residue, the leucine at position 384 to a valine residue, and the “CFLLC” sequence at positions 524-528 (SEQ ID NO: 99) to “SLVLL” (SEQ ID NO: 98). As shown in FIGS. 7D and 7E, purified extracts from N. benthamiana plants agroinfiltrated with Construct #3925 exhibited an approximate 400% increase in hemagglutination titer as compared to extracts from N. benthamiana plants agroinfiltrated with CysTM A/S. Australia/1/16 Mutant H3 (Construct #3924).

### N382A+L384V+CysTM A/Bangkok/3007/15 Mutant H3

N382A+L384V+CysTM A/Bangkok/3007/15 Mutant H3 (Construct #3905) was constructed by mutating the asparagine at position 382 of wildtype A/Bangkok/3007/15 to an alanine residue, the leucine at position 384 to a valine residue, and the “CFLLC” sequence at positions 524-528 (SEQ ID NO: 99) to “SLVLL” (SEQ ID NO: 98). As shown in FIG. 7E, purified extracts from N. benthamiana plants agroinfiltrated with Construct #3905 exhibited an approximate 400% increase in hemagglutination titer as compared to extracts from N. benthamiana plants agroinfiltrated with CysTM A/Bangkok/3007/15 Mutant H3 (Construct #3904).

The one or more than one mutations described herein specifically increase influenza HA protein production and VLP yield in plants. It was observed that mutations at other positions significantly reduced, or had no significant effect, on influenza HA protein accumulation or VLP production in plant cells.

The increased hemagglutination titers achieved with the influenza HA proteins comprising the one or more than one mutation described herein was also observed to be specific to influenza H3 HAs. Similar enhancements were not observed in plants agroinfiltrated with constructs encoding mutant influenza HAs derived from non-H3 strains.

For example, F393D A/Indonesia/5/2005 Mutant H5 was constructed by mutating the phenylalanine at position 393 of wildtype A/Indonesia/5/2005 H5 to aspartic acid (Construct #3680). As shown in FIG. 3, purified extracts from N. benthamiana plants agroinfiltrated with Construct #3680 exhibited an approximate 98% reduction in hemagglutination titer as compared to extracts from N. benthamiana plants agroinfiltrated with wildtype A/Indonesia/5/2005 H5 (Construct #2295).

Similarly, purified extracts from N. benthamiana plants agroinfiltrated with F392D A/Egypt/N04915/2014 Mutant H5 (Construct #3690), exhibited an approximate 99% reduction in hemagglutination titer as compared to extracts from N. benthamiana plants agroinfiltrated with wildtype A/Egypt/N04915/2014 H5 (Construct #3690) (see FIG. 3).

TABLE 4
Examples of constructs that have been prepared as described herein.
Sequences are provided in Example 4 and the sequence listing.

| Construct Name | TMCT | Construct # | FIG. | Primer 1 | Primer 2 | Primer 3 |
|---|---|---|---|---|---|---|
| H3 A-HK-4801-14 | — | 3340 | 8A | SEQ ID NO: 15 | SEQ ID NO: 17 | — |
| H3 A-HK-4801-14 | CysTm | 3341 | 8B | SEQ ID NO: 15 | SEQ ID NO: 18 | SEQ ID NO: 19 |
| H3 A-HK-4801-14 (N382A + L384V) | CysTm | 3375 | 8C | SEQ ID NO: 15 | SEQ ID NO: 22 | SEQ ID NO: 23 |
| H3 A-Minn-40-15 | CysTM | 3914 | 8D | SEQ ID NO: 15 | SEQ ID NO: 18 | SEQ ID NO: 19 |
| H3 A-Minn-40-15 (N382A + L384V) | CysTM | 3915 | 8E | SEQ ID NO: 15 | SEQ ID NO: 28 | SEQ ID NO: 29 |
| H3 A-SAus-1-16 | CysTM | 3924 | 8F | SEQ ID NO: 15 | SEQ ID NO: 18 | SEQ ID NO: 19 |
| H3 A-SAus-1-16 (N382A + L384V) | CysTM | 3925 | 8G | SEQ ID NO: 15 | SEQ ID NO: 28 | SEQ ID NO: 29 |
| H3 A-Bang-3007-15 | CysTM | 3904 | 8H | SEQ ID NO: 15 | SEQ ID NO: 18 | SEQ ID NO: 19 |
| H3 A-Bang-3007-15 (N382A + L384V) | CysTM | 3905 | 8I | SEQ ID NO: 15 | SEQ ID NO: 22 | SEQ ID NO: 23 |
| H3 A-Swi-9715293-13 | Wt | 2801 | 8J | SEQ ID NO: 15 | SEQ ID NO: 17 | — |
| H3 A-Swi-9715293-13 (F392D) | Wt | 3045 | 4A | SEQ ID NO: 15 | SEQ ID NO: 40 | SEQ ID NO: 41 |
| H3 A-Swi-9715293-13 (L431M) | Wt | 3022 | 4B | SEQ ID NO: 15 | SEQ ID NO: 44 | SEQ ID NO: 45 |
| H3 A-Swi-9715293-13 | CysTM | 2811 | 8K | SEQ ID NO: 15 | SEQ ID NO: 18 | SEQ ID NO: 19 |
| H3 A-Swi-9715293-13 (N382A) | CysTM | 3063 | 8L | SEQ ID NO: 15 | SEQ ID NO: 50 | SEQ ID NO: 51 |
| H3 A-Swi-9715293-13 (L384V) | CysTM | 3074 | 8M | SEQ ID NO: 15 | SEQ ID NO: 54 | SEQ ID NO: 55 |
| H3 A-Swi-9715293-13 (F392D) | CysTM | 3085 | 8N | SEQ ID NO: 15 | SEQ ID NO: 40 | SEQ ID NO: 41 |
| H3 A-Swi-9715293-13 (L431M) | CysTM | 3062 | 8O | SEQ ID NO: 15 | SEQ ID NO: 44 | SEQ ID NO: 45 |
| H5 A-Indo-5-05 | Wt | 2295 | 9A | SEQ ID NO: 15 | SEQ ID NO: 67 | — |
| H5 A-Indo-5-05 (F393D) | Wt | 3680 | 9B | SEQ ID NO: 15 | SEQ ID NO: 70 | SEQ ID NO: 71 |
| H5 A-Egypt-N04915-14 | Wt | 3645 | 9C | SEQ ID NO: 15 | SEQ ID NO: 74 | — |
| H5 A-Egypt-N04915-14 (F392D) | Wt | 3690 | 9D | SEQ ID NO: 15 | SEQ ID NO: 77 | SEQ ID NO: 78 |
| H3 A-Swi-9715293-13 (N382A) | Wt | 3023 | 4C | SEQ ID NO: 15 | SEQ ID NO: 50 | SEQ ID NO: 51 |
| H3 A-Swi-9715293-13 (L384V) | Wt | 3034 | 4D | SEQ ID NO: 15 | SEQ ID NO: 54 | SEQ ID NO: 55 |
| H3 A-Penn-09-15 | Wt | 3312 | 8P | SEQ ID NO: 15 | SEQ ID NO: 17 | — |
| H3 A-Penn-09-15 | CysTM | 3313 | 8Q | SEQ ID NO: 15 | SEQ ID NO: 18 | SEQ ID NO: 19 |
| H3 A-Penn-09-15 (N382A) | CysTM | 3314 | 8R | SEQ ID NO: 15 | SEQ ID NO: 50 | SEQ ID NO: 51 |
| H3 A-Penn-09-15 (L384V) | CysTM | 3315 | 8S | SEQ ID NO: 15 | SEQ ID NO: 54 | SEQ ID NO: 55 |

| Construct Name | Primer 4 | Template for PCR | Resulting Gene | Resulting Protein |
|---|---|---|---|---|
| H3 A-HK-4801-14 | — | SEQ ID NO: 1 | SEQ ID NO: 1 | SEQ ID NO: 2 |
| H3 A-HK-4801-14 | SEQ ID NO: 17 | SEQ ID NO: 1 | SEQ ID NO: 20 | SEQ ID NO: 21 |
| H3 A-HK-4801-14 (N382A + L384V) | SEQ ID NO: 17 | SEQ ID NO: 20 | SEQ ID NO: 24 | SEQ ID NO: 25 |
| H3 A-Minn-40-15 | SEQ ID NO: 17 | SEQ ID NO: 3 | SEQ ID NO: 26 | SEQ ID NO: 27 |
| H3 A-Minn-40-15 (N382A + L384V) | SEQ ID NO: 17 | SEQ ID NO: 26 | SEQ ID NO: 30 | SEQ ID NO: 31 |
| H3 A-SAus-1-16 | SEQ ID NO: 17 | SEQ ID NO: 5 | SEQ ID NO: 32 | SEQ ID NO: 33 |
| H3 A-SAus-1-16 (N382A + L384V) | SEQ ID NO: 17 | SEQ ID NO: 32 | SEQ ID NO: 34 | SEQ ID NO: 35 |
| H3 A-Bang-3007-15 | SEQ ID NO: 17 | SEQ ID NO: 7 | SEQ ID NO: 36 | SEQ ID NO: 37 |
| H3 A-Bang-3007-15 (N382A + L384V) | SEQ ID NO: 17 | SEQ ID NO: 36 | SEQ ID NO: 38 | SEQ ID NO: 39 |
| H3 A-Swi-9715293-13 | — | SEQ ID NO: 9 | SEQ ID NO: 9 | SEQ ID NO: 10 |
| H3 A-Swi-9715293-13 (F392D) | SEQ ID NO: 17 | SEQ ID NO: 9 | SEQ ID NO: 42 | SEQ ID NO: 43 |
| H3 A-Swi-9715293-13 (L431M) | SEQ ID NO: 17 | SEQ ID NO: 9 | SEQ ID NO: 46 | SEQ ID NO: 47 |
| H3 A-Swi-9715293-13 | SEQ ID NO: 17 | SEQ ID NO: 9 | SEQ ID NO: 48 | SEQ ID NO: 49 |
| H3 A-Swi-9715293-13 (N382A) | SEQ ID NO: 17 | SEQ ID NO: 48 | SEQ ID NO: 52 | SEQ ID NO: 53 |
| H3 A-Swi-9715293-13 (L384V) | SEQ ID NO: 17 | SEQ ID NO: 48 | SEQ ID NO: 56 | SEQ ID NO: 57 |
| H3 A-Swi-9715293-13 (F392D) | SEQ ID NO: 17 | SEQ ID NO: 48 | SEQ ID NO: 58 | SEQ ID NO: 59 |
| H3 A-Swi-9715293-13 (L431M) | SEQ ID NO: 17 | SEQ ID NO: 48 | SEQ ID NO: 60 | SEQ ID NO: 61 |
| H5 A-Indo-5-05 | — | SEQ ID NO: 68 | SEQ ID NO: 68 | SEQ ID NO: 69 |
| H5 A-Indo-5-05 (F393D) | SEQ ID NO: 67 | SEQ ID NO: 68 | SEQ ID NO: 72 | SEQ ID NO: 73 |
| H5 A-Egypt-N04915-14 | — | SEQ ID NO: 75 | SEQ ID NO: 75 | SEQ ID NO: 76 |
| H5 A-Egypt-N04915-14 (F392D) | SEQ ID NO: 74 | SEQ ID NO: 75 | SEQ ID NO: 79 | SEQ ID NO: 80 |
| H3 A-Swi-9715293-13 (N382A) | SEQ ID NO: 17 | SEQ ID NO: 9 | SEQ ID NO: 81 | SEQ ID NO: 82 |
| H3 A-Swi-9715293-13 (L384V) | SEQ ID NO: 17 | SEQ ID NO: 9 | SEQ ID NO: 83 | SEQ ID NO: 84 |
| H3 A-Penn-09-15 | — | SEQ ID NO: 13 | SEQ ID NO: 13 | SEQ ID NO: 14 |
| H3 A-Penn-09-15 | SEQ ID NO: 17 | SEQ ID NO: 13 | SEQ ID NO: 85 | SEQ ID NO: 86 |
| H3 A-Penn-09-15 (N382A) | SEQ ID NO: 17 | SEQ ID NO: 85 | SEQ ID NO: 87 | SEQ ID NO: 88 |
| H3 A-Penn-09-15 (L384V) | SEQ ID NO: 17 | SEQ ID NO: 85 | SEQ ID NO: 89 | SEQ ID NO: 90 |

### Example 3: Hemagglutination Titer, Post-Density Gradient Yields and Full Process Yields

A Summary of the measured Hemagglutination Titer is given in Tables 5 and 6A. Hemagglutination Titer were measured as described in Example 2. The relative hemagglutination titer were obtained by comparing the hemagglutination titer of the mutated or modified HA protein to either wildtype HA (Table 5) or the CysTM mutant HA protein (Table 6A).

A Summary of the measured Post-Density Gradient Yields is given in Tables A 5B and C5B. Post-Density Gradient Yields were measured as described in Example 2. Relative yields were obtained by comparing the HAO band intensity from the mutated or modified HA protein to either the HAO band intensity of wildtype HA (Table A 51B) or the HAO band intensity of CysTM mutant HA protein (Table 61B).

A Summary of the measured Full Process Yield is given in Table 6C. Full Process Yield were obtained as described above in Example 2. Relative yields were obtained by comparing the protein yield from the mutated or modified HA protein to either the protein yield of wildtype HA or the protein yield of CysTM mutant HA protein (Table 6C).

TABLE 5
Summary of relative Hemagglutination Titer for H3. Numbering
is in accordance with H3 A/Hongkong/4801/14.

| | WT | N382A | L384V | F392D | L431M |
|---|---|---|---|---|---|
| H3 A/Switzerland/9715293/13 | 100% | 130% | 200% | 140% | 170% |

TABLE 6A
Summary of relative Hemagglutination Titer for H3. Numbering
is in accordance with H3 A/Hongkong/4801/14.

| | | | | | | | N382A + |
|---|---|---|---|---|---|---|---|
| | | | N382A + | L384V + | F392D + | L431M + | L384V + |
| | WT | CysTM | CysTM | CysTM | CysTM | CysTM | CysTM |
| H3 A/Hongkong/4801/14 | | 100% | | | | | 400% |
| H3 A/Minnesota/40/15 | | 100% | | | | | 500% |
| H3 A/S.Aus/1/16 | | 100% | | | | | 300-500% |
| H3 A/Bangkok/3007/15 | | 100% | | | | | 500% |
| H3 A/Switzerland/9715293/13 | 100% | 160% | 260% | 270% | 200-270% | 250% | 480% |
| H3 A/Mississippi/16/16 | | 100% | | | | | 100% |

TABLE 6B
Summary of Post-Density Gradient Yields for H3 (expressed in
%). Numbering is in accordance with H3 A/Hongkong/4801/14.

| | | | N382A + | L384V + | F392D + | L431M + |
|---|---|---|---|---|---|---|
| | WT | CysTM | CysTM | CysTM | CysTM | CysTM |
| H3 A/Switzerland/9715293/13 | 93% | 100% | 129% | 150% | 100% | 100% |

TABLE 6C
Summary of relative Full Process Yield for H3. Numbering
is in accordance with H3 A/Hongkong/4801/14.

| | | | | | N382A + |
|---|---|---|---|---|---|
| | | | N382A + | L384V + | L384V + |
| | WT | CysTM | CysTM | CysTM | CysTM |
| H3 A/Hongkong/4801/14 | | 100% | 135% | 135% | 150% |
| H3 A/Minnesota/40/15 | | 100% | | | 280% |
| H3 A/S.Aus/1/16 | | 100% | | | 395% |
| H3 A/Switzerland/9715293/13 | 100% | 196% | | | |
| H3 A/Mississippi/16/16 | | 100% | | | 152% |

TABLE 7
Summary of relative Hemagglutination Titer for H5.
Numbering in accordance with H5 A/Indonesia/5/2005

| | WT | N383A | F393D |
|---|---|---|---|
| H5 A/Indonesia/5/2005 | 100% | 50% | 2% |
| H5 A/Egypt/N04915/2014 | 100% | 50% | 1% |

### Example 4: Sequences

The following sequences were used (also see Table 4):

PDI-H3 HK DNA
(SEQ ID NO: 1)
ATGGCGAAAAACGTTGCGATTTTCGGCTTATTGTTTTCTCTTCTTGTGTTGGTTCCTTCTCAGATCTTC
GCGCAAAAAATTCCTGGAAATGACAATAGCACGGCAACGCTGTGCCTTGGGCATCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGACTGGAGTCACTCAAAACGGAACAAGTTCT
GCTTGCATAAGGAGATCTAGTAGTAGTTTCTTTAGTAGATTAAATTGGTTGACCCACTTAAACTACACA
TACCCAGCATTGAACGTGACTATGCCAAACAATGAACAATTTGACAAATTGTACATTTGGGGGGTTCAC
CACCCGGGTACGGACAAGGACCAAATCTTCCTGTATGCTCAATCATCAGGAAGAATCACAGTATCTACC
AAAAGAAGCCAACAAGCTGTAATCCCAAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCATAGCACTCTGAAATTGGCAACAGGAATGCGA
AATGTACCAGAGAAACAAACTAGAGGCATATTTGGTGCAATAGCGGGTTTCATAGAAAATGGTTGGGAG
GGAATGGTGGATGGTTGGTACGGTTTCAGGCATCAAAATTCTGAGGGAAGAGGACAAGCAGCAGATCTC
AAAAGCACTCAAGCAGCAATCGATCAAATCAATGGGAAGCTGAATCGATTGATCGGGAAAACCAACGAG
AAATTCCATCAGATTGAAAAAGAATTCTCAGAAGTAGAAGGAAGAATTCAGGACCTTGAGAAATATGTT
GAGGACACTAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATAAGA
AATGGAACTTATGACCACAATGTGTACAGGGATGAAGCATTAAACAACCGGTTCCAGATCAAGGGAGTT
GAGCTGAAGTCAGGGTACAAAGATTGGATCCTATGGATTTCCTTTGCCATATCATGTTTTTTGCTTTGT
GTTGCTTTGTTGGGGTTCATCATGTGGGCCTGCCAAAAGGGCAACATTAGGTGCAACATTTGCATTTGA
PDI-H3 HK AA
(SEQ ID NO: 2)
MAKNVAIFGLLFSLLVLVPSQIFAQKIPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWTGVTQNGTSSACIRRSSSSFFSRLNWLTHLNYTYPALNVTMPNNEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKHSTLKLATGMR
NVPEKQTRGIFGAIAGFIENGWEGMVDGWYGFRHQNSEGRGQAADLKSTQAAIDQINGKLNRLIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHNVYRDEALNNRFQIKGVELKSGYKDWILWISFAISCFLLC
VALLGFIMWACQKGNIRCNICI
PDI-H3 Minn DNA
(SEQ ID NO: 3)
ATGGCGAAAAACGTTGCGATTTTCGGCTTATTGTTTTCTCTTCTTGTGTTGGTTCCTTCTCAGATCTTC
GCGCAAAAAATTCCTGGAAATGACAATAGCACGGCAACGCTGTGCCTTGGGCACCATGCAGTACCAAAC
GGAACGATAGTGAAAACAATCACAAATGACCGAATTGAAGTTACTAATGCTACTGAGTTGGTTCAGAAT
TCCTCAATAGGTGAAATATGCGACAGTCCTCATCAGATCCTTGATGGAGAGAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGACTGGAGTCACTCAAAACGGAACAAGTTCT
GCTTGCATAAGGAGATCTAGTAGTAGTTTCTTTAGTAGATTAAATTGGTTGACCCACTTAAACTACACA
TATCCAGCATTGAACGTGACTATGCCAAACAAGGAACAATTTGACAAATTGTACATTTGGGGGGTTCAC
CACCCGGGTACGGACAAGGACCAAATCTTCCTGTATGCTCAATCATCAGGAAGAATCACAGTATCTACC
AAAAGAAGCCAACAAGCTGTAATCCCAAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCATAGCACTCTGAAATTGGCAACAGGAATGCGA
AATGTACCAGAGAAACAAACTAGAGGCATATTTGGCGCAATAGCGGGTTTCATAGAAAATGGTTGGGAG
GGAATGGTGGATGGTTGGTACGGTTTCAGGCATCAAAATTCTGAGGGAAGAGGACAAGCAGCAGATCTC
AAAAGCACTCAAGCAGCAATCGATCAAATCAATGGGAAGCTGAATCGGTTGATCGGGAAAACCAACGAG
AAATTCCATCAGATTGAAAAAGAATTCTCAGAAGTAGAAGGAAGAGTTCAAGACCTTGAGAAATATGTT
GAGGACACTAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGAAATGGTTGTTTCAAAATATACCACAAATGTGACAATGCCTGCATAGGATCAATAAGA
AATGAAACTTATGACCACAATGTGTACAGGGATGAAGCATTAAACAACCGGTTCCAGATCAAGGGAGTT
GAGCTGAAGTCAGGGTACAAAGATTGGATCCTATGGATTTCCTTTGCCATATCATGTTTTTTGCTTTGT
GTTGCTTTGTTGGGGTTCATCATGTGGGCCTGCCAAAAGGGCAACATTAGATGCAACATTTGCATTTGA
PDI-H3 Minn AA
(SEQ ID NO: 4)
MAKNVAIFGLLFSLLVLVPSQIFAQKIPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWTGVTQNGTSSACIRRSSSSFFSRLNWLTHLNYTYPALNVTMPNKEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKHSTLKLATGMR
NVPEKQTRGIFGAIAGFIENGWEGMVDGWYGFRHQNSEGRGQAADLKSTQAAIDQINGKLNRLIGKTNE
KFHQIEKEFSEVEGRVQDLEKYVEDTKIDLWSYNAELLVALENQHTIDLTDSEMNKLFEKTKKQLRENA
EDMGNGCFKIYHKCDNACIGSIRNETYDHNVYRDEALNNRFQIKGVELKSGYKDWILWISFAISCFLLC
VALLGFIMWACQKGNIRCNICI
PDI-H3 SAus DNA
(SEQ ID NO: 5)
ATGGCGAAAAACGTTGCGATTTTCGGCTTATTGTTTTCTCTTCTTGTGTTGGTTCCTTCTCAGATCTTC
GCGCAAAAAATTCCTGGAAATGACAATAGCACGGCAACGCTGTGCCTTGGGCACCATGCAGTACCAAAC
GGAACGATAGTGAAAACAATCACAAATGACCGAATTGAAGTTACTAATGCTACTGAGTTGGTTCAGAAT
TCCTCAATAGGTGAAATATGCGACAGTCCTCATCAGATCCTTGATGGAGAGAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAAAAATGAAAGCTTCAATTGGACTGGAGTCACTCAAAACGGAACAAGTTCT
GCTTGCATAAGGAGATCTAGTAGTAGTTTCTTTAGTAGATTAAATTGGTTGACCCACTTAAACTACACA
TATCCAGCATTGAACGTGACTATGCCAAACAAGGAACAATTTGACAAATTGTACATTTGGGGGGTTCAC
CACCCGGGTACGGACAAGGACCAAATCTTCCTGTATGCTCAATCATCAGGAAGAATCACAGTATCTACC
AAAAGAAGCCAACAAGCTGTAATCCCAAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCATAGCACTCTGAAATTGGCAACAGGAATGCGA
AATGTACCAGAGAAACAAACTAGAGGCATATTTGGCGCAATAGCGGGTTTCATAGAAAATGGTTGGGAG
GGAATGGTGGATGGTTGGTACGGTTTCAGGCATCAAAATTCTGAGGGAAGAGGACAAGCAGCAGATCTC
AAAAGCACTCAAGCAGCAATCGATCAAATCAATGGGAAGCTGAATCGGTTGATCGGGAAAACCAACGAG
AAATTCCATCAGATTGAAAAAGAATTCTCAGAAGTAGAAGGAAGAGTTCAAGACCTTGAGAAATATGTT
GAGGACACTAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGAAATGGTTGTTTCAAAATATACCACAAATGTGACAATGCCTGCATAGGATCAATAAGA
AATGAAACTTATGACCACAATGTGTACAGGGATGAAGCATTAAACAACCGGTTCCAGATCAAGGGAGTT
GAGCTGAAGTCAGGGTACAAAGATTGGATCCTATGGATTTCCTTTGCCATATCATGTTTTTTGCTTTGT
GTTGCTTTGTTGGGGTTCATCATGTGGGCCTGCCAAAAGGGCAACATTAGATGCAACATTTGCATTTGA
PDI-H3 SAus AA
(SEQ ID NO: 6)
MAKNVAIFGLLFSLLVLVPSQIFAQKIPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFKNESFNWTGVTQNGTSSACIRRSSSSFFSRLNWLTHLNYTYPALNVTMPNKEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKHSTLKLATGMR
NVPEKQTRGIFGAIAGFIENGWEGMVDGWYGFRHQNSEGRGQAADLKSTQAAIDQINGKLNRLIGKTNE
KFHQIEKEFSEVEGRVQDLEKYVEDTKIDLWSYNAELLVALENQHTIDLTDSEMNKLFEKTKKQLRENA
EDMGNGCFKIYHKCDNACIGSIRNETYDHNVYRDEALNNRFQIKGVELKSGYKDWILWISFAISCFLLC
VALLGFIMWACQKGNIRCNICI
PDI-H3 Bang DNA
(SEQ ID NO: 7)
ATGGCGAAAAACGTTGCGATTTTCGGCTTATTGTTTTCTCTTCTTGTGTTGGTTCCTTCTCAGATCTTC
GCGCAAAAAATTCCTGGAAATGACAATAGCACGGCAACGCTGTGCCTTGGGCACCATGCAGTACCAAAC
GGAACGATAGTGAAAACAATCACAAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGACTGGAGTCACTCAAAACGGAACAAGTTCT
GCTTGCATAAGGAAATCTAGTAGTAGTTTCTTTAGTAGATTAAATTGGTTGACCCACTTAAACTACACA
TACCCAGCATTGAACGTGACTGTGCCAAACAATGAACAATTTGACAAATTGTACATTTGGGGGGTTCAC
CACCCGGGTACGGACAAGGACCAAATCTTCCTGTATGCTCGATCATCAGGAAGAATCACAGTATCTACC
AAAAGAAGCCAACAAGCTGTAATCCCAAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCATAGCACTCTGAAATTGGCAACAGGAATGCGA
AATGTACCAGAGAAACAAACTAGAGGCATATTTGGCGCAATAGCGGGTTTCATAGAAAATGGTTGGGAG
GGAATGGTGGATGGTTGGTACGGTTTCAGGCATCAAAATTCTGAGGGAAGAGGACAAGCAGCAGATCTC
AAAAGCACTCAAGCAGCAATCGATCAAATCAATGGGAAGCTGAATCGATTGATCGGGAAAACCAACGAG
AAATTCCATCAGATTGAAAAAGAATTCTCAGAAGTAGAAGGAAGAATTCAGGACCTTGAGAAATATGTT
GAGGACACTAAAATAGATCTCTGGTCATACAACGCGGAGCTTCTTGTTGCCCTGGAGAATCAACATACA
ATTGATCTAACTGACTCAGAAATGAACAAACTGTTTGAAAAAACAAAGAAGCAACTGAGGGAAAATGCT
GAGGATATGGGAAATGGTTGTTTCAAAATATACCACAAATGTGACAATGCCTGCATAGGATCAATAAGA
AATGGAACTTATGACCACAATGTGTACAGGGATGAAGCATTAAACAACCGGTTCCAGATCAAGGGAGTT
GAGCTGAAGTCAGGGTACAAAGATTGGATCCTATGGATTTCCTTTGCCATATCATGTTTTTTGCTTTGT
GTTGCTTTGTTGGGGTTCATCATGTGGGCCTGCCAAAAGGGCAACATTAGGTGCAACATTTGCATTTGA
PDI-H3 Bang AA
(SEQ ID NO: 8)
MAKNVAIFGLLFSLLVLVPSQIFAQKIPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWTGVTQNGTSSACIRKSSSSFFSRLNWLTHLNYTYPALNVTVPNNEQFDKLYIWGVH
HPGTDKDQIFLYARSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKHSTLKLATGMR
NVPEKQTRGIFGAIAGFIENGWEGMVDGWYGFRHQNSEGRGQAADLKSTQAAIDQINGKLNRLIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHNVYRDEALNNRFQIKGVELKSGYKDWILWISFAISCFLLC
VALLGFIMWACQKGNIRCNICI
PDI-H3 Switz DNA
(SEQ ID NO: 9)
ATGGCGAAAAACGTTGCGATTTTCGGCTTATTGTTTTCTCTTCTTGTGTTGGTTCCTTCTCAGATCTTC
GCGCAAAAACTTCCTGGAAATGACAATAGCACGGCAACGCTGTGCCTTGGGCACCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGGCTGGAGTCACTCAAAACGGAACAAGTTCT
TCTTGCATAAGGGGATCTAATAGTAGTTTCTTTAGTAGATTAAATTGGTTGACCCACTTAAACTCCAAA
TACCCAGCATTAAACGTGACTATGCCAAACAATGAACAATTTGACAAATTGTACATTTGGGGGGTTCAC
CACCCGGGTACGGACAAGGACCAAATCTTCCTGTATGCACAATCATCAGGAAGAATCACAGTATCTACC
AAAAGAAGCCAACAAGCTGTAATCCCGAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCAAAGCACTCTGAAATTGGCAACAGGAATGCGA
AATGTACCAGAGAGACAAACTAGAGGCATATTTGGCGCAATAGCGGGTTTCATAGAAAATGGTTGGGAG
GGAATGGTGGATGGTTGGTACGGCTTCAGGCATCAAAATTCTGAGGGAAGAGGACAAGCAGCAGATCTC
AAAAGCACTCAAGCAGCAATCGATCAAATCAATGGGAAGCTGAATCGATTGATCGGGAAAACCAACGAG
AAATTCCATCAGATTGAAAAAGAATTCTCAGAAGTAGAAGGGAGAATTCAGGACCTTGAGAAATATGTT
GAGGACACAAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATCAGA
AATGGAACTTATGACCACGATGTATACAGGGATGAAGCATTAAACAACCGGTTCCAGATCAAGGGAGTT
GAGCTGAAGTCAGGGTACAAAGATTGGATCCTATGGATTTCCTTTGCCATATCATGTTTTTTGCTTTGT
GTTGCTTTGTTGGGGTTCATCATGTGGGCCTGCCAAAAGGGCAACATTAGGTGCAACATTTGCATTTGA
PDI-H3 Switz AA
(SEQ ID NO: 10)
MAKNVAIFGLLFSLLVLVPSQIFAQKLPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWAGVTQNGTSSSCIRGSNSSFFSRLNWLTHLNSKYPALNVTMPNNEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKQSTLKLATGMR
NVPERQTRGIFGAIAGFIENGWEGMVDGWYGFRHQNSEGRGQAADLKSTQAAIDQINGKLNRLIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHDVYRDEALNNRFQIKGVELKSGYKDWILWISFAISCFLLC
VALLGFIMWACQKGNIRCNICI
PDI-H3 Miss DNA
(SEQ ID NO: 11)
ATGGCGAAAAACGTTGCGATTTTCGGCTTATTGTTTTCTCTTCTTGTGTTGGTTCCTTCTCAGATCTTC
GCGCAAAAACTTCCTGGAAATGACAATAGCACGGCAACGCTGTGCCTTGGGCACCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AGAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGGCTGGAGTCACTCAAAACGGAACAAGTTCT
TCTTGCATAAGGGGATCTAATAGTAGTTTCTTTAGTAGATTAAATTGGTTGACCCACTTAAACTCCAAA
TACCCAGCATTAAACGTGACTATGCCAAACAATGAACAATTTGACAAATTGTACATTTGGGGGGTTCAC
CACCCGGGTACGGACAAGGACCAAATCTTCCTGTATGCAAAACCATCAGGAAGAATCACAGTATCTACC
AAAAGAAGCCAACAAGCTGTAATCCCGAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCAAAACACTCTGAAATTGGCAACAGGAATGCGA
AATGTACCAGAGAGACAAACTAGAGGCATATTTGGCGCAATAGCGGGTTTCATAGAAAATGGTTGGGAG
GGAATGGTGGATGGTTGGTACGGCTTCAGGCATCAAAATTCTGAGGGAAGAGGACAAGCAGCAGATCTC
AAAAGCACTCAAGCAGCAATCGATCAAATCAATGGGAAGCTGAATCGATTGATCGGGAAAACCAACGAG
AAATTCCATCAGATTGAAAAAGAATTCTCAGAAGTAGAAGGGAGAATTCAGGACCTTGAGAAATATGTT
GAGGACACAAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATCAGA
AATGGAACTTATGACCACGATGTATACAGGGATGAAGCATTAAACAACCGGTTCCAGATCAAGGGAGTT
GAGCTGAAGTCAGGGTACAAAGATTGGATCCTATGGATTTCCTTTGCCATATCATGTTTTTTGCTTTGT
GTTGCTTTGTTGGGGTTCATCATGTGGGCCTGCCAAAAGGGCAACATTAGGTGCAACATTTGCATTTGA
PDI-H3 Miss AA
(SEQ ID NO: 12)
MAKNVAIFGLLFSLLVLVPSQIFAQKLPGNDNSTATLCLGHHAVPNGTIVKTITNDRIEVTNATELVQN
SSIGEICDSPHQILDGENCTLIDALLGDPQCDGFQNKKWDLFVERSRAYSNCYPYDVPDYASLRSLVAS
SGTLEFNNESFNWAGVTQNGTSSSCIRGSNSSFFSRLNWLTHLNSKYPALNVTMPNNEQFDKLYIWGVH
HPGTDKDQIFLYAKPSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKQNTLKLATGMR
NVPERQTRGIFGAIAGFIENGWEGMVDGWYGFRHQNSEGRGQAADLKSTQAAIDQINGKLNRLIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHDVYRDEALNNRFQIKGVELKSGYKDWILWISFAISCFLLC
VALLGFIMWACQKGNIRCNICI
PDI-H3 Penn DNA
(SEQ ID NO: 13)
ATGGCGAAAAACGTTGCGATTTTCGGCTTATTGTTTTCTCTTCTTGTGTTGGTTCCTTCTCAGATCTTC
GCGCAAAAACTTCCTGGAAATGACAATAGCACGGCAACGCTGTGCCTTGGGCACCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAAAAAACTGCACTCTAATAGAT
GCTCTATTGGGAGACCCTCAGTGTGATGGCTTTCAAAATAAGAGATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACGATGAAAGCTTCAATTGGGCTGGAGTCACTCAAAACGGAACAAGTTCT
GCTTGCATAAGGGGATCTAATAGTAGTTTCTTTAGTAGATTAAATTGGTTGACCCACTCAAACTTCAAA
TACCCAGCATTGAACGTGACTATGCCAAACAATGAACAATTTGACAAATTGTACATTTGGGGGGTTCAC
CACCCGGGTACAGACAAGGACCAAATCTTCCTGTACGCTCAATCATCAGGAAGAATCACAGTATCTACC
AAAAGAAGCCAACAAGCTGTAATCCCGAATATCGGATCTAGACCCAGAATAAGGAATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACAAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCAAAGCACTCTGAAATTGGCAACAGGAATGCGA
AATGTACCAGAGAAACAAACTAGAGGCATATTTGGCGCAATAGCGGGTTTCATAGAAAATGGTTGGGAG
GGAATGAAGGATGGTTGGTACGGTTTCAGGCATCAAAATTCTGAGGGAAGAGGACAAGCAGCAGATCTC
AAAAGCACTCAAGCAGCAATCGATCAAATCAATGGGAAGCTGAATCGATTGATCGGGAAAACCAACGAG
AAATTCCATCAGATTGAAAAAGAATTCTCAGAAGTAGAAGGGAGAATTCAGGACCTTGAGAAATATGTT
GAGGACACAAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATCAGA
AATGGAACTTATGACCACGATGTATACAGGGATGAAGCTTTAAACAACCGGTTCCAGATCAAGGGAGTT
GAGCTGAAGTCAGGGTACAAAGATTGGATCCTATGGATTTCCTTTGCCATATCATGTTTTTTGCTTTGT
GTTGCTTTGTTGGGGTTCATCATGTGGGCCTGCCAAAAGGGCAACATTAGGTGCAACATTTGCATTTGA
PDI-H3 Penn AA
(SEQ ID NO: 14)
MAKNVAIFGLLFSLLVLVPSQIFAQKLPGNDNSTATLCLGHHAVPNGTIVKTITNDRIEVTNATELVQN
SSIGEICDSPHQILDGKNCTLIDALLGDPQCDGFQNKRWDLFVERSKAYSNCYPYDVPDYASLRSLVAS
SGTLEFNDESFNWAGVTQNGTSSACIRGSNSSFFSRLNWLTHSNFKYPALNVTMPNNEQFDKLYIWGVH
HPGTDKDQIFLYAQSSGRITVSTKRSQQAVIPNIGSRPRIRNIPSRISIYWTIVKPGDILLINSTGNLI
APRGYFKIQSGKSSIMRSDAPIGKCKSECITPNGSIPNDKPFQNVNRITYGACPRYVKQSTLKLATGMR
NVPEKQTRGIFGAIAGFIENGWEGMKDGWYGFRHQNSEGRGQAADLKSTQAAIDQINGKLNRLIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHDVYRDEALNNRFQIKGVELKSGYKDWILWISFAISCFLLC
VALLGFIMWACQKGNIRCNICI
IF-CPMV(fl5′UTR)_SpPDI.c
(SEQ ID NO: 15)
TCGTGCTTCGGCACCAGTACAATGGCGAAAAACGTTGCGATTTTCGGCT
IF-H1cTMCT.S1-4r
(SEQ ID NO: 16)
ACTAAAGAAAATAGGCCTTTAAATACATATTCTACACTGTAGAGAC
IF-H3A-Ala.r
(SEQ ID NO: 17)
ACTAAAGAAAATAGGCCTTCAAATGCAAATGTTGCACCTAATGTTGCCCTTTTGG
H3_Swi_SLVLL.r
(SEQ ID NO: 18)
AGCAACTAACAGTACAAGGGATGATATGGCAAAGGAAATCCATAGGATCCA
H3_Swi_SLVLL.c
(SEQ ID NO: 19)
TCCTTTGCCATATCATCCCTTGTACTGTTAGTTGCTTTGTTGGGGTTCATCAT
PDI-H3 HK-CysTm DNA
(SEQ ID NO: 20)
ATGGCGAAAAACGTTGCGATTTTCGGCTTATTGTTTTCTCTTCTTGTGTTGGTTCCTTCTCAGATCTTC
GCGCAAAAAATTCCTGGAAATGACAATAGCACGGCAACGCTGTGCCTTGGGCATCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGACTGGAGTCACTCAAAACGGAACAAGTTCT
GCTTGCATAAGGAGATCTAGTAGTAGTTTCTTTAGTAGATTAAATTGGTTGACCCACTTAAACTACACA
TACCCAGCATTGAACGTGACTATGCCAAACAATGAACAATTTGACAAATTGTACATTTGGGGGGTTCAC
CACCCGGGTACGGACAAGGACCAAATCTTCCTGTATGCTCAATCATCAGGAAGAATCACAGTATCTACC
AAAAGAAGCCAACAAGCTGTAATCCCAAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCATAGCACTCTGAAATTGGCAACAGGAATGCGA
AATGTACCAGAGAAACAAACTAGAGGCATATTTGGTGCAATAGCGGGTTTCATAGAAAATGGTTGGGAG
GGAATGGTGGATGGTTGGTACGGTTTCAGGCATCAAAATTCTGAGGGAAGAGGACAAGCAGCAGATCTC
AAAAGCACTCAAGCAGCAATCGATCAAATCAATGGGAAGCTGAATCGATTGATCGGGAAAACCAACGAG
AAATTCCATCAGATTGAAAAAGAATTCTCAGAAGTAGAAGGAAGAATTCAGGACCTTGAGAAATATGTT
GAGGACACTAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATAAGA
AATGGAACTTATGACCACAATGTGTACAGGGATGAAGCATTAAACAACCGGTTCCAGATCAAGGGAGTT
GAGCTGAAGTCAGGGTACAAAGATTGGATCCTATGGATTTCCTTTGCCATATCATCCCTTGTACTGTTA
GTTGCTTTGTTGGGGTTCATCATGTGGGCCTGCCAAAAGGGCAACATTAGGTGCAACATTTGCATTTGA
PDI-H3 HK-CysTm AA
(SEQ ID NO: 21)
MAKNVAIFGLLFSLLVLVPSQIFAQKIPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWTGVTQNGTSSACIRRSSSSFFSRLNWLTHLNYTYPALNVTMPNNEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKHSTLKLATGMR
NVPEKQTRGIFGAIAGFIENGWEGMVDGWYGFRHQNSEGRGQAADLKSTQAAIDQINGKLNRLIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHNVYRDEALNNRFQIKGVELKSGYKDWILWISFAISSLVLL
VALLGFIMWACQKGNIRCNICI*
H3_HK480114(N382A + L384V).r
(SEQ ID NO: 22)
TTGGTTTTCCCGATCACTCGAGCCAGCTTCCCATTGATTTGATCGATT
H3_HK480114(N382A + L384V).c
(SEQ ID NO: 23)
GGGAAGCTGGCTCGAGTGATCGGGAAAACCAACGAGAAATTCCATCAG
PDI-H3 HK-N382A + L384V-CysTm DNA
(SEQ ID NO: 24)
ATGGCGAAAAACGTTGCGATTTTCGGCTTATTGTTTTCTCTTCTTGTGTTGGTTCCTTCTCAGATCTTC
GCGCAAAAAATTCCTGGAAATGACAATAGCACGGCAACGCTGTGCCTTGGGCATCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGACTGGAGTCACTCAAAACGGAACAAGTTCT
GCTTGCATAAGGAGATCTAGTAGTAGTTTCTTTAGTAGATTAAATTGGTTGACCCACTTAAACTACACA
TACCCAGCATTGAACGTGACTATGCCAAACAATGAACAATTTGACAAATTGTACATTTGGGGGGTTCAC
CACCCGGGTACGGACAAGGACCAAATCTTCCTGTATGCTCAATCATCAGGAAGAATCACAGTATCTACC
AAAAGAAGCCAACAAGCTGTAATCCCAAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCATAGCACTCTGAAATTGGCAACAGGAATGCGA
AATGTACCAGAGAAACAAACTAGAGGCATATTTGGTGCAATAGCGGGTTTCATAGAAAATGGTTGGGAG
GGAATGGTGGATGGTTGGTACGGTTTCAGGCATCAAAATTCTGAGGGAAGAGGACAAGCAGCAGATCTC
AAAAGCACTCAAGCAGCAATCGATCAAATCAATGGGAAGCTGGCTCGAGTGATCGGGAAAACCAACGAG
AAATTCCATCAGATTGAAAAAGAATTCTCAGAAGTAGAAGGAAGAATTCAGGACCTTGAGAAATATGTT
GAGGACACTAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATAAGA
AATGGAACTTATGACCACAATGTGTACAGGGATGAAGCATTAAACAACCGGTTCCAGATCAAGGGAGTT
GAGCTGAAGTCAGGGTACAAAGATTGGATCCTATGGATTTCCTTTGCCATATCATCCCTTGTACTGTTA
GTTGCTTTGTTGGGGTTCATCATGTGGGCCTGCCAAAAGGGCAACATTAGGTGCAACATTTGCATTTGA
PDI-H3 HK-N382A + L384V-CysTm AA
(SEQ ID NO: 25)
MAKNVAIFGLLFSLLVLVPSQIFAQKIPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWTGVTQNGTSSACIRRSSSSFFSRLNWLTHLNYTYPALNVTMPNNEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKHSTLKLATGMR
NVPEKQTRGIFGAIAGFIENGWEGMVDGWYGFRHQNSEGRGQAADLKSTQAAIDQINGKLARVIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHNVYRDEALNNRFQIKGVELKSGYKDWILWISFAISSLVLL
VALLGFIMWACQKGNIRCNICI*
PDI-H3 Minn-CysTm DNA
(SEQ ID NO: 26)
ATGGCGAAAAACGTTGCGATTTTCGGCTTATTGTTTTCTCTTCTTGTGTTGGTTCCTTCTCAGATCTTC
GCGCAAAAAATTCCTGGAAATGACAATAGCACGGCAACGCTGTGCCTTGGGCACCATGCAGTACCAAAC
GGAACGATAGTGAAAACAATCACAAATGACCGAATTGAAGTTACTAATGCTACTGAGTTGGTTCAGAAT
TCCTCAATAGGTGAAATATGCGACAGTCCTCATCAGATCCTTGATGGAGAGAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGACTGGAGTCACTCAAAACGGAACAAGTTCT
GCTTGCATAAGGAGATCTAGTAGTAGTTTCTTTAGTAGATTAAATTGGTTGACCCACTTAAACTACACA
TATCCAGCATTGAACGTGACTATGCCAAACAAGGAACAATTTGACAAATTGTACATTTGGGGGGTTCAC
CACCCGGGTACGGACAAGGACCAAATCTTCCTGTATGCTCAATCATCAGGAAGAATCACAGTATCTACC
AAAAGAAGCCAACAAGCTGTAATCCCAAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCATAGCACTCTGAAATTGGCAACAGGAATGCGA
AATGTACCAGAGAAACAAACTAGAGGCATATTTGGCGCAATAGCGGGTTTCATAGAAAATGGTTGGGAG
GGAATGGTGGATGGTTGGTACGGTTTCAGGCATCAAAATTCTGAGGGAAGAGGACAAGCAGCAGATCTC
AAAAGCACTCAAGCAGCAATCGATCAAATCAATGGGAAGCTGAATCGGTTGATCGGGAAAACCAACGAG
AAATTCCATCAGATTGAAAAAGAATTCTCAGAAGTAGAAGGAAGAGTTCAAGACCTTGAGAAATATGTT
GAGGACACTAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGAAATGGTTGTTTCAAAATATACCACAAATGTGACAATGCCTGCATAGGATCAATAAGA
AATGAAACTTATGACCACAATGTGTACAGGGATGAAGCATTAAACAACCGGTTCCAGATCAAGGGAGTT
GAGCTGAAGTCAGGGTACAAAGATTGGATCCTATGGATTTCCTTTGCCATATCATCCCTTGTACTGTTA
GTTGCTTTGTTGGGGTTCATCATGTGGGCCTGCCAAAAGGGCAACATTAGATGCAACATTTGCATTTGA
PDI-H3 Minn-CysTm AA
(SEQ ID NO: 27)
MAKNVAIFGLLFSLLVLVPSQIFAQKIPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWTGVTQNGTSSACIRRSSSSFFSRLNWLTHLNYTYPALNVTMPNKEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKHSTLKLATGMR
NVPEKQTRGIFGAIAGFIENGWEGMVDGWYGFRHQNSEGRGQAADLKSTQAAIDQINGKLNRLIGKTNE
KFHQIEKEFSEVEGRVQDLEKYVEDTKIDLWSYNAELLVALENQHTIDLTDSEMNKLFEKTKKQLRENA
EDMGNGCFKIYHKCDNACIGSIRNETYDHNVYRDEALNNRFQIKGVELKSGYKDWILWISFAISSLVLL
VALLGFIMWACQKGNIRCNICI*
H3Minn(N382A + L384V).r
(SEQ ID NO: 28)
GTTGGTTTTCCCGATCACCCGAGCCAGCTTCCCATTGATTTGATCGATTGCTGCTTGAGT
H3Minn(N382A + L384V).c
(SEQ ID NO: 29)
AATCAATGGGAAGCTGGCTCGGGTGATCGGGAAAACCAACGAGAAATTCCATCAGA
PDI-H3 Minn-N382A + L384V-CysTm DNA
(SEQ ID NO: 30)
ATGGCGAAAAACGTTGCGATTTTCGGCTTATTGTTTTCTCTTCTTGTGTTGGTTCCTTCTCAGATCTTC
GCGCAAAAAATTCCTGGAAATGACAATAGCACGGCAACGCTGTGCCTTGGGCACCATGCAGTACCAAAC
GGAACGATAGTGAAAACAATCACAAATGACCGAATTGAAGTTACTAATGCTACTGAGTTGGTTCAGAAT
TCCTCAATAGGTGAAATATGCGACAGTCCTCATCAGATCCTTGATGGAGAGAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGACTGGAGTCACTCAAAACGGAACAAGTTCT
GCTTGCATAAGGAGATCTAGTAGTAGTTTCTTTAGTAGATTAAATTGGTTGACCCACTTAAACTACACA
TATCCAGCATTGAACGTGACTATGCCAAACAAGGAACAATTTGACAAATTGTACATTTGGGGGGTTCAC
CACCCGGGTACGGACAAGGACCAAATCTTCCTGTATGCTCAATCATCAGGAAGAATCACAGTATCTACC
AAAAGAAGCCAACAAGCTGTAATCCCAAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCATAGCACTCTGAAATTGGCAACAGGAATGCGA
AATGTACCAGAGAAACAAACTAGAGGCATATTTGGCGCAATAGCGGGTTTCATAGAAAATGGTTGGGAG
GGAATGGTGGATGGTTGGTACGGTTTCAGGCATCAAAATTCTGAGGGAAGAGGACAAGCAGCAGATCTC
AAAAGCACTCAAGCAGCAATCGATCAAATCAATGGGAAGCTGGCTCGGGTGATCGGGAAAACCAACGAG
AAATTCCATCAGATTGAAAAAGAATTCTCAGAAGTAGAAGGAAGAGTTCAAGACCTTGAGAAATATGTT
GAGGACACTAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGAAATGGTTGTTTCAAAATATACCACAAATGTGACAATGCCTGCATAGGATCAATAAGA
AATGAAACTTATGACCACAATGTGTACAGGGATGAAGCATTAAACAACCGGTTCCAGATCAAGGGAGTT
GAGCTGAAGTCAGGGTACAAAGATTGGATCCTATGGATTTCCTTTGCCATATCATCCCTTGTACTGTTA
GTTGCTTTGTTGGGGTTCATCATGTGGGCCTGCCAAAAGGGCAACATTAGATGCAACATTTGCATTTGA
PDI-H3 Minn-N382A + L384V-CysTm AA
(SEQ ID NO: 31)
MAKNVAIFGLLFSLLVLVPSQIFAQKIPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWTGVTQNGTSSACIRRSSSSFFSRLNWLTHLNYTYPALNVTMPNKEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKHSTLKLATGMR
NVPEKQTRGIFGAIAGFIENGWEGMVDGWYGFRHQNSEGRGQAADLKSTQAAIDQINGKLARVIGKTNE
KFHQIEKEFSEVEGRVQDLEKYVEDTKIDLWSYNAELLVALENQHTIDLTDSEMNKLFEKTKKQLRENA
EDMGNGCFKIYHKCDNACIGSIRNETYDHNVYRDEALNNRFQIKGVELKSGYKDWILWISFAISSLVLL
VALLGFIMWACQKGNIRCNICI*
PDI-H3 SAus-CysTm DNA
(SEQ ID NO: 32)
ATGGCGAAAAACGTTGCGATTTTCGGCTTATTGTTTTCTCTTCTTGTGTTGGTTCCTTCTCAGATCTTC
GCGCAAAAAATTCCTGGAAATGACAATAGCACGGCAACGCTGTGCCTTGGGCACCATGCAGTACCAAAC
GGAACGATAGTGAAAACAATCACAAATGACCGAATTGAAGTTACTAATGCTACTGAGTTGGTTCAGAAT
TCCTCAATAGGTGAAATATGCGACAGTCCTCATCAGATCCTTGATGGAGAGAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAAAAATGAAAGCTTCAATTGGACTGGAGTCACTCAAAACGGAACAAGTTCT
GCTTGCATAAGGAGATCTAGTAGTAGTTTCTTTAGTAGATTAAATTGGTTGACCCACTTAAACTACACA
TATCCAGCATTGAACGTGACTATGCCAAACAAGGAACAATTTGACAAATTGTACATTTGGGGGGTTCAC
CACCCGGGTACGGACAAGGACCAAATCTTCCTGTATGCTCAATCATCAGGAAGAATCACAGTATCTACC
AAAAGAAGCCAACAAGCTGTAATCCCAAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCATAGCACTCTGAAATTGGCAACAGGAATGCGA
AATGTACCAGAGAAACAAACTAGAGGCATATTTGGCGCAATAGCGGGTTTCATAGAAAATGGTTGGGAG
GGAATGGTGGATGGTTGGTACGGTTTCAGGCATCAAAATTCTGAGGGAAGAGGACAAGCAGCAGATCTC
AAAAGCACTCAAGCAGCAATCGATCAAATCAATGGGAAGCTGAATCGGTTGATCGGGAAAACCAACGAG
AAATTCCATCAGATTGAAAAAGAATTCTCAGAAGTAGAAGGAAGAGTTCAAGACCTTGAGAAATATGTT
GAGGACACTAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGAAATGGTTGTTTCAAAATATACCACAAATGTGACAATGCCTGCATAGGATCAATAAGA
AATGAAACTTATGACCACAATGTGTACAGGGATGAAGCATTAAACAACCGGTTCCAGATCAAGGGAGTT
GAGCTGAAGTCAGGGTACAAAGATTGGATCCTATGGATTTCCTTTGCCATATCATCCCTTGTACTGTTA
GTTGCTTTGTTGGGGTTCATCATGTGGGCCTGCCAAAAGGGCAACATTAGATGCAACATTTGCATTTGA
PDI-H3 SAus-CysTm AA
(SEQ ID NO: 33)
MAKNVAIFGLLFSLLVLVPSQIFAQKIPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFKNESFNWTGVTQNGTSSACIRRSSSSFFSRLNWLTHLNYTYPALNVTMPNKEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKHSTLKLATGMR
NVPEKQTRGIFGAIAGFIENGWEGMVDGWYGFRHQNSEGRGQAADLKSTQAAIDQINGKLNRLIGKTNE
KFHQIEKEFSEVEGRVQDLEKYVEDTKIDLWSYNAELLVALENQHTIDLTDSEMNKLFEKTKKQLRENA
EDMGNGCFKIYHKCDNACIGSIRNETYDHNVYRDEALNNRFQIKGVELKSGYKDWILWISFAISSLVLL
VALLGFIMWACQKGNIRCNICI*
PDI-H3 SAus-N382A + L384V-CysTm DNA
(SEQ ID NO: 34)
ATGGCGAAAAACGTTGCGATTTTCGGCTTATTGTTTTCTCTTCTTGTGTTGGTTCCTTCTCAGATCTTC
GCGCAAAAAATTCCTGGAAATGACAATAGCACGGCAACGCTGTGCCTTGGGCACCATGCAGTACCAAAC
GGAACGATAGTGAAAACAATCACAAATGACCGAATTGAAGTTACTAATGCTACTGAGTTGGTTCAGAAT
TCCTCAATAGGTGAAATATGCGACAGTCCTCATCAGATCCTTGATGGAGAGAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAAAAATGAAAGCTTCAATTGGACTGGAGTCACTCAAAACGGAACAAGTTCT
GCTTGCATAAGGAGATCTAGTAGTAGTTTCTTTAGTAGATTAAATTGGTTGACCCACTTAAACTACACA
TATCCAGCATTGAACGTGACTATGCCAAACAAGGAACAATTTGACAAATTGTACATTTGGGGGGTTCAC
CACCCGGGTACGGACAAGGACCAAATCTTCCTGTATGCTCAATCATCAGGAAGAATCACAGTATCTACC
AAAAGAAGCCAACAAGCTGTAATCCCAAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCATAGCACTCTGAAATTGGCAACAGGAATGCGA
AATGTACCAGAGAAACAAACTAGAGGCATATTTGGCGCAATAGCGGGTTTCATAGAAAATGGTTGGGAG
GGAATGGTGGATGGTTGGTACGGTTTCAGGCATCAAAATTCTGAGGGAAGAGGACAAGCAGCAGATCTC
AAAAGCACTCAAGCAGCAATCGATCAAATCAATGGGAAGCTGGCTCGGGTGATCGGGAAAACCAACGAG
AAATTCCATCAGATTGAAAAAGAATTCTCAGAAGTAGAAGGAAGAGTTCAAGACCTTGAGAAATATGTT
GAGGACACTAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGAAATGGTTGTTTCAAAATATACCACAAATGTGACAATGCCTGCATAGGATCAATAAGA
AATGAAACTTATGACCACAATGTGTACAGGGATGAAGCATTAAACAACCGGTTCCAGATCAAGGGAGTT
GAGCTGAAGTCAGGGTACAAAGATTGGATCCTATGGATTTCCTTTGCCATATCATCCCTTGTACTGTTA
GTTGCTTTGTTGGGGTTCATCATGTGGGCCTGCCAAAAGGGCAACATTAGATGCAACATTTGCATTTGA
PDI-H3 SAus-N382A + L384V-CysTm AA
(SEQ ID NO: 35)
MAKNVAIFGLLFSLLVLVPSQIFAQKIPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFKNESFNWTGVTQNGTSSACIRRSSSSFFSRLNWLTHLNYTYPALNVTMPNKEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKHSTLKLATGMR
NVPEKQTRGIFGAIAGFIENGWEGMVDGWYGFRHQNSEGRGQAADLKSTQAAIDQINGKLARVIGKTNE
KFHQIEKEFSEVEGRVQDLEKYVEDTKIDLWSYNAELLVALENQHTIDLTDSEMNKLFEKTKKQLRENA
EDMGNGCFKIYHKCDNACIGSIRNETYDHNVYRDEALNNRFQIKGVELKSGYKDWILWISFAISSLVLL
VALLGFIMWACQKGNIRCNICI*
PDI-H3 Bang-CysTm DNA
(SEQ ID NO: 36)
ATGGCGAAAAACGTTGCGATTTTCGGCTTATTGTTTTCTCTTCTTGTGTTGGTTCCTTCTCAGATCTTC
GCGCAAAAAATTCCTGGAAATGACAATAGCACGGCAACGCTGTGCCTTGGGCACCATGCAGTACCAAAC
GGAACGATAGTGAAAACAATCACAAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGACTGGAGTCACTCAAAACGGAACAAGTTCT
GCTTGCATAAGGAAATCTAGTAGTAGTTTCTTTAGTAGATTAAATTGGTTGACCCACTTAAACTACACA
TACCCAGCATTGAACGTGACTGTGCCAAACAATGAACAATTTGACAAATTGTACATTTGGGGGGTTCAC
CACCCGGGTACGGACAAGGACCAAATCTTCCTGTATGCTCGATCATCAGGAAGAATCACAGTATCTACC
AAAAGAAGCCAACAAGCTGTAATCCCAAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCATAGCACTCTGAAATTGGCAACAGGAATGCGA
AATGTACCAGAGAAACAAACTAGAGGCATATTTGGCGCAATAGCGGGTTTCATAGAAAATGGTTGGGAG
GGAATGGTGGATGGTTGGTACGGTTTCAGGCATCAAAATTCTGAGGGAAGAGGACAAGCAGCAGATCTC
AAAAGCACTCAAGCAGCAATCGATCAAATCAATGGGAAGCTGAATCGATTGATCGGGAAAACCAACGAG
AAATTCCATCAGATTGAAAAAGAATTCTCAGAAGTAGAAGGAAGAATTCAGGACCTTGAGAAATATGTT
GAGGACACTAAAATAGATCTCTGGTCATACAACGCGGAGCTTCTTGTTGCCCTGGAGAATCAACATACA
ATTGATCTAACTGACTCAGAAATGAACAAACTGTTTGAAAAAACAAAGAAGCAACTGAGGGAAAATGCT
GAGGATATGGGAAATGGTTGTTTCAAAATATACCACAAATGTGACAATGCCTGCATAGGATCAATAAGA
AATGGAACTTATGACCACAATGTGTACAGGGATGAAGCATTAAACAACCGGTTCCAGATCAAGGGAGTT
GAGCTGAAGTCAGGGTACAAAGATTGGATCCTATGGATTTCCTTTGCCATATCATCCCTTGTACTGTTA
GTTGCTTTGTTGGGGTTCATCATGTGGGCCTGCCAAAAGGGCAACATTAGGTGCAACATTTGCATTTGA
PDI-H3 Bang-CysTm AA
(SEQ ID NO: 37)
MAKNVAIFGLLFSLLVLVPSQIFAQKIPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWTGVTQNGTSSACIRKSSSSFFSRLNWLTHLNYTYPALNVTVPNNEQFDKLYIWGVH
HPGTDKDQIFLYARSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKHSTLKLATGMR
NVPEKQTRGIFGAIAGFIENGWEGMVDGWYGFRHQNSEGRGQAADLKSTQAAIDQINGKLNRLIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHNVYRDEALNNRFQIKGVELKSGYKDWILWISFAISSLVLL
VALLGFIMWACQKGNIRCNICI*
PDI-H3 Bang-N382A + L384V-CysTm DNA
(SEQ ID NO: 38)
ATGGCGAAAAACGTTGCGATTTTCGGCTTATTGTTTTCTCTTCTTGTGTTGGTTCCTTCTCAGATCTTC
GCGCAAAAAATTCCTGGAAATGACAATAGCACGGCAACGCTGTGCCTTGGGCACCATGCAGTACCAAAC
GGAACGATAGTGAAAACAATCACAAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGACTGGAGTCACTCAAAACGGAACAAGTTCT
GCTTGCATAAGGAAATCTAGTAGTAGTTTCTTTAGTAGATTAAATTGGTTGACCCACTTAAACTACACA
TACCCAGCATTGAACGTGACTGTGCCAAACAATGAACAATTTGACAAATTGTACATTTGGGGGGTTCAC
CACCCGGGTACGGACAAGGACCAAATCTTCCTGTATGCTCGATCATCAGGAAGAATCACAGTATCTACC
AAAAGAAGCCAACAAGCTGTAATCCCAAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCATAGCACTCTGAAATTGGCAACAGGAATGCGA
AATGTACCAGAGAAACAAACTAGAGGCATATTTGGCGCAATAGCGGGTTTCATAGAAAATGGTTGGGAG
GGAATGGTGGATGGTTGGTACGGTTTCAGGCATCAAAATTCTGAGGGAAGAGGACAAGCAGCAGATCTC
AAAAGCACTCAAGCAGCAATCGATCAAATCAATGGGAAGCTGGCTCGAGTGATCGGGAAAACCAACGAG
AAATTCCATCAGATTGAAAAAGAATTCTCAGAAGTAGAAGGAAGAATTCAGGACCTTGAGAAATATGTT
GAGGACACTAAAATAGATCTCTGGTCATACAACGCGGAGCTTCTTGTTGCCCTGGAGAATCAACATACA
ATTGATCTAACTGACTCAGAAATGAACAAACTGTTTGAAAAAACAAAGAAGCAACTGAGGGAAAATGCT
GAGGATATGGGAAATGGTTGTTTCAAAATATACCACAAATGTGACAATGCCTGCATAGGATCAATAAGA
AATGGAACTTATGACCACAATGTGTACAGGGATGAAGCATTAAACAACCGGTTCCAGATCAAGGGAGTT
GAGCTGAAGTCAGGGTACAAAGATTGGATCCTATGGATTTCCTTTGCCATATCATCCCTTGTACTGTTA
GTTGCTTTGTTGGGGTTCATCATGTGGGCCTGCCAAAAGGGCAACATTAGGTGCAACATTTGCATTTGA
PDI-H3 Bang-N382A + L384V-CysTm AA
(SEQ ID NO: 39)
MAKNVAIFGLLFSLLVLVPSQIFAQKIPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWTGVTQNGTSSACIRKSSSSFFSRLNWLTHLNYTYPALNVTVPNNEQFDKLYIWGVH
HPGTDKDQIFLYARSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKHSTLKLATGMR
NVPEKQTRGIFGAIAGFIENGWEGMVDGWYGFRHQNSEGRGQAADLKSTQAAIDQINGKLARVIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHNVYRDEALNNRFQIKGVELKSGYKDWILWISFAISSLVLL
VALLGFIMWACQKGNIRCNICI*
H3_Swi(F392D).r
(SEQ ID NO: 40)
TTTTCAATCTGATGGTCTTTCTCGTTGGTTTTCCCGATCAATCGATTCAGCTTC
H3_Swi(F392D).c
(SEQ ID NO: 41)
TCGGGAAAACCAACGAGAAAGACCATCAGATTGAAAAAGAATTCTCAG
PDI-H3 Switz-F392D DNA
(SEQ ID NO: 42)
ATGGCGAAAAACGTTGCGATTTTCGGCTTATTGTTTTCTCTTCTTGTGTTGGTTCCTTCTCAGATCTTC
GCGCAAAAACTTCCTGGAAATGACAATAGCACGGCAACGCTGTGCCTTGGGCACCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGGCTGGAGTCACTCAAAACGGAACAAGTTCT
TCTTGCATAAGGGGATCTAATAGTAGTTTCTTTAGTAGATTAAATTGGTTGACCCACTTAAACTCCAAA
TACCCAGCATTAAACGTGACTATGCCAAACAATGAACAATTTGACAAATTGTACATTTGGGGGGTTCAC
CACCCGGGTACGGACAAGGACCAAATCTTCCTGTATGCACAATCATCAGGAAGAATCACAGTATCTACC
AAAAGAAGCCAACAAGCTGTAATCCCGAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCAAAGCACTCTGAAATTGGCAACAGGAATGCGA
AATGTACCAGAGAGACAAACTAGAGGCATATTTGGCGCAATAGCGGGTTTCATAGAAAATGGTTGGGAG
GGAATGGTGGATGGTTGGTACGGCTTCAGGCATCAAAATTCTGAGGGAAGAGGACAAGCAGCAGATCTC
AAAAGCACTCAAGCAGCAATCGATCAAATCAATGGGAAGCTGAATCGATTGATCGGGAAAACCAACGAG
AAAGACCATCAGATTGAAAAAGAATTCTCAGAAGTAGAAGGGAGAATTCAGGACCTTGAGAAATATGTT
GAGGACACAAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATCAGA
AATGGAACTTATGACCACGATGTATACAGGGATGAAGCATTAAACAACCGGTTCCAGATCAAGGGAGTT
GAGCTGAAGTCAGGGTACAAAGATTGGATCCTATGGATTTCCTTTGCCATATCATGTTTTTTGCTTTGT
GTTGCTTTGTTGGGGTTCATCATGTGGGCCTGCCAAAAGGGCAACATTAGGTGCAACATTTGCATTTGA
PDI-H3 Switz-F392D AA
(SEQ ID NO: 43)
MAKNVAIFGLLFSLLVLVPSQIFAQKLPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWAGVTQNGTSSSCIRGSNSSFFSRLNWLTHLNSKYPALNVTMPNNEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKQSTLKLATGMR
NVPERQTRGIFGAIAGFIENGWEGMVDGWYGFRHQNSEGRGQAADLKSTQAAIDQINGKLNRLIGKTNE
KDHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHDVYRDEALNNRFQIKGVELKSGYKDWILWISFAISCFLLC
VALLGFIMWACQKGNIRCNICI*
H3_Swi(L431M).r
(SEQ ID NO: 44)
TTGTATGTTGGTTCTCCATGGCAACAAGAAGCTCCGCGTTGTATGACCAG
H3_Swi(L431M).c
(SEQ ID NO: 45)
GGAGCTTCTTGTTGCCATGGAGAACCAACATACAATTGATCTAACTGACTCAGA
PDI-H3 Switz-L431M DNA
(SEQ ID NO: 46)
ATGGCGAAAAACGTTGCGATTTTCGGCTTATTGTTTTCTCTTCTTGTGTTGGTTCCTTCTCAGATCTTC
GCGCAAAAACTTCCTGGAAATGACAATAGCACGGCAACGCTGTGCCTTGGGCACCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGGCTGGAGTCACTCAAAACGGAACAAGTTCT
TCTTGCATAAGGGGATCTAATAGTAGTTTCTTTAGTAGATTAAATTGGTTGACCCACTTAAACTCCAAA
TACCCAGCATTAAACGTGACTATGCCAAACAATGAACAATTTGACAAATTGTACATTTGGGGGGTTCAC
CACCCGGGTACGGACAAGGACCAAATCTTCCTGTATGCACAATCATCAGGAAGAATCACAGTATCTACC
AAAAGAAGCCAACAAGCTGTAATCCCGAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCAAAGCACTCTGAAATTGGCAACAGGAATGCGA
AATGTACCAGAGAGACAAACTAGAGGCATATTTGGCGCAATAGCGGGTTTCATAGAAAATGGTTGGGAG
GGAATGGTGGATGGTTGGTACGGCTTCAGGCATCAAAATTCTGAGGGAAGAGGACAAGCAGCAGATCTC
AAAAGCACTCAAGCAGCAATCGATCAAATCAATGGGAAGCTGAATCGATTGATCGGGAAAACCAACGAG
AAATTCCATCAGATTGAAAAAGAATTCTCAGAAGTAGAAGGGAGAATTCAGGACCTTGAGAAATATGTT
GAGGACACAAAAATAGATCTCTGGTCATACAACGCGGAGCTTCTTGTTGCCATGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATCAGA
AATGGAACTTATGACCACGATGTATACAGGGATGAAGCATTAAACAACCGGTTCCAGATCAAGGGAGTT
GAGCTGAAGTCAGGGTACAAAGATTGGATCCTATGGATTTCCTTTGCCATATCATGTTTTTTGCTTTGT
GTTGCTTTGTTGGGGTTCATCATGTGGGCCTGCCAAAAGGGCAACATTAGGTGCAACATTTGCATTTGA
PDI-H3 Switz-L431M AA
(SEQ ID NO: 47)
MAKNVAIFGLLFSLLVLVPSQIFAQKLPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWAGVTQNGTSSSCIRGSNSSFFSRLNWLTHLNSKYPALNVTMPNNEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKQSTLKLATGMR
NVPERQTRGIFGAIAGFIENGWEGMVDGWYGFRHQNSEGRGQAADLKSTQAAIDQINGKLNRLIGKTNE
KFHQIEKEFSEVEGRIQDLEKYVEDTKIDLWSYNAELLVAMENQHTIDLTDSEMNKLFEKTKKQLRENA
EDMGNGCFKIYHKCDNACIGSIRNGTYDHDVYRDEALNNRFQIKGVELKSGYKDWILWISFAISCFLLC
VALLGFIMWACQKGNIRCNICI
PDI-H3 Switz-CysTm DNA
(SEQ ID NO: 48)
ATGGCGAAAAACGTTGCGATTTTCGGCTTATTGTTTTCTCTTCTTGTGTTGGTTCCTTCTCAGATCTTC
GCGCAAAAACTTCCTGGAAATGACAATAGCACGGCAACGCTGTGCCTTGGGCACCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGGCTGGAGTCACTCAAAACGGAACAAGTTCT
TCTTGCATAAGGGGATCTAATAGTAGTTTCTTTAGTAGATTAAATTGGTTGACCCACTTAAACTCCAAA
TACCCAGCATTAAACGTGACTATGCCAAACAATGAACAATTTGACAAATTGTACATTTGGGGGGTTCAC
CACCCGGGTACGGACAAGGACCAAATCTTCCTGTATGCACAATCATCAGGAAGAATCACAGTATCTACC
AAAAGAAGCCAACAAGCTGTAATCCCGAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCAAAGCACTCTGAAATTGGCAACAGGAATGCGA
AATGTACCAGAGAGACAAACTAGAGGCATATTTGGCGCAATAGCGGGTTTCATAGAAAATGGTTGGGAG
GGAATGGTGGATGGTTGGTACGGCTTCAGGCATCAAAATTCTGAGGGAAGAGGACAAGCAGCAGATCTC
AAAAGCACTCAAGCAGCAATCGATCAAATCAATGGGAAGCTGAATCGATTGATCGGGAAAACCAACGAG
AAATTCCATCAGATTGAAAAAGAATTCTCAGAAGTAGAAGGGAGAATTCAGGACCTTGAGAAATATGTT
GAGGACACAAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATCAGA
AATGGAACTTATGACCACGATGTATACAGGGATGAAGCATTAAACAACCGGTTCCAGATCAAGGGAGTT
GAGCTGAAGTCAGGGTACAAAGATTGGATCCTATGGATTTCCTTTGCCATATCATCCCTTGTACTGTTA
GTTGCTTTGTTGGGGTTCATCATGTGGGCCTGCCAAAAGGGCAACATTAGGTGCAACATTTGCATTTGA
PDI-H3 Switz-CysTm AA
(SEQ ID NO: 49)
MAKNVAIFGLLFSLLVLVPSQIFAQKLPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWAGVTQNGTSSSCIRGSNSSFFSRLNWLTHLNSKYPALNVTMPNNEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKQSTLKLATGMR
NVPERQTRGIFGAIAGFIENGWEGMVDGWYGFRHQNSEGRGQAADLKSTQAAIDQINGKLNRLIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHDVYRDEALNNRFQIKGVELKSGYKDWILWISFAISSLVLL
VALLGFIMWACQKGNIRCNICI*
H3_Swi(N382A).r
(SEQ ID NO: 50)
TTTCCCGATCAATCGAGCCAGCTTCCCATTGATTTGATCGATTGCTGC
H3_Swi(N382A).c
(SEQ ID NO: 51)
TCAAATCAATGGGAAGCTGGCTCGATTGATCGGGAAAACCAACGAGAAATTCCA
PDI-H3 Switz-N382A-CysTm DNA
(SEQ ID NO: 52)
ATGGCGAAAAACGTTGCGATTTTCGGCTTATTGTTTTCTCTTCTTGTGTTGGTTCCTTCTCAGATCTTC
GCGCAAAAACTTCCTGGAAATGACAATAGCACGGCAACGCTGTGCCTTGGGCACCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGGCTGGAGTCACTCAAAACGGAACAAGTTCT
TCTTGCATAAGGGGATCTAATAGTAGTTTCTTTAGTAGATTAAATTGGTTGACCCACTTAAACTCCAAA
TACCCAGCATTAAACGTGACTATGCCAAACAATGAACAATTTGACAAATTGTACATTTGGGGGGTTCAC
CACCCGGGTACGGACAAGGACCAAATCTTCCTGTATGCACAATCATCAGGAAGAATCACAGTATCTACC
AAAAGAAGCCAACAAGCTGTAATCCCGAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCAAAGCACTCTGAAATTGGCAACAGGAATGCGA
AATGTACCAGAGAGACAAACTAGAGGCATATTTGGCGCAATAGCGGGTTTCATAGAAAATGGTTGGGAG
GGAATGGTGGATGGTTGGTACGGCTTCAGGCATCAAAATTCTGAGGGAAGAGGACAAGCAGCAGATCTC
AAAAGCACTCAAGCAGCAATCGATCAAATCAATGGGAAGCTGGCTCGATTGATCGGGAAAACCAACGAG
AAATTCCATCAGATTGAAAAAGAATTCTCAGAAGTAGAAGGGAGAATTCAGGACCTTGAGAAATATGTT
GAGGACACAAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATCAGA
AATGGAACTTATGACCACGATGTATACAGGGATGAAGCATTAAACAACCGGTTCCAGATCAAGGGAGTT
GAGCTGAAGTCAGGGTACAAAGATTGGATCCTATGGATTTCCTTTGCCATATCATCCCTTGTACTGTTA
GTTGCTTTGTTGGGGTTCATCATGTGGGCCTGCCAAAAGGGCAACATTAGGTGCAACATTTGCATTTGA
PDI-H3 Switz-N382A-CysTm AA
(SEQ ID NO: 53)
MAKNVAIFGLLFSLLVLVPSQIFAQKLPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWAGVTQNGTSSSCIRGSNSSFFSRLNWLTHLNSKYPALNVTMPNNEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKQSTLKLATGMR
NVPERQTRGIFGAIAGFIENGWEGMVDGWYGFRHQNSEGRGQAADLKSTQAAIDQINGKLARLIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHDVYRDEALNNRFQIKGVELKSGYKDWILWISFAISSLVLL
VALLGFIMWACQKGNIRCNICI*
H3_Swi(L384V).r
(SEQ ID NO: 54)
TTGGTTTTCCCGATCACTCGATTCAGCTTCCCATTGATTTGATCGATT
H3_Swi(L384V).c
(SEQ ID NO: 55)
GGGAAGCTGAATCGAGTGATCGGGAAAACCAACGAGAAATTCCATCAG
PDI-H3 Switz-L384V-CysTm DNA
(SEQ ID NO: 56)
ATGGCGAAAAACGTTGCGATTTTCGGCTTATTGTTTTCTCTTCTTGTGTTGGTTCCTTCTCAGATCTTC
GCGCAAAAACTTCCTGGAAATGACAATAGCACGGCAACGCTGTGCCTTGGGCACCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGGCTGGAGTCACTCAAAACGGAACAAGTTCT
TCTTGCATAAGGGGATCTAATAGTAGTTTCTTTAGTAGATTAAATTGGTTGACCCACTTAAACTCCAAA
TACCCAGCATTAAACGTGACTATGCCAAACAATGAACAATTTGACAAATTGTACATTTGGGGGGTTCAC
CACCCGGGTACGGACAAGGACCAAATCTTCCTGTATGCACAATCATCAGGAAGAATCACAGTATCTACC
AAAAGAAGCCAACAAGCTGTAATCCCGAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCAAAGCACTCTGAAATTGGCAACAGGAATGCGA
AATGTACCAGAGAGACAAACTAGAGGCATATTTGGCGCAATAGCGGGTTTCATAGAAAATGGTTGGGAG
GGAATGGTGGATGGTTGGTACGGCTTCAGGCATCAAAATTCTGAGGGAAGAGGACAAGCAGCAGATCTC
AAAAGCACTCAAGCAGCAATCGATCAAATCAATGGGAAGCTGAATCGAGTGATCGGGAAAACCAACGAG
AAATTCCATCAGATTGAAAAAGAATTCTCAGAAGTAGAAGGGAGAATTCAGGACCTTGAGAAATATGTT
GAGGACACAAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATCAGA
AATGGAACTTATGACCACGATGTATACAGGGATGAAGCATTAAACAACCGGTTCCAGATCAAGGGAGTT
GAGCTGAAGTCAGGGTACAAAGATTGGATCCTATGGATTTCCTTTGCCATATCATCCCTTGTACTGTTA
GTTGCTTTGTTGGGGTTCATCATGTGGGCCTGCCAAAAGGGCAACATTAGGTGCAACATTTGCATTTGA
PDI-H3 Switz-L384V-CysTm AA
(SEQ ID NO: 57)
MAKNVAIFGLLFSLLVLVPSQIFAQKLPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWAGVTQNGTSSSCIRGSNSSFFSRLNWLTHLNSKYPALNVTMPNNEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKQSTLKLATGMR
NVPERQTRGIFGAIAGFIENGWEGMVDGWYGFRHQNSEGRGQAADLKSTQAAIDQINGKLNRVIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHDVYRDEALNNRFQIKGVELKSGYKDWILWISFAISSLVLL
VALLGFIMWACQKGNIRCNICI*
PDI-H3 Switz-F392D-CysTm DNA
(SEQ ID NO: 58)
ATGGCGAAAAACGTTGCGATTTTCGGCTTATTGTTTTCTCTTCTTGTGTTGGTTCCTTCTCAGATCTTC
GCGCAAAAACTTCCTGGAAATGACAATAGCACGGCAACGCTGTGCCTTGGGCACCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGGCTGGAGTCACTCAAAACGGAACAAGTTCT
TCTTGCATAAGGGGATCTAATAGTAGTTTCTTTAGTAGATTAAATTGGTTGACCCACTTAAACTCCAAA
TACCCAGCATTAAACGTGACTATGCCAAACAATGAACAATTTGACAAATTGTACATTTGGGGGGTTCAC
CACCCGGGTACGGACAAGGACCAAATCTTCCTGTATGCACAATCATCAGGAAGAATCACAGTATCTACC
AAAAGAAGCCAACAAGCTGTAATCCCGAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCAAAGCACTCTGAAATTGGCAACAGGAATGCGA
AATGTACCAGAGAGACAAACTAGAGGCATATTTGGCGCAATAGCGGGTTTCATAGAAAATGGTTGGGAG
GGAATGGTGGATGGTTGGTACGGCTTCAGGCATCAAAATTCTGAGGGAAGAGGACAAGCAGCAGATCTC
AAAAGCACTCAAGCAGCAATCGATCAAATCAATGGGAAGCTGAATCGATTGATCGGGAAAACCAACGAG
AAAGACCATCAGATTGAAAAAGAATTCTCAGAAGTAGAAGGGAGAATTCAGGACCTTGAGAAATATGTT
GAGGACACAAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATCAGA
AATGGAACTTATGACCACGATGTATACAGGGATGAAGCATTAAACAACCGGTTCCAGATCAAGGGAGTT
GAGCTGAAGTCAGGGTACAAAGATTGGATCCTATGGATTTCCTTTGCCATATCATCCCTTGTACTGTTA
GTTGCTTTGTTGGGGTTCATCATGTGGGCCTGCCAAAAGGGCAACATTAGGTGCAACATTTGCATTTGA
PDI-H3 Switz-F392D-CysTm AA
(SEQ ID NO: 59)
MAKNVAIFGLLFSLLVLVPSQIFAQKLPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWAGVTQNGTSSSCIRGSNSSFFSRLNWLTHLNSKYPALNVTMPNNEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKQSTLKLATGMR
NVPERQTRGIFGAIAGFIENGWEGMVDGWYGFRHQNSEGRGQAADLKSTQAAIDQINGKLNRLIGKTNE
KDHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHDVYRDEALNNRFQIKGVELKSGYKDWILWISFAISSLVLL
VALLGFIMWACQKGNIRCNICI*
PDI-H3 Switz-L431M-CysTm DNA
(SEQ ID NO: 60)
ATGGCGAAAAACGTTGCGATTTTCGGCTTATTGTTTTCTCTTCTTGTGTTGGTTCCTTCTCAGATCTTC
GCGCAAAAACTTCCTGGAAATGACAATAGCACGGCAACGCTGTGCCTTGGGCACCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGGCTGGAGTCACTCAAAACGGAACAAGTTCT
TCTTGCATAAGGGGATCTAATAGTAGTTTCTTTAGTAGATTAAATTGGTTGACCCACTTAAACTCCAAA
TACCCAGCATTAAACGTGACTATGCCAAACAATGAACAATTTGACAAATTGTACATTTGGGGGGTTCAC
CACCCGGGTACGGACAAGGACCAAATCTTCCTGTATGCACAATCATCAGGAAGAATCACAGTATCTACC
AAAAGAAGCCAACAAGCTGTAATCCCGAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCAAAGCACTCTGAAATTGGCAACAGGAATGCGA
AATGTACCAGAGAGACAAACTAGAGGCATATTTGGCGCAATAGCGGGTTTCATAGAAAATGGTTGGGAG
GGAATGGTGGATGGTTGGTACGGCTTCAGGCATCAAAATTCTGAGGGAAGAGGACAAGCAGCAGATCTC
AAAAGCACTCAAGCAGCAATCGATCAAATCAATGGGAAGCTGAATCGATTGATCGGGAAAACCAACGAG
AAATTCCATCAGATTGAAAAAGAATTCTCAGAAGTAGAAGGGAGAATTCAGGACCTTGAGAAATATGTT
GAGGACACAAAAATAGATCTCTGGTCATACAACGCGGAGCTTCTTGTTGCCATGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATCAGA
AATGGAACTTATGACCACGATGTATACAGGGATGAAGCATTAAACAACCGGTTCCAGATCAAGGGAGTT
GAGCTGAAGTCAGGGTACAAAGATTGGATCCTATGGATTTCCTTTGCCATATCATCCCTTGTACTGTTA
GTTGCTTTGTTGGGGTTCATCATGTGGGCCTGCCAAAAGGGCAACATTAGGTGCAACATTTGCATTTGA
PDI-H3 Switz-L431M-CysTm AA
(SEQ ID NO: 61)
MAKNVAIFGLLFSLLVLVPSQIFAQKLPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWAGVTQNGTSSSCIRGSNSSFFSRLNWLTHLNSKYPALNVTMPNNEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKQSTLKLATGMR
NVPERQTRGIFGAIAGFIENGWEGMVDGWYGFRHQNSEGRGQAADLKSTQAAIDQINGKLNRLIGKTNE
KFHQIEKEFSEVEGRIQDLEKYVEDTKIDLWSYNAELLVAMENQHTIDLTDSEMNKLFEKTKKQLRENA
EDMGNGCFKIYHKCDNACIGSIRNGTYDHDVYRDEALNNRFQIKGVELKSGYKDWILWISFAISSLVLL
VALLGFIMWACQKGNIRCNICI*
Cloning vector 1190 from left to right T-DNA
(SEQ ID NO: 62)
TGGCAGGATATATTGTGGTGTAAACAAATTGACGCTTAGACAACTTAATAACACATTGCGGACGTTTTT
AATGTACTGAATTAACGCCGAATCCCGGGCTGGTATATTTATATGTTGTCAAATAACTCAAAAACCATA
AAAGTTTAAGTTAGCAAGTGTGTACATTTTTACTTGAACAAAAATATTCACCTACTACTGTTATAAATC
ATTATTAAACATTAGAGTAAAGAAATATGGATGATAAGAACAAGAGTAGTGATATTTTGACAACAATTT
TGTTGCAACATTTGAGAAAATTTTGTTGTTCTCTCTTTTCATTGGTCAAAAACAATAGAGAGAGAAAAA
GGAAGAGGGAGAATAAAAACATAATGTGAGTATGAGAGAGAAAGTTGTACAAAAGTTGTACCAAAATAG
TTGTACAAATATCATTGAGGAATTTGACAAAAGCTACACAAATAAGGGTTAATTGCTGTAAATAAATAA
GGATGACGCATTAGAGAGATGTACCATTAGAGAATTTTTGGCAAGTCATTAAAAAGAAAGAATAAATTA
TTTTTAAAATTAAAAGTTGAGTCATTTGATTAAACATGTGATTATTTAATGAATTGATGAAAGAGTTGG
ATTAAAGTTGTATTAGTAATTAGAATTTGGTGTCAAATTTAATTTGACATTTGATCTTTTCCTATATAT
TGCCCCATAGAGTCAGTTAACTCATTTTTATATTTCATAGATCAAATAAGAGAAATAACGGTATATTAA
TCCCTCCAAAAAAAAAAAACGGTATATTTACTAAAAAATCTAAGCCACGTAGGAGGATAACAGGATCCC
CGTAGGAGGATAACATCCAATCCAACCAATCACAACAATCCTGATGAGATAACCCACTTTAAGCCCACG
CATCTGTGGCACATCTACATTATCTAAATCACACATTCTTCCACACATCTGAGCCACACAAAAACCAAT
CCACATCTTTATCACCCATTCTATAAAAAATCACACTTTGTGAGTCTACACTTTGATTCCCTTCAAACA
CATACAAAGAGAAGAGACTAATTAATTAATTAATCATCTTGAGAGAAAATGGAACGAGCTATACAAGGA
AACGACGCTAGGGAACAAGCTAACAGTGAACGTTGGGATGGAGGATCAGGAGGTACCACTTCTCCCTTC
AAACTTCCTGACGAAAGTCCGAGTTGGACTGAGTGGCGGCTACATAACGATGAGACGAATTCGAATCAA
GATAATCCCCTTGGTTTCAAGGAAAGCTGGGGTTTCGGGAAAGTTGTATTTAAGAGATATCTCAGATAC
GACAGGACGGAAGCTTCACTGCACAGAGTCCTTGGATCTTGGACGGGAGATTCGGTTAACTATGCAGCA
TCTCGATTTTTCGGTTTCGACCAGATCGGATGTACCTATAGTATTCGGTTTCGAGGAGTTAGTATCACC
GTTTCTGGAGGGTCGCGAACTCTTCAGCATCTCTGTGAGATGGCAATTCGGTCTAAGCAAGAACTGCTA
CAGCTTGCCCCAATCGAAGTGGAAAGTAATGTATCAAGAGGATGCCCTGAAGGTACTCAAACCTTCGAA
AAAGAAAGCGAGTAAGTTAAAATGCTTCTTCGTCTCCTATTTATAATATGGTTTGTTATTGTTAATTTT
GTTCTTGTAGAAGAGCTTAATTAATCGTTGTTGTTATGAAATACTATTTGTATGAGATGAACTGGTGTA
ATGTAATTCATTTACATAAGTGGAGTCAGAATCAGAATGTTTCCTCCATAACTAACTAGACATGAAGAC
CTGCCGCGTACAATTGTCTTATATTTGAACAACTAAAATTGAACATCTTTTGCCACAACTTTATAAGTG
GTTAATATAGCTCAAATATATGGTCAAGTTCAATAGATTAATAATGGAAATATCAGTTATCGAAATTCA
TTAACAATCAACTTAACGTTATTAACTACTAATTTTATATCATCCCCTTTGATAAATGATAGTACACCA
ATTAGGAAGGAGCATGCTCGCCTAGGAGATTGTCGTTTCCCGCCTTCAGTTTGCAAGCTGCTCTAGCCG
TGTAGCCAATACGCAAACCGCCTCTCCCCGCGCGTTGGGAATTACTAGCGCGTGTCGACAAGCTTGCAT
GCCGGTCAACATGGTGGAGCACGACACACTTGTCTACTCCAAAAATATCAAAGATACAGTCTCAGAAGA
CCAAAGGGCAATTGAGACTTTTCAACAAAGGGTAATATCCGGAAACCTCCTCGGATTCCATTGCCCAGC
TATCTGTCACTTTATTGTGAAGATAGTGGAAAAGGAAGGTGGCTCCTACAAATGCCATCATTGCGATAA
AGGAAAGGCCATCGTTGAAGATGCCTCTGCCGACAGTGGTCCCAAAGATGGACCCCCACCCACGAGGAG
CATCGTGGAAAAAGAAGACGTTCCAACCACGTCTTCAAAGCAAGTGGATTGATGTGATAACATGGTGGA
GCACGACACACTTGTCTACTCCAAAAATATCAAAGATACAGTCTCAGAAGACCAAAGGGCAATTGAGAC
TTTTCAACAAAGGGTAATATCCGGAAACCTCCTCGGATTCCATTGCCCAGCTATCTGTCACTTTATTGT
GAAGATAGTGGAAAAGGAAGGTGGCTCCTACAAATGCCATCATTGCGATAAAGGAAAGGCCATCGTTGA
AGATGCCTCTGCCGACAGTGGTCCCAAAGATGGACCCCCACCCACGAGGAGCATCGTGGAAAAAGAAGA
CGTTCCAACCACGTCTTCAAAGCAAGTGGATTGATGTGATATCTCCACTGACGTAAGGGATGACGCACA
ATCCCACTATCCTTCGCAAGACCCTTCCTCTATATAAGGAAGTTCATTTCATTTGGAGAGGTATTAAAA
TCTTAATAGGTTTTGATAAAAGCGAACGTGGGGAAACCCGAACCAAACCTTCTTCTAAACTCTCTCTCA
TCTCTCTTAAAGCAAACTTCTCTCTTGTCTTTCTTGCGTGAGCGATCTTCAACGTTGTCAGATCGTGCT
TCGGCACCGCGGATGGCGAAAAACGTTGCGATTTTCGGCTTATTGTTTTCTCTTCTTGTGTTGGTTCCT
TCTCAGATCTTCGCCTGCAGGCTCCTCAGCCAAAACGACACCCCCATCTGTCTATCCACTGGCCCCTGG
ATCTGCTGCCCAAACTAACTCCATGGTGACCCTGGGATGCCTGGTCAAGGGCTATTTCCCTGAGCCAGT
GACAGTGACCTGGAACTCTGGATCCCTGTCCAGCGGTGTGCACACCTTCCCAGCTGTCCTGCAGTCTGA
CCTCTACACTCTGAGCAGCTCAGTGACTGTCCCCTCCAGCACCTGGCCCAGCGAGACCGTCACCTGCAA
CGTTGCCCACCCGGCCAGCAGCACCAAGGTGGACAAGAAAATTGTGCCCAGGGATTGTGGTTGTAAGCC
TTGCATATGTACAGTCCCAGAAGTATCATCTGTCTTCATCTTCCCCCCAAAGCCCAAGGATGTGCTCAC
CATTACTCTGACTCCTAAGGTCACGTGTGTTGTGGTAGACATCAGCAAGGATGATCCCGAGGTCCAGTT
CAGCTGGTTTGTAGATGATGTGGAGGTGCACACAGCTCAGACGCAACCCCGGGAGGAGCAGTTCAACAG
CACTTTCCGCTCAGTCAGTGAACTTCCCATCATGCACCAGGACTGGCTCAATGGCAAGGAGCGATCGCT
CACCATCACCATCACCATCACCATCACCATTAAAGGCCTATTTTCTTTAGTTTGAATTTACTGTTATTC
GGTGTGCATTTCTATGTTTGGTGAGCGGTTTTCTGTGCTCAGAGTGTGTTTATTTTATGTAATTTAATT
TCTTTGTGAGCTCCTGTTTAGCAGGTCGTCCCTTCAGCAAGGACACAAAAAGATTTTAATTTTATTAAA
AAAAAAAAAAAAAAAGACCGGGAATTCGATATCAAGCTTATCGACCTGCAGATCGTTCAAACATTTGGC
AATAAAGTTTCTTAAGATTGAATCCTGTTGCCGGTCTTGCGATGATTATCATATAATTTCTGTTGAATT
ACGTTAAGCATGTAATAATTAACATGTAATGCATGACGTTATTTATGAGATGGGTTTTTATGATTAGAG
TCCCGCAATTATACATTTAATACGCGATAGAAAACAAAATATAGCGCGCAAACTAGGATAAATTATCGC
GCGCGGTGTCATCTATGTTACTAGATCTCTAGAGTCTCAAGCTTGGCGCGCCCACGTGACTAGTGGCAC
TGGCCGTCGTTTTACAACGTCGTGACTGGGAAAACCCTGGCGTTACCCAACTTAATCGCCTTGCAGCAC
ATCCCCCTTTCGCCAGCTGGCGTAATAGCGAAGAGGCCCGCACCGATCGCCCTTCCCAACAGTTGCGCA
GCCTGAATGGCGAATGCTAGAGCAGCTTGAGCTTGGATCAGATTGTCGTTTCCCGCCTTCAGTTTAAAC
TATCAGTGTTTGACAGGATATATTGGCGGGTAAACCTAAGAGAAAAGAGCGTTTA
Construct 1314 from 2X35S prom to NOS term
(SEQ ID NO: 63)
GTCAACATGGTGGAGCACGACACACTTGTCTACTCCAAAAATATCAAAGATACAGTCTCAGAAGACCAA
AGGGCAATTGAGACTTTTCAACAAAGGGTAATATCCGGAAACCTCCTCGGATTCCATTGCCCAGCTATC
TGTCACTTTATTGTGAAGATAGTGGAAAAGGAAGGTGGCTCCTACAAATGCCATCATTGCGATAAAGGA
AAGGCCATCGTTGAAGATGCCTCTGCCGACAGTGGTCCCAAAGATGGACCCCCACCCACGAGGAGCATC
GTGGAAAAAGAAGACGTTCCAACCACGTCTTCAAAGCAAGTGGATTGATGTGATAACATGGTGGAGCAC
GACACACTTGTCTACTCCAAAAATATCAAAGATACAGTCTCAGAAGACCAAAGGGCAATTGAGACTTTT
CAACAAAGGGTAATATCCGGAAACCTCCTCGGATTCCATTGCCCAGCTATCTGTCACTTTATTGTGAAG
ATAGTGGAAAAGGAAGGTGGCTCCTACAAATGCCATCATTGCGATAAAGGAAAGGCCATCGTTGAAGAT
GCCTCTGCCGACAGTGGTCCCAAAGATGGACCCCCACCCACGAGGAGCATCGTGGAAAAAGAAGACGTT
CCAACCACGTCTTCAAAGCAAGTGGATTGATGTGATATCTCCACTGACGTAAGGGATGACGCACAATCC
CACTATCCTTCGCAAGACCCTTCCTCTATATAAGGAAGTTCATTTCATTTGGAGAGGTATTAAAATCTT
AATAGGTTTTGATAAAAGCGAACGTGGGGAAACCCGAACCAAACCTTCTTCTAAACTCTCTCTCATCTC
TCTTAAAGCAAACTTCTCTCTTGTCTTTCTTGCGTGAGCGATCTTCAACGTTGTCAGATCGTGCTTCGG
CACCAGTACAATGGCGAAAAACGTTGCGATTTTCGGCTTATTGTTTTCTCTTCTTGTGTTGGTTCCTTC
TCAGATCTTCGCTGACACATTATGTATAGGTTATCATGCGAACAATTCAACAGACACTGTAGACACAGT
ACTAGAAAAGAATGTAACAGTAACACACTCTGTTAACCTTCTAGAAGACAAGCATAACGGGAAACTATG
CAAACTAAGAGGGGTAGCCCCATTGCATTTGGGTAAATGTAACATTGCTGGCTGGATCCTGGGAAATCC
AGAGTGTGAATCACTCTCCACAGCAAGCTCATGGTCCTACATTGTGGAAACACCTAGTTCAGACAATGG
AACGTGTTACCCAGGAGATTTCATCGATTATGAGGAGCTAAGAGAGCAATTGAGCTCAGTGTCATCATT
TGAAAGGTTTGAGATATTCCCCAAGACAAGTTCATGGCCCAATCATGACTCGAACAAAGGTGTAACGGC
AGCATGTCCTCATGCTGGAGCAAAAAGCTTCTACAAAAATTTAATATGGCTAGTTAAAAAAGGAAATTC
ATACCCAAAGCTCAGCAAATCCTACATTAATGATAAAGGGAAAGAAGTCCTCGTGCTATGGGGCATTCA
CCATCCATCTACTAGTGCTGACCAACAAAGTCTCTATCAGAATGCAGATGCATATGTTTTTGTGGGGTC
ATCAAGATACAGCAAGAAGTTCAAGCCGGAAATAGCAATAAGACCCAAAGTGAGGGATCAAGAAGGGAG
AATGAACTATTACTGGACACTAGTAGAGCCGGGAGACAAAATAACATTCGAAGCAACTGGAAATCTAGT
GGTACCGAGATATGCATTCGCAATGGAAAGAAATGCTGGATCTGGTATTATCATTTCAGATACACCAGT
CCACGATTGCAATACAACTTGTCAAACACCCAAGGGTGCTATAAACACCAGCCTCCCATTTCAGAATAT
ACATCCGATCACAATTGGAAAATGTCCAAAATATGTAAAAAGCACAAAATTGAGACTGGCCACAGGATT
GAGGAATATCCCGTCTATTCAATCTAGAGGACTATTTGGGGCCATTGCCGGTTTCATTGAAGGGGGGTG
GACAGGGATGGTAGATGGATGGTACGGTTATCACCATCAAAATGAGCAGGGGTCAGGATATGCAGCCGA
CCTGAAGAGCACACAGAATGCCATTGACGAGATTACTAACAAAGTAAATTCTGTTATTGAAAAGATGAA
TACACAGTTCACAGCAGTAGGTAAAGAGTTCAACCACCTGGAAAAAAGAATAGAGAATTTAAATAAAAA
AGTTGATGATGGTTTCCTGGACATTTGGACTTACAATGCCGAACTGTTGGTTCTATTGGAAAATGAAAG
AACTTTGGACTACCACGATTCAAATGTGAAGAACTTATATGAAAAGGTAAGAAGCCAGCTAAAAAACAA
TGCCAAGGAAATTGGAAACGGCTGCTTTGAATTTTACCACAAATGCGATAACACGTGCATGGAAAGTGT
CAAAAATGGGACTTATGACTACCCAAAATACTCAGAGGAAGCAAAATTAAACAGAGAAGAAATAGATGG
GGTAAAGCTGGAATCAACAAGGATTTACCAGATTTTGGCGATCTATTCAACTGTCGCCAGTTCATTGGT
ACTGGTAGTCTCCCTGGGGGCAATCAGTTTCTGGATGTGCTCTAATGGGTCTCTACAGTGTAGAATATG
TATTTAAAGGCCTATTTTCTTTAGTTTGAATTTACTGTTATTCGGTGTGCATTTCTATGTTTGGTGAGC
GGTTTTCTGTGCTCAGAGTGTGTTTATTTTATGTAATTTAATTTCTTTGTGAGCTCCTGTTTAGCAGGT
CGTCCCTTCAGCAAGGACACAAAAAGATTTTAATTTTATTAAAAAAAAAAAAAAAAAAGACCGGGAATT
CGATATCAAGCTTATCGACCTGCAGATCGTTCAAACATTTGGCAATAAAGTTTCTTAAGATTGAATCCT
GTTGCCGGTCTTGCGATGATTATCATATAATTTCTGTTGAATTACGTTAAGCATGTAATAATTAACATG
TAATGCATGACGTTATTTATGAGATGGGTTTTTATGATTAGAGTCCCGCAATTATACATTTAATACGCG
ATAGAAAACAAAATATAGCGCGCAAACTAGGATAAATTATCGCGCGCGGTGTCATCTATGTTACTAGAT
Construct 2980 from 2X35S prom to NOS term
(SEQ ID NO: 64)
GTCAACATGGTGGAGCACGACACACTTGTCTACTCCAAAAATATCAAAGATACAGTCTCAGAAGACCAA
AGGGCAATTGAGACTTTTCAACAAAGGGTAATATCCGGAAACCTCCTCGGATTCCATTGCCCAGCTATC
TGTCACTTTATTGTGAAGATAGTGGAAAAGGAAGGTGGCTCCTACAAATGCCATCATTGCGATAAAGGA
AAGGCCATCGTTGAAGATGCCTCTGCCGACAGTGGTCCCAAAGATGGACCCCCACCCACGAGGAGCATC
GTGGAAAAAGAAGACGTTCCAACCACGTCTTCAAAGCAAGTGGATTGATGTGATAACATGGTGGAGCAC
GACACACTTGTCTACTCCAAAAATATCAAAGATACAGTCTCAGAAGACCAAAGGGCAATTGAGACTTTT
CAACAAAGGGTAATATCCGGAAACCTCCTCGGATTCCATTGCCCAGCTATCTGTCACTTTATTGTGAAG
ATAGTGGAAAAGGAAGGTGGCTCCTACAAATGCCATCATTGCGATAAAGGAAAGGCCATCGTTGAAGAT
GCCTCTGCCGACAGTGGTCCCAAAGATGGACCCCCACCCACGAGGAGCATCGTGGAAAAAGAAGACGTT
CCAACCACGTCTTCAAAGCAAGTGGATTGATGTGATATCTCCACTGACGTAAGGGATGACGCACAATCC
CACTATCCTTCGCAAGACCCTTCCTCTATATAAGGAAGTTCATTTCATTTGGAGAGGTATTAAAATCTT
AATAGGTTTTGATAAAAGCGAACGTGGGGAAACCCGAACCAAACCTTCTTCTAAACTCTCTCTCATCTC
TCTTAAAGCAAACTTCTCTCTTGTCTTTCTTGCGTGAGCGATCTTCAACGTTGTCAGATCGTGCTTCGG
CACCAGTACAATGGCGAAAAACGTTGCGATTTTCGGCTTATTGTTTTCTCTTCTTGTGTTGGTTCCTTC
TCAGATCTTCGCGGACACATTATGTATAGGTTATCATGCGAACAATTCAACAGACACTGTAGACACAGT
ACTAGAAAAGAATGTAACAGTAACACACTCTGTTAACCTTCTAGAAGACAAGCATAACGGGAAACTATG
CAAACTAAGAGGGGTAGCCCCATTGCATTTGGGTAAATGTAACATTGCTGGCTGGATCCTGGGAAATCC
AGAGTGTGAATCACTCTCCACAGCAAGCTCATGGTCCTACATTGTGGAAACACCTAGTTCAGACAATGG
AACGTGTTACCCAGGAGATTTCATCGATTATGAGGAGCTAAGAGAGCAATTGAGCTCAGTGTCATCATT
TGAAAGGTTTGAGATATTCCCCAAGACAAGTTCATGGCCCAATCATGACTCGAACAAAGGTGTAACGGC
AGCATGTCCTCATGCTGGAGCAAAAAGCTTCTACAAAAATTTAATATGGCTAGTTAAAAAAGGAAATTC
ATACCCAAAGCTCAGCAAATCCTACATTAATGATAAAGGGAAAGAAGTCCTCGTGCTATGGGGCATTCA
CCATCCATCTACTAGTGCTGACCAACAAAGTCTCTATCAGAATGCAGATGCATATGTTTTTGTGGGGTC
ATCAAGATACAGCAAGAAGTTCAAGCCGGAAATAGCAATAAGACCCAAAGTGAGGGATCAAGAAGGGAG
AATGAACTATTACTGGACACTAGTAGAGCCGGGAGACAAAATAACATTCGAAGCAACTGGAAATCTAGT
GGTACCGAGATATGCATTCGCAATGGAAAGAAATGCTGGATCTGGTATTATCATTTCAGATACACCAGT
CCACGATTGCAATACAACTTGTCAAACACCCAAGGGTGCTATAAACACCAGCCTCCCATTTCAGAATAT
ACATCCGATCACAATTGGAAAATGTCCAAAATATGTAAAAAGCACAAAATTGAGACTGGCCACAGGATT
GAGGAATATCCCGTCTATTCAATCTAGAGGACTATTTGGGGCCATTGCCGGTTTCATTGAAGGGGGGTG
GACAGGGATGGTAGATGGATGGTACGGTTATCACCATCAAAATGAGCAGGGGTCAGGATATGCAGCCGA
CCTGAAGAGCACACAGAATGCCATTGACGAGATTACTAACAAAGTAAATTCTGTTATTGAAAAGATGAA
TACACAGGACACAGCAGTAGGTAAAGAGTTCAACCACCTGGAAAAAAGAATAGAGAATTTAAATAAAAA
AGTTGATGATGGTTTCCTGGACATTTGGACTTACAATGCCGAACTGTTGGTTCTATTGGAAAATGAAAG
AACTTTGGACTACCACGATTCAAATGTGAAGAACTTATATGAAAAGGTAAGAAGCCAGCTAAAAAACAA
TGCCAAGGAAATTGGAAACGGCTGCTTTGAATTTTACCACAAATGCGATAACACGTGCATGGAAAGTGT
CAAAAATGGGACTTATGACTACCCAAAATACTCAGAGGAAGCAAAATTAAACAGAGAAGAAATAGATGG
GGTAAAGCTGGAATCAACAAGGATTTACCAGATTTTGGCGATCTATTCAACTGTCGCCAGTTCATTGGT
ACTGGTAGTCTCCCTGGGGGCAATCAGTTTCTGGATGTGCTCTAATGGGTCTCTACAGTGTAGAATATG
TATTTAAAGGCCTATTTTCTTTAGTTTGAATTTACTGTTATTCGGTGTGCATTTCTATGTTTGGTGAGC
GGTTTTCTGTGCTCAGAGTGTGTTTATTTTATGTAATTTAATTTCTTTGTGAGCTCCTGTTTAGCAGGT
CGTCCCTTCAGCAAGGACACAAAAAGATTTTAATTTTATTAAAAAAAAAAAAAAAAAAGACCGGGAATT
CGATATCAAGCTTATCGACCTGCAGATCGTTCAAACATTTGGCAATAAAGTTTCTTAAGATTGAATCCT
GTTGCCGGTCTTGCGATGATTATCATATAATTTCTGTTGAATTACGTTAAGCATGTAATAATTAACATG
TAATGCATGACGTTATTTATGAGATGGGTTTTTATGATTAGAGTCCCGCAATTATACATTTAATACGCG
ATAGAAAACAAAATATAGCGCGCAAACTAGGATAAATTATCGCGCGCGGTGTCATCTATGTTACTAGAT
Construct 2995 from 2X35S prom to NOS term
(SEQ ID NO: 65)
GTCAACATGGTGGAGCACGACACACTTGTCTACTCCAAAAATATCAAAGATACAGTCTCAGAAGACCAA
AGGGCAATTGAGACTTTTCAACAAAGGGTAATATCCGGAAACCTCCTCGGATTCCATTGCCCAGCTATC
TGTCACTTTATTGTGAAGATAGTGGAAAAGGAAGGTGGCTCCTACAAATGCCATCATTGCGATAAAGGA
AAGGCCATCGTTGAAGATGCCTCTGCCGACAGTGGTCCCAAAGATGGACCCCCACCCACGAGGAGCATC
GTGGAAAAAGAAGACGTTCCAACCACGTCTTCAAAGCAAGTGGATTGATGTGATAACATGGTGGAGCAC
GACACACTTGTCTACTCCAAAAATATCAAAGATACAGTCTCAGAAGACCAAAGGGCAATTGAGACTTTT
CAACAAAGGGTAATATCCGGAAACCTCCTCGGATTCCATTGCCCAGCTATCTGTCACTTTATTGTGAAG
ATAGTGGAAAAGGAAGGTGGCTCCTACAAATGCCATCATTGCGATAAAGGAAAGGCCATCGTTGAAGAT
GCCTCTGCCGACAGTGGTCCCAAAGATGGACCCCCACCCACGAGGAGCATCGTGGAAAAAGAAGACGTT
CCAACCACGTCTTCAAAGCAAGTGGATTGATGTGATATCTCCACTGACGTAAGGGATGACGCACAATCC
CACTATCCTTCGCAAGACCCTTCCTCTATATAAGGAAGTTCATTTCATTTGGAGAGGTATTAAAATCTT
AATAGGTTTTGATAAAAGCGAACGTGGGGAAACCCGAACCAAACCTTCTTCTAAACTCTCTCTCATCTC
TCTTAAAGCAAACTTCTCTCTTGTCTTTCTTGCGTGAGCGATCTTCAACGTTGTCAGATCGTGCTTCGG
CACCAGTACAATGGCGAAAAACGTTGCGATTTTCGGCTTATTGTTTTCTCTTCTTGTGTTGGTTCCTTC
TCAGATCTTCGCGGACACATTATGTATAGGTTATCATGCGAACAATTCAACAGACACTGTAGACACAGT
ACTAGAAAAGAATGTAACAGTAACACACTCTGTTAACCTTCTAGAAGACAAGCATAACGGGAAACTATG
CAAACTAAGAGGGGTAGCCCCATTGCATTTGGGTAAATGTAACATTGCTGGCTGGATCCTGGGAAATCC
AGAGTGTGAATCACTCTCCACAGCAAGCTCATGGTCCTACATTGTGGAAACACCTAGTTCAGACAATGG
AACGTGTTACCCAGGAGATTTCATCGATTATGAGGAGCTAAGAGAGCAATTGAGCTCAGTGTCATCATT
TGAAAGGTTTGAGATATTCCCCAAGACAAGTTCATGGCCCAATCATGACTCGAACAAAGGTGTAACGGC
AGCATGTCCTCATGCTGGAGCAAAAAGCTTCTACAAAAATTTAATATGGCTAGTTAAAAAAGGAAATTC
ATACCCAAAGCTCAGCAAATCCTACATTAATGATAAAGGGAAAGAAGTCCTCGTGCTATGGGGCATTCA
CCATCCATCTACTAGTGCTGACCAACAAAGTCTCTATCAGAATGCAGATGCATATGTTTTTGTGGGGTC
ATCAAGATACAGCAAGAAGTTCAAGCCGGAAATAGCAATAAGACCCAAAGTGAGGGATCAAGAAGGGAG
AATGAACTATTACTGGACACTAGTAGAGCCGGGAGACAAAATAACATTCGAAGCAACTGGAAATCTAGT
GGTACCGAGATATGCATTCGCAATGGAAAGAAATGCTGGATCTGGTATTATCATTTCAGATACACCAGT
CCACGATTGCAATACAACTTGTCAAACACCCAAGGGTGCTATAAACACCAGCCTCCCATTTCAGAATAT
ACATCCGATCACAATTGGAAAATGTCCAAAATATGTAAAAAGCACAAAATTGAGACTGGCCACAGGATT
GAGGAATATCCCGTCTATTCAATCTAGAGGACTATTTGGGGCCATTGCCGGTTTCATTGAAGGGGGGTG
GACAGGGATGGTAGATGGATGGTACGGTTATCACCATCAAAATGAGCAGGGGTCAGGATATGCAGCCGA
CCTGAAGAGCACACAGAATGCCATTGACGAGATTACTAACAAAGTAAATTCTGTTATTGAAAAGATGAA
TACACAGGACACAGCAGTAGGTAAAGAGTTCAACCACCTGGAAAAAAGAATAGAGAATTTAAATAAAAA
AGTTGATGATGGTTTCCTGGACATTTGGACTTACAATGCCGAACTGTTGGTTCTAATGGAAAATGAAAG
AACTTTGGACTACCACGATTCAAATGTGAAGAACTTATATGAAAAGGTAAGAAGCCAGCTAAAAAACAA
TGCCAAGGAAATTGGAAACGGCTGCTTTGAATTTTACCACAAATGCGATAACACGTGCATGGAAAGTGT
CAAAAATGGGACTTATGACTACCCAAAATACTCAGAGGAAGCAAAATTAAACAGAGAAGAAATAGATGG
GGTAAAGCTGGAATCAACAAGGATTTACCAGATTTTGGCGATCTATTCAACTGTCGCCAGTTCATTGGT
ACTGGTAGTCTCCCTGGGGGCAATCAGTTTCTGGATGTGCTCTAATGGGTCTCTACAGTGTAGAATATG
TATTTAAAGGCCTATTTTCTTTAGTTTGAATTTACTGTTATTCGGTGTGCATTTCTATGTTTGGTGAGC
GGTTTTCTGTGCTCAGAGTGTGTTTATTTTATGTAATTTAATTTCTTTGTGAGCTCCTGTTTAGCAGGT
CGTCCCTTCAGCAAGGACACAAAAAGATTTTAATTTTATTAAAAAAAAAAAAAAAAAAGACCGGGAATT
CGATATCAAGCTTATCGACCTGCAGATCGTTCAAACATTTGGCAATAAAGTTTCTTAAGATTGAATCCT
GTTGCCGGTCTTGCGATGATTATCATATAATTTCTGTTGAATTACGTTAAGCATGTAATAATTAACATG
TAATGCATGACGTTATTTATGAGATGGGTTTTTATGATTAGAGTCCCGCAATTATACATTTAATACGCG
ATAGAAAACAAAATATAGCGCGCAAACTAGGATAAATTATCGCGCGCGGTGTCATCTATGTTACTAGAT
Cloning vector 3556 from left to right T-DNA
(SEQ ID NO: 66)
TGGCAGGATATATTGTGGTGTAAACAAATTGACGCTTAGACAACTTAATAACACATTGCGGACGTTTTT
AATGTACTGAATTAACGCCGAATCCCGGGCTGGTATATTTATATGTTGTCAAATAACTCAAAAACCATA
AAAGTTTAAGTTAGCAAGTGTGTACATTTTTACTTGAACAAAAATATTCACCTACTACTGTTATAAATC
ATTATTAAACATTAGAGTAAAGAAATATGGATGATAAGAACAAGAGTAGTGATATTTTGACAACAATTT
TGTTGCAACATTTGAGAAAATTTTGTTGTTCTCTCTTTTCATTGGTCAAAAACAATAGAGAGAGAAAAA
GGAAGAGGGAGAATAAAAACATAATGTGAGTATGAGAGAGAAAGTTGTACAAAAGTTGTACCAAAATAG
TTGTACAAATATCATTGAGGAATTTGACAAAAGCTACACAAATAAGGGTTAATTGCTGTAAATAAATAA
GGATGACGCATTAGAGAGATGTACCATTAGAGAATTTTTGGCAAGTCATTAAAAAGAAAGAATAAATTA
TTTTTAAAATTAAAAGTTGAGTCATTTGATTAAACATGTGATTATTTAATGAATTGATGAAAGAGTTGG
ATTAAAGTTGTATTAGTAATTAGAATTTGGTGTCAAATTTAATTTGACATTTGATCTTTTCCTATATAT
TGCCCCATAGAGTCAGTTAACTCATTTTTATATTTCATAGATCAAATAAGAGAAATAACGGTATATTAA
TCCCTCCAAAAAAAAAAAACGGTATATTTACTAAAAAATCTAAGCCACGTAGGAGGATAACAGGATCCC
CGTAGGAGGATAACATCCAATCCAACCAATCACAACAATCCTGATGAGATAACCCACTTTAAGCCCACG
CATCTGTGGCACATCTACATTATCTAAATCACACATTCTTCCACACATCTGAGCCACACAAAAACCAAT
CCACATCTTTATCACCCATTCTATAAAAAATCACACTTTGTGAGTCTACACTTTGATTCCCTTCAAACA
CATACAAAGAGAAGAGACTAATTAATTAATTAATCATCTTGAGAGAAAATGGAACGAGCTATACAAGGA
AACGACGCTAGGGAACAAGCTAACAGTGAACGTTGGGATGGAGGATCAGGAGGTACCACTTCTCCCTTC
AAACTTCCTGACGAAAGTCCGAGTTGGACTGAGTGGCGGCTACATAACGATGAGACGAATTCGAATCAA
GATAATCCCCTTGGTTTCAAGGAAAGCTGGGGTTTCGGGAAAGTTGTATTTAAGAGATATCTCAGATAC
GACAGGACGGAAGCTTCACTGCACAGAGTCCTTGGATCTTGGACGGGAGATTCGGTTAACTATGCAGCA
TCTCGATTTTTCGGTTTCGACCAGATCGGATGTACCTATAGTATTCGGTTTCGAGGAGTTAGTATCACC
GTTTCTGGAGGGTCGCGAACTCTTCAGCATCTCTGTGAGATGGCAATTCGGTCTAAGCAAGAACTGCTA
CAGCTTGCCCCAATCGAAGTGGAAAGTAATGTATCAAGAGGATGCCCTGAAGGTACTCAAACCTTCGAA
AAAGAAAGCGAGTAAGTTAAAATGCTTCTTCGTCTCCTATTTATAATATGGTTTGTTATTGTTAATTTT
GTTCTTGTAGAAGAGCTTAATTAATCGTTGTTGTTATGAAATACTATTTGTATGAGATGAACTGGTGTA
ATGTAATTCATTTACATAAGTGGAGTCAGAATCAGAATGTTTCCTCCATAACTAACTAGACATGAAGAC
CTGCCGCGTACAATTGTCTTATATTTGAACAACTAAAATTGAACATCTTTTGCCACAACTTTATAAGTG
GTTAATATAGCTCAAATATATGGTCAAGTTCAATAGATTAATAATGGAAATATCAGTTATCGAAATTCA
TTAACAATCAACTTAACGTTATTAACTACTAATTTTATATCATCCCCTTTGATAAATGATAGTACACCA
ATTAGGAAGGAGCATGCTCGCCTAGGAGATTGTCGTTTCCCGCCTTCAGTTTGCAAGCTGCTCTAGCCG
TGTAGCCAATACGCAAACCGCCTCTCCCCGCGCGTTGGGAATTACTAGCGCGTGTCGACACGCGTGGCG
CGCCCTGGTATATTTATATGTTGTCAAATAACTCAAAAACCATAAAAGTTTAAGTTAGCAAGTGTGTAC
ATTTTTACTTGAACAAAAATATTCACCTACTACTGTTATAAATCATTATTAAACATTAGAGTAAAGAAA
TATGGATGATAAGAACAAGAGTAGTGATATTTTGACAACAATTTTGTTGCAACATTTGAGAAAATTTTG
TTGTTCTCTCTTTTCATTGGTCAAAAACAATAGAGAGAGAAAAAGGAAGAGGGAGAATAAAAACATAAT
GTGAGTATGAGAGAGAAAGTTGTACAAAAGTTGTACCAAAATAGTTGTACAAATATCATTGAGGAATTT
GACAAAAGCTACACAAATAAGGGTTAATTGCTGTAAATAAATAAGGATGACGCATTAGAGAGATGTACC
ATTAGAGAATTTTTGGCAAGTCATTAAAAAGAAAGAATAAATTATTTTTAAAATTAAAAGTTGAGTCAT
TTGATTAAACATGTGATTATTTAATGAATTGATGAAAGAGTTGGATTAAAGTTGTATTAGTAATTAGAA
TTTGGTGTCAAATTTAATTTGACATTTGATCTTTTCCTATATATTGCCCCATAGAGTCAGTTAACTCAT
TTTTATATTTCATAGATCAAATAAGAGAAATAACGGTATATTAATCCCTCCAAAAAAAAAAAACGGTAT
ATTTACTAAAAAATCTAAGCCACGTAGGAGGATAACAGGATCCCCGTAGGAGGATAACATCCAATCCAA
CCAATCACAACAATCCTGATGAGATAACCCACTTTAAGCCCACGCATCTGTGGCACATCTACATTATCT
AAATCACACATTCTTCCACACATCTGAGCCACACAAAAACCAATCCACATCTTTATCACCCATTCTATA
AAAAATCACACTTTGTGAGTCTACACTTTGATTCCCTTCAAACACATACAAAGAGAAGAGACTAATTAA
TTAATTAATCATCTTGAGAGAAAATGAGTCTTCTAACCGAGGTCGAAACGCCTATCAGAAACGAATGGG
GGTGCAGATGCAACGATTCAAGTGATCCTCTTGTTGTTGCCGCAAGTATAATTGGGATTGTGCACCTGA
TATTGTGGATTATTGATCGCCTTTTTTCCAAAAGCATTTATCGTATCTTTAAACACGGTTTAAAAAGAG
GGCCTTCTACGGAAGGAGTACCAGAGTCTATGAGGGAAGAATATCGAGAGGAACAGCAGAATGCTGTGG
ATGCTGACGATGGTCATTTTGTCAGCATAGAGCTGGAGTAAGAGCTCTAAGTTAAAATGCTTCTTCGTC
TCCTATTTATAATATGGTTTGTTATTGTTAATTTTGTTCTTGTAGAAGAGCTTAATTAATCGTTGTTGT
TATGAAATACTATTTGTATGAGATGAACTGGTGTAATGTAATTCATTTACATAAGTGGAGTCAGAATCA
GAATGTTTCCTCCATAACTAACTAGACATGAAGACCTGCCGCGTACAATTGTCTTATATTTGAACAACT
AAAATTGAACATCTTTTGCCACAACTTTATAAGTGGTTAATATAGCTCAAATATATGGTCAAGTTCAAT
AGATTAATAATGGAAATATCAGTTATCGAAATTCATTAACAATCAACTTAACGTTATTAACTACTAATT
TTATATCATCCCCTTTGATAAATGATAGTACACCAATTAGGAAGGAACTAGGAGATTGTCGTTTCCCGC
CTTCAGTTTGCAAGCTGCTCTAGCCGTGTAGCCAATACGCAAACCGCCTCTCCCCGCGCGTTGGGAATT
ACTAGCGCGTGTCGACAAGCTTGCATGCCGGTCAACATGGTGGAGCACGACACACTTGTCTACTCCAAA
AATATCAAAGATACAGTCTCAGAAGACCAAAGGGCAATTGAGACTTTTCAACAAAGGGTAATATCCGGA
AACCTCCTCGGATTCCATTGCCCAGCTATCTGTCACTTTATTGTGAAGATAGTGGAAAAGGAAGGTGGC
TCCTACAAATGCCATCATTGCGATAAAGGAAAGGCCATCGTTGAAGATGCCTCTGCCGACAGTGGTCCC
AAAGATGGACCCCCACCCACGAGGAGCATCGTGGAAAAAGAAGACGTTCCAACCACGTCTTCAAAGCAA
GTGGATTGATGTGATAACATGGTGGAGCACGACACACTTGTCTACTCCAAAAATATCAAAGATACAGTC
TCAGAAGACCAAAGGGCAATTGAGACTTTTCAACAAAGGGTAATATCCGGAAACCTCCTCGGATTCCAT
TGCCCAGCTATCTGTCACTTTATTGTGAAGATAGTGGAAAAGGAAGGTGGCTCCTACAAATGCCATCAT
TGCGATAAAGGAAAGGCCATCGTTGAAGATGCCTCTGCCGACAGTGGTCCCAAAGATGGACCCCCACCC
ACGAGGAGCATCGTGGAAAAAGAAGACGTTCCAACCACGTCTTCAAAGCAAGTGGATTGATGTGATATC
TCCACTGACGTAAGGGATGACGCACAATCCCACTATCCTTCGCAAGACCCTTCCTCTATATAAGGAAGT
TCATTTCATTTGGAGAGGTATTAAAATCTTAATAGGTTTTGATAAAAGCGAACGTGGGGAAACCCGAAC
CAAACCTTCTTCTAAACTCTCTCTCATCTCTCTTAAAGCAAACTTCTCTCTTGTCTTTCTTGCGTGAGC
GATCTTCAACGTTGTCAGATCGTGCTTCGGCACCGCGGATGGCGAAAAACGTTGCGATTTTCGGCTTAT
TGTTTTCTCTTCTTGTGTTGGTTCCTTCTCAGATCTTCGCCTGCAGGCTCCTCAGCCAAAACGACACCC
CCATCTGTCTATCCACTGGCCCCTGGATCTGCTGCCCAAACTAACTCCATGGTGACCCTGGGATGCCTG
GTCAAGGGCTATTTCCCTGAGCCAGTGACAGTGACCTGGAACTCTGGATCCCTGTCCAGCGGTGTGCAC
ACCTTCCCAGCTGTCCTGCAGTCTGACCTCTACACTCTGAGCAGCTCAGTGACTGTCCCCTCCAGCACC
TGGCCCAGCGAGACCGTCACCTGCAACGTTGCCCACCCGGCCAGCAGCACCAAGGTGGACAAGAAAATT
GTGCCCAGGGATTGTGGTTGTAAGCCTTGCATATGTACAGTCCCAGAAGTATCATCTGTCTTCATCTTC
CCCCCAAAGCCCAAGGATGTGCTCACCATTACTCTGACTCCTAAGGTCACGTGTGTTGTGGTAGACATC
AGCAAGGATGATCCCGAGGTCCAGTTCAGCTGGTTTGTAGATGATGTGGAGGTGCACACAGCTCAGACG
CAACCCCGGGAGGAGCAGTTCAACAGCACTTTCCGCTCAGTCAGTGAACTTCCCATCATGCACCAGGAC
TGGCTCAATGGCAAGGAGCGATCGCTCACCATCACCATCACCATCACCATCACCATTAAAGGCCTATTT
TCTTTAGTTTGAATTTACTGTTATTCGGTGTGCATTTCTATGTTTGGTGAGCGGTTTTCTGTGCTCAGA
GTGTGTTTATTTTATGTAATTTAATTTCTTTGTGAGCTCCTGTTTAGCAGGTCGTCCCTTCAGCAAGGA
CACAAAAAGATTTTAATTTTATTAAAAAAAAAAAAAAAAAAGACCGGGAATTCGATATCAAGCTTATCG
ACCTGCAGATCGTTCAAACATTTGGCAATAAAGTTTCTTAAGATTGAATCCTGTTGCCGGTCTTGCGAT
GATTATCATATAATTTCTGTTGAATTACGTTAAGCATGTAATAATTAACATGTAATGCATGACGTTATT
TATGAGATGGGTTTTTATGATTAGAGTCCCGCAATTATACATTTAATACGCGATAGAAAACAAAATATA
GCGCGCAAACTAGGATAAATTATCGCGCGCGGTGTCATCTATGTTACTAGATCTCTAGAGTCTCAAGCT
TGGCGCGCCCACGTGACTAGTGGCACTGGCCGTCGTTTTACAACGTCGTGACTGGGAAAACCCTGGCGT
TACCCAACTTAATCGCCTTGCAGCACATCCCCCTTTCGCCAGCTGGCGTAATAGCGAAGAGGCCCGCAC
CGATCGCCCTTCCCAACAGTTGCGCAGCCTGAATGGCGAATGCTAGAGCAGCTTGAGCTTGGATCAGAT
TGTCGTTTCCCGCCTTCAGTTTAAACTATCAGTGTTTGACAGGATATATTGGCGGGTAAACCTAAGAGA
AAAGAGCGTTTA
IF-H5ITMCT.s1-4r
(SEQ ID NO: 67)
ACTAAAGAAAATAGGCCTTTAAATGCAAATTCTGCATTGTAACGATCCAT
PDI-H5 Indo DNA
(SEQ ID NO: 68)
ATGGCGAAAAACGTTGCGATTTTCGGCTTATTGTTTTCTCTTCTTGTGTTGGTTCCTTCTCAGATCTTC
GCCGATCAGATTTGCATTGGTTACCATGCAAACAATTCAACAGAGCAGGTTGACACAATCATGGAAAAG
AACGTTACTGTTACACATGCCCAAGACATACTGGAAAAGACACACAACGGGAAGCTCTGCGATCTAGAT
GGAGTGAAGCCTCTAATTTTAAGAGATTGTAGTGTAGCTGGATGGCTCCTCGGGAACCCAATGTGTGAC
GAATTCATCAATGTACCGGAATGGTCTTACATAGTGGAGAAGGCCAATCCAACCAATGACCTCTGTTAC
CCAGGGAGTTTCAACGACTATGAAGAACTGAAACACCTATTGAGCAGAATAAACCATTTTGAGAAAATT
CAAATCATCCCCAAAAGTTCTTGGTCCGATCATGAAGCCTCATCAGGAGTTAGCTCAGCATGTCCATAC
CTGGGAAGTCCCTCCTTTTTTAGAAATGTGGTATGGCTTATCAAAAAGAACAGTACATACCCAACAATA
AAGAAAAGCTACAATAATACCAACCAAGAGGATCTTTTGGTACTGTGGGGAATTCACCATCCTAATGAT
GCGGCAGAGCAGACAAGGCTATATCAAAACCCAACCACCTATATTTCCATTGGGACATCAACACTAAAC
CAGAGATTGGTACCAAAAATAGCTACTAGATCCAAAGTAAACGGGCAAAGTGGAAGGATGGAGTTCTTC
TGGACAATTTTAAAACCTAATGATGCAATCAACTTCGAGAGTAATGGAAATTTCATTGCTCCAGAATAT
GCATACAAAATTGTCAAGAAAGGGGACTCAGCAATTATGAAAAGTGAATTGGAATATGGTAACTGCAAC
ACCAAGTGTCAAACTCCAATGGGGGCGATAAACTCTAGTATGCCATTCCACAACATACACCCTCTCACC
ATCGGGGAATGCCCCAAATATGTGAAATCAAACAGATTAGTCCTTGCAACAGGGCTCAGAAATAGCCCT
CAAAGAGAGAGCAGAAGAAAAAAGAGAGGACTATTTGGAGCTATAGCAGGTTTTATAGAGGGAGGATGG
CAGGGAATGGTAGATGGTTGGTATGGGTACCACCATAGCAATGAGCAGGGGAGTGGGTACGCTGCAGAC
AAAGAATCCACTCAAAAGGCAATAGATGGAGTCACCAATAAGGTCAACTCAATCATTGACAAAATGAAC
ACTCAGTTTGAGGCCGTTGGAAGGGAATTTAATAACTTAGAAAGGAGAATAGAGAATTTAAACAAGAAG
ATGGAAGACGGGTTTCTAGATGTCTGGACTTATAATGCCGAACTTCTGGTTCTCATGGAAAATGAGAGA
ACTCTAGACTTTCATGACTCAAATGTTAAGAACCTCTACGACAAGGTCCGACTACAGCTTAGGGATAAT
GCAAAGGAGCTGGGTAACGGTTGTTTCGAGTTCTATCACAAATGTGATAATGAATGTATGGAAAGTATA
AGAAACGGAACGTACAACTATCCGCAGTATTCAGAAGAAGCAAGATTAAAAAGAGAGGAAATAAGTGGG
GTAAAATTGGAATCAATAGGAACTTACCAAATACTGTCAATTTATTCAACAGTGGCGAGTTCCCTAGCA
CTGGCAATCATGATGGCTGGTCTATCTTTATGGATGTGCTCCAATGGATCGTTACAATGCAGAATTTGC
ATTTAA
PDI-H5 Indo AA
(SEQ ID NO: 69)
MAKNVAIFGLLFSLLVLVPSQIFADQICIGYHANNSTEQVDTIMEKNVTVTHAQDILEKTHNGKLCDLD
GVKPLILRDCSVAGWLLGNPMCDEFINVPEWSYIVEKANPTNDLCYPGSFNDYEELKHLLSRINHFEKI
QIIPKSSWSDHEASSGVSSACPYLGSPSFFRNVVWLIKKNSTYPTIKKSYNNTNQEDLLVLWGIHHPND
AAEQTRLYQNPTTYISIGTSTLNQRLVPKIATRSKVNGQSGRMEFFWTILKPNDAINFESNGNFIAPEY
AYKIVKKGDSAIMKSELEYGNCNTKCQTPMGAINSSMPFHNIHPLTIGECPKYVKSNRLVLATGLRNSP
QRESRRKKRGLFGAIAGFIEGGWQGMVDGWYGYHHSNEQGSGYAADKESTQKAIDGVTNKVNSIIDKMN
TQFEAVGREFNNLERRIENLNKKMEDGFLDVWTYNAELLVLMENERTLDFHDSNVKNLYDKVRLQLRDN
AKELGNGCFEFYHKCDNECMESIRNGTYNYPQYSEEARLKREEISGVKLESIGTYQILSIYSTVASSLA
LAIMMAGLSLWMCSNGSLQCRICI*
H5Ind(F393D).r
(SEQ ID NO: 70)
CTTCCAACGGCCTCGTCCTGAGTGTTCATTTTGTCAATGATTGAGTTGA
H5Ind(F393D).c
(SEQ ID NO: 71)
CAAAATGAACACTCAGGACGAGGCCGTTGGAAGGGAATTTAATAACTTA
PDI-H5 Indo-F393D DNA
(SEQ ID NO: 72)
ATGGCGAAAAACGTTGCGATTTTCGGCTTATTGTTTTCTCTTCTTGTGTTGGTTCCTTCTCAGATCTTC
GCGGATCAGATTTGCATTGGTTACCATGCAAACAATTCAACAGAGCAGGTTGACACAATCATGGAAAAG
AACGTTACTGTTACACATGCCCAAGACATACTGGAAAAGACACACAACGGGAAGCTCTGCGATCTAGAT
GGAGTGAAGCCTCTAATTTTAAGAGATTGTAGTGTAGCTGGATGGCTCCTCGGGAACCCAATGTGTGAC
GAATTCATCAATGTACCGGAATGGTCTTACATAGTGGAGAAGGCCAATCCAACCAATGACCTCTGTTAC
CCAGGGAGTTTCAACGACTATGAAGAACTGAAACACCTATTGAGCAGAATAAACCATTTTGAGAAAATT
CAAATCATCCCCAAAAGTTCTTGGTCCGATCATGAAGCCTCATCAGGAGTTAGCTCAGCATGTCCATAC
CTGGGAAGTCCCTCCTTTTTTAGAAATGTGGTATGGCTTATCAAAAAGAACAGTACATACCCAACAATA
AAGAAAAGCTACAATAATACCAACCAAGAGGATCTTTTGGTACTGTGGGGAATTCACCATCCTAATGAT
GCGGCAGAGCAGACAAGGCTATATCAAAACCCAACCACCTATATTTCCATTGGGACATCAACACTAAAC
CAGAGATTGGTACCAAAAATAGCTACTAGATCCAAAGTAAACGGGCAAAGTGGAAGGATGGAGTTCTTC
TGGACAATTTTAAAACCTAATGATGCAATCAACTTCGAGAGTAATGGAAATTTCATTGCTCCAGAATAT
GCATACAAAATTGTCAAGAAAGGGGACTCAGCAATTATGAAAAGTGAATTGGAATATGGTAACTGCAAC
ACCAAGTGTCAAACTCCAATGGGGGCGATAAACTCTAGTATGCCATTCCACAACATACACCCTCTCACC
ATCGGGGAATGCCCCAAATATGTGAAATCAAACAGATTAGTCCTTGCAACAGGGCTCAGAAATAGCCCT
CAAAGAGAGAGCAGAAGAAAAAAGAGAGGACTATTTGGAGCTATAGCAGGTTTTATAGAGGGAGGATGG
CAGGGAATGGTAGATGGTTGGTATGGGTACCACCATAGCAATGAGCAGGGGAGTGGGTACGCTGCAGAC
AAAGAATCCACTCAAAAGGCAATAGATGGAGTCACCAATAAGGTCAACTCAATCATTGACAAAATGAAC
ACTCAGGACGAGGCCGTTGGAAGGGAATTTAATAACTTAGAAAGGAGAATAGAGAATTTAAACAAGAAG
ATGGAAGACGGGTTTCTAGATGTCTGGACTTATAATGCCGAACTTCTGGTTCTCATGGAAAATGAGAGA
ACTCTAGACTTTCATGACTCAAATGTTAAGAACCTCTACGACAAGGTCCGACTACAGCTTAGGGATAAT
GCAAAGGAGCTGGGTAACGGTTGTTTCGAGTTCTATCACAAATGTGATAATGAATGTATGGAAAGTATA
AGAAACGGAACGTACAACTATCCGCAGTATTCAGAAGAAGCAAGATTAAAAAGAGAGGAAATAAGTGGG
GTAAAATTGGAATCAATAGGAACTTACCAAATACTGTCAATTTATTCAACAGTGGCGAGTTCCCTAGCA
CTGGCAATCATGATGGCTGGTCTATCTTTATGGATGTGCTCCAATGGATCGTTACAATGCAGAATTTGC
ATTTAA
PDI-H5 Indo-F393D AA
(SEQ ID NO: 73)
MAKNVAIFGLLFSLLVLVPSQIFADQICIGYHANNSTEQVDTIMEKNVTVTHAQDILEKTHNGKLCDLD
GVKPLILRDCSVAGWLLGNPMCDEFINVPEWSYIVEKANPTNDLCYPGSFNDYEELKHLLSRINHFEKI
QIIPKSSWSDHEASSGVSSACPYLGSPSFFRNVVWLIKKNSTYPTIKKSYNNTNQEDLLVLWGIHHPND
AAEQTRLYQNPTTYISIGTSTLNQRLVPKIATRSKVNGQSGRMEFFWTILKPNDAINFESNGNFIAPEY
AYKIVKKGDSAIMKSELEYGNCNTKCQTPMGAINSSMPFHNIHPLTIGECPKYVKSNRLVLATGLRNSP
QRESRRKKRGLFGAIAGFIEGGWQGMVDGWYGYHHSNEQGSGYAADKESTQKAIDGVTNKVNSIIDKMN
TQDEAVGREFNNLERRIENLNKKMEDGFLDVWTYNAELLVLMENERTLDFHDSNVKNLYDKVRLQLRDN
AKELGNGCFEFYHKCDNECMESIRNGTYNYPQYSEEARLKREEISGVKLESIGTYQILSIYSTVASSLA
LAIMMAGLSLWMCSNGSLQCRICI*
IF-H5_Egy.r
(SEQ ID NO: 74)
ACTAAAGAAAATAGGCCTTTAAATGCAAATTCTGCATTGTAGCGATCCATT
PDI-H5 Egypt DNA
(SEQ ID NO: 75)
ATGGCGAAAAACGTTGCGATTTTCGGCTTATTGTTTTCTCTTCTTGTGTTGGTTCCTTCTCAGATCTTC
GCGGATCAGATTTGCATTGGTTACCATGCAAACAACTCGACAGAGCAGGTTGACACAATAATGGAAAAG
AATGTCACTGTTACACACGCCCAAGACATACTGGAAAAGACACACAACGGGAAACTCTGCAATCTAGAT
GGAGTGAAGCCTCTCATTTTGAGAGATTGTAGTGTAGCTGGATGGCTCCTCGGGAACCCAATGTGCGAT
GAATTCCTCAATGTGCCGGAATGGTCTTACATAGTGGAGAAAATCAATCCAGCCAATGACCTCTGTTAT
CCAGGGAATTTCAACGACTATGAAGAACTGAAACACCTATTGAGCAGAATAAACCATTTTGAGAAAATT
CAGATCATTCCCAAAGATTCTTGGTCAGATCATGAAGCCTCGGGAGTGAGCTCAGCATGCCCATACCAA
GGAAGATCCTCCTTTTTTAGAAATGTTGTATGGCTTACCAAAAAGAACGATGCATACCCAACAATAAAG
AAAAGTTACAATAATACTAACCAAGAAGATCTTTTGGTACTATGGGGGATTCACCATCCAAATGATGCT
GCAGAGCAGACAAGGCTTTATCAAAACCCAACTACCTATATCTCCGTTGGGACATCAACACTAAACCAG
AGATTGGTACCCAAAATAGCTACTAGATCTAAGGTAAACGGGCAAAGTGGAAGGATGGAGTTCTTTTGG
ACAATTTTAAAATCGAATGATGCAATAAACTTTGAGAGCAATGGAAACTTCATTGCTCCAGAAAATGCA
TACAAAATTGTCAAGAAAGGAGATTCAACAATTATGAAAAGTGAGTTGGAATATAGTAACTGCAACACC
AAGTGTCAGACTCCAATAGGGGCGATAAACTCCAGTATGCCATTCCACAACATCCACCCTCTCACCATC
GGGGAATGCCCCAAATATGTGAAATCAAACAGATTAGTCCTTGCTACTGGGCTCAGGAATAGCCCTCAA
GGAGAGAAAAGAAGAAAAAAGAGAGGACTATTCGGAGCCATAGCAGGCTTTATAGAGGGAGGATGGCAG
GGAATGGTAGATGGTTGGTATGGGTACCACCATAGCAACGAGCAGGGGAGTGGGTACGCTGCAGACAAA
GAATCCACTCAAAGGGCTATAGATGGAGTCACCAATAAGGTCAATTCGATCATTGACAAAATGAACACT
CAGTTTGAGGCTGTTGGAAGGGAATTTAATAACTTAGAAAGGAGAATAGAAAATTTAAACAAGAAGATG
GAAGACGGATTCCTAGATGTCTGGACTTATAATGCTGAACTTCTGGTTCTCATGGAAAATGAGAGAACT
CTAGACTTTCATGACTCAAATGTCAAGAATCTTTATGACAAGGTCCGACTACAGCTTAGGGATAATGCA
AAGGAGCTTGGTAACGGTTGTTTCGAGTTCTATCACAGATGTGATAATGAATGTATGGAAAGTGTAAGA
AACGGAACGTATGACTACCCTCAATATTCAGAAGAAGCAAGATTAAAAAGAGAGGAAATAAGTGGAGTA
AAATTGGAGTCAATAGGAACTTACCAAATACTGTCAATTTATTCAACAGTGGCGAGCTCCCTAGCACTG
GCAATCATGGTGGCTGGTCTATCTTTATGGATGTGCTCCAATGGATCGCTACAATGCAGAATTTGCATT
TAA
PDI-H5 Egypt AA
(SEQ ID NO: 76)
MAKNVAIFGLLFSLLVLVPSQIFADQICIGYHANNSTEQVDTIMEKNVTVTHAQDILEKTHNGKLCNLD
GVKPLILRDCSVAGWLLGNPMCDEFLNVPEWSYIVEKINPANDLCYPGNFNDYEELKHLLSRINHFEKI
QIIPKDSWSDHEASGVSSACPYQGRSSFFRNVVWLTKKNDAYPTIKKSYNNTNQEDLLVLWGIHHPNDA
AEQTRLYQNPTTYISVGTSTLNQRLVPKIATRSKVNGQSGRMEFFWTILKSNDAINFESNGNFIAPENA
YKIVKKGDSTIMKSELEYSNCNTKCQTPIGAINSSMPFHNIHPLTIGECPKYVKSNRLVLATGLRNSPQ
GEKRRKKRGLFGAIAGFIEGGWQGMVDGWYGYHHSNEQGSGYAADKESTQRAIDGVTNKVNSIIDKMNT
QFEAVGREFNNLERRIENLNKKMEDGFLDVWTYNAELLVLMENERTLDFHDSNVKNLYDKVRLQLRDNA
KELGNGCFEFYHRCDNECMESVRNGTYDYPQYSEEARLKREEISGVKLESIGTYQILSIYSTVASSLAL
AIMVAGLSLWMCSNGSLQCRICI*
H5Egy(F392D).r
(SEQ ID NO: 77)
CTTCCAACAGCCTCGTCCTGAGTGTTCATTTTGTCAATGATCGAATTGA
H5Egy(F392D).c
(SEQ ID NO: 78)
CAAAATGAACACTCAGGACGAGGCTGTTGGAAGGGAATTTAATAACTTA
PDI-H5 Egypt-F392D DNA
(SEQ ID NO: 79)
ATGGCGAAAAACGTTGCGATTTTCGGCTTATTGTTTTCTCTTCTTGTGTTGGTTCCTTCTCAGATCTTC
GCGGATCAGATTTGCATTGGTTACCATGCAAACAACTCGACAGAGCAGGTTGACACAATAATGGAAAAG
AATGTCACTGTTACACACGCCCAAGACATACTGGAAAAGACACACAACGGGAAACTCTGCAATCTAGAT
GGAGTGAAGCCTCTCATTTTGAGAGATTGTAGTGTAGCTGGATGGCTCCTCGGGAACCCAATGTGCGAT
GAATTCCTCAATGTGCCGGAATGGTCTTACATAGTGGAGAAAATCAATCCAGCCAATGACCTCTGTTAT
CCAGGGAATTTCAACGACTATGAAGAACTGAAACACCTATTGAGCAGAATAAACCATTTTGAGAAAATT
CAGATCATTCCCAAAGATTCTTGGTCAGATCATGAAGCCTCGGGAGTGAGCTCAGCATGCCCATACCAA
GGAAGATCCTCCTTTTTTAGAAATGTTGTATGGCTTACCAAAAAGAACGATGCATACCCAACAATAAAG
AAAAGTTACAATAATACTAACCAAGAAGATCTTTTGGTACTATGGGGGATTCACCATCCAAATGATGCT
GCAGAGCAGACAAGGCTTTATCAAAACCCAACTACCTATATCTCCGTTGGGACATCAACACTAAACCAG
AGATTGGTACCCAAAATAGCTACTAGATCTAAGGTAAACGGGCAAAGTGGAAGGATGGAGTTCTTTTGG
ACAATTTTAAAATCGAATGATGCAATAAACTTTGAGAGCAATGGAAACTTCATTGCTCCAGAAAATGCA
TACAAAATTGTCAAGAAAGGAGATTCAACAATTATGAAAAGTGAGTTGGAATATAGTAACTGCAACACC
AAGTGTCAGACTCCAATAGGGGCGATAAACTCCAGTATGCCATTCCACAACATCCACCCTCTCACCATC
GGGGAATGCCCCAAATATGTGAAATCAAACAGATTAGTCCTTGCTACTGGGCTCAGGAATAGCCCTCAA
GGAGAGAAAAGAAGAAAAAAGAGAGGACTATTCGGAGCCATAGCAGGCTTTATAGAGGGAGGATGGCAG
GGAATGGTAGATGGTTGGTATGGGTACCACCATAGCAACGAGCAGGGGAGTGGGTACGCTGCAGACAAA
GAATCCACTCAAAGGGCTATAGATGGAGTCACCAATAAGGTCAATTCGATCATTGACAAAATGAACACT
CAGGACGAGGCTGTTGGAAGGGAATTTAATAACTTAGAAAGGAGAATAGAAAATTTAAACAAGAAGATG
GAAGACGGATTCCTAGATGTCTGGACTTATAATGCTGAACTTCTGGTTCTCATGGAAAATGAGAGAACT
CTAGACTTTCATGACTCAAATGTCAAGAATCTTTATGACAAGGTCCGACTACAGCTTAGGGATAATGCA
AAGGAGCTTGGTAACGGTTGTTTCGAGTTCTATCACAGATGTGATAATGAATGTATGGAAAGTGTAAGA
AACGGAACGTATGACTACCCTCAATATTCAGAAGAAGCAAGATTAAAAAGAGAGGAAATAAGTGGAGTA
AAATTGGAGTCAATAGGAACTTACCAAATACTGTCAATTTATTCAACAGTGGCGAGCTCCCTAGCACTG
GCAATCATGGTGGCTGGTCTATCTTTATGGATGTGCTCCAATGGATCGCTACAATGCAGAATTTGCATT
TAA
PDI-H5 Egypt-F392D AA
(SEQ ID NO: 80)
MAKNVAIFGLLFSLLVLVPSQIFADQICIGYHANNSTEQVDTIMEKNVTVTHAQDILEKTHNGKLCNLD
GVKPLILRDCSVAGWLLGNPMCDEFLNVPEWSYIVEKINPANDLCYPGNFNDYEELKHLLSRINHFEKI
QIIPKDSWSDHEASGVSSACPYQGRSSFFRNVVWLTKKNDAYPTIKKSYNNTNQEDLLVLWGIHHPNDA
AEQTRLYQNPTTYISVGTSTLNQRLVPKIATRSKVNGQSGRMEFFWTILKSNDAINFESNGNFIAPENA
YKIVKKGDSTIMKSELEYSNCNTKCQTPIGAINSSMPFHNIHPLTIGECPKYVKSNRLVLATGLRNSPQ
GEKRRKKRGLFGAIAGFIEGGWQGMVDGWYGYHHSNEQGSGYAADKESTQRAIDGVTNKVNSIIDKMNT
QDEAVGREFNNLERRIENLNKKMEDGFLDVWTYNAELLVLMENERTLDFHDSNVKNLYDKVRLQLRDNA
KELGNGCFEFYHRCDNECMESVRNGTYDYPQYSEEARLKREEISGVKLESIGTYQILSIYSTVASSLAL
AIMVAGLSLWMCSNGSLQCRICI*
PDI-H3 Switz-N382A DNA
(SEQ ID NO: 81)
ATGGCGAAAAACGTTGCGATTTTCGGCTTATTGTTTTCTCTTCTTGTGTTGGTTCCTTCTCAGATCTTC
GCGCAAAAACTTCCTGGAAATGACAATAGCACGGCAACGCTGTGCCTTGGGCACCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGGCTGGAGTCACTCAAAACGGAACAAGTTCT
TCTTGCATAAGGGGATCTAATAGTAGTTTCTTTAGTAGATTAAATTGGTTGACCCACTTAAACTCCAAA
TACCCAGCATTAAACGTGACTATGCCAAACAATGAACAATTTGACAAATTGTACATTTGGGGGGTTCAC
CACCCGGGTACGGACAAGGACCAAATCTTCCTGTATGCACAATCATCAGGAAGAATCACAGTATCTACC
AAAAGAAGCCAACAAGCTGTAATCCCGAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCAAAGCACTCTGAAATTGGCAACAGGAATGCGA
AATGTACCAGAGAGACAAACTAGAGGCATATTTGGCGCAATAGCGGGTTTCATAGAAAATGGTTGGGAG
GGAATGGTGGATGGTTGGTACGGCTTCAGGCATCAAAATTCTGAGGGAAGAGGACAAGCAGCAGATCTC
AAAAGCACTCAAGCAGCAATCGATCAAATCAATGGGAAGCTGGCTCGATTGATCGGGAAAACCAACGAG
AAATTCCATCAGATTGAAAAAGAATTCTCAGAAGTAGAAGGGAGAATTCAGGACCTTGAGAAATATGTT
GAGGACACAAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATCAGA
AATGGAACTTATGACCACGATGTATACAGGGATGAAGCATTAAACAACCGGTTCCAGATCAAGGGAGTT
GAGCTGAAGTCAGGGTACAAAGATTGGATCCTATGGATTTCCTTTGCCATATCATGTTTTTTGCTTTGT
GTTGCTTTGTTGGGGTTCATCATGTGGGCCTGCCAAAAGGGCAACATTAGGTGCAACATTTGCATTTGA
PDI-H3 Switz-N382A AA
(SEQ ID NO: 82)
MAKNVAIFGLLFSLLVLVPSQIFAQKLPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWAGVTQNGTSSSCIRGSNSSFFSRLNWLTHLNSKYPALNVTMPNNEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKQSTLKLATGMR
NVPERQTRGIFGAIAGFIENGWEGMVDGWYGFRHQNSEGRGQAADLKSTQAAIDQINGKLARLIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHDVYRDEALNNRFQIKGVELKSGYKDWILWISFAISCFLLC
VALLGFIMWACQKGNIRCNICI*
PDI-H3 Switz-L384V DNA
(SEQ ID NO: 83)
ATGGCGAAAAACGTTGCGATTTTCGGCTTATTGTTTTCTCTTCTTGTGTTGGTTCCTTCTCAGATCTTC
GCGCAAAAACTTCCTGGAAATGACAATAGCACGGCAACGCTGTGCCTTGGGCACCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAGAAAACTGCACACTAATAGAT
GCTCTATTGGGAGACCCTCAGTGTGATGGCTTTCAAAATAAGAAATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACAATGAAAGCTTCAATTGGGCTGGAGTCACTCAAAACGGAACAAGTTCT
TCTTGCATAAGGGGATCTAATAGTAGTTTCTTTAGTAGATTAAATTGGTTGACCCACTTAAACTCCAAA
TACCCAGCATTAAACGTGACTATGCCAAACAATGAACAATTTGACAAATTGTACATTTGGGGGGTTCAC
CACCCGGGTACGGACAAGGACCAAATCTTCCTGTATGCACAATCATCAGGAAGAATCACAGTATCTACC
AAAAGAAGCCAACAAGCTGTAATCCCGAATATCGGATCTAGACCCAGAATAAGGGATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACGAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCAAAGCACTCTGAAATTGGCAACAGGAATGCGA
AATGTACCAGAGAGACAAACTAGAGGCATATTTGGCGCAATAGCGGGTTTCATAGAAAATGGTTGGGAG
GGAATGGTGGATGGTTGGTACGGCTTCAGGCATCAAAATTCTGAGGGAAGAGGACAAGCAGCAGATCTC
AAAAGCACTCAAGCAGCAATCGATCAAATCAATGGGAAGCTGAATCGAGTGATCGGGAAAACCAACGAG
AAATTCCATCAGATTGAAAAAGAATTCTCAGAAGTAGAAGGGAGAATTCAGGACCTTGAGAAATATGTT
GAGGACACAAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATCAGA
AATGGAACTTATGACCACGATGTATACAGGGATGAAGCATTAAACAACCGGTTCCAGATCAAGGGAGTT
GAGCTGAAGTCAGGGTACAAAGATTGGATCCTATGGATTTCCTTTGCCATATCATGTTTTTTGCTTTGT
GTTGCTTTGTTGGGGTTCATCATGTGGGCCTGCCAAAAGGGCAACATTAGGTGCAACATTTGCATTTGA
PDI-H3 Switz-L384V AA
(SEQ ID NO: 84)
MAKNVAIFGLLFSLLVLVPSQIFAQKLPGNDNSTATLCLGHHAVPNGTIVKTITNDRIEVTNATELVQN
SSIGEICDSPHQILDGENCTLIDALLGDPQCDGFQNKKWDLFVERSKAYSNCYPYDVPDYASLRSLVAS
SGTLEFNNESFNWAGVTQNGTSSSCIRGSNSSFFSRLNWLTHLNSKYPALNVTMPNNEQFDKLYIWGVH
HPGTDKDQIFLYAQSSGRITVSTKRSQQAVIPNIGSRPRIRDIPSRISIYWTIVKPGDILLINSTGNLI
APRGYFKIRSGKSSIMRSDAPIGKCKSECITPNGSIPNDKPFQNVNRITYGACPRYVKQSTLKLATGMR
NVPERQTRGIFGAIAGFIENGWEGMVDGWYGFRHQNSEGRGQAADLKSTQAAIDQINGKLNRVIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHDVYRDEALNNRFQIKGVELKSGYKDWILWISFAISCFLLC
VALLGFIMWACQKGNIRCNICI*
PDI-H3 Penn-CysTm DNA
(SEQ ID NO: 85)
ATGGCGAAAAACGTTGCGATTTTCGGCTTATTGTTTTCTCTTCTTGTGTTGGTTCCTTCTCAGATCTTC
GCGCAAAAACTTCCTGGAAATGACAATAGCACGGCAACGCTGTGCCTTGGGCACCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAAAAAACTGCACTCTAATAGAT
GCTCTATTGGGAGACCCTCAGTGTGATGGCTTTCAAAATAAGAGATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACGATGAAAGCTTCAATTGGGCTGGAGTCACTCAAAACGGAACAAGTTCT
GCTTGCATAAGGGGATCTAATAGTAGTTTCTTTAGTAGATTAAATTGGTTGACCCACTCAAACTTCAAA
TACCCAGCATTGAACGTGACTATGCCAAACAATGAACAATTTGACAAATTGTACATTTGGGGGGTTCAC
CACCCGGGTACAGACAAGGACCAAATCTTCCTGTACGCTCAATCATCAGGAAGAATCACAGTATCTACC
AAAAGAAGCCAACAAGCTGTAATCCCGAATATCGGATCTAGACCCAGAATAAGGAATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACAAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCAAAGCACTCTGAAATTGGCAACAGGAATGCGA
AATGTACCAGAGAAACAAACTAGAGGCATATTTGGCGCAATAGCGGGTTTCATAGAAAATGGTTGGGAG
GGAATGAAGGATGGTTGGTACGGTTTCAGGCATCAAAATTCTGAGGGAAGAGGACAAGCAGCAGATCTC
AAAAGCACTCAAGCAGCAATCGATCAAATCAATGGGAAGCTGAATCGATTGATCGGGAAAACCAACGAG
AAATTCCATCAGATTGAAAAAGAATTCTCAGAAGTAGAAGGGAGAATTCAGGACCTTGAGAAATATGTT
GAGGACACAAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATCAGA
AATGGAACTTATGACCACGATGTATACAGGGATGAAGCTTTAAACAACCGGTTCCAGATCAAGGGAGTT
GAGCTGAAGTCAGGGTACAAAGATTGGATCCTATGGATTTCCTTTGCCATATCATCCCTTGTACTGTTA
GTTGCTTTGTTGGGGTTCATCATGTGGGCCTGCCAAAAGGGCAACATTAGGTGCAACATTTGCATTTGA
PDI-H3 Penn-CysTm AA
(SEQ ID NO: 86)
MAKNVAIFGLLFSLLVLVPSQIFAQKLPGNDNSTATLCLGHHAVPNGTIVKTITNDRIEVTNATELVQN
SSIGEICDSPHQILDGKNCTLIDALLGDPQCDGFQNKRWDLFVERSKAYSNCYPYDVPDYASLRSLVAS
SGTLEFNDESFNWAGVTQNGTSSACIRGSNSSFFSRLNWLTHSNFKYPALNVTMPNNEQFDKLYIWGVH
HPGTDKDQIFLYAQSSGRITVSTKRSQQAVIPNIGSRPRIRNIPSRISIYWTIVKPGDILLINSTGNLI
APRGYFKIQSGKSSIMRSDAPIGKCKSECITPNGSIPNDKPFQNVNRITYGACPRYVKQSTLKLATGMR
NVPEKQTRGIFGAIAGFIENGWEGMKDGWYGFRHQNSEGRGQAADLKSTQAAIDQINGKLNRLIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHDVYRDEALNNRFQIKGVELKSGYKDWILWISFAISSLVLL
VALLGFIMWACQKGNIRCNICI*
PDI-H3 Penn-N382A-CysTm DNA
(SEQ ID NO: 87)
ATGGCGAAAAACGTTGCGATTTTCGGCTTATTGTTTTCTCTTCTTGTGTTGGTTCCTTCTCAGATCTTC
GCGCAAAAACTTCCTGGAAATGACAATAGCACGGCAACGCTGTGCCTTGGGCACCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAAAAAACTGCACTCTAATAGAT
GCTCTATTGGGAGACCCTCAGTGTGATGGCTTTCAAAATAAGAGATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACGATGAAAGCTTCAATTGGGCTGGAGTCACTCAAAACGGAACAAGTTCT
GCTTGCATAAGGGGATCTAATAGTAGTTTCTTTAGTAGATTAAATTGGTTGACCCACTCAAACTTCAAA
TACCCAGCATTGAACGTGACTATGCCAAACAATGAACAATTTGACAAATTGTACATTTGGGGGGTTCAC
CACCCGGGTACAGACAAGGACCAAATCTTCCTGTACGCTCAATCATCAGGAAGAATCACAGTATCTACC
AAAAGAAGCCAACAAGCTGTAATCCCGAATATCGGATCTAGACCCAGAATAAGGAATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACAAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCAAAGCACTCTGAAATTGGCAACAGGAATGCGA
AATGTACCAGAGAAACAAACTAGAGGCATATTTGGCGCAATAGCGGGTTTCATAGAAAATGGTTGGGAG
GGAATGAAGGATGGTTGGTACGGTTTCAGGCATCAAAATTCTGAGGGAAGAGGACAAGCAGCAGATCTC
AAAAGCACTCAAGCAGCAATCGATCAAATCAATGGGAAGCTGGCTCGATTGATCGGGAAAACCAACGAG
AAATTCCATCAGATTGAAAAAGAATTCTCAGAAGTAGAAGGGAGAATTCAGGACCTTGAGAAATATGTT
GAGGACACAAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATCAGA
AATGGAACTTATGACCACGATGTATACAGGGATGAAGCTTTAAACAACCGGTTCCAGATCAAGGGAGTT
GAGCTGAAGTCAGGGTACAAAGATTGGATCCTATGGATTTCCTTTGCCATATCATCCCTTGTACTGTTA
GTTGCTTTGTTGGGGTTCATCATGTGGGCCTGCCAAAAGGGCAACATTAGGTGCAACATTTGCATTTGA
PDI-H3 Penn-N382A-CysTm AA
(SEQ ID NO: 88)
MAKNVAIFGLLFSLLVLVPSQIFAQKLPGNDNSTATLCLGHHAVPNGTIVKTITNDRIEVTNATELVQN
SSIGEICDSPHQILDGKNCTLIDALLGDPQCDGFQNKRWDLFVERSKAYSNCYPYDVPDYASLRSLVAS
SGTLEFNDESFNWAGVTQNGTSSACIRGSNSSFESRLNWLTHSNFKYPALNVTMPNNEQFDKLYIWGVH
HPGTDKDQIFLYAQSSGRITVSTKRSQQAVIPNIGSRPRIRNIPSRISIYWTIVKPGDILLINSTGNLI
APRGYFKIQSGKSSIMRSDAPIGKCKSECITPNGSIPNDKPFQNVNRITYGACPRYVKQSTLKLATGMR
NVPEKQTRGIFGAIAGFIENGWEGMKDGWYGFRHQNSEGRGQAADLKSTQAAIDQINGKLARLIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHDVYRDEALNNRFQIKGVELKSGYKDWILWISFAISSLVLL
VALLGFIMWACQKGNIRCNICI*
PDI-H3 Penn-L384V-CysTm DNA
(SEQ ID NO: 89)
ATGGCGAAAAACGTTGCGATTTTCGGCTTATTGTTTTCTCTTCTTGTGTTGGTTCCTTCTCAGATCTTC
GCGCAAAAACTTCCTGGAAATGACAATAGCACGGCAACGCTGTGCCTTGGGCACCATGCAGTACCAAAC
GGAACGATAGTGAAAACAATCACGAATGACCGAATTGAAGTTACTAATGCTACTGAGCTGGTTCAGAAT
TCCTCAATAGGTGAAATATGCGACAGTCCTCATCAGATCCTTGATGGAAAAAACTGCACTCTAATAGAT
GCTCTATTGGGAGACCCTCAGTGTGATGGCTTTCAAAATAAGAGATGGGACCTTTTTGTTGAACGAAGC
AAAGCCTACAGCAACTGTTACCCTTATGATGTGCCGGATTATGCCTCCCTTAGGTCACTAGTTGCCTCA
TCCGGCACACTGGAGTTTAACGATGAAAGCTTCAATTGGGCTGGAGTCACTCAAAACGGAACAAGTTCT
GCTTGCATAAGGGGATCTAATAGTAGTTTCTTTAGTAGATTAAATTGGTTGACCCACTCAAACTTCAAA
TACCCAGCATTGAACGTGACTATGCCAAACAATGAACAATTTGACAAATTGTACATTTGGGGGGTTCAC
CACCCGGGTACAGACAAGGACCAAATCTTCCTGTACGCTCAATCATCAGGAAGAATCACAGTATCTACC
AAAAGAAGCCAACAAGCTGTAATCCCGAATATCGGATCTAGACCCAGAATAAGGAATATCCCTAGCAGA
ATAAGCATCTATTGGACAATAGTAAAACCGGGAGACATACTTTTGATTAACAGCACAGGGAATCTAATT
GCTCCTAGGGGTTACTTCAAAATACAAAGTGGGAAAAGCTCAATAATGAGATCAGATGCACCCATTGGC
AAATGCAAGTCTGAATGCATCACTCCAAATGGAAGCATTCCCAATGACAAACCATTCCAAAATGTAAAC
AGGATCACATACGGGGCCTGTCCCAGATATGTTAAGCAAAGCACTCTGAAATTGGCAACAGGAATGCGA
AATGTACCAGAGAAACAAACTAGAGGCATATTTGGCGCAATAGCGGGTTTCATAGAAAATGGTTGGGAG
GGAATGAAGGATGGTTGGTACGGTTTCAGGCATCAAAATTCTGAGGGAAGAGGACAAGCAGCAGATCTC
AAAAGCACTCAAGCAGCAATCGATCAAATCAATGGGAAGCTGAATCGAGTGATCGGGAAAACCAACGAG
AAATTCCATCAGATTGAAAAAGAATTCTCAGAAGTAGAAGGGAGAATTCAGGACCTTGAGAAATATGTT
GAGGACACAAAAATAGATCTCTGGTCATACAACGCGGAGCTTCTTGTTGCCCTGGAGAACCAACATACA
ATTGATCTAACTGACTCAGAAATGAACAAACTGTTTGAAAAAACAAAGAAGCAACTGAGGGAAAATGCT
GAGGATATGGGCAATGGTTGTTTCAAAATATACCACAAATGTGACAATGCCTGCATAGGATCAATCAGA
AATGGAACTTATGACCACGATGTATACAGGGATGAAGCTTTAAACAACCGGTTCCAGATCAAGGGAGTT
GAGCTGAAGTCAGGGTACAAAGATTGGATCCTATGGATTTCCTTTGCCATATCATCCCTTGTACTGTTA
GTTGCTTTGTTGGGGTTCATCATGTGGGCCTGCCAAAAGGGCAACATTAGGTGCAACATTTGCATTTGA
PDI-H3 Penn-L384V-CysTm AA
(SEQ ID NO: 90)
MAKNVAIFGLLFSLLVLVPSQIFAQKLPGNDNSTATLCLGHHAVPNGTIVKTITNDRIEVTNATELVQN
SSIGEICDSPHQILDGKNCTLIDALLGDPQCDGFQNKRWDLFVERSKAYSNCYPYDVPDYASLRSLVAS
SGTLEFNDESFNWAGVTQNGTSSACIRGSNSSFESRLNWLTHSNFKYPALNVTMPNNEQFDKLYIWGVH
HPGTDKDQIFLYAQSSGRITVSTKRSQQAVIPNIGSRPRIRNIPSRISIYWTIVKPGDILLINSTGNLI
APRGYFKIQSGKSSIMRSDAPIGKCKSECITPNGSIPNDKPFQNVNRITYGACPRYVKQSTLKLATGMR
NVPEKQTRGIFGAIAGFIENGWEGMKDGWYGFRHQNSEGRGQAADLKSTQAAIDQINGKLNRVIGKTNE
KFHQIEKEFSEVEGRIQDLEKYVEDTKIDLWSYNAELLVALENQHTIDLTDSEMNKLFEKTKKQLRENA
EDMGNGCFKIYHKCDNACIGSIRNGTYDHDVYRDEALNNRFQIKGVELKSGYKDWILWISFAISSLVLL
VALLGFIMWACQKGNIRCNICI*
A/Bangkok/3007/15 (H3N2)(aa)
(SEQ ID NO: 91)
QKIPGNDNSTATLCLGHHAVPNGTIVKTITNDRIEVTNATELVQNSSIGEICDSPHQILDGENCTLIDA
LLGDPQCDGFQNKKWDLFVERSKAYSNCYPYDVPDYASLRSLVASSGTLEFNNESFNWTGVTQNGTSSA
CIRKSSSSFESRLNWLTHLNYTYPALNVTVPNNEQFDKLYIWGVHHPGTDKDQIFLYARSSGRITVSTK
RSQQAVIPNIGSRPRIRDIPSRISIYWTIVKPGDILLINSTGNLIAPRGYFKIRSGKSSIMRSDAPIGK
CKSECITPNGSIPNDKPFQNVNRITYGACPRYVKHSTLKLATGMRNVPEKQTRGIFGAIAGFIENGWEG
MVDGWYGFRHQNSEGRGQAADLKSTQAAIDQINGKLNRLIGKTNEKFHQIEKEFSEVEGRIQDLEKYVE
DTKIDLWSYNAELLVALENQHTIDLTDSEMNKLFEKTKKQLRENAEDMGNGCFKIYHKCDNACIGSIRN
GTYDHNVYRDEALNNRFQIKGVELKSGYKDWILWISFAISCFLLCVALLGFIMWACQKGNIRCNICI
A/Hongkong/4801/14 (H3N2)(aa)
(SEQ ID NO: 92)
QKIPGNDNSTATLCLGHHAVPNGTIVKTITNDRIEVTNATELVQNSSIGEICDSPHQILDGENCTLIDA
LLGDPQCDGFQNKKWDLFVERSKAYSNCYPYDVPDYASLRSLVASSGTLEFNNESFNWTGVTQNGTSSA
CIRRSSSSFFSRLNWLTHLNYTYPALNVTMPNNEQFDKLYIWGVHHPGTDKDQIFLYAQSSGRITVSTK
RSQQAVIPNIGSRPRIRDIPSRISIYWTIVKPGDILLINSTGNLIAPRGYFKIRSGKSSIMRSDAPIGK
CKSECITPNGSIPNDKPFQNVNRITYGACPRYVKHSTLKLATGMRNVPEKQTRGIFGAIAGFIENGWEG
MVDGWYGFRHQNSEGRGQAADLKSTQAAIDQINGKLNRLIGKTNEKFHQIEKEFSEVEGRIQDLEKYVE
DTKIDLWSYNAELLVALENQHTIDLTDSEMNKLFEKTKKQLRENAEDMGNGCFKIYHKCDNACIGSIRN
GTYDHNVYRDEALNNRFQIKGVELKSGYKDWILWISFAISCFLLCVALLGFIMWACQKGNIRCNICI
A/Minnesota/40/15 (H3N2)(aa)
(SEQ ID NO: 93)
QKIPGNDNSTATLCLGHHAVPNGTIVKTITNDRIEVTNATELVQNSSIGEICDSPHQILDGENCTLIDA
LLGDPQCDGFQNKKWDLFVERSKAYSNCYPYDVPDYASLRSLVASSGTLEFNNESFNWTGVTQNGTSSA
CIRRSSSSFFSRLNWLTHLNYTYPALNVTMPNKEQFDKLYIWGVHHPGTDKDQIFLYAQSSGRITVSTK
RSQQAVIPNIGSRPRIRDIPSRISIYWTIVKPGDILLINSTGNLIAPRGYFKIRSGKSSIMRSDAPIGK
CKSECITPNGSIPNDKPFQNVNRITYGACPRYVKHSTLKLATGMRNVPEKQTRGIFGAIAGFIENGWEG
MVDGWYGFRHQNSEGRGQAADLKSTQAAIDQINGKLNRLIGKTNEKFHQIEKEFSEVEGRVQDLEKYVE
DTKIDLWSYNAELLVALENQHTIDLTDSEMNKLFEKTKKQLRENAEDMGNGCFKIYHKCDNACIGSIRN
ETYDHNVYRDEALNNRFQIKGVELKSGYKDWILWISFAISCFLLCVALLGFIMWACQKGNIRCNICI
A/South Australia/1/16 (H3N2)(aa)
(SEQ ID NO: 94)
QKIPGNDNSTATLCLGHHAVPNGTIVKTITNDRIEVTNATELVQNSSIGEICDSPHQILDGENCTLIDA
LLGDPQCDGFQNKKWDLFVERSKAYSNCYPYDVPDYASLRSLVASSGTLEFKNESFNWTGVTQNGTSSA
CIRRSSSSFFSRLNWLTHLNYTYPALNVTMPNKEQFDKLYIWGVHHPGTDKDQIFLYAQSSGRITVSTK
RSQQAVIPNIGSRPRIRDIPSRISIYWTIVKPGDILLINSTGNLIAPRGYFKIRSGKSSIMRSDAPIGK
CKSECITPNGSIPNDKPFQNVNRITYGACPRYVKHSTLKLATGMRNVPEKQTRGIFGAIAGFIENGWEG
MVDGWYGFRHQNSEGRGQAADLKSTQAAIDQINGKLNRLIGKTNEKFHQIEKEFSEVEGRVQDLEKYVE
DTKIDLWSYNAELLVALENQHTIDLTDSEMNKLFEKTKKQLRENAEDMGNGCFKIYHKCDNACIGSIRN
ETYDHNVYRDEALNNRFQIKGVELKSGYKDWILWISFAISCFLLCVALLGFIMWACQKGNIRCNICI
A/Pennsylvania/09/15 (H3N2)(aa)
(SEQ ID NO: 95)
QKLPGNDNSTATLCLGHHAVPNGTIVKTITNDRIEVTNATELVQNSSIGEICDSPHQILDGKNCTLIDA
LLGDPQCDGFQNKRWDLFVERSKAYSNCYPYDVPDYASLRSLVASSGTLEFNDESFNWAGVTQNGTSSA
CIRGSNSSFFSRLNWLTHSNFKYPALNVTMPNNEQFDKLYIWGVHHPGTDKDQIFLYAQSSGRITVSTK
RSQQAVIPNIGSRPRIRNIPSRISIYWTIVKPGDILLINSTGNLIAPRGYFKIQSGKSSIMRSDAPIGK
CKSECITPNGSIPNDKPFQNVNRITYGACPRYVKQSTLKLATGMRNVPEKQTRGIFGAIAGFIENGWEG
MKDGWYGFRHQNSEGRGQAADLKSTQAAIDQINGKLNRLIGKTNEKFHQIEKEFSEVEGRIQDLEKYVE
DTKIDLWSYNAELLVALENQHTIDLTDSEMNKLFEKTKKQLRENAEDMGNGCFKIYHKCDNACIGSIRN
GTYDHDVYRDEALNNRFQIKGVELKSGYKDWILWISFAISCFLLCVALLGFIMWACQKGNIRCNICI
A/Switzerland/9715293/13 (H3N2)(aa)
(SEQ ID NO: 96)
QKLPGNDNSTATLCLGHHAVPNGTIVKTITNDRIEVTNATELVQNSSIGEICDSPHQILDGENCTLIDA
LLGDPQCDGFQNKKWDLFVERSKAYSNCYPYDVPDYASLRSLVASSGTLEFNNESFNWAGVTQNGTSSS
CIRGSNSSFFSRLNWLTHLNSKYPALNVTMPNNEQFDKLYIWGVHHPGTDKDQIFLYAQSSGRITVSTK
RSQQAVIPNIGSRPRIRDIPSRISIYWTIVKPGDILLINSTGNLIAPRGYFKIRSGKSSIMRSDAPIGK
CKSECITPNGSIPNDKPFQNVNRITYGACPRYVKQSTLKLATGMRNVPERQTRGIFGAIAGFIENGWEG
MVDGWYGFRHQNSEGRGQAADLKSTQAAIDQINGKLNRLIGKTNEKFHQIEKEFSEVEGRIQDLEKYVE
DTKIDLWSYNAELLVALENQHTIDLTDSEMNKLFEKTKKQLRENAEDMGNGCFKIYHKCDNACIGSIRN
GTYDHDVYRDEALNNRFQIKGVELKSGYKDWILWISFAISCFLLCVALLGFIMWACQKGNIRCNICI
A/Mississippi/16/16 (H3N2)(aa)
(SEQ ID NO: 97)
QKLPGNDNSTATLCLGHHAVPNGTIVKTITNDRIEVTNATELVQNSSIGEICDSPHQILDGENCTLIDA
LLGDPQCDGFQNKKWDLFVERSRAYSNCYPYDVPDYASLRSLVASSGTLEFNNESFNWAGVTQNGTSSS
CIRGSNSSFFSRLNWLTHLNSKYPALNVTMPNNEQFDKLYIWGVHHPGTDKDQIFLYAKPSGRITVSTK
RSQQAVIPNIGSRPRIRDIPSRISIYWTIVKPGDILLINSTGNLIAPRGYFKIRSGKSSIMRSDAPIGK
CKSECITPNGSIPNDKPFQNVNRITYGACPRYVKQNTLKLATGMRNVPERQTRGIFGAIAGFIENGWEG
MVDGWYGFRHQNSEGRGQAADLKSTQAAIDQINGKLNRLIGKTNEKFHQIEKEFSEVEGRIQDLEKYVE
DTKIDLWSYNAELLVALENQHTIDLTDSEMNKLFEKTKKQLRENAEDMGNGCFKIYHKCDNACIGSIRN
GTYDHDVYRDEALNNRFQIKGVELKSGYKDWILWISFAISCFLLCVALLGFIMWACQKGNIRCNICI
CysTM sequence (aa)
(SEQ ID NO: 98)
SLVLL
Native TMCT (aa)
SEQ ID NO: 99)
CFLLC
CysTM sequence (nt)
(SEQ ID NO: 100)
TCCCTTGTACTGTTA
Native TMCT (nt)
SEQ ID NO: 101)
TGTTTTTTGCTTTGT
A/Hong Kong/4801/14 (H3N2 (nt)-(Epiflu accession no EPI539576)
(SEQ ID NO: 102)
ATGAAGACTATCATTGCTTTGAGCTACATTCTATGTCTGGTTTTCGCTCAAAAAATTCCTGGAAATGAC
AATAGCACGGCAACGCTGTGCCTTGGGCACCATGCAGTACCAAACGGAACGATAGTGAAAACAATCACG
AATGACCGAATTGAAGTTACTAATGCTACTGAGCTGGTTCAGAATTCCTCAATAGGTGAAATATGCGAC
AGTCCTCATCAGATCCTTGATGGAGAAAACTGCACACTAATAGATGCTCTATTGGGAGACCCTCAGTGT
GATGGCTTTCAAAATAAGAAATGGGACCTTTTTGTTGAACGAAGCAAAGCCTACAGCAACTGTTACCCT
TATGATGTGCCGGATTATGCCTCCCTTAGGTCACTAGTTGCCTCATCCGGCACACTGGAGTTTAACAAT
GAAAGCTTCAATTGGACTGGAGTCACTCAAAACGGAACAAGTTCTGCTTGCATAAGGAGATCTAGTAGT
AGTTTCTTTAGTAGATTAAATTGGTTGACCCACTTAAACTACACATACCCAGCATTGAACGTGACTATG
CCAAACAATGAACAATTTGACAAATTGTACATTTGGGGGGTTCACCACCCGGGTACGGACAAGGACCAA
ATCTTCCTGTATGCTCAATCATCAGGAAGAATCACAGTATCTACCAAAAGAAGCCAACAAGCTGTAATC
CCAAATATCGGATCTAGACCCAGAATAAGGGATATCCCTAGCAGAATAAGCATCTATTGGACAATAGTA
AAACCGGGAGACATACTTTTGATTAACAGCACAGGGAATCTAATTGCTCCTAGGGGTTACTTCAAAATA
CGAAGTGGGAAAAGCTCAATAATGAGATCAGATGCACCCATTGGCAAATGCAAGTCTGAATGCATCACT
CCAAATGGAAGCATTCCCAATGACAAACCATTCCAAAATGTAAACAGGATCACATACGGGGCCTGTCCC
AGATATGTTAAGCATAGCACTCTGAAATTGGCAACAGGAATGCGAAATGTACCAGAGAAACAAACTAGA
GGCATATTTGGCGCAATAGCGGGTTTCATAGAAAATGGTTGGGAGGGAATGGTGGATGGTTGGTACGGT
TTCAGGCATCAAAATTCTGAGGGAAGAGGACAAGCAGCAGATCTCAAAAGCACTCAAGCAGCAATCGAT
CAAATCAATGGGAAGCTGAATCGATTGATCGGGAAAACCAACGAGAAATTCCATCAGATTGAAAAAGAA
TTCTCAGAAGTAGAAGGAAGAATTCAGGACCTTGAGAAATATGTTGAGGACACTAAAATAGATCTCTGG
TCATACAACGCGGAGCTTCTTGTTGCCCTGGAGAACCAACATACAATTGATCTAACTGACTCAGAAATG
AACAAACTGTTTGAAAAAACAAAGAAGCAACTGAGGGAAAATGCTGAGGATATGGGCAATGGTTGTTTC
AAAATATACCACAAATGTGACAATGCCTGCATAGGATCAATAAGAAATGGAACTTATGACCACAATGTG
TACAGGGATGAAGCATTAAACAACCGGTTCCAGATCAAGGGAGTTGAGCTGAAGTCAGGGTACAAAGAT
TGGATCCTATGGATTTCCTTTGCCATATCATGTTTTTTGCTTTGTGTTGCTTTGTTGGGGTTCATCATG
TGGGCCTGCCAAAAGGGCAACATTAGGTGCAACATTTGCATTTGA
Construct 2801 from 2X35S prom to NOS term
(SEQ ID NO: 103)
GTCAACATGGTGGAGCACGACACACTTGTCTACTCCAAAAATATCAAAGATACAGTCTCAGAAGACCAA
AGGGCAATTGAGACTTTTCAACAAAGGGTAATATCCGGAAACCTCCTCGGATTCCATTGCCCAGCTATC
TGTCACTTTATTGTGAAGATAGTGGAAAAGGAAGGTGGCTCCTACAAATGCCATCATTGCGATAAAGGA
AAGGCCATCGTTGAAGATGCCTCTGCCGACAGTGGTCCCAAAGATGGACCCCCACCCACGAGGAGCATC
GTGGAAAAAGAAGACGTTCCAACCACGTCTTCAAAGCAAGTGGATTGATGTGATAACATGGTGGAGCAC
GACACACTTGTCTACTCCAAAAATATCAAAGATACAGTCTCAGAAGACCAAAGGGCAATTGAGACTTTT
CAACAAAGGGTAATATCCGGAAACCTCCTCGGATTCCATTGCCCAGCTATCTGTCACTTTATTGTGAAG
ATAGTGGAAAAGGAAGGTGGCTCCTACAAATGCCATCATTGCGATAAAGGAAAGGCCATCGTTGAAGAT
GCCTCTGCCGACAGTGGTCCCAAAGATGGACCCCCACCCACGAGGAGCATCGTGGAAAAAGAAGACGTT
CCAACCACGTCTTCAAAGCAAGTGGATTGATGTGATATCTCCACTGACGTAAGGGATGACGCACAATCC
CACTATCCTTCGCAAGACCCTTCCTCTATATAAGGAAGTTCATTTCATTTGGAGAGGTATTAAAATCTT
AATAGGTTTTGATAAAAGCGAACGTGGGGAAACCCGAACCAAACCTTCTTCTAAACTCTCTCTCATCTC
TCTTAAAGCAAACTTCTCTCTTGTCTTTCTTGCGTGAGCGATCTTCAACGTTGTCAGATCGTGCTTCGG
CACCAGTACAATGGCGAAAAACGTTGCGATTTTCGGCTTATTGTTTTCTCTTCTTGTGTTGGTTCCTTC
TCAGATCTTCGCGCAAAAACTTCCTGGAAATGACAATAGCACGGCAACGCTGTGCCTTGGGCACCATGC
AGTACCAAACGGAACGATAGTGAAAACAATCACGAATGACCGAATTGAAGTTACTAATGCTACTGAGCT
GGTTCAGAATTCCTCAATAGGTGAAATATGCGACAGTCCTCATCAGATCCTTGATGGAGAAAACTGCAC
ACTAATAGATGCTCTATTGGGAGACCCTCAGTGTGATGGCTTTCAAAATAAGAAATGGGACCTTTTTGT
TGAACGAAGCAAAGCCTACAGCAACTGTTACCCTTATGATGTGCCGGATTATGCCTCCCTTAGGTCACT
AGTTGCCTCATCCGGCACACTGGAGTTTAACAATGAAAGCTTCAATTGGGCTGGAGTCACTCAAAACGG
AACAAGTTCTTCTTGCATAAGGGGATCTAATAGTAGTTTCTTTAGTAGATTAAATTGGTTGACCCACTT
AAACTCCAAATACCCAGCATTAAACGTGACTATGCCAAACAATGAACAATTTGACAAATTGTACATTTG
GGGGGTTCACCACCCGGGTACGGACAAGGACCAAATCTTCCTGTATGCACAATCATCAGGAAGAATCAC
AGTATCTACCAAAAGAAGCCAACAAGCTGTAATCCCGAATATCGGATCTAGACCCAGAATAAGGGATAT
CCCTAGCAGAATAAGCATCTATTGGACAATAGTAAAACCGGGAGACATACTTTTGATTAACAGCACAGG
GAATCTAATTGCTCCTAGGGGTTACTTCAAAATACGAAGTGGGAAAAGCTCAATAATGAGATCAGATGC
ACCCATTGGCAAATGCAAGTCTGAATGCATCACTCCAAATGGAAGCATTCCCAATGACAAACCATTCCA
AAATGTAAACAGGATCACATACGGGGCCTGTCCCAGATATGTTAAGCAAAGCACTCTGAAATTGGCAAC
AGGAATGCGAAATGTACCAGAGAGACAAACTAGAGGCATATTTGGCGCAATAGCGGGTTTCATAGAAAA
TGGTTGGGAGGGAATGGTGGATGGTTGGTACGGCTTCAGGCATCAAAATTCTGAGGGAAGAGGACAAGC
AGCAGATCTCAAAAGCACTCAAGCAGCAATCGATCAAATCAATGGGAAGCTGAATCGATTGATCGGGAA
AACCAACGAGAAATTCCATCAGATTGAAAAAGAATTCTCAGAAGTAGAAGGGAGAATTCAGGACCTTGA
GAAATATGTTGAGGACACAAAAATAGATCTCTGGTCATACAACGCGGAGCTTCTTGTTGCCCTGGAGAA
CCAACATACAATTGATCTAACTGACTCAGAAATGAACAAACTGTTTGAAAAAACAAAGAAGCAACTGAG
GGAAAATGCTGAGGATATGGGCAATGGTTGTTTCAAAATATACCACAAATGTGACAATGCCTGCATAGG
ATCAATCAGAAATGGAACTTATGACCACGATGTATACAGGGATGAAGCATTAAACAACCGGTTCCAGAT
CAAGGGAGTTGAGCTGAAGTCAGGGTACAAAGATTGGATCCTATGGATTTCCTTTGCCATATCATGTTT
TTTGCTTTGTGTTGCTTTGTTGGGGTTCATCATGTGGGCCTGCCAAAAGGGCAACATTAGGTGCAACAT
TTGCATTTGAAGGCCTATTTTCTTTAGTTTGAATTTACTGTTATTCGGTGTGCATTTCTATGTTTGGTG
AGCGGTTTTCTGTGCTCAGAGTGTGTTTATTTTATGTAATTTAATTTCTTTGTGAGCTCCTGTTTAGCA
GGTCGTCCCTTCAGCAAGGACACAAAAAGATTTTAATTTTATTAAAAAAAAAAAAAAAAAAGACCGGGA
ATTCGATATCAAGCTTATCGACCTGCAGATCGTTCAAACATTTGGCAATAAAGTTTCTTAAGATTGAAT
CCTGTTGCCGGTCTTGCGATGATTATCATATAATTTCTGTTGAATTACGTTAAGCATGTAATAATTAAC
ATGTAATGCATGACGTTATTTATGAGATGGGTTTTTATGATTAGAGTCCCGCAATTATACATTTAATAC
GCGATAGAAAACAAAATATAGCGCGCAAACTAGGATAAATTATCGCGCGCGGTGTCATCTATGTTACTA
GAT
Construct 3023 from 2X35S prom to NOS term
(SEQ ID NO: 104)
GTCAACATGGTGGAGCACGACACACTTGTCTACTCCAAAAATATCAAAGATACAGTCTCAGAAGACCAA
AGGGCAATTGAGACTTTTCAACAAAGGGTAATATCCGGAAACCTCCTCGGATTCCATTGCCCAGCTATC
TGTCACTTTATTGTGAAGATAGTGGAAAAGGAAGGTGGCTCCTACAAATGCCATCATTGCGATAAAGGA
AAGGCCATCGTTGAAGATGCCTCTGCCGACAGTGGTCCCAAAGATGGACCCCCACCCACGAGGAGCATC
GTGGAAAAAGAAGACGTTCCAACCACGTCTTCAAAGCAAGTGGATTGATGTGATAACATGGTGGAGCAC
GACACACTTGTCTACTCCAAAAATATCAAAGATACAGTCTCAGAAGACCAAAGGGCAATTGAGACTTTT
CAACAAAGGGTAATATCCGGAAACCTCCTCGGATTCCATTGCCCAGCTATCTGTCACTTTATTGTGAAG
ATAGTGGAAAAGGAAGGTGGCTCCTACAAATGCCATCATTGCGATAAAGGAAAGGCCATCGTTGAAGAT
GCCTCTGCCGACAGTGGTCCCAAAGATGGACCCCCACCCACGAGGAGCATCGTGGAAAAAGAAGACGTT
CCAACCACGTCTTCAAAGCAAGTGGATTGATGTGATATCTCCACTGACGTAAGGGATGACGCACAATCC
CACTATCCTTCGCAAGACCCTTCCTCTATATAAGGAAGTTCATTTCATTTGGAGAGGTATTAAAATCTT
AATAGGTTTTGATAAAAGCGAACGTGGGGAAACCCGAACCAAACCTTCTTCTAAACTCTCTCTCATCTC
TCTTAAAGCAAACTTCTCTCTTGTCTTTCTTGCGTGAGCGATCTTCAACGTTGTCAGATCGTGCTTCGG
CACCAGTACAATGGCGAAAAACGTTGCGATTTTCGGCTTATTGTTTTCTCTTCTTGTGTTGGTTCCTTC
TCAGATCTTCGCGCAAAAACTTCCTGGAAATGACAATAGCACGGCAACGCTGTGCCTTGGGCACCATGC
AGTACCAAACGGAACGATAGTGAAAACAATCACGAATGACCGAATTGAAGTTACTAATGCTACTGAGCT
GGTTCAGAATTCCTCAATAGGTGAAATATGCGACAGTCCTCATCAGATCCTTGATGGAGAAAACTGCAC
ACTAATAGATGCTCTATTGGGAGACCCTCAGTGTGATGGCTTTCAAAATAAGAAATGGGACCTTTTTGT
TGAACGAAGCAAAGCCTACAGCAACTGTTACCCTTATGATGTGCCGGATTATGCCTCCCTTAGGTCACT
AGTTGCCTCATCCGGCACACTGGAGTTTAACAATGAAAGCTTCAATTGGGCTGGAGTCACTCAAAACGG
AACAAGTTCTTCTTGCATAAGGGGATCTAATAGTAGTTTCTTTAGTAGATTAAATTGGTTGACCCACTT
AAACTCCAAATACCCAGCATTAAACGTGACTATGCCAAACAATGAACAATTTGACAAATTGTACATTTG
GGGGGTTCACCACCCGGGTACGGACAAGGACCAAATCTTCCTGTATGCACAATCATCAGGAAGAATCAC
AGTATCTACCAAAAGAAGCCAACAAGCTGTAATCCCGAATATCGGATCTAGACCCAGAATAAGGGATAT
CCCTAGCAGAATAAGCATCTATTGGACAATAGTAAAACCGGGAGACATACTTTTGATTAACAGCACAGG
GAATCTAATTGCTCCTAGGGGTTACTTCAAAATACGAAGTGGGAAAAGCTCAATAATGAGATCAGATGC
ACCCATTGGCAAATGCAAGTCTGAATGCATCACTCCAAATGGAAGCATTCCCAATGACAAACCATTCCA
AAATGTAAACAGGATCACATACGGGGCCTGTCCCAGATATGTTAAGCAAAGCACTCTGAAATTGGCAAC
AGGAATGCGAAATGTACCAGAGAGACAAACTAGAGGCATATTTGGCGCAATAGCGGGTTTCATAGAAAA
TGGTTGGGAGGGAATGGTGGATGGTTGGTACGGCTTCAGGCATCAAAATTCTGAGGGAAGAGGACAAGC
AGCAGATCTCAAAAGCACTCAAGCAGCAATCGATCAAATCAATGGGAAGCTGGCTCGATTGATCGGGAA
AACCAACGAGAAATTCCATCAGATTGAAAAAGAATTCTCAGAAGTAGAAGGGAGAATTCAGGACCTTGA
GAAATATGTTGAGGACACAAAAATAGATCTCTGGTCATACAACGCGGAGCTTCTTGTTGCCCTGGAGAA
CCAACATACAATTGATCTAACTGACTCAGAAATGAACAAACTGTTTGAAAAAACAAAGAAGCAACTGAG
GGAAAATGCTGAGGATATGGGCAATGGTTGTTTCAAAATATACCACAAATGTGACAATGCCTGCATAGG
ATCAATCAGAAATGGAACTTATGACCACGATGTATACAGGGATGAAGCATTAAACAACCGGTTCCAGAT
CAAGGGAGTTGAGCTGAAGTCAGGGTACAAAGATTGGATCCTATGGATTTCCTTTGCCATATCATGTTT
TTTGCTTTGTGTTGCTTTGTTGGGGTTCATCATGTGGGCCTGCCAAAAGGGCAACATTAGGTGCAACAT
TTGCATTTGAAGGCCTATTTTCTTTAGTTTGAATTTACTGTTATTCGGTGTGCATTTCTATGTTTGGTG
AGCGGTTTTCTGTGCTCAGAGTGTGTTTATTTTATGTAATTTAATTTCTTTGTGAGCTCCTGTTTAGCA
GGTCGTCCCTTCAGCAAGGACACAAAAAGATTTTAATTTTATTAAAAAAAAAAAAAAAAAAGACCGGGA
ATTCGATATCAAGCTTATCGACCTGCAGATCGTTCAAACATTTGGCAATAAAGTTTCTTAAGATTGAAT
CCTGTTGCCGGTCTTGCGATGATTATCATATAATTTCTGTTGAATTACGTTAAGCATGTAATAATTAAC
ATGTAATGCATGACGTTATTTATGAGATGGGTTTTTATGATTAGAGTCCCGCAATTATACATTTAATAC
GCGATAGAAAACAAAATATAGCGCGCAAACTAGGATAAATTATCGCGCGCGGTGTCATCTATGTTACTA
GAT
Construct 2811 from 2X35S prom to NOS term
(SEQ ID NO: 105)
GTCAACATGGTGGAGCACGACACACTTGTCTACTCCAAAAATATCAAAGATACAGTCTCAGAAGACCAA
AGGGCAATTGAGACTTTTCAACAAAGGGTAATATCCGGAAACCTCCTCGGATTCCATTGCCCAGCTATC
TGTCACTTTATTGTGAAGATAGTGGAAAAGGAAGGTGGCTCCTACAAATGCCATCATTGCGATAAAGGA
AAGGCCATCGTTGAAGATGCCTCTGCCGACAGTGGTCCCAAAGATGGACCCCCACCCACGAGGAGCATC
GTGGAAAAAGAAGACGTTCCAACCACGTCTTCAAAGCAAGTGGATTGATGTGATAACATGGTGGAGCAC
GACACACTTGTCTACTCCAAAAATATCAAAGATACAGTCTCAGAAGACCAAAGGGCAATTGAGACTTTT
CAACAAAGGGTAATATCCGGAAACCTCCTCGGATTCCATTGCCCAGCTATCTGTCACTTTATTGTGAAG
ATAGTGGAAAAGGAAGGTGGCTCCTACAAATGCCATCATTGCGATAAAGGAAAGGCCATCGTTGAAGAT
GCCTCTGCCGACAGTGGTCCCAAAGATGGACCCCCACCCACGAGGAGCATCGTGGAAAAAGAAGACGTT
CCAACCACGTCTTCAAAGCAAGTGGATTGATGTGATATCTCCACTGACGTAAGGGATGACGCACAATCC
CACTATCCTTCGCAAGACCCTTCCTCTATATAAGGAAGTTCATTTCATTTGGAGAGGTATTAAAATCTT
AATAGGTTTTGATAAAAGCGAACGTGGGGAAACCCGAACCAAACCTTCTTCTAAACTCTCTCTCATCTC
TCTTAAAGCAAACTTCTCTCTTGTCTTTCTTGCGTGAGCGATCTTCAACGTTGTCAGATCGTGCTTCGG
CACCAGTACAATGGCGAAAAACGTTGCGATTTTCGGCTTATTGTTTTCTCTTCTTGTGTTGGTTCCTTC
TCAGATCTTCGCGCAAAAACTTCCTGGAAATGACAATAGCACGGCAACGCTGTGCCTTGGGCACCATGC
AGTACCAAACGGAACGATAGTGAAAACAATCACGAATGACCGAATTGAAGTTACTAATGCTACTGAGCT
GGTTCAGAATTCCTCAATAGGTGAAATATGCGACAGTCCTCATCAGATCCTTGATGGAGAAAACTGCAC
ACTAATAGATGCTCTATTGGGAGACCCTCAGTGTGATGGCTTTCAAAATAAGAAATGGGACCTTTTTGT
TGAACGAAGCAAAGCCTACAGCAACTGTTACCCTTATGATGTGCCGGATTATGCCTCCCTTAGGTCACT
AGTTGCCTCATCCGGCACACTGGAGTTTAACAATGAAAGCTTCAATTGGGCTGGAGTCACTCAAAACGG
AACAAGTTCTTCTTGCATAAGGGGATCTAATAGTAGTTTCTTTAGTAGATTAAATTGGTTGACCCACTT
AAACTCCAAATACCCAGCATTAAACGTGACTATGCCAAACAATGAACAATTTGACAAATTGTACATTTG
GGGGGTTCACCACCCGGGTACGGACAAGGACCAAATCTTCCTGTATGCACAATCATCAGGAAGAATCAC
AGTATCTACCAAAAGAAGCCAACAAGCTGTAATCCCGAATATCGGATCTAGACCCAGAATAAGGGATAT
CCCTAGCAGAATAAGCATCTATTGGACAATAGTAAAACCGGGAGACATACTTTTGATTAACAGCACAGG
GAATCTAATTGCTCCTAGGGGTTACTTCAAAATACGAAGTGGGAAAAGCTCAATAATGAGATCAGATGC
ACCCATTGGCAAATGCAAGTCTGAATGCATCACTCCAAATGGAAGCATTCCCAATGACAAACCATTCCA
AAATGTAAACAGGATCACATACGGGGCCTGTCCCAGATATGTTAAGCAAAGCACTCTGAAATTGGCAAC
AGGAATGCGAAATGTACCAGAGAGACAAACTAGAGGCATATTTGGCGCAATAGCGGGTTTCATAGAAAA
TGGTTGGGAGGGAATGGTGGATGGTTGGTACGGCTTCAGGCATCAAAATTCTGAGGGAAGAGGACAAGC
AGCAGATCTCAAAAGCACTCAAGCAGCAATCGATCAAATCAATGGGAAGCTGAATCGATTGATCGGGAA
AACCAACGAGAAATTCCATCAGATTGAAAAAGAATTCTCAGAAGTAGAAGGGAGAATTCAGGACCTTGA
GAAATATGTTGAGGACACAAAAATAGATCTCTGGTCATACAACGCGGAGCTTCTTGTTGCCCTGGAGAA
CCAACATACAATTGATCTAACTGACTCAGAAATGAACAAACTGTTTGAAAAAACAAAGAAGCAACTGAG
GGAAAATGCTGAGGATATGGGCAATGGTTGTTTCAAAATATACCACAAATGTGACAATGCCTGCATAGG
ATCAATCAGAAATGGAACTTATGACCACGATGTATACAGGGATGAAGCATTAAACAACCGGTTCCAGAT
CAAGGGAGTTGAGCTGAAGTCAGGGTACAAAGATTGGATCCTATGGATTTCCTTTGCCATATCATCCCT
TGTACTGTTAGTTGCTTTGTTGGGGTTCATCATGTGGGCCTGCCAAAAGGGCAACATTAGGTGCAACAT
TTGCATTTGAAGGCCTATTTTCTTTAGTTTGAATTTACTGTTATTCGGTGTGCATTTCTATGTTTGGTG
AGCGGTTTTCTGTGCTCAGAGTGTGTTTATTTTATGTAATTTAATTTCTTTGTGAGCTCCTGTTTAGCA
GGTCGTCCCTTCAGCAAGGACACAAAAAGATTTTAATTTTATTAAAAAAAAAAAAAAAAAAGACCGGGA
ATTCGATATCAAGCTTATCGACCTGCAGATCGTTCAAACATTTGGCAATAAAGTTTCTTAAGATTGAAT
CCTGTTGCCGGTCTTGCGATGATTATCATATAATTTCTGTTGAATTACGTTAAGCATGTAATAATTAAC
ATGTAATGCATGACGTTATTTATGAGATGGGTTTTTATGATTAGAGTCCCGCAATTATACATTTAATAC
GCGATAGAAAACAAAATATAGCGCGCAAACTAGGATAAATTATCGCGCGCGGTGTCATCTATGTTACTA
GAT

All citations are hereby incorporated by reference.

The present invention has been described with regard to one or more embodiments. However, it will be apparent to persons skilled in the art that a number of variations and modifications can be made without departing from the scope of the invention as defined in the claims.

1. A modified influenza H3 hemagglutinin (HA) protein comprising an amino acid sequence of a wildtype influenza H3 HA protein and comprising at least one amino acid substitution, said at least one amino acid substitution consisting of an amino acid substitution corresponding to position 382 of reference sequence SEQ ID NO: 92, and optionally one or more amino acid substitution corresponding to positions 384, 392 and/or 431 of reference sequence SEQ ID NO: 92, wherein:
the amino acid substitution corresponding to position 382 of reference sequence SEQ ID NO: 92 is to an alanine or a conserved substitution of alanine;
the amino acid substitution corresponding to position 384 of reference sequence SEQ ID NO: 92 is to a valine or a conserved substitution of valine;
the amino acid substitution corresponding to position 392 of reference sequence SEQ ID NO: 92 is to an aspartic acid or a conserved substitution of aspartic acid; and
the amino acid substitution corresponding to position 431 of reference sequence SEQ ID NO: 92 is to a methionine or a conserved substitution of methionine.

2. The modified influenza H3 HA of claim 1, wherein:
the conserved substitution of alanine is serine, glycine, threonine, cysteine, or valine;
the conserved substitution of valine is isoleucine, methionine, alanine, or threonine;
the conserved substitution of aspartic acid is glutamic acid, glutamine, or serine; and/or
the conserved substitution of methionine is isoleucine, glutamine, valine or phenylalanine.

3. The modified influenza H3 HA protein of claim 1, wherein the HA protein comprising an amino acid substitution corresponding to position 382 of reference sequence SEQ ID NO: 92, wherein the amino acid substitution is to alanine or a conserved substitution of alanine.

4. The modified influenza H3 HA protein of claim 3, wherein the conserved substitution of alanine is serine, glycine, threonine, cysteine, or valine.

5. The modified influenza H3 HA protein of claim 1, wherein the HA protein comprising an amino acid substitution corresponding to position 384 of reference sequence SEQ ID NO: 92, wherein the amino acid substitution is to valine or a conserved substitution of valine.

6. The modified influenza H3 HA protein of claim 5, wherein the conserved substitution of valine is isoleucine, methionine, alanine, or threonine.

7. The modified influenza H3 HA protein of claim 1, wherein the HA protein comprising an amino acid substitution corresponding to positions 382 and 384 of reference sequence SEQ ID NO: 92, wherein the amino acid substitution corresponding to position 382 of reference sequence SEQ ID NO: 92 is to alanine or a conserved substitution of alanine and the amino acid substitution corresponding to position 384 of reference sequence SEQ ID NO: 92 is to valine or a conserved substitution of valine.

8. The modified influenza H3 HA protein of claim 7, wherein the conserved substitution of alanine is serine, glycine, threonine, cysteine, or valine, and the conserved substitution of valine is isoleucine, methionine, alanine, or threonine.

9. The modified influenza H3 HA protein of claim 1, wherein the HA protein comprising an amino acid substitution corresponding to positions 382, 384 and 392 of reference sequence SEQ ID NO: 92, wherein:
the amino acid substitution corresponding to position 382 of reference sequence SEQ ID NO: 92 is to alanine or a conserved substitution of alanine;
the amino acid substitution corresponding to position 384 of reference sequence SEQ ID NO: 92 is to valine or a conserved substitution of valine; and
the amino acid substitution corresponding to position 392 of reference sequence SEQ ID NO: 92 is to an aspartic acid or a conserved substitution of aspartic acid.

10. The modified influenza H3 HA protein of claim 9, wherein
the conserved substitution of alanine is serine, glycine, threonine, cysteine, or valine;
the conserved substitution of valine is isoleucine, methionine, alanine, or threonine; and
the conserved substitution of aspartic acid is glutamic acid, glutamine or serine.

11. The modified influenza H3 HA protein of claim 1, wherein the HA protein comprising an amino acid substitution corresponding to position 382, 384 and 431 of reference sequence SEQ ID NO: 92, wherein:
the amino acid substitution corresponding to position 382 of reference sequence SEQ ID NO: 92 is to alanine or a conserved substitution of alanine;
the amino acid substitution corresponding to position 384 of reference sequence SEQ ID NO: 92 is to valine or a conserved substitution of valine; and
the amino acid substitution corresponding to position 431 of reference sequence SEQ ID NO: 92 is to a methionine or a conserved substitution of methionine.

12. The modified influenza H3 HA protein of claim 11, wherein:
the conserved substitution of alanine is serine, glycine, threonine, cysteine, or valine;
the conserved substitution of valine is isoleucine, methionine, alanine, or threonine; and
the conserved substitution of methionine is isoleucine, glutamine, valine or phenylalanine.

13. The modified influenza H3 HA protein of claim 1, wherein the HA protein comprising an amino acid substitution corresponding to positions 382, 384, 392 and 431 of reference sequence SEQ ID NO: 92, wherein:
the amino acid substitution corresponding to position 382 of reference sequence SEQ ID NO: 92 is to alanine or a conserved substitution of alanine;
the amino acid substitution corresponding to position 384 of reference sequence SEQ ID NO: 92 is to valine or a conserved substitution of valine;
the amino acid substitution corresponding to position 392 of reference sequence SEQ ID NO: 92 is to an aspartic acid or a conserved substitution of aspartic acid; and
the amino acid substitution corresponding to position 431 of reference sequence SEQ ID NO: 92 is to a methionine or a conserved substitution of methionine.

14. The modified influenza H3 HA protein of claim 13, wherein
the conserved substitution of alanine is serine, glycine, threonine, cysteine, or valine;
the conserved substitution of valine is isoleucine, methionine, alanine, or threonine;
the conserved substitution of aspartic acid is glutamic acid, glutamine or serine; and
the conserved substitution of methionine is isoleucine, glutamine, valine or phenylalanine.

15. A virus-like particle (VLP) comprising the modified influenza H3 hemagglutinin (HA) protein of claim 1.

16. A virus-like particle (VLP) comprising the modified influenza H3 hemagglutinin (HA) protein of claim 3.

17. A virus-like particle (VLP) comprising the modified influenza H3 hemagglutinin (HA) protein of claim 5.

18. A virus-like particle (VLP) comprising the modified influenza H3 hemagglutinin (HA) protein of claim 7.

19. A virus-like particle (VLP) comprising the modified influenza H3 hemagglutinin (HA) protein of claim 9.

20. A virus-like particle (VLP) comprising the modified influenza H3 hemagglutinin (HA) protein of claim 11.

21. A virus-like particle (VLP) comprising the modified influenza H3 hemagglutinin (HA) protein of claim 13.
